SEQUENCE LISTING (128 sequences)

SEQ ID NO: 1 - DNA, 20 bp, synthetic construct
  source 1..20: mol_type = other DNA
acatggtacaggaggttcta

SEQ ID NO: 2 - DNA, 20 bp, synthetic construct
  source 1..20: mol_type = other DNA
tagaacctcctgtaccatgt

SEQ ID NO: 3 - RNA, 20 nt, synthetic construct
  source 1..20: mol_type = other RNA
acatggtacaggaggttcta

SEQ ID NO: 4 - DNA, 20 bp, synthetic construct
  source 1..20: mol_type = other DNA
cacatggtacaggaggttct

SEQ ID NO: 5 - DNA, 20 bp, synthetic construct
  source 1..20: mol_type = other DNA
agaacctcctgtaccatgtg

SEQ ID NO: 6 - RNA, 20 nt, synthetic construct
  source 1..20: mol_type = other RNA
cacatggtacaggaggttct

SEQ ID NO: 7 - DNA, 20 bp, synthetic construct
  source 1..20: mol_type = other DNA
agccgaccacatggtacagg

SEQ ID NO: 8 - DNA, 20 bp, synthetic construct
  source 1..20: mol_type = other DNA
cctgtaccatgtggtcggct

SEQ ID NO: 9 - RNA, 20 nt, synthetic construct
  source 1..20: mol_type = other RNA
agccgaccacatggtacagg

SEQ ID NO: 10 - DNA, 20 bp, synthetic construct
  source 1..20: mol_type = other DNA
ctagaacctcctgtaccatg

SEQ ID NO: 11 - DNA, 20 bp, synthetic construct
  source 1..20: mol_type = other DNA
catggtacaggaggttctag

SEQ ID NO: 12 - RNA, 20 nt, synthetic construct
  source 1..20: mol_type = other RNA
ctagaacctcctgtaccatg

SEQ ID NO: 13 - DNA, 20 bp, synthetic construct
  source 1..20: mol_type = other DNA
gtccaggtcgttcttctgac

SEQ ID NO: 14 - DNA, 20 bp, synthetic construct
  source 1..20: mol_type = other DNA
gtcagaagaacgacctggac

SEQ ID NO: 15 - RNA, 20 nt, synthetic construct
  source 1..20: mol_type = other RNA
gtccaggtcgttcttctgac

SEQ ID NO: 16 - DNA, 20 bp, synthetic construct
  source 1..20: mol_type = other DNA
tgccagtcagaagaacgacc

SEQ ID NO: 17 - DNA, 20 bp, synthetic construct
  source 1..20: mol_type = other DNA
ggtcgttcttctgactggca

SEQ ID NO: 18 - RNA, 20 nt, synthetic construct
  source 1..20: mol_type = other RNA
tgccagtcagaagaacgacc

SEQ ID NO: 19 - DNA, 20 bp, synthetic construct
  source 1..20: mol_type = other DNA
gcatcagtgccactgcgtcc

SEQ ID NO: 20 - DNA, 20 bp, synthetic construct
  source 1..20: mol_type = other DNA
ggacgcagtggcactgatgc

SEQ ID NO: 21 - RNA, 20 nt, synthetic construct
  source 1..20: mol_type = other RNA
gcatcagtgccactgcgtcc

SEQ ID NO: 22 - DNA, 20 bp, synthetic construct
  source 1..20: mol_type = other DNA
gaagaacgacctggacgcag

SEQ ID NO: 23 - DNA, 20 bp, synthetic construct
  source 1..20: mol_type = other DNA
ctgcgtccaggtcgttcttc

SEQ ID NO: 24 - RNA, 20 nt, synthetic construct
  source 1..20: mol_type = other RNA
gaagaacgacctggacgcag

SEQ ID NO: 25 - DNA, 200 bp, synthetic construct
  source 1..200: mol_type = other DNA
tgcctctcccacatgtgacccttacctacactctctggggacccccagtgttgcgcctttgtctctttgcctttgtccttaccctagagcctgctctatcatgtggtcggctggaccgactggaaccttgccctgaaccccgaaggaggacccaattgggtgcgtaactttgtcgacagtcccatcattgtagacatcac

SEQ ID NO: 26 - DNA, 200 bp, synthetic construct
  source 1..200: mol_type = other DNA
tgcctctcccacatgtgacccttacctacactctctggggacccccagtgttgcgcctttgtctctttgcctttgtccttaccctagagcctcctgtaccatgtggtcggctggaccgactggaaccttgccctgaaccccgaaggaggacccaattgggtgcgtaactttgtcgacagtcccatcattgtagacatcac

SEQ ID NO: 27 - DNA, 200 bp, synthetic construct
  source 1..200: mol_type = other DNA
tgatgggactgtcgacaaagttacgcacccaattgggtcctccttcggggttcagggcaaggttccagtcggtccagccgaccacgtgatagagcaggctctagggtaaggacaaaggcaaagagacaaaggcgcaacactgggggtccccagagagtgtaggtaagggtcacatgtgggagaggcagctgtgggtaggt

SEQ ID NO: 28 - DNA, 200 bp, synthetic construct
  source 1..200: mol_type = other DNA
tgatgggactgtcgacaaagttacgcacccaattgggtcctccttcggggttcagggcaaggttccagtcggtccagccgaccacatggtacaggaggctctagggtaaggacaaaggcaaagagacaaaggcgcaacactgggggtccccagagagtgtaggtaagggtcacatgtgggagaggcagctgtgggtaggt

SEQ ID NO: 29 - DNA, 200 bp, synthetic construct
  source 1..200: mol_type = other DNA
gggaggtaccccgagggactctgaccatctgttcccacattcagcaagttcattcctgagggctcccagagagtggggctggttgctagccagaaaaatgatccggacgcagtggcactgatgcatcccgatggctctgctgttgtggtcgtgctaaaccggtgagggcaatggtgaggtctgggaagtgggctgaagac

SEQ ID NO: 30 - DNA, 200 bp, synthetic construct
  source 1..200: mol_type = other DNA
caacgctgtcttcagcccacttcccagacctcaccattgccctcaccggtttagcacgaccacaacagcagagccatcgggatgcatgagggcgacggcatccgggtcgttcttctgactggcaaccagccccactctctgggagccctcaggaatgaacttgctgaatgtgggaacagatggtcagagtccctcggggt

SEQ ID NO: 31 - DNA, 20 bp, synthetic construct
  source 1..20: mol_type = other DNA
tgacatcaattattatacat

SEQ ID NO: 32 - DNA, 20 bp, synthetic construct
  source 1..20: mol_type = other DNA
atgtataataattgatgtca

SEQ ID NO: 33 - RNA, 20 nt, synthetic construct
  source 1..20: mol_type = other RNA
tgacatcaattattatacat

SEQ ID NO: 34 - DNA, 20 bp, synthetic construct
  source 1..20: mol_type = other DNA
ttttgcagtttatcaggatg

SEQ ID NO: 35 - DNA, 20 bp, synthetic construct
  source 1..20: mol_type = other DNA
catcctgataaactgcaaaa

SEQ ID NO: 36 - RNA, 20 nt, synthetic construct
  source 1..20: mol_type = other RNA
ttttgcagtttatcaggatg

SEQ ID NO: 37 - DNA, 20 bp, synthetic construct
  source 1..20: mol_type = other DNA
gtagagcggaggcaggaggc

SEQ ID NO: 38 - DNA, 20 bp, synthetic construct
  source 1..20: mol_type = other DNA
gcctcctgcctccgctctac

SEQ ID NO: 39 - RNA, 20 nt, synthetic construct
  source 1..20: mol_type = other RNA
gtagagcggaggcaggaggc

SEQ ID NO: 40 - DNA, 20 bp, synthetic construct
  source 1..20: mol_type = other DNA
ttcacattgattttttggca

SEQ ID NO: 41 - DNA, 20 bp, synthetic construct
  source 1..20: mol_type = other DNA
tgccaaaaaatcaatgtgaa

SEQ ID NO: 42 - RNA, 20 nt, synthetic construct
  source 1..20: mol_type = other RNA
ttcacattgattttttggca

SEQ ID NO: 43 - DNA, 200 bp, synthetic construct
  source 1..200: mol_type = other DNA
tctaggactttataaaagatcactttttatttatgcacagggtggaacaagatggattatcaagtgtcaagtccaatctatgacatcaattattatacgatcgcatcggagccctgccaaaaaatcaatgtgaagcaaatcgcagcccgcctcctgcctccgctctactcactggtgttcatctttggttttgtgggcaa

SEQ ID NO: 44 - DNA, 200 bp, synthetic construct
  source 1..200: mol_type = other DNA
ccagaaggggacagtaagaaggaaaaacaggtcagagatggccaggttgagcaggtagatgtcagtcatgctcttcagccttttgcagtttatcaggcgatcgatgaggatgaccagcatgttgcccacaaaaccaaagatgaacaccagtgagtagagcggaggcaggaggcgggctgcgatttgcttcacattgattt

SEQ ID NO: 45 - DNA, 200 bp, synthetic construct
  source 1..200: mol_type = other DNA
atgctcttcagccttttgcagtttatcaggatgaggatgaccagcatgttgcccacaaaaccaaagatgaacaccagtgagtagagcggaggcaggacgatcgggcgggctgcgatttgcttcacattgattttttggcagggctccgatgtataataattgatgtcatagattggacttgacacttgataatccatctt

SEQ ID NO: 46 - DNA, 200 bp, synthetic construct
  source 1..200: mol_type = other DNA
ggatgaccagcatgttgcccacaaaaccaaagatgaacaccagtgagtagagcggaggcaggaggcgggctgcgatttgcttcacattgattttttgcgatcggcagggctccgatgtataataattgatgtcatagattggacttgacacttgataatccatcttgttccaccctgtgcataaataaaaagtgatcttt

SEQ ID NO: 47 - DNA, 17246 bp, Homo sapiens
  source 1..17246: mol_type = genomic DNA
ccgtgttatccaggatggtctcaatctcctgacctcgtgatctgcccgcctcggcctcccaaagtgctgggattacaggcatgagtcaccgtgcccgggcaatttttgtattttttagtagagacagggtttcacccttttggccaggctggtcttgaactcctgacctcaagtgatccacccgcctcggcctcccaaaaggatttattttttgaaaccagttccacagctctcagcttggtccacttatctgtcctccccaagcttcagctgtcacttgttaaacatgtataataatagtacttcaggccgggcacggtggcttgcacctgtaatcccagcactgtgggaagctgaggtgggtggatcacctgaggtcgggagttcgagaccagcctggctaacatggtgaaaccctatctctactaaaaatacaaaaattagccaggtgtggtggagcgcgcctgtaattccagctactaacagagaggctaaggcaggagaatcgcttgaacctggaaagcaggggttgccgtgagccaagatcatgccactgcactccagcctgggtgacagagacacactccatctcaaaaacacaaacaaacaaacaaaaaacatgtataataacagtacttcagccataggcattgtactcgaagatgctgagaaaggagacagtggccgggcaaggctgttcacacctataatcccagcactttgggaggccaaggcaggtggatcacctgaggtcaggagttcaagaccagcctagccaacatggtgaaacccccatctctactaaaaattgaaaaattagctgggcctggtggtggacgcctgtaatcccagctactagggaggctgaggcaggagaatcgcttgaacctgggaggcggaggttgcagtgagctgagatcgtaccactgcactccggcctgggcaacacagtgagactccatctcaaaaaaataagaaaggagatagtactggggaacgctcagcactgtgcgccaggtgctgaacaacaccactgcagtccttgttgtggtggattgtaccatctagttgctggctaatatggacagagatgctggccctttgattggggatggagcgtgggagctgtgaaagctcctctgggcttgagttcccacaggagggtgggcgtgtccacagaacacttccactcactccctgtctccctttctctcttctccccagctgacttcagggacctttataccaaagtgcttgaggaagaagctgcttctgtttcctctgcagatacaggtcaggcatgtggtttgcgccccagggatggggattgggcatggctgcccagccccctctccaccctacaataccattctcttatctctgtctctctgcagggctctgctctgaagcctgcctcttccgcctagcccgctgcccttcccccaagttgctacgtgcccggtcagccgagaaacggcgccctgtgcccaccttccaaaaagttcccctgccctcgggccctgcacctgcccactccctgggggacctaaagggcagctggccaggtcggggcctggtcactcgtttcctccagatatccaggaaagccccagaccccagtgggactggagctcatggacataagcaggtaggaattcggggagccaggaaagatgtttgggaaagcgtggagcttcagattgagccttattgatgatgccctttcttgtgtccctgtccaggtgccccggagcctgtggggccggcctggccgagagagcctccaccttcgcagctgcggagatctgagctctagctcttccctgcggcgtctcctgtctggccgcaggctggagcgtggtacccgcccccacagcctcagcctcaacgggggcagccgggagactgggctctgacctaggcttcttgtcacactgaacacatccagccacaggcaccagctggttgggaccagcagcccccagcatcctcttgcactggctggcacaaaaagaaacctgctgtataccccccaaagtgtccctttccctcctacctctggggtctcttgctgcttgcctctgctgctctggactgggagagcttctgtcctgtgctgcatgggtatttagactgtgggggagatgccccttcttatagcactggaggaggaaaacaaattcttgtccccctcagaatgagagtggctctttctgatttgcaagggcactatggtcagggcaaaggcatggcccaggtgtttaagtacagggtgacgtgtgcctatgcaatggggtggtaaggcaggcacgaagagtccaaaaaatctaggtggcctctcagctctgccacctctagctgcatgaccttgggcaagctatgtaaccccaattgcctgctccattaaagactgtgaaggtagaatgtttgtaaagctcttaacagtatgtaagccttcaataaatttcagttttccccttgttttcttgatcattctctgtcaccagtgaaatttgttctagtgtctctcatatttaagaaaactctttcaggactgggtatggtggctcacacctataatcctagcactttgggaggccgaagcaagaggatcgcctgagcctaggaattcaagaccagcctgggcaacatagtgagaccctgtctctacaaaaaacaaaaaattagccaggcatggtgggacacgcctgtagtcccaactactcaggtggctaaggtgagaggatcacttgagcttgggaagtccaggctgcagtaagctgtgattgagccactgcactacagcctgggcaacagagcaagaccatgtctcaaaaaaaaaaaaaaaagaaaaaagaaactttcaagacactctttccaaccactaattgtaactctgctcctccttttcacagcaataggttttctttttcttccctccactgttaaacatccattctctcctcacccacccccatcagactccttcccctatctttccacagccactgctctgaccaaactttccagtgaccacagtggtgtcagacccagtgaccatttctctgcctgcatctcacttgacctcgaggcagcaattaatacccataatcagcatcttcttgaatttgtccctttgaaaagggaaatattggctcttctactttgtcctgctgaactgcttaacattggagggccccagggccctcacctaagccctctttcctacctccactctttctataggtggccctactactaaagtccatggctttaaataccatctttctatgtgttaatccataactccagccttgacctcccatgagcgccatccaactcagcatgtctgcttggatgtctaatgggcatttcagattcaacatggccacaactgaactcttgattcccaccccagcaccggttatttttccactgttcccatctcaatggcacctccattacccatttgcacattccaaaagctcaggaaccatggtgacttcttttcccatatccaacacaaccaatcctatcctgaattcatccacatcccaccacctccccagctacctagctccagccatcctctctccacaacctctgaatcagtctttcacttttcccagcaatccattctccactcagcaaaatgatgataaagcacgtcacatcaaggctctgcctcaatttaatggcttcccattgtatttagaatcatctccaaactcccagagactatggtcagctacaatctggcccaccttctgttccagccaaatttcctcacagcacaaggacgtttgcacctgctgttttccaagcatgaaacccttggcccctatatctggtgctatcacctaatatcaggttttagctccattctcaccatttcagtgagcacccaatccccatcgcagtcattctatcacatagccatgtttttttttgtttgtttgtttcattttgtctttttttgagacagggtcttgctttgttacccaggctggagtgcagtggtgtgatttgggctcactgcaaccttccacctcctgggttcaagcaattctcctgcctcagcctcccgagtagctgggattacaggcgcccgtccccatgcccgcccagctaaattttgtatttttagaagagatagggtttcaccatgttggccaggcgggtctcaaactcctgacctcaagtaatccgcctgcctcggtctcccaaagtgctgggattacaggtgtgactcaccgcgcctggccacatacccatggtttcagcatgtatcactatctaaaattattatttttgtttatatatctgtgtcgtcccatagaatgttaaggtcccaagatcagaaacttgctcattgcagtgggtctaacactcagtaggtcctcaacaaacattcgttaagatactaaagtggcagggtggggccctgtaaacagcttcaggaccctgtgcttgtaggggcaacgtggtgccctccaaggaagacagggaggtgggaggagcactgcccagagatggcgtcaggctgcaagacttcttgaataattcagcatcataacaacccagcctcaggaagggatagggcacggccaggacgaaacattaggaggcgatggacaatgggattcccacggggcagcttctgcgcactggacgttccctaacctgaggctctctaaagaggaaggttaggaatcctctgagcttcggtgggctggactcactgtgggaattcaatcgcccccatccaccaacagtgtgctggcgggaaaacgccgacacgcatgcgtagttctcgcgccggctcctctctctctctctctctctctcgctcgctctctcgctctctcgctctctctcgctcgctctctcgctctcgctctctctctctctccggctcgccagcgacacttgttcgttcaacttgaccaatgagacttgaggaagggctctgagtcccgcctctgcatgagtgaccgtctcttttccaatccaggtcccgccccgactccccagggctgcttttctcgcggctgcgggtggtcgggctgcatcctgccttcagagtcttactgcgcggggccccagtctccagtcccgcccaggcgcctttgcaggctgcggtgggatttcgttttgcctccggttggggctgctgtttctcttcgccgacggtaggcgtaatgaatatttcgacctttggatcttagctgtcccctccctgcgttcgcacttaacctttttcaccattattattattattgttattattattattttttgagggagtctcgccctgtcgcccaggctggagtgtaatggcgccttcttggctcactgcaacctccgcctcccgggttcaggcgattctccgacctcagcctcccaagtacgtgggattacaggcacccgccaccacgcacggctaattttttgtatcttttagtagagacggggtttcaccatgttggtcaggctggtctccaattcctgacctcgtgatccgcccgcctcggcctgccaaacagctgtgattataggcgtgagccaccgcgcccggccaaccatcattattatttttaacggtaaggatggtcagattttactaatgaagaagagattataaaatcttcaagtctttatatccacttgctttttgaggggtggagtgggaagaaggttatgtaattcatacgttcttcagacatgtgacaaacattcacggagcccggcgacgagcgtcggggttgggattcgcactggagctgcagatgggtgccaggatggactggtccctaccctccgcttgaacctaggaggcggaggttgcagtgaaccgagatcgtgccactgcactccagcctgggtgacagagatactccgtctcaaaaaaaaaaacaaaacaaaaaacaagcggactgggcgcagtgcctcaccctgtaatcccagcactttgcaaagccaaggcgggaggatcctttgagtttaggagtttgagaccaacctgcgcaacacagtaagaccccgtctctacaaaaaatacagaaattagccaggtgtggtggtgtgcgcctatagtcccagctattctggaggctgaggtgggaggattgcttattctggaggcagaggttgcactgagccgaaatcaagctactacactccatccagggcaacatacggagaccctgtctcaaacaaacaaacaaaaaattgctcagtacctggccaaaaaagaagaggctcactatgcagaggggaagtggaaggagatgtttggacttctaaactcaatagagcaggagaggcaaatgtagaatgtgctcaggaaatatctgtgagatgaatgaacttgagggaagtaaggtactagatattacctgccctacccagaacaaatcctgtgcaatgtttccttgaaaagtgagaagtctggaaggggtggctactgacatagtgaagcaactagttcaattctacaacttgacagctacccctgtgccaggctatctacgaggatacttagaatgcataagacattccttcaaggaactccaggaacagaggcctgacatgttgcaatgtttagtgtcaagcagtgtactagagacacattatcacactcaaacctcacaacaattctgtgaggtaggagttatcactccccttttatagatgaaacagaggcttagagtgattgatttattgaaagtcaaacagccagtaaatggtgtagccaggattccaaacttgctgtctcactgagactgtacttaattactggagggaccgggtgtggtggctcattgctataatcccaacaccttgggaggctgaggctggtggatcacctgaggtcaggggttcgagaccagcctggccaacatggtgaaaccccatctctactaaaaatacaaaaattagctgggcatggtggtgggctcctgtaatcccagctactcaggaggctgaggcagggcaattgcttgagccgagatcacactgcactccagcctgggcaacagggcaagactctgtctcaaaaccaaaaaaaaaaaaattactggaggaacctagaagaagaaatgatcaattttgcttggagtgtatctagaaagacttcactgagatcatttaaagaacaaaaaggatggctggggtccagcgcagtggctcatgcctgtaatcccagcactttcggataccaaggcagcagatcacctgaggtccagagtttcagaccagcctggccaacatagtgaaaccccatctctactaaaaataaaaaaattagctgagcatgttggagggcacctgtaatcccagctacttgggaggctgaggcaggagaatcactcgaacccaggaggtggaggttgcagtgagccaagatcacgccactgcactccagcctgggcaacagagtgagactctgtctcaaaaaacaacaacaacaaaaaatacaaacaagagacaagtagttcccaggtgcctaccaagtggtcaggcactgcacttacctcactgactgcagtaaccaccctttgaggttgtggcattgcctccattttccaggcaaggaaatgggctgagagctgggattagtcaggtcatgactgtgtgtgccactcccgctaaatctcatttgatgtggttcatgaggccacaccatggacagcttcctccttgtgtccactgaggatatggctttgtacaacactttggtttttgaacgactttacaaacctccctgtcttgtgaggaaggaagaacagttattaccatctgcatctgatgatgaaacaagggacgctgcagaggagccgcactgaccactccctccctccagtcctgtcatcccactgccagtgtcccaccctcttgtgccctgcacttcactggctaataacccccctcactttttcctctgtgaagccatcctggataattccccacccacgaatggtccctcctcatctcagagagctctccatgcacacctgttaccgtttctgtctttatctgtaaatatctgtgtgtctgacttccatgcctcacacacctctatagggcaaagactgtcttaaacatcttggtagtgtcagtattttgcacagtgaagtttttttttttaaattatatcagctttatttgtacctttttgacatttctatcaaaaaagaagtgtgcctgctgtggttcccatcctctgggatttaggagcctctaccccattctccatgcaaatctgtgttctaggctcttcctaaagttgtcacccatacatgccctccagagttttatagggcatataatctgtaacagatgagaggaagccaattgccctttagaaatatggctgtgattgcctcacttcctgtgtcatgtgacgctcctagtcatcacatgacccatccacatcgggaagccggaattacttgcagggctaacctagtgcctatagctaaggcaggtacctgcatccttgtttttgtttagtggatcctctatccttcagagactctggaacccctgtggtcttctcttcatctaatgaccctgaggggatggagttttcaagtccttccagagaggtaagagagagagctcccaatcagcattgtcacagtgcttctggaatcctggcactggaatttaatgaatgacagactctctttgaatccagggccatcatggctctttgagcaaggcacagatggagggaggggtcgaagttgaaatgggtgggaagagtggtggggagcatcctgatttggggtgggcagagagttgtcatcagaagggttgcagggagagctgcacccaggtttctgtgggccttgtcctaatgaatgtgggagaccgggccatgggcacccaaaggcagctaagccctgcccaggagagtagttgaggggtggagaggggcttgcttttcagtcattcctcattctgtcctcaggaatgtcccaagcctttgagtagggtaagcatcatggctggcagcctcacaggattgcttctacttcaggcagtgtcgtgggcatcaggtgagtgagtcaaggcagtggggaggtagcacagagcctcccttctgcctcatagtcctttggtagccttccagtaagctggtggtagacttttagtaggtgctcaataaatccttttgagtgactgagaccaactttggggtgaggattttgttttttttcttttgaaacagagtcttactctgttgcctgggctggagtgcagtggtgcaattttggctcattccaacctctgcctcccagattcaagcgattctcttgcttcagcttcccaggtagctgggattacaggcggccaccactacgcccagctaatttttgtatttttagtagagacggggtttcaccatgctggcaaggcaggtctcaaactcctcacctcaggtgatccgcccacctcggcctcctaaagtgctaggattacaggtgtgagcccctgcgcccggccaaggggtgaggaattttgaaaccgtgttcagtctctcctagcagatgtgtccattctccatgtcttcatcagacctcactctgcttgtactccctccctcccaggtgcccgcccctgcatccctaaaagcttcggctacagctcggtggtgtgtgtctgcaatgccacatactgtgactcctttgaccccccgacctttcctgcccttggtaccttcagccgctatgagagtacacgcagtgggcgacggatggagctgagtatggggcccatccaggctaatcacacgggcacaggtaaccattacacccctcaccccctgggccaggctgggtcctcctagaggtaaatggtgtcagtgatcaccatggagtttcccgctgggtactgatacccttattccctgtggatgtcctcaggcctgctactgaccctgcagccagaacagaagttccagaaagtgaagggatttggaggggccatgacagatgctgctgctctcaacatccttgccctgtcaccccctgcccaaaatttgctacttaaatcgtacttctctgaagaaggtgaggaggaaggggacaagatgacatagagccattgaaacttttcgtttttcttttctttttttaaaatttttttgaggcagaatctcactctgcccattctgtcggcgagacaggagtgcagtggtgtgatctcccctcacagcaacctctgcctcccaggctatagtgattctcctgcctcagcctcctgagtagctggaattataggcgtgcgccactaccacctggctaatttttgtatttttagtagagacagggtttcatcatgttgaccaggctagtcttaaactcctgacctcaaatgatatacctgccttggcctcccgaagtgctggaattacaagtgtgagccaccgagcccagcagacacttttcttttttctttttttttttttgagacagagtctcgcactgtcacccaggctggagtgcagtggcacaatctcagctcactgcaacctccacctcccgggttcaggtgattctcctgtctcagcctctcgagtacctgggattacaggtgcctgccaccacgcccggctaattttttgtatttttagtagagacagggtttcactatgttggccaggatgattgcgaactcctgacctcgtgatctgcccacatcggcctcccaaagtgctgggattacatgcgtgagccactgacacttttctttgccctttctttggaccctgacttctgcccatccctgacatttggttcctgttttaatgccctgtgaaataagatttcaccgcctatcatctgctaactgctacggactcaggctcagaaaggcctgcgcttcacccaggtgccagcctccacaggttccaacccaggagcccaagttccctttggccctgactcagacactattaggactggcaagtgataagcagagtcccatactctcctattgactcggactaccatatcttgatcatccttttctgtaggaatcggatataacatcatccgggtacccatggccagctgtgacttctccatccgcacctacacctatgcagacacccctgatgatttccagttgcacaacttcagcctcccagaggaagataccaagctcaaggtaggcattctagctttttcaggccctgagggccctgatgtctgggggttgagaaactgtagggtaggtctgcttgtacagacattttgtcccctgctgttttgtcctgggggtgggagggtggaggctaatggctgaaccggatgcactggttgggctagtatgtgttccaactctgggtgcttctctcttcactacctttgtctctagatacccctgattcaccgagccctgcagttggcccagcgtcccgtttcactccttgccagcccctggacatcacccacttggctcaagaccaatggagcggtgaatgggaaggggtcactcaagggacagcccggagacatctaccaccagacctgggccagatactttgtgaagtaagggatcagcaaggatgtgggatcaggactggcctcccatttagccatgctgatctgtgtcccaaccctcaacctagttccacttccagatctgcctgtcctcagctcacctttctaccttctgggcctttcagccttgggcctgtcaatcttgcccactccatcaggcttcctgttctctcggtctggcccactttctttttatttttcttcttttttttttttttgagaaggagtctctctctctgtcacccaggctggagtgctgtggcgccatcttcactcactgtaacctctgcctcctgagttcaagcaattctcctgcctcagccttccaagtagctgggattataggcgcctgccaccaggcccagctgatttttctatttttagtagagacggggtttcgccaggctgttctcgaactcctgaactcaagtgatccacctgcctcggcttcccaaagtgctgggattacaggtgtgagccaccacacccagctggtctggtccactttcttggccggatcattcatgacctttctcttgccaggttcctggatgcctatgctgagcacaagttacagttctgggcagtgacagctgaaaatgagccttctgctgggctgttgagtggataccccttccagtgcctgggcttcacccctgaacatcagcgagacttcattgcccgtgacctaggtcctaccctcgccaacagtactcaccacaatgtccgcctactcatgctggatgaccaacgcttgctgctgccccactgggcaaaggtggtaaggcctggacctccatggtgctccagtgaccttcaaatccagcatccaaatgactggctcccaaacttagagcgatttctctacccaactatggattcctagagcaccattcccctggacctccagggtgccatggatcccacagttgtcgcttgaaacctttctaggggctgggcgaggtggctcactcatgcaaacccagcactttgggaagccgaggcgggtgatcacctgaggtcaggagtttaagaccaccctggccaacgtgttgaaaccctgtgtctactaaaatacaaaaaaaaaaaattatctgggcatgatggtgggtgtctgtaatcccagctactcaggaggctgagaagggagaatcagttgaacccgggagatggtggttgcggtgagccgagatcgcgccactgcactccagcctgggaggctgagcgagactccatctcgaaacaaaacaaaacaaaactatctaggctgggggtggtggttcatgtatgtatgtgtatatacatatatatgtgtttatatgtatatatatatacacacacacacatacatacacacacatacacacacaaattagctgggtgtggcacccgtgtagtcccagctactcaggaggctaatgtgggaggatcagttgaccctaggaagtcaaggctgcagtgagtcgtgattgcgccactgtactccagcccgagtgacagagtgacatcctgtctcaaaaacaaaaaaaaatctccccaaacctctctagttgcattcttcccgtcacccaactccaggattcctacaacaggaactagaagttccagaagcctgtgtgcaaggtccaggatcagttgctcttcctttgcaggtactgacagacccagaagcagctaaatatgttcatggcattgctgtacattggtacctggactttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacggtaagccaccccagtctcccttcctgcaaagcagacctcagacctcttactagtttcaccaaagactgacagaagcccttcctgtccagctttccccagctagcctgcccttttgagcaactctggggaaccatgattccctatcttccctttccttcacaggtctgcacacctcattgccccttttgcaactactgaggcacttgcagctgcctcagacttctcagctccccttgagatgcctggatcttcacacccccaactccttagctactaaggaatgtgcccctcacagggctgacctacccacagctgcctctcccacatgtgacccttacctacactctctggggacccccagtgttgcgcctttgtctctttgcctttgtccttaccctagaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcaggtgagtggagggcgggcacccccattccataccaggcctatcatctcctacatcggatggcttacatcactctacaccacgagggagcaggaaggtgttcagggtggaacctcggaagaggcacacccatccccttttgcaccatggaggcaggaagtgactaggtagcaacagaaaaccccaatgcctgaggctggactgcgatgcagaaaagcagggtcagtgcccagcagcatggctccaggcctagagagccagggcagagcctctgcaggagttatggggtgggtccgtgggtgggtgacttcttagatgagggtttcatgggaggtaccccgagggactctgaccatctgttcccacattcagcaagttcattcctgagggctcccagagagtggggctggttgccagtcagaagaacgacctggacgcagtggcactgatgcatcccgatggctctgctgttgtggtcgtgctaaaccggtgagggcaatggtgaggtctgggaagtgggctgaagacagcgttgggggccttggcaggatcacactctcagcttctcctccctgctccctagctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttatgtgaacatgcgctgtgtgctgcttgctttggaaactgggcctgggtccaggcctagggtgagctcactgtccgtacaaacacaagatcagggctgagggtaaggaaaagaagagactaggaaagctgggcccaaaactggagactgtttgtctttcctggagatgcagaactgggcccgtggagcagcagtgtcagcatcagggcggaagccttaaagcagcagcgggtgtgcccaggcacccagatgattcctatggcaccagccaggaaaaatggcagctcttaaaggagaaaatgtttgagcccagtcagtgtgagtggctttattctgggtggcagcaccccgtgtccggctgtaccaacaacgaggaggcacgggggcctctggaatgcatgagagtagaaaaaccagtcttgggagcgtgaggacaaatcattcctcttcatcctcctcagccatgcccagggtccgggtgcctggggcccgagcaggcgttgcccgctggatggagacaatgccgctgagcaaggcgtagcccaccatggctgccagtcctgccagcacagataggatctggttccggcgccggtatggctcctcctcagtctctgggcctgctggtgtctggcgttgcggtggtacctcagctgagggtcaaggaaggaaggtgtgttaggagaactagttcttggatccctgcccactctccccagggctgcccctcccatctgccccttacctccatcccaggggaagtagagactgagaatgtgggtacaataggcacagaggttgtgcagcccacgcaggtggacctgcagcttcccactgggcagctttgcctgcagcagcagggccaagtagctgaagacgaaggcgtccaaggaggcagggctggagcagagagagaagggtgggatggaggagaaccactggggtagaaggggtaaagatggagctggaggaagagtcagccttgggaggtgggctctgggcagcaggcggccaccagggaaggacaggacacacagttctagacctggtatggggagagatccccaggtggcgccagcctggccctgaatagggctctatcccagggctgcataaagggcacactcagtgccccacagctcttcaggcccttcctgtgcctggctgccctcccaccctacccttttgtacctctgagaaggctctggccccacgcacagccccactgtcaccagggccagtatctgtctcagggacctcctatccagagcctgagccagccccagccccagccccagctccagctgctccatctgaacctgtatcttcttccaagccacccattaccctcttggagtcagactcacgcatctccaaagaagaacttttgagagcccaggcgctgagagagcagggtcagacactcccgagcctctcggtacagctgtaggggcgacacaggtaggcttgcagctgcgggaacagtgccacctccgcacctaagcactcccattcctggccagcatccttggggctcatctcatacaatagcccccggtctcagagctacctccttctccagctcttcctcgtcctcaggcctgtgctccccagtcagcagctgtagccgttccatgtactgccgctgcatgcggccaggcaggaagaagttgaggggaaagggcatagcctctgcataccacttccgggtcacttctacgtagttcttggtgtctatccaaaaagtatgtacctggattgggtgggcaggaagaaacaggcaggtctgagccagtgcacctgtctgattcaaggtgggcttctgacctccatgctctcctgagtctctgtgtgggtctgtgtgttcccgtcccctccccggctggccatggatgctgggaggtctgggcacactcaccagcaccgggatcaacttctcctccaggagagacatgaaggccagggtgtctgccccttgctgagctgacagatcataatcagcattgtacttctgtggaggaaatatccatggcgtggacgctggggagctgcaagggcacttcaccagggaggaaggagtcctgtctggtacccccctcactggcctctgagtgcagtggaggtacagcaaggaacttttcctgccaaggcccccttgcctgggcccagccagtagcctgttgctgttggcaaaaagcctgggccttggagcccgctggccgtcaaggtcctgggcccattgagaagaaggaagaaaggttgggccgcaaactaggagcagctcccagaatttccatggaaagctggaacaa

SEQ ID NO: 48 - protein, 536 aa, Homo sapiens
MEFSSPSREECPKPLSRVSIMAGSLTGLLLLQAVSWASGARPCIPKSFGYSSVVCVCNATYCDSFDPPTFPALGTFSRYESTRSGRRMELSMGPIQANHTGTGLLLTLQPEQKFQKVKGFGGAMTDAAALNILALSPPAQNLLLKSYFSEEGIGYNIIRVPMASCDFSIRTYTYADTPDDFQLHNFSLPEEDTKLKIPLIHRALQLAQRPVSLLASPWTSPTWLKTNGAVNGKGSLKGQPGDIYHQTWARYFVKFLDAYAEHKLQFWAVTAENEPSAGLLSGYPFQCLGFTPEHQRDFIARDLGPTLANSTHHNVRLLMLDDQRLLLPHWAKVVLTDPEAAKYVHGIAVHWYLDFLAPAKATLGETHRLFPNTMLFASEACVGSKFWEQSVRLGSWDRGMQYSHSIITNLLYHVVGWTDWNLALNPEGGPNWVRNFVDSPIIVDITKDTFYKQPMFYHLGHFSKFIPEGSQRVGLVASQKNDLDAVALMHPDGSAVVVVLNRSSKDVPLTIKDPAVGFLETISPGYSIHTYLWRRQ

SEQ ID NO: 49 - DNA, 13065 bp, Homo sapiens
  source 1..13065: mol_type = genomic DNA
ttgaaagaagtgggtaaaatgctatcaaatagcatcacatggtatggagaaatcttttgtgaagggaagagtcgaccaaggtggcaaattgcattgtcatcttattttaagaaattgccacagccacccccagctttagcaaccaccaccctgatcagtaagcagccatcaacatcaaaacaagaccgccatcctcttcagcaaaaacactatgacttgctgaaggctcagatgatggttagcatttttagcaatacaatatttttaattaaggtatgcacattggtttttctgacataatactattgcatacttaatagactacagtataggataaacacaacttttatatgcactgggaaaccaaaaaggttatttttgagatatttgctttactgtggtggtctgaagctgaactcacaatctcaccaaggtgtgcctgaacctctttagctaactggccactgccacagtccactctgtgttggtcaagatgccccagagtggcaggcacactgtgtggtcacatccaagggcctagatatggtgggggctccaaatggatctagatatgtgagatctctctttgatttgacttcttccaacccaccattttctgggtgctgggctcatctcacccagaaagtaggacccaatgtgacagttcctgcccagttccctcctgtggtagccacttgacccaggggcactcttgatccttgcagcctcacttacacaccctatctctacccctattaactctctccaatccccactccccctgctcagcttgtctgctgcccagtgggggccccacccatgctggcctctccttttgcaagtccccattcctcatatggtttcttcagagcccctttctttggctttgaggagagatgccctcactcgcttccccaccaatcctgcccacttctacaatccattcattatcctaattgcctccgtatacagactggagtgagaggagttgatgtgatgggtgtggatacagggctggtgctgtcatcttctagtaagccctgggagaggtgtctgagcccaggtgtcagtggttttctttggaactgtgagtgcataacacttctttgccttcagccttaggccatagttgctagttctgggacaaccagaaaagccctacataatctcgtgttatgtgcagagctgagtatagagctccaggtatgatctgactcacttaagatcacagtgagtctattgtattgttgaactgttagcttagacatctgttactgtacctacatggcactagcctcacgcctagacaccgatctgaaagaaatcccctaaatgcatagagaagacttctcagctgagctaaggggctcccaccaggtttgagcctatctaatgaatccatgaggtagacagcctgcacatgtccacttggtttgatgaattgcacaaatccctatgggggatgtggttcatgggctgggaagtgggttaccctgggaaaggtctacaggacagaggcagggatggagacaacagcatggtgagttcccaacccacccacgatgataggtgtctgaggcagaaggtaaagaggctgtcacctggtgggtgtcataagactcaagtgtcattgttgaggcacatgggtaacaaagcgtggcactggatgggggtagattcttcctatttctgtgaggatcagggggactccctggctctcctgctaaaggtggctctagggacaggaagagtgtacttcttgacagggatgtcagagcactgatggtgacaatcagtgtgacactgctcacatgactgaacaaccgagaagagcccgactgtctactgaacaacgggaagagcccgactgtcaatgacggagctctgttaaatatagttaaggctattttgttgaatgaatgaagccagacaggaaagaggacagtatctttaatccatttatagaagttaaagacaggcttatttaatctctatgaagacagagtggcccttacctctgggtggagcaaaaggcaccttctgaagtgatagggatgttccttatcatcttgatccggagtggtagttacatgcatgtgtgcatatcaaaactcaccaagctgtaccactaagtgtgttcttcctcaataaaaataataaagaactacacttataaagaattttttaataatataggaaaatgtctacactataatctttagctaaaaaaaaaaaaaaaagaagccgcctacagaatggtatatgcatgagaacaattaatcgaaaagtgcatgggaaaagtcaggattgaaacatcatgttttaaaagacattgttttgatactgtgagaatgtacctaagtttttccttttttctgtttttcccaattttatacaatgagcatgtgttggttttataattagacattttgtttgtttggtttggttttgagacacagcttgctgtcacccaggttggagtgcaatggcccaatcttggttcactgcaacctccatctcctgggttcaagagattctcccacttcagcctcctgagtagctgggactataggggcgcaccaccacatccagctaattttgtgtatttttagtagagatggggtttcaccatgctggccaggttggtctcaaactcctgacctcaagttatccactcgccttggcttcccaaagtgctgggattataggcatgagccaccgcacttggcctagacatttgtttttaaaaataaaagattcatttgctctttttacagcccgtctcactgttgactgatattgaccaggagtcaactcaggccccagggattttcacaacagctgctgtatggcagggtttctgctcactgtgctcatgtagttggcccttgcacccaaagtgaataattaacattctccccatcctgttgacgatgctctgaaaatatggtccagaaatggtgtgagcaaggagacagcaaagcaatgcttggaacataggtgcagtgactagacatggggcagctgtttaaagacaaaaaggccccaaaaaggagggatggcacgaaacaccctccaatatgggcatggagtctagagtgacaaagtgatcaaaagttcatttcctatggggtgtccgaatgtacttaataataaaaagagaacaagagccatgcaaactgagagggacaaagtagaaagagtagcagacaccaagcaactaagtcacagcatgataagctgctagcttgttgtcattattgtatccagaacaacatttcatttaaatgctgaagaatttcccatgggtccccactttcttgtgaatccttgggctgaacccccctgtcctgagtggttactagaacacacctctggaccagaaacacaaaagtggagtaacgcacactgcaaagctgtgcttccttgtttcagcctgtgaatcctcaccttgtttcccatctagcctatatttttcaaactaacttggccatagaatcatgtagtatttagggtggaagctgccccaggtctagcacgtcatttaacagatgaggaaatggaagcttgggcagtggaagtatcttgccgaggtcacacagcaagtcagcagcacagcgtgtgtgactccgagcctgctccgctagcccacattgccctctgggggtgagtatgtcttcacatcctccaataccctaatgacagacaaacagaacatggcaaagcctcagctctgcatggtgaaagtaagaaccagcaattgccacaaacagaaatacagtgttggtccggcagcctccgggggttctgcacaagtggattaccagtgaatacaaggctatctatctttcgaaaaaccaaagttgtatttatgctatctattttctataaaattttatattaatttatttgttacctatttttgaactctttcaaaagcacactttatatttccctgcttaaacagtcccccgagggtgggtgcccaaaaggctctacacttgttatcattccctctccaccacaggcatattgagtaagtttgtatttgggtttttttaaaacctccactctacagttaagaaaactaaggcacagagcttcaataatttggtcagagccaagtagcagtaatgaagctggaggttaaacccagcagcatgactgcagttcttaatcaatgccttttgaattgcacatatgggatgaactagaacattttctcgatgattcgctgtccttgttatgattatgttactgagctctgttgtagcacagacatatgtccctatatggggcgggggtgggggtgtcttgatcgctgggctatttctatactgttctggcttttcccaagcagtcatttctttctattctccaagcaccagcaattagctttaccttttcagcttctagtttgctgaaactaatctgctatagacagagactccggtgaaccaattttattaggatttgatcaaataaactctctctgacaaaggactgctgaaagagtaactaagagtttgatgtttactgagtgcatagtatgtgctagatgctggccgtggatgcctcatagaatcctcccaacaactcatgaaatgactactgtcattcagcccaatacccagacgagaaagctgagggtaagacaggtttcaagcttggcagtctgactacagaggccactggcttagcccctgggttagtctgcctctgtaggattgggggcacgtaattttgctgtttggggtctcatttgccttcttagagatcacaagccaaagctttttattctagagccaaggtcacggaagcccagagggcatcttgtggctcgggagtagctctctgctgtcttctcagctctgctgacaatacttgagattttcagatgtcaccaaccgccaagagagcttgatatgactgtatatagtatagtcataaagaacctgaacttgaccatatacttatgtcatgtggaaaatttctcatagcttcagatagattatatctggagtgaagaatcctgccacctatgtatctggcatagtgtgagtcctcataaatgcttactggtttgaagggcaacaaaatagtgaacagagtgaaaatccccactaagatcctgggtccagaaaaagatgggaaacctgtttagctcacccgtgagcccatagttaaaactctttagacaacaggttgtttccgtttacagagaacaataatattgggtggtgagcatctgtgtgggggttggggtgggataggggatacggggagagtggagaaaaaggggacacagggttaatgtgaagtccaggatccccctctacatttaaagttggtttaagttggctttaattaatagcaactcttaagataatcagaattttcttaaccttttagccttactgttgaaaagccctgtgatcttgtacaaatcatttgcttcttggatagtaatttcttttactaaaatgtgggcttttgactagatgaatgtaaatgttcttctagctctgatatcctttattctttatattttctaacagattctgtgtagtgggatgagcagagaacaaaaacaaaataatccagtgagaaaagcccgtaaataaaccttcagaccagagatctattctctagcttattttaagctcaacttaaaaagaagaactgttctctgattcttttcgccttcaatacacttaatgatttaactccaccctccttcaaaagaaacagcatttcctacttttatactgtctatatgattgatttgcacagctcatctggccagaagagctgagacatccgttcccctacaagaaactctccccggtaagtaacctctcagctgcttggcctgttagttagcttctgagatgagtaaaagactttacaggaaacccatagaagacatttggcaaacaccaagtgctcatacaattatcttaaaatataatctttaagataaggaaagggtcacagtttggaatgagtttcagacggttataacatcaaagatacaaaacatgattgtgagtgaaagactttaaagggagcaatagtattttaataactaacaatccttacctctcaaaagaaagatttgcagagagatgagtcttagctgaaatcttgaaatcttatcttctgctaaggagaactaaaccctctccagtgagatgccttctgaatatgtgcccacaagaagttgtgtctaagtctggttctcttttttctttttcctccagacaagagggaagcctaaaaatggtcaaaattaatattaaattacaaacgccaaataaaattttcctctaatatatcagtttcatggcacagttagtatataattctttatggttcaaaattaaaaatgagcttttctaggggcttctctcagctgcctagtctaaggtgcagggagtttgagactcacagggtttaataagagaaaattctcagctagagcagctgaacttaaatagactaggcaagacagctggttataagactaaactacccagaatgcatgacattcatctgtggtggcagacgaaacattttttattatattatttcttgggtatgtatgacaactcttaattgtggcaactcagaaactacaaacacaaacttcacagaaaatgtgaggattttacaattggctgttgtcatctatgaccttccctgggacttgggcacccggccatttcactctgactacatcatgtcaccaaacatctgatggtcttgccttttaattctcttttcgaggactgagagggagggtagcatggtagttaagagtgcaggcttcccgcattcaaaatcggttgcttactagctgtgtggctttgagcaagttactcaccctctctgtgcttcaaggtccttgtctgcaaaatgtgaaaaatatttcctgcctcataaggttgccctaaggattaaatgaatgaatgggtatgatgcttagaacagtgattggcatccagtatgtgccctcgaggcctcttaattattactggcttgctcatagtgcatgttctttgtgggctaactctagcgtcaataaaaatgttaagactgagttgcagccgggcatggtggctcatgcctgtaatcccagcattctaggaggctgaggcaggaggatcgcttgagcccaggagttcgagaccagcctgggcaacatagtgtgatcttgtatctataaaaataaacaaaattagcttggtgtggtggcgcctgtagtccccagccacttggaggggtgaggtgagaggattgcttgagcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgaggcttatcttcaccatcatgattgtttattttctcttctgggctccctacaacattgtccttctcctgaacaccttccaggaattctttggcctgaataattgcagtagctctaacaggttggaccaagctatgcaggtgacagagactcttgggatgacgcactgctgcatcaaccccatcatctatgcctttgtcggggagaagttcagaaactacctcttagtcttcttccaaaagcacattgccaaacgcttctgcaaatgctgttctattttccagcaagaggctcccgagcgagcaagctcagtttacacccgatccactggggagcaggaaatatctgtgggcttgtgacacggactcaagtgggctggtgacccagtcagagttgtgcacatggcttagttttcatacacagcctgggctgggggtggggtgggagaggtcttttttaaaaggaagttactgttatagagggtctaagattcatccatttatttggcatctgtttaaagtagattagatcttttaagcccatcaattatagaaagccaaatcaaaatatgttgatgaaaaatagcaacctttttatctccccttcacatgcatcaagttattgacaaactctcccttcactccgaaagttccttatgtatatttaaaagaaagcctcagagaattgctgattcttgagtttagtgatctgaacagaaataccaaaattatttcagaaatgtacaactttttacctagtacaaggcaacatataggttgtaaatgtgtttaaaacaggtctttgtcttgctatggggagaaaagacatgaatatgattagtaaagaaatgacacttttcatgtgtgatttcccctccaaggtatggttaataagtttcactgacttagaaccaggcgagagacttgtggcctgggagagctggggaagcttcttaaatgagaaggaatttgagttggatcatctattgctggcaaagacagaagcctcactgcaagcactgcatgggcaagcttggctgtagaaggagacagagctggttgggaagacatggggaggaaggacaaggctagatcatgaagaaccttgacggcattgctccgtctaagtcatgagctgagcagggagatcctggttggtgttgcagaaggtttactctgtggccaaaggagggtcaggaaggatgagcatttagggcaaggagaccaccaacagccctcaggtcagggtgaggatggcctctgctaagctcaaggcgtgaggatgggaaggagggaggtattcgtaaggatgggaaggagggaggtattcgtgcagcatatgaggatgcagagtcagcagaactggggtggatttgggttggaagtgagggtcagagaggagtcagagagaatccctagtcttcaagcagattggagaaacccttgaaaagacatcaagcacagaaggaggaggaggaggtttaggtcaagaagaagatggattggtgtaaaaggatgggtctggtttgcagagcttgaacacagtctcacccagactccaggctgtctttcactgaatgcttctgacttcatagatttccttcccatcccagctgaaatactgaggggtctccaggaggagactagatttatgaatacacgaggtatgaggtctaggaacatacttcagctcacacatgagatctaggtgaggattgattacctagtagtcatttcatgggttgttgggaggattctatgaggcaaccacaggcagcatttagcacatactacacattcaataagcatcaaactcttagttactcattcagggatagcactgagcaaagcattgagcaaaggggtcccatagaggtgagggaagcctgaaaaactaagatgctgcctgcccagtgcacacaagtgtaggtatcattttctgcatttaaccgtcaataggcaaaggggggaagggacatattcatttggaaataagctgccttgagccttaaaacccacaaaagtacaatttaccagcctccgtatttcagactgaatgggggtggggggggcgccttaggtacttattccagatgccttctccagacaaaccagaagcaacagaaaaaatcgtctctccctccctttgaaatgaatataccccttagtgtttgggtatattcatttcaaagggagagagagaggtttttttctgttctgtctcatatgattgtgcacatacttgagactgttttgaatttgggggatggctaaaaccatcatagtacaggtaaggtgagggaatagtaagtggtgagaactactcagggaatgaaggtgtcagaataataagaggtgctactgactttctcagcctctgaatatgaacggtgagcattgtggctgtcagcaggaagcaacgaagggaaatgtctttccttttgctcttaagttgtggagagtgcaacagtagcataggaccctaccctctgggccaagtcaaagacattctgacatcttagtatttgcatattcttatgtatgtgaaagttacaaattgcttgaaagaaaatatgcatctaataaaaaacaccttctaaaataattcattatattcttgctctttcagtcaagtgtacatttagagaatagcacataaaactgccagagcattttataagcagctgttttcttccttagtgtgtgtgcatgtgtgtgtgatgtatacaaagagagagataattgtatttttgtattttcttttaaataatttttaaaattgacccttttcctgagacaaattgccagaatagtttgtatttagagatggtacctctaagagtaaggttgctggttgctgagcaattgacttgaaaacttttaaaattcaaattttaattccactactcaaaagaattgccatgttttaaaaaagagaattggtgccataagttagttgtctatgtttgaaaatgaagaagatatgcaacgtcatggcctggtcacttacccgcagccctgagttgtaggcacatcatatgtgagaatgaggatgcttttctttcatttaaaatccctccccaaaacttggctctaattgcagtcatgacaatcatgtacatttggatttatgtgcacgagtctcttaccctgagagaggacaggtgctacaggtggaggggacccgtctgggtcacgttcacattttgaacatgctggttttcagtcactgcacactcatctcccagcacaggtcatgggcagcagatgcaaaagctgcccgtggtcctatttggaggtgcatgaaatgagcagaagacagaacagcttgatctgactagaagggcagcttgtccctaccaagacttgaaggattgcctttcatctgttagggtaaaaggtagaatgaaccaaggaagggcaggagggggctggggttagggtagaaggaaggggccatggagaagggagatccatcccataggaggaaggcagtgcggcagggaggtttgaaggtatcagcttttgtggctgacatacatgcagtcatgtcaattgctcgtttttccttttccatcttattaaatgtcttccaacgttagcacgaagaaaagctatttgcagtgttgccagcctttccagagcccgtccccattacctccccaggcccatgcctttactccttggagtttcaactcacgaccttcaggatctgactttattcaccaactctggggtgaacgtaccttctgtctccacccagaggtctctatcaaagaggagattgcatgccatggataaagtcaaagtagaggtgactgtccttaggaagagtaatgtgaaaattcataaactgggattctgtttacattttgtactccaggggttcttagtttaaatcgctctgaataaattaagatgcaatggcatttcaactgttatgattaaatttacaaatcatttattttctatcacggggagagatagagctccaaatgcaaacataactgctcaagtgttaacacttataatgaaaacataagaattaccaccaactaccctgggggctagaagcagaaatgtgaaccagaaaacaaatcatgaactttcctttttttttttgagatggagtctcgctctgttgcccaggctggagtgcaatggtgcgatctcggctcactgcaaccactgcctcccgggttcaagcaattctcctgcctcagcctcctgagtagctgggactacaggcatgcaccaccacgcctgggtaattttttgtatttttagtagagacagggtttcaccgtattagccaggatgctctcgatctcctgacctcgtgatctgcccgcctcggcctcccaccgaagtgctgggattacaggcatgagccactgtgcccggccaacaaatcatgaactttctaactgcagttccttgtagcttgttaacacatccacttacttattgtcagagtacgtggagattttccacaaccctcggggataaggctgaacagaagaggcaaaaacgtgaaaacatttcgatagctcctatactttgaaataaaattcactgtaaaagttgcttgtatttttccaaaac

SEQ ID NO: 50 - DNA, 1649 bp, synthetic construct
  source 1..1649: mol_type = other DNA
actttctggctccagccaaagccaccctaggggagacacaccgcctgttccccaacaccatgctctttgcctcagaggcctgtgtgggctccaagttctgggagcagagtgtgcggctaggctcctgggatcgagggatgcagtacagccacagcatcatcacggtaagccaccccagtctcccttcctgcaaagcagacctcagacctcttactagtttcaccaaagactgacagaagcccttcctgtccagctttccccagctagcctgcccttttgagcaactctggggaaccatgattccctatcttccctttccttcacaggtctgcacacctcattgccccttttgcaactactgaggcacttgcagctgcctcagacttctcagctccccttgagatgcctggatcttcacacccccaactccttagctactaaggaatgtgcccctcacagggctgacctacccacagctgcctctcccacatgtgacccttacctacactctctggggacccccagtgttgcgcctttgtctctttgcctttgtccttaccctagaacctcctgtaccatgtggtcggctggaccgactggaaccttgccctgaaccccgaaggaggacccaattgggtgcgtaactttgtcgacagtcccatcattgtagacatcaccaaggacacgttttacaaacagcccatgttctaccaccttggccacttcaggtgagtggagggcgggcacccccattccataccaggcctatcatctcctacatcggatggcttacatcactctacaccacgagggagcaggaaggtgttcagggtggaacctcggaagaggcacacccatccccttttgcaccatggaggcaggaagtgactaggtagcaacagaaaaccccaatgcctgaggctggactgcgatgcagaaaagcagggtcagtgcccagcagcatggctccaggcctagagagccagggcagagcctctgcaggagttatggggtgggtccgtgggtgggtgacttcttagatgagggtttcatgggaggtaccccgagggactctgaccatctgttcccacattcagcaagttcattcctgagggctcccagagagtggggctggttgccagtcagaagaacgacctggacgcagtggcactgatgcatcccgatggctctgctgttgtggtcgtgctaaaccggtgagggcaatggtgaggtctgggaagtgggctgaagacagcgttgggggccttggcaggatcacactctcagcttctcctccctgctccctagctcctctaaggatgtgcctcttaccatcaaggatcctgctgtgggcttcctggagacaatctcacctggctactccattcacacctacctgtggcgtcgccagtgatggagcagatactcaaggaggcactgggctcagcctgggcattaaagggacagagtcagctcacacgctgtctgtgactaaagagggcacagcagggccagtgtgagcttacagcgacgtaagcccaggggcaatggtttgggtgactcactttcccctctaggtggtgccaggggctggaggcccctagaaaaagatcagtaagccccagtgtccccccagcccccatgcttat

SEQ ID NO: 51 - DNA, 200 bp, synthetic construct
  source 1..200: mol_type = other DNA
tgcctctcccacatgtgacccttacctacactctctggggacccccagtgttgcgcctttgtctctttgcctttgtccttaccctagaacctcctgtaccatgtggtcggctggaccgactggaaccttgccctgaaccccgaaggaggacccaattgggtgcgtaactttgtcgacagtcccatcattgtagacatcac

SEQ ID NO: 52 - DNA, 200 bp, synthetic construct
  source 1..200: mol_type = other DNA
tgcctctcccacatgtgacccttacctacactctctggggacccccagtgttgcgcctttgtctctttgcctttgtccttaccctagaacctgctctatcatgtggtcggctggaccgactggaaccttgccctgaaccccgaaggaggacccaattgggtgcgtaactttgtcgacagtcccatcattgtagacatcac

SEQ ID NO: 53 - DNA, 200 bp, synthetic construct
  source 1..200: mol_type = other DNA
tgatgggactgtcgacaaagttacgcacccaattgggtcctccttcggggttcagggcaaggttccagtcggtccagccgaccacatggtacaggaggttctagggtaaggacaaaggcaaagagacaaaggcgcaacactgggggtccccagagagtgtaggtaagggtcacatgtgggagaggcagctgtgggtaggt

SEQ ID NO: 54 - DNA, 200 bp, synthetic construct
  source 1..200: mol_type = other DNA
tgatgggactgtcgacaaagttacgcacccaattgggtcctccttcggggttcagggcaaggttccagtcggtccagccgaccacgtgatagagcaggttctagggtaaggacaaaggcaaagagacaaaggcgcaacactgggggtccccagagagtgtaggtaagggtcacatgtgggagaggcagctgtgggtaggt

SEQ ID NO: 55 - DNA, 200 bp, synthetic construct
  source 1..200: mol_type = other DNA
gggaggtaccccgagggactctgaccatctgttcccacattcagcaagttcattcctgagggctcccagagagtggggctggttgccagtcagaagaacgacctggacgcagtggcactgatgcatcccgatggctctgctgttgtggtcgtgctaaaccggtgagggcaatggtgaggtctgggaagtgggctgaagac

SEQ ID NO: 56 - DNA, 200 bp, synthetic construct
  source 1..200: mol_type = other DNA
gggaggtaccccgagggactctgaccatctgttcccacattcagcaagttcattcctgagggctcccagagagtggggctggttgctagccagaaaaatgatctggacgcagtggcactgatgcatcccgatggctctgctgttgtggtcgtgctaaaccggtgagggcaatggtgaggtctgggaagtgggctgaagac

SEQ ID NO: 57 - DNA, 200 bp, synthetic construct; identical to SEQ ID NO: 30
  source 1..200: mol_type = other DNA

SEQ ID NO: 58 - DNA, 23 bp, synthetic construct
  source 1..23: mol_type = other DNA
gggacatggtacaggaggttcta

SEQ ID NO: 59 - DNA, 23 bp, synthetic construct
  source 1..23: mol_type = other DNA
ctagaacctcctgtaccatgtgg

SEQ ID NO: 60 - DNA, 23 bp, synthetic construct
  source 1..23: mol_type = other DNA
tgccagtcagaagaacgacctgg

SEQ ID NO: 61 - DNA, 23 bp, synthetic construct
  source 1..23: mol_type = other DNA
agggcatcagtgccactgcgtcc

SEQ ID NO: 62 - DNA, 23 bp, synthetic construct
  source 1..23: mol_type = other DNA
gggacgtgatagagcaggctcta

SEQ ID NO: 63 - DNA, 23 bp, synthetic construct
  source 1..23: mol_type = other DNA
tgctagccagaaaaatgatccgg

SEQ ID NO: 64 - DNA, 35 bp, synthetic construct
  source 1..35: mol_type = other DNA
ccgaccacgtgatagagcaggctctagggtaagga

SEQ ID NO: 65 - DNA, 33 bp, synthetic construct
  source 1..33: mol_type = other DNA
ccgaccatggtacaggaggctctagggtaagga

SEQ ID NO: 66 - DNA, 33 bp, synthetic construct
  source 1..33: mol_type = other DNA
tccttaccctagagcctgctctatcatgtggtc

SEQ ID NO: 67 - DNA, 33 bp, synthetic construct
  source 1..33: mol_type = other DNA
tccttaccctagagcctcctgtaccatgtggtc

SEQ ID NO: 68 - DNA, 33 bp, synthetic construct
  source 1..33: mol_type = other DNA
tggttgctagccagaagaatgatccggacgcag

SEQ ID NO: 69 - DNA, 32 bp, synthetic construct
  source 1..32: mol_type = other DNA
gatgcatgagggcgacggcatccgggtcgttc

SEQ ID NO: 70 - DNA, 33 bp, synthetic construct
  source 1..33: mol_type = other DNA
cccacgacgtgatacaggaggttctagggtaag

SEQ ID NO: 71 - DNA, 28 bp, synthetic construct
  source 1..28: mol_type = other DNA
tcgtggctagccagaagaatgacctgga

SEQ ID NO: 72 - DNA, 200 bp, synthetic construct
  source 1..200: mol_type = other DNA
tgatgggactgtcgacaaagttacgcacccaattgggtcctccttcggggttcagggcaaggttccagtcggtccagcccacgacgtgatacaggaggttctagggtaaggacaaaggcaaagagacaaaggcgcaacactgggggtccccagagagtgtaggtaagggtcacatgtgggagaggcagctgtgggtaggt

SEQ ID NO: 73 - DNA, 200 bp, synthetic construct
  source 1..200: mol_type = other DNA
gggaggtaccccgagggactctgaccatctgttcccacattcagcaagttcattcctgagggctcccagagagtggggctcgtggctagccagaagaatgacctggacgcagtggcactgatgcatcccgatggctctgctgttgtggtcgtgctaaaccggtgagggcaatggtgaggtctgggaagtgggctgaagac

SEQ ID NO: 74 - DNA, 42 bp, synthetic construct
  source 1..42: mol_type = other DNA
ctttgtccttaccctagaacctcctgtaccatgtggtcggct

SEQ ID NO: 75 - DNA, 42 bp, synthetic construct
  source 1..42: mol_type = other DNA
agccgaccacatggtacaggaggttctagggtaaggacaaag

SEQ ID NO: 76 - protein, 8 aa, synthetic construct
NLLYHVVG

SEQ ID NO: 77 - DNA, 61 bp, synthetic construct
  source 1..61: mol_type = other DNA
agtggggctggttgccagtcagaagaacgacctggacgcagtggcactgatgcatcccgat

SEQ ID NO: 78 - DNA, 61 bp, synthetic construct
  source 1..61: mol_type = other DNA
atcgggatgcatcagtgccactgcgtccaggtcgttcttctgactggcaaccagccccact

SEQ ID NO: 79 - protein, 20 aa, synthetic construct
VGLVASQKNDLDAVALMHPD

SEQ ID NO: 80 - DNA, 39 bp, synthetic construct
  source 1..39: mol_type = other DNA
tgtccttaccctagagcctgctctatcatgtggtcggct

SEQ ID NO: 81 - DNA, 39 bp, synthetic construct
  source 1..39: mol_type = other DNA
agccgaccacatgatagagcaggctctagggtaaggaca

SEQ ID NO: 82 - protein, 8 aa, synthetic construct
SLLYHVVG

SEQ ID NO: 83 - DNA, 71 bp, synthetic construct
  source 1..71: mol_type = other DNA
tctttgcctttgtccttaccctagagcctgctctatcacgtggtcggctggaccgactggaaccttgccct

SEQ ID NO: 84 - DNA, 70 bp, synthetic construct
  source 1..70: mol_type = other DNA
agggcaaggttccagtcggtccagccgaccacgtgatagagcaggcctagggtaaggacaaaggcaaaga

SEQ ID NO: 85 - protein, 16 aa, synthetic construct
SLLYHVVGWTDWNLAL

SEQ ID NO: 86 - DNA, 46 bp, synthetic construct
  source 1..46: mol_type = other DNA
gagagtggggctggttgctagccagaagaatgatccggacgcagtg

SEQ ID NO: 87 - DNA, 46 bp, synthetic construct
  source 1..46: mol_type = other DNA
cactgcgtccggatcattcttctggctagcaaccagccccactctc

SEQ ID NO: 88 - protein, 15 aa, synthetic construct
RVGLVASQKNDPDAV

SEQ ID NO: 89 - DNA, 35 bp, synthetic construct
  source 1..35: mol_type = other DNA
aggaatgggatctcggacgagatagtgcaccagcc

SEQ ID NO: 90 - DNA, 35 bp, synthetic construct
  source 1..35: mol_type = other DNA
aggaatgggatcttggaggacatagtgcagcaccc

SEQ ID NO: 91 - DNA, 97 bp, synthetic construct
  source 1..97: mol_type = other DNA
ttgtcccacagatatccagaaccctgaccctgccgtgtaccagctgagagactctaaatccagtgacaagtctgtctgcctattcaccgattttgat

SEQ ID NO: 92 - DNA, 97 bp, synthetic construct
  source 1..97: mol_type = other DNA
atcaaaatcggtgaataggcagacagacttgtcactggatttagagtctctcagctggtacacggcagggtcagggttctggatatctgtgggacaa

SEQ ID NO: 93 - DNA, 2245 bp, synthetic construct
  source 1..2245: mol_type = other DNA
gcccgggatggtccaggctgcagtgagccatgatcgtgccactgcactccagcctgggcgacagagtgagaccctgtctcacaacaacaacaacaacaacaaaaaggctgagctgcaccatgcttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggccgcggccgcgggctagcggatccccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatgaatgcaattgttgttgttaataaaggaaatttattttcattgcaatagtgtgttggaattttttgtgtctctcacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcatttt

SEQ ID NO: 94 - DNA, 20 bp, synthetic construct
  source 1..20: mol_type = other DNA
gccagtagccagccccgtcc

SEQ ID NO: 95 - DNA, 1999 bp, synthetic construct
  source 1..1999: mol_type = other DNA
ttgacccagtttcttaaaattgttgtcaaagcttcattcactccatggtgctatagagcacaagattttatttggtgagatggtgctttcatgaattcccccaacagagccaagctctccatctagtggacagggaagctagcagcaaaccttcccttcactacaaaacttcattgcttggccaaaaagagagttaattcaatgtagacatctatgtaggcaattaaaaacctattgatgtataaaacagtttgcattcatggagggcaactaaatacattctaggactttataaaagatcactttttatttatgcacagggtggaacaagatggattatcaagtgtcaagtccaatctatgacatcaattattatagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggccgcggccgcgggctagcggatccccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatgaatgcaattgttgttgttaataaaggaaatttattttcattgcaatagtgtgttggaattttttgtgtctctcacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaactcttgacagggctctattttataggcttcttctctggaatcttcttcatcatcctcctgacaatcgataggtacctggctgtcgtccatgctgtgtttgctttaaaagccaggacg

SEQ ID NO: 96 - DNA, 1999 bp, synthetic construct
  source 1..1999: mol_type = other DNA
atgcaggggaacggggatgcaggggaacggggctcagtctgaagagcagagccaggaacccctgtagggaaggggcaggagagccaggggcatgagatggtggacgaggaagggggacagggaagcctgagcgcctctcctgggcttgccaaggactcaaacccagaagcccagagcagggccttagggaagcgggaccctgctctgggcggaggaatatgtcccagatagcactggggactctttaaggaaagaaggatggagaaagagaaagggagtagaggcggccacgacctggtgaacacctaggacgcaccattctcacaaagggagttttccacacggacacccccctcctcaccacagccctgccaggatgagagacacaaaaaattccaacacactattgcaatgaaaataaatttcctttattaacaacaacaattgcattcattttacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccaggatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccaggatgggcaccaccccggtgaacagctcctcgcccttgctcaccatggtggcgaccggtggggatccgctagcccgcggccgcggcccgggcgactcagtctgtcggaggactggcgcgccgagtgaggggttgtgagctcttttatagagctcgggaagcagaagcgcgcgaacagaagcgagaagcaggctgattggttaattcaaataaggcgcagggtcatttcaggtccttgggggagcctggaaacatctgatgggtcttaagaaactgctgagggttgggccatatctggggaccatctgttcttggcctcgggccggggccgaaactgcggtgaccatctgttcttggccccgggccggggccgaaactgctcaccgcagatatcctgtttggcccaacgttagctattttcatgtacccgcccttgatctgaacttctctattcttggtttggtatttttccatgccttgcaaaatggcgttaccggggctggctactggccttatctcacaggtaaaactgacgcacggaggaacaatataaattggggactagaaaggtgaagagccaaagttagaactcaggaccaacttattctgattttgtttttccaaactgcttctcctcttgggaagtgtaaggaagctgcagcaccaggatcagtgaaacgcaccagacagccgcgtcagagcagctcaggttctgggagagggtagcgcagggtggccactgagaaccgggcaggtcacgcatcccccccttccctcccaccccctgccaagctctccctcccaggatcctctctggctccatcgtaagcaaaccttagaggttctggcaaggagagagatggctccagga

SEQ ID NO: 97 - DNA, 20 bp, synthetic construct
  source 1..20: mol_type = other DNA
tcagggttctggatatctgt

SEQ ID NO: 98 - DNA, 20 bp, synthetic construct
  source 1..20: mol_type = other DNA
agagtctctcagctggtaca

SEQ ID NO: 99 - DNA, 20 bp, synthetic construct
  source 1..20: mol_type = other DNA
agcggttgcagagaccccat

SEQ ID NO: 100 - DNA, 20 bp, synthetic construct
  source 1..20: mol_type = other DNA
cataccagctgagccgtccg

SEQ ID NO: 101 - DNA, 20 bp, synthetic construct
  source 1..20: mol_type = other DNA
catagctgatggtacccccc

SEQ ID NO: 102 - DNA, 1350 bp, synthetic construct
  source 1..1350: mol_type = other DNA
ggtagctaggagttccaggactcagtttcccctttgagcctcctttagcgactaaagcttgaagccccacgcatctcgactctcgcgcacaccgcccttgttgggctcaggggcggggcgccgcccccggaagtacttccccttaaaggctggggcctgccggaaatggcgcagcggcagggaggggctcttcacccagtccggcagttgaagctcggcgctcgggttacccctgcagcgacgccccctggtcccacagataccactgctgctcccgccctttcgctcctcggccgcgcaatgggcggatcgggtgggactagtggcagcaagggcgaggagctgttcaccggggtggtgcccatcctggtcgagctggacggcgacgtaaacggccacaagttcagcgtgcgcggcgagggcgagggcgatgccaccaacggcaagctgaccctgaagttcatctgcaccaccggcaagctgcccgtgccctggcccaccctcgtgaccaccctgacctacggcgtgcagtgcttcagccgctaccccgaccacatgaagcgccacgacttcttcaagtccgccatgcccgaaggctacgtccaggagcgcaccatcagcttcaaggacgacggcacctacaagacccgcgccgaggtgaagttcgagggcgacaccctggtgaaccgcatcgagctgaagggcatcgacttcaaggaggacggcaacatcctggggcacaagctggagtacaacttcaacagccacaacgtctatatcaccgccgacaagcagaagaacggcatcaaggccaacttcaagatccgccacaacgtggaggacggcagcgtgcagctcgccgaccactaccagcagaacacccccatcggcgacggccccgtgctgctgcccgacaaccactacctgagcacccagtccgtgctgagcaaagaccccaacgagaagcgcgatcacatggtcctgctggagttcgtgaccgccgccgggatcactggaaccggtgctggaagtggtacacgcgacgacgagtacgactacctctttaaaggtgaggccatgggctctcgcactctacacagtcctcgttcggggacccgggccactcccggtggaccctcgtgccggccacccctgcactgatataggcctccctcagcccttcctttttgtgcggttccgtctcctacccagctcagcctcttctcccccgctcagacaggggtccccatcacatgccgctctctgagcgacctctccataggccttcgctggcctcagagcccctccctgcgtgtccttcccctggcggactgccttctcccacatcgt

SEQ ID NO: 103 - DNA, 1804 bp, synthetic construct
  source 1..1804: mol_type = other DNA
gagtccagagtgctaaccattacaccatggaaccgccacgcatgtgtagctgccttcggctgtctaatcctcagagaaccccgcccccatccacaaacccaccactcacaggcggtcccgcctggttccagcgagccgcttccggcacggtagctcgagaaatgagcaagcggccactaagactatggtagctaggagttccaggactcagtttcccctttgagcctcctttagcgactaaagcttgaagccccacgcatctcgactctcgcgcacaccgcccttgttgggctcaggggcggggcgccgcccccggaagtacttccccttaaaggctggggcctgccggaaatggcgcagcggcagggaggggctcttcacccagtccggcagttgaagctcggcgctcgggttacccctgcagcgacgccccctggtcccacagataccactgctgctcccgccctttcgctcctcggccgcgcaatgggcacccgcgagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatgaatgcaattgttgttgttaataaaggaaatttattttcattgcaatagtgtgttggaattttttgtgtctctcacgacgagtacgactacctctttaaaggtgaggccatgggctctcgcactctacacagtcctcgttcggggacccgggccactcccggtggaccctcgtgccggccacccctgcactgatataggcctccctcagcccttcctttttgtgcggttccgtctcctacccagctcagcctcttctcccccgctcagacaggggtccccatcacatgccgctctctgagcgacctctccataggccttcgctggcctcagagcccctccctgcgtgtccttcccctggcggactgccttctcccacatcgtcgaattcctttccccgggttctacggccccgccgctcctcccaccatctctcttttcgggtgtagcgccccctccccctcggcgtacacccttcccagctcgcgtcctctcccgaagcccctctgacgggttcttcgcttccctcttggccttgccttcggtgcagactcccattacaggtctttttcttatc

SEQ ID NO: 104 - DNA, 2245 bp, synthetic construct
  source 1..2245: mol_type = other DNA
gagtccagagtgctaaccattacaccatggaaccgccacgcatgtgtagctgccttcggctgtctaatcctcagagaaccccgcccccatccacaaacccaccactcacaggcggtcccgcctggttccagcgagccgcttccggcacggtagctcgagaaatgagcaagcggccactaagactatggtagctaggagttccaggactcagtttcccctttgagcctcctttagcgactaaagcttgaagccccacgcatctcgactctcgcgcacaccgcccttgttgggctcaggggcggggcgccgcccccggaagtacttccccttaaaggctggggcctgccggaaatggcgcagcggcagggaggggctcttcacccagtccggcagttgaagctcggcgctcgggttacccctgcagcgacgccccctggtcccacagataccactgctgctcccgccctttcgctcctcggccgcgcaatgggcacccgcgagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggccgcggccgcgggctagcggatccccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatgaatgcaattgttgttgttaataaaggaaatttattttcattgcaatagtgtgttggaattttttgtgtctctcacgacgagtacgactacctctttaaaggtgaggccatgggctctcgcactctacacagtcctcgttcggggacccgggccactcccggtggaccctcgtgccggccacccctgcactgatataggcctccctcagcccttcctttttgtgcggttccgtctcctacccagctcagcctcttctcccccgctcagacaggggtccccatcacatgccgctctctgagcgacctctccataggccttcgctggcctcagagcccctccctgcgtgtccttcccctggcggactgccttctcccacatcgtcgaattcctttccccgggttctacggccccgccgctcctcccaccatctctcttttcgggtgtagcgccccctccccctcggcgtacacccttcccagctcgcgtcctctcccgaagcccctctgacgggttcttcgcttccctcttggccttgccttcggtgcagactcccattacaggtctttttcttatc

SEQ ID NO: 105 - DNA, 2491 bp, synthetic construct
  source 1..2491: mol_type = other DNA
atgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagtgaataattgagccaccatggctctgcccgtcacagctctgctgctgcctctggccctgctgctgcacgccgccagacctcaggtgcagctcgtgcagagcggcgctgaggtgaagaaacctggcagcagcgtgaaggtgagctgcaaggcctccggctacaccttcaccgactacaacatgcactgggtgaggcaagcccctggccagggactggagtggatcggctacatctacccttacaacggcggcacaggctacaaccagaagttcaagtccaaggccaccatcaccgccgatgagtccaccaataccgcctacatggagctcagcagcctgaggtccgaggacacagccgtctactactgcgccaggggcaggcccgctatggactactggggccagggcaccctggtgacagtgagctctggtggcggcggatccggcggcggcggcagcggcggcggcggctccgacattcagatgacccagagccctagcagcctgagcgcttccgtgggagacagggtgaccatcacatgcagggcctccgagagcgtggacaattacggcatcagcttcatgaactggttccagcagaagcccggcaaggcccccaaactgctgatctatgccgccagcaatcagggctccggcgtgcctagcaggttttccggcagcggcagcggcaccgactttaccctgaccatctccagcctgcagcctgacgatttcgccacctactactgccagcagagcaaggaggtgccttggacctttggacagggcacaaaggtggagatcaagtccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatcctgataaaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaac

SEQ ID NO: 106 - DNA, 3271 bp, synthetic construct
  source 1..3271: mol_type = other DNA
atgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagtgaataattgagccaccatggctctgcccgtcacagctctgctgctgcctctggccctgctgctgcacgccgccagacctcaggtgcagctcgtgcagagcggcgctgaggtgaagaaacctggcagcagcgtgaaggtgagctgcaaggcctccggctacaccttcaccgactacaacatgcactgggtgaggcaagcccctggccagggactggagtggatcggctacatctacccttacaacggcggcacaggctacaaccagaagttcaagtccaaggccaccatcaccgccgatgagtccaccaataccgcctacatggagctcagcagcctgaggtccgaggacacagccgtctactactgcgccaggggcaggcccgctatggactactggggccagggcaccctggtgacagtgagctctggtggcggcggatccggcggcggcggcagcggcggcggcggctccgacattcagatgacccagagccctagcagcctgagcgcttccgtgggagacagggtgaccatcacatgcagggcctccgagagcgtggacaattacggcatcagcttcatgaactggttccagcagaagcccggcaaggcccccaaactgctgatctatgccgccagcaatcagggctccggcgtgcctagcaggttttccggcagcggcagcggcaccgactttaccctgaccatctccagcctgcagcctgacgatttcgccacctactactgccagcagagcaaggaggtgccttggacctttggacagggcacaaaggtggagatcaagtccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatccggtggcagcggtgaaggaaggggctctttgcttacttgtggagatgttgaggaaaatccaggacccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaac

SEQ ID NO: 107 - DNA, 4462 bp, synthetic construct
  source 1..4462: mol_type = other DNA
atgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagtgaataattgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcctcgaggccaccatggctctgcccgtcacagctctgctgctgcctctggccctgctgctgcacgccgccagacctcaggtgcagctcgtgcagagcggcgctgaggtgaagaaacctggcagcagcgtgaaggtgagctgcaaggcctccggctacaccttcaccgactacaacatgcactgggtgaggcaagcccctggccagggactggagtggatcggctacatctacccttacaacggcggcacaggctacaaccagaagttcaagtccaaggccaccatcaccgccgatgagtccaccaataccgcctacatggagctcagcagcctgaggtccgaggacacagccgtctactactgcgccaggggcaggcccgctatggactactggggccagggcaccctggtgacagtgagctctggtggcggcggatccggcggcggcggcagcggcggcggcggctccgacattcagatgacccagagccctagcagcctgagcgcttccgtgggagacagggtgaccatcacatgcagggcctccgagagcgtggacaattacggcatcagcttcatgaactggttccagcagaagcccggcaaggcccccaaactgctgatctatgccgccagcaatcagggctccggcgtgcctagcaggttttccggcagcggcagcggcaccgactttaccctgaccatctccagcctgcagcctgacgatttcgccacctactactgccagcagagcaaggaggtgccttggacctttggacagggcacaaaggtggagatcaagtccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatccggtggcagcggtgaaggaaggggctctttgcttacttgtggagatgttgaggaaaatccaggacccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaac

SEQ ID NO: 108 - DNA, 2490 bp, synthetic construct
  source 1..2490: mol_type = other DNA
acataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgttgaataattgagccaccatggctctgcccgtcacagctctgctgctgcctctggccctgctgctgcacgccgccagacctcaggtgcagctcgtgcagagcggcgctgaggtgaagaaacctggcagcagcgtgaaggtgagctgcaaggcctccggctacaccttcaccgactacaacatgcactgggtgaggcaagcccctggccagggactggagtggatcggctacatctacccttacaacggcggcacaggctacaaccagaagttcaagtccaaggccaccatcaccgccgatgagtccaccaataccgcctacatggagctcagcagcctgaggtccgaggacacagccgtctactactgcgccaggggcaggcccgctatggactactggggccagggcaccctggtgacagtgagctctggtggcggcggatccggcggcggcggcagcggcggcggcggctccgacattcagatgacccagagccctagcagcctgagcgcttccgtgggagacagggtgaccatcacatgcagggcctccgagagcgtggacaattacggcatcagcttcatgaactggttccagcagaagcccggcaaggcccccaaactgctgatctatgccgccagcaatcagggctccggcgtgcctagcaggttttccggcagcggcagcggcaccgactttaccctgaccatctccagcctgcagcctgacgatttcgccacctactactgccagcagagcaaggaggtgccttggacctttggacagggcacaaaggtggagatcaagtccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatcctgataaaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaac

SEQ ID NO: 109 - DNA, 3270 bp, synthetic construct
  source 1..3270: mol_type = other DNA
acataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgttgaataattgagccaccatggctctgcccgtcacagctctgctgctgcctctggccctgctgctgcacgccgccagacctcaggtgcagctcgtgcagagcggcgctgaggtgaagaaacctggcagcagcgtgaaggtgagctgcaaggcctccggctacaccttcaccgactacaacatgcactgggtgaggcaagcccctggccagggactggagtggatcggctacatctacccttacaacggcggcacaggctacaaccagaagttcaagtccaaggccaccatcaccgccgatgagtccaccaataccgcctacatggagctcagcagcctgaggtccgaggacacagccgtctactactgcgccaggggcaggcccgctatggactactggggccagggcaccctggtgacagtgagctctggtggcggcggatccggcggcggcggcagcggcggcggcggctccgacattcagatgacccagagccctagcagcctgagcgcttccgtgggagacagggtgaccatcacatgcagggcctccgagagcgtggacaattacggcatcagcttcatgaactggttccagcagaagcccggcaaggcccccaaactgctgatctatgccgccagcaatcagggctccggcgtgcctagcaggttttccggcagcggcagcggcaccgactttaccctgaccatctccagcctgcagcctgacgatttcgccacctactactgccagcagagcaaggaggtgccttggacctttggacagggcacaaaggtggagatcaagtccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatccggtggcagcggtgaaggaaggggctctttgcttacttgtggagatgttgaggaaaatccaggacccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaac

SEQ ID NO: 110 - DNA, 4461 bp, synthetic construct
  source 1..4461: mol_type = other DNA
acataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgttgaataattgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagctagcctcgaggccaccatggctctgcccgtcacagctctgctgctgcctctggccctgctgctgcacgccgccagacctcaggtgcagctcgtgcagagcggcgctgaggtgaagaaacctggcagcagcgtgaaggtgagctgcaaggcctccggctacaccttcaccgactacaacatgcactgggtgaggcaagcccctggccagggactggagtggatcggctacatctacccttacaacggcggcacaggctacaaccagaagttcaagtccaaggccaccatcaccgccgatgagtccaccaataccgcctacatggagctcagcagcctgaggtccgaggacacagccgtctactactgcgccaggggcaggcccgctatggactactggggccagggcaccctggtgacagtgagctctggtggcggcggatccggcggcggcggcagcggcggcggcggctccgacattcagatgacccagagccctagcagcctgagcgcttccgtgggagacagggtgaccatcacatgcagggcctccgagagcgtggacaattacggcatcagcttcatgaactggttccagcagaagcccggcaaggcccccaaactgctgatctatgccgccagcaatcagggctccggcgtgcctagcaggttttccggcagcggcagcggcaccgactttaccctgaccatctccagcctgcagcctgacgatttcgccacctactactgccagcagagcaaggaggtgccttggacctttggacagggcacaaaggtggagatcaagtccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatccggtggcagcggtgaaggaaggggctctttgcttacttgtggagatgttgaggaaaatccaggacccgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtgataaaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaac

SEQ ID NO: 111 - DNA, 2998 bp, synthetic construct
  source 1..2998: mol_type = other DNA
gagtccagagtgctaaccattacaccatggaaccgccacgcatgtgtagctgccttcggctgtctaatcctcagagaaccccgcccccatccacaaacccaccactcacaggcggtcccgcctggttccagcgagccgcttccggcacggtagctcgagaaatgagcaagcggccactaagactatggtagctaggagttccaggactcagtttcccctttgagcctcctttagcgactaaagcttgaagccccacgcatctcgactctcgcgcacaccgcccttgttgggctcaggggcggggcgccgcccccggaagtacttccccttaaaggctggggcctgccggaaatggcgcagcggcagggaggggctcttcacccagtccggcagttgaagctcggcgctcgggttacccctgcagcgacgccccctggtcccacagataccactgctgctcccgccctttcgctcctcggccgcgcaatgggcacccgcgagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggccgcggccgcgggctagcggatccccaccggtcgccaccatggctctgcccgtcacagctctgctgctgcctctggccctgctgctgcacgccgccagacctcaggtgcagctcgtgcagagcggcgctgaggtgaagaaacctggcagcagcgtgaaggtgagctgcaaggcctccggctacaccttcaccgactacaacatgcactgggtgaggcaagcccctggccagggactggagtggatcggctacatctacccttacaacggcggcacaggctacaaccagaagttcaagtccaaggccaccatcaccgccgatgagtccaccaataccgcctacatggagctcagcagcctgaggtccgaggacacagccgtctactactgcgccaggggcaggcccgctatggactactggggccagggcaccctggtgacagtgagctctggtggcggcggatccggcggcggcggcagcggcggcggcggctccgacattcagatgacccagagccctagcagcctgagcgcttccgtgggagacagggtgaccatcacatgcagggcctccgagagcgtggacaattacggcatcagcttcatgaactggttccagcagaagcccggcaaggcccccaaactgctgatctatgccgccagcaatcagggctccggcgtgcctagcaggttttccggcagcggcagcggcaccgactttaccctgaccatctccagcctgcagcctgacgatttcgccacctactactgccagcagagcaaggaggtgccttggacctttggacagggcacaaaggtggagatcaagtccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatcctgataaaatgaatgcaattgttgttgttaataaaggaaatttattttcattgcaatagtgtgttggaattttttgtgtctctcacgacgagtacgactacctctttaaaggtgaggccatgggctctcgcactctacacagtcctcgttcggggacccgggccactcccggtggaccctcgtgccggccacccctgcactgatataggcctccctcagcccttcctttttgtgcggttccgtctcctacccagctcagcctcttctcccccgctcagacaggggtccccatcacatgccgctctctgagcgacctctccataggccttcgctggcctcagagcccctccctgcgtgtccttcccctggcggactgccttctcccacatcgtcgaattcctttccccgggttctacggccccgccgctcctcccaccatctctcttttcgggtgtagcgccccctccccctcggcgtacacccttcccagctcgcgtcctctcccgaagcccctctgacgggttcttcgcttccctcttggccttgccttcggtgcagactcccattacaggtctttttcttatc

SEQ ID NO: 112 - DNA, 2998 bp, synthetic construct
  source 1..2998: mol_type = other DNA
tagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccggtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggccgcggccgcgggctagcggatccccaccggtcgccaccatggctctgcccgtcacagctctgctgctgcctctggccctgctgctgcacgccgccagacctcaggtgcagctcgtgcagagcggcgctgaggtgaagaaacctggcagcagcgtgaaggtgagctgcaaggcctccggctacaccttcaccgactacaacatgcactgggtgaggcaagcccctggccagggactggagtggatcggctacatctacccttacaacggcggcacaggctacaaccagaagttcaagtccaaggccaccatcaccgccgatgagtccaccaataccgcctacatggagctcagcagcctgaggtccgaggacacagccgtctactactgcgccaggggcaggcccgctatggactactggggccagggcaccctggtgacagtgagctctggtggcggcggatccggcggcggcggcagcggcggcggcggctccgacattcagatgacccagagccctagcagcctgagcgcttccgtgggagacagggtgaccatcacatgcagggcctccgagagcgtggacaattacggcatcagcttcatgaactggttccagcagaagcccggcaaggcccccaaactgctgatctatgccgccagcaatcagggctccggcgtgcctagcaggttttccggcagcggcagcggcaccgactttaccctgaccatctccagcctgcagcctgacgatttcgccacctactactgccagcagagcaaggaggtgccttggacctttggacagggcacaaaggtggagatcaagtccggagccgccgccatcgaagtgatgtacccccctccctacctggataacgagaagagcaacggcaccatcatccacgtgaagggaaagcacctgtgtcccagccccctgtttcccggccctagcaagcccttctgggtgctggtggtggtcggcggagtgctggcctgctacagcctcctggtgaccgtggccttcatcatcttctgggtgaggagcaagaggtccaggctgctgcacagcgactacatgaatatgacccccagaaggcccggccccaccagaaagcactatcagccctacgccccccccagggactttgccgcctacaggagcagggtgaagttcagcagatccgccgatgcccctgcttaccagcagggccagaaccagctgtataacgagctgaacctgggcaggagggaggaatacgacgtgctggataagaggaggggaagggaccccgagatgggcggaaagcccaggaggaagaacccccaggagggcctgtacaatgagctgcagaaagacaagatggccgaggcctacagcgagatcggcatgaagggcgagaggaggaggggcaagggccatgacggcctgtaccaaggcctgtccaccgccaccaaggatacctacgacgccctgcacatgcaggccctgcctcccaggggatcctgataaaatgaatgcaattgttgttgttaataaaggaaatttattttcattgcaatagtgtgttggaattttttgtgtctctcatcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgttcccctgcatcccccttcccctgcatcccccagaggccccaggccacctacttggcctggaccccacgagaggccaccccagccctgtctaccaggctgccttttgggtggattctcctccaactgtggggtg

SEQ ID NO: 113 - DNA, 5124 bp, synthetic construct
  source 1..5124: mol_type = other DNA
tttggttttggctttcactggagtctgcaacaagaactggcatcatgctgcccatttcccgcctctccccacccagacccctgcctcagggacgcctgtcctcagcccagccctcagctgcagccaggccttcagcctccgtaacccccgctcagggtccccaccccctgcagccctgtccctccaggatgcatggccttgtcctgtgtgggggtggccgagagcactgccccagccctgggtaccttgggcaggaagctggcagaggccagggctgccattcaaacaggggcaggtggttttgccaggaggaagttgacagttcaacttcaaacatgggtgacgcaggccccacactgcctgctccccgtcccacccctccctgagcacgccaccccgccctctccctctctgagagcgagatacccggccagacaccctcacctgcggtgcccagctgcccaggctgaggcaagagaaggccagaaaccatgcccatgtaaataaataaggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggaattcgacgccaccatggagttcggcctgagctggctgttcctggtggccatcctgaagggcgtgcagtgcatcgacgccatgggcaacatccagctggtgcagagcggccccgagctgaagaagcccggcgagaccgtgaagatcagctgcaaggccagcggctacaccttcaccaactacggcatgaactgggtgaagcaggcccccggcaagggcctgaggtggatgggctggatcaacacccacaccggcgagcccacctacgccgacgacttcaagggcaggttcgccttcagcctggagaccagcgccagcaccgcctacctgcagatcaacaacctgaagaacgaggacaccgccacctacttctgcaccaggaggggctacgactggtacttcgacgtgtggggcgccggcaccaccgtgaccgtgagcagcggcggcggcggcagcggcggcggcggcagcggcggcggcggcagcgacatcaagatgacccagagccccagcagcatgtacgccagcctgggcgagagggtgaccatcacctgcaaggccagccaggacatcaacagctacctgagctggttccaccacaagcccggcaagagccccaagaccctgatctacagggccaacaggctggtggacggcgtgcccagcaggttcagcggcagcggcagcggccaggactacagcctgaccatcagcagcctggactacgaggacatgggcatctactactgccagcagtacgacgagagcccctggaccttcggcggcggcaccaagctggagatgaagggcagcggcgaccccgccgagcccaagagccccgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagaaggaccccaagttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagggccacgaacttctctctgttaaagcaagcaggagacgtggaagaaaaccccggtcctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatgaatgcaattgttgttgttaataaaggaaatttattttcattgcaatagtgtgttggaattttttgtgtctctcagggtctctgcaaccgctggccaccttgtacctgctggggatgctgggtgagtacccctcccaggtgtcctgcgaacacccgggctcgctccagtgcaaggaaggagttcccagttttacccaaggctgactctgggatccacatgtcagccctctggagcgttgtggagatttggggccactgggatccctgcctgcccccactaagccgcagcttggccctctgtcctgcatgtcccacccgccaggagcacaaccttgcctctctcatgcgctgttgagaaccctgctttacccttccagtgcaagagagactgcaggggggacccgcatttgatggggcccagacaacttgattcctaggctgagttggattttagcagagcattcaggcctccctctgcgaggtcccccactgacagcccagcctttacttggtcgcctccagagacatggaaactcgccgtctccgaggcagctctgatgatgctctggacagac

SEQ ID NO: 114 - DNA, 5124 bp, synthetic construct
  source 1..5124: mol_type = other DNA
ttgaggctgcagtgagctgtgatcatgccactgcactccagcctgggtgaggagagtgatatcctgtctcaaaaagtaataataataataattgatggttacattattacaaaggttagcatgagggaatttgaggtagggagtaatggaactggtgtataccttgattgtgatggtggtcacacacatctatatatgtgacattcacggaatcatgcagtaaagaaaaatcaatttcacgtctgttcattttaaaagtaacgttttttaagaagaaaaaaaatcgatagttgcagcccactagatagaattcatatcactcaggggttccaacctggagtatgaaaattcctgtccctaaaacccatgatagtggatagggggaggcagaaagggccattgctcgggctgtgggtgggtgagctggggagaagggagagagtgggaggtttcacttcctgaccctcctctcttctttctgcagtcgcttcctgcctcggtaaataaataaggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggaattcgacgccaccatggagttcggcctgagctggctgttcctggtggccatcctgaagggcgtgcagtgcatcgacgccatgggcaacatccagctggtgcagagcggccccgagctgaagaagcccggcgagaccgtgaagatcagctgcaaggccagcggctacaccttcaccaactacggcatgaactgggtgaagcaggcccccggcaagggcctgaggtggatgggctggatcaacacccacaccggcgagcccacctacgccgacgacttcaagggcaggttcgccttcagcctggagaccagcgccagcaccgcctacctgcagatcaacaacctgaagaacgaggacaccgccacctacttctgcaccaggaggggctacgactggtacttcgacgtgtggggcgccggcaccaccgtgaccgtgagcagcggcggcggcggcagcggcggcggcggcagcggcggcggcggcagcgacatcaagatgacccagagccccagcagcatgtacgccagcctgggcgagagggtgaccatcacctgcaaggccagccaggacatcaacagctacctgagctggttccaccacaagcccggcaagagccccaagaccctgatctacagggccaacaggctggtggacggcgtgcccagcaggttcagcggcagcggcagcggccaggactacagcctgaccatcagcagcctggactacgaggacatgggcatctactactgccagcagtacgacgagagcccctggaccttcggcggcggcaccaagctggagatgaagggcagcggcgaccccgccgagcccaagagccccgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagaaggaccccaagttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagggccacgaacttctctctgttaaagcaagcaggagacgtggaagaaaaccccggtcctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatgaatgcaattgttgttgttaataaaggaaatttattttcattgcaatagtgtgttggaattttttgtgtctctcaacggctcagctggtatgacccaggtaaggaagagccacatggagaaaggcctggggcagggggagagtggggctgtggtttcatcaggccatcggggacctctcgatgaagccatcacttctgccagagtgaaccccaccctatagagagagtgaaccccagcatacacacaggcacatagatgcagacactgcacattaagatgctcacatgcaggtgggtgccctcgacagccgtaaatcacccacaaatgccagatctcatgataattattatgacccgctcaccatgcacagaagacatcccagctcataaatgtaccttgcaaagtcttatttcccacccaatcctgacagatgctccatggtcaaagatgtttagagcggagtctgcagagagaggccgcagactgatggtaaagtgtgtggaacgtccagccttagacgttggagtttagtcgtagaggctgtttcccaaatagggttccatggagcatgttg

SEQ ID NO: 115 - DNA, 5124 bp, synthetic construct
  source 1..5124: mol_type = other DNA
gcactcatgccaggagctcctggtcctctcaaggctgctggctgcccccggccctccccacaccacccattcctccctcaccagagtgtctcattgcagaaccccagaagacaacacctccaacgacaaggcccccgcccaccacaactccagagcccacaggtaagaggattctgaaccccccacagggagtcagagctagcaaataaaaacccaggatgcccagttacattggaatttctgacaaaggtggaaatgtttagtattggtgtgttctacgcaatatttgggaccccatcacctcccaaggctaagcgttagtcagtagttgtccacaagttggggccaaacagcaaggagtgcccaggaagccctcggcgctcagggtggctccccctcctgctctctcctctcctagctcctcccaggctgcagctggtggcacagtctggcggccagcactgtgccggcgtggtggagttctacagcggcagcctgggtaaataaataaggctccggtgcccgtcagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgacgtacggaattcgacgccaccatggagttcggcctgagctggctgttcctggtggccatcctgaagggcgtgcagtgcatcgacgccatgggcaacatccagctggtgcagagcggccccgagctgaagaagcccggcgagaccgtgaagatcagctgcaaggccagcggctacaccttcaccaactacggcatgaactgggtgaagcaggcccccggcaagggcctgaggtggatgggctggatcaacacccacaccggcgagcccacctacgccgacgacttcaagggcaggttcgccttcagcctggagaccagcgccagcaccgcctacctgcagatcaacaacctgaagaacgaggacaccgccacctacttctgcaccaggaggggctacgactggtacttcgacgtgtggggcgccggcaccaccgtgaccgtgagcagcggcggcggcggcagcggcggcggcggcagcggcggcggcggcagcgacatcaagatgacccagagccccagcagcatgtacgccagcctgggcgagagggtgaccatcacctgcaaggccagccaggacatcaacagctacctgagctggttccaccacaagcccggcaagagccccaagaccctgatctacagggccaacaggctggtggacggcgtgcccagcaggttcagcggcagcggcagcggccaggactacagcctgaccatcagcagcctggactacgaggacatgggcatctactactgccagcagtacgacgagagcccctggaccttcggcggcggcaccaagctggagatgaagggcagcggcgaccccgccgagcccaagagccccgacaagacccacacctgccccccctgccccgcccccgagctgctgggcggccccagcgtgttcctgttcccccccaagcccaaggacaccctgatgatcagcaggacccccgaggtgacctgcgtggtggtggacgtgagccacgaggaccccgaggtgaagttcaactggtacgtggacggcgtggaggtgcacaacgccaagaccaagcccagggaggagcagtacaacagcacctacagggtggtgagcgtgctgaccgtgctgcaccaggactggctgaacggcaaggagtacaagtgcaaggtgagcaacaaggccctgcccgcccccatcgagaagaccatcagcaaggccaagggccagcccagggagccccaggtgtacaccctgccccccagcagggacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgagccccggcaagaaggaccccaagttctgggtgctggtggtggtgggcggcgtgctggcctgctacagcctgctggtgaccgtggccttcatcatcttctgggtgaggagcaagaggagcaggctgctgcacagcgactacatgaacatgacccccaggaggcccggccccaccaggaagcactaccagccctacgccccccccagggacttcgccgcctacaggagcagggtgaagttcagcaggagcgccgacgcccccgcctaccagcagggccagaaccagctgtacaacgagctgaacctgggcaggagggaggagtacgacgtgctggacaagaggaggggcagggaccccgagatgggcggcaagcccaggaggaagaacccccaggagggcctgtacaacgagctgcagaaggacaagatggccgaggcctacagcgagatcggcatgaagggcgagaggaggaggggcaagggccacgacggcctgtaccagggcctgagcaccgccaccaaggacacctacgacgccctgcacatgcaggccctgccccccagggccacgaacttctctctgttaaagcaagcaggagacgtggaagaaaaccccggtcctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatgaatgcaattgttgttgttaataaaggaaatttattttcattgcaatagtgtgttggaattttttgtgtctctcagggtaccatcagctatgaggcccaggacaagacccaggacctggagaacttcctctgcaacaacctccagtgtggctccttcttgaagcatctgccagagactgaggcaggcagagcccaagacccaggggagccacgggaacaccagcccttgccaatccaatggaagatccagaactcaagctgtacctccctggagcattgcttcaggaaaatcaagccccagaaaagtggccgagttcttgccctcctttgctcaggtaagtgagacctggccaagccccatgacaccttctgctgccctaggtggggtcacagagcatcccagaaggtcagggaacatgtgtgcagcacagggcactatggagaatacaagggaagtggaggcctggtcttggcctctaagaggtaacaagggttggggtggggaggatgcatccacactcaatgccttggtaatctctgcaaagctacacaccccaagcccaaaggaaccgctg

SEQ ID NO: 116 - DNA, 2245 bp, synthetic construct
  source 1..2245: mol_type = other DNA
atgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggccgcggccgcgggctagcggatccccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatgaatgcaattgttgttgttaataaaggaaatttattttcattgcaatagtgtgttggaattttttgtgtctctcagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagaggg

SEQ ID NO: 117 - DNA, 2983 bp, synthetic construct
  source 1..2983: mol_type = other DNA
atgtgatagatttcccaacttaatgccaacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatgaatgcaattgttgttgttaataaaggaaatttattttcattgcaatagtgtgttggaattttttgtgtctctcagatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagaggg

SEQ ID NO: 118 - DNA, 2245 bp, synthetic construct
  source 1..2245: mol_type = other DNA
aacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtggtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggccgcggccgcgggctagcggatccccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatgaatgcaattgttgttgttaataaaggaaatttattttcattgcaatagtgtgttggaattttttgtgtctctcataccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaa

SEQ ID NO: 119 - DNA, 2983 bp, synthetic construct
  source 1..2983: mol_type = other DNA
aacataccataaacctcccattctgctaatgcccagcctaagttggggagaccactccagattccaagatgtacagtttgctttgctgggcctttttcccatgcctgcctttactctgccagagttatattgctggggttttgaagaagatcctattaaataaaagaataagcagtattattaagtagccctgcatttcaggtttccttgagtggcaggccaggcctggccgtgaacgttcactgaaatcatggcctcttggccaagattgatagcttgtgcctgtccctgagtcccagtccatcacgagcagctggtttctaagatgctatttcccgtataaagcatgagaccgtgacttgccagccccacagagccccgcccttgtccatcactggcatctggactccagcctgggttggggcaaagagggaaatgagatcatgtcctaaccctgatcctcttgtcccacagatatccagaaccctgaccctgccgtgggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccactgagtaccgggcgccgtccaggcacctcgattagttctcgtgcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatgaatgcaattgttgttgttaataaaggaaatttattttcattgcaatagtgtgttggaattttttgtgtctctcataccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccaggtaagggcagctttggtgccttcgcaggctgtttccttgcttcaggaatggccaggttctgcccagagctctggtcaatgatgtctaaaactcctctgattggtggtctcggccttatccattgccaccaaaaccctctttttactaagaaacagtgagccttgttctggcagtccagagaatgacacgggaaaaaagcagatgaagagaaggtggcaggagagggcacgtggcccagcctcagtctctccaa

SEQ ID NO: 120 - DNA, 4245 bp, synthetic construct
  source 1..4245: mol_type = other DNA
cagtctggtctatctgcctggccctggccattgtcactttgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggctgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccggtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggccgcggccgcgggctagcggatccccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatgaatgcaattgttgttgttaataaaggaaatttattttcattgcaatagtgtgttggaattttttgtgtctctcatcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgttcccctgcatcccccttcccctgcatcccccagaggccccaggccacctacttggcctggaccccacgagaggccaccccagccctgtctaccaggctgccttttgggtggattctcctccaactgtggggtgactgcttggcaaactcactcttcggggtatcccaggaggcctggagcattggggtgggctggggttcagagaggagggattcccttctcaggttacgtggccaagaagcaggggagctgggtttgggtcaggtctgggtgtggggtgaccagcttatgctgtttgcccaggacagcctagttttagcgctgaaaccctcagtcctaggaaaacagggatggttggtcactgtctctgggtgactcttgattcccggccagtttctccacctggggctgtgtttctcgtcctgcatccttctccaggcaggtccccaagcatcgcccccctgctgtggctgttcccaagttcttagggtaccccacgtgggtttatcaaccacttggtgaggctggtaccctgcccccattcctgcaccccaattgccttagtggctggggggttgggggctagagtaggaggggctggagccaggattcttagggctgaacagagaagagctgggggcctgggctcctgggtttgagagaggaggggctggggcctggactcctgggtccgagggaggaggggctggggcctggactcctgggtctgagggtggagggactgggggcctggactcctgggtccgagggaggaggggctggggcctggactcgtgggtctgagggaggaggggctgggggcctggacttctgggtcttagggaggcggggctgggcctggacccctgggtctgaatggggagaggctgggggcctggactccttcatctgagggcggaagggctggggcctggcctcctgggttgaatggggaggggttgggcctggactctggagtccctggtgcccaggcctcaggcatctttcacagggatgcctgtactgggcaggtccttgaaagggaaaggcccattgctctccttgcccccctcccctatcgccatgacaactgggtggaaataaacgagccgagttcatcccgttcccagggc

SEQ ID NO: 121 - DNA, 1929 bp, synthetic construct
  source 1..1929: mol_type = other DNA
aaggcgattaagttgggtaacgccagggtttgacatcaattattatacatcggatagggcgaattggagctcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggccgcggccgcgggctagcggatccccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatgaatgcaattgttgttgttaataaaggaaatttattttcattgcaatagtgtgttggaattttttgtgtctctcaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctctgacatcaattattatacatcggaagatcctttgatcttttctacggggtctg

SEQ ID NO: 122 - DNA, 1876 bp, synthetic construct
  source 1..1876: mol_type = other DNA
aaggcgattaagttgggtaacgccagggtttgacatcaattattatacatcggatagggcgaattggagctcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggccgcggccgcgggctagcggatccccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatgaatgcaattgttgttgttaataaaggaaatttattttcattgcaatagtgtgttggaattttttgtgtctctcaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctc

SEQ ID NO: 123 - DNA, 1929 bp, synthetic construct
  source 1..1929: mol_type = other DNA
aaggcgattaagttgggtaacgccagggttgccagtagccagccccgtcctggatagggcgaattggagctcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggccgcggccgcgggctagcggatccccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatgaatgcaattgttgttgttaataaaggaaatttattttcattgcaatagtgtgttggaattttttgtgtctctcaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctcgccagtagccagccccgtcctggaagatcctttgatcttttctacggggtctg

SEQ ID NO: 124 - DNA, 1876 bp, synthetic construct
  source 1..1876: mol_type = other DNA
aaggcgattaagttgggtaacgccagggttgccagtagccagccccgtcctggatagggcgaattggagctcggatccctatacagttgaagtcggaagtttacatacacttaagttggagtcattaaaactcgtttttcaactactccacaaatttcttgttaacaaacaatagttttggcaagtcagttaggacatctactttgtgcatgacacaagtcatttttccaacaattgtttacagacagattatttcacttataattcactgtatcacaattccagtgggtcagaagtttacatacactaagttgactgtgcctttaaacagcttggaaaattgtaacgccattttgcaaggcatggaaaaataccaaaccaagaatagagaagttcagatcaagggcgggtacatgaaaatagctaacgttgggccaaacaggatatctgcggtgagcagtttcggccccggcccggggccaagaacagatggtcaccgcagtttcggccccggcccgaggccaagaacagatggtccccagatatggcccaaccctcagcagtttcttaagacccatcagatgtttccaggctcccccaaggacctgaaatgaccctgcgccttatttgaattaaccaatcagcctgcttctcgcttctgttcgcgcgcttctgcttcccgagctctataaaagagctcacaacccctcactcggcgcgccagtcctccgacagactgagtcgcccgggccgcggccgcgggctagcggatccccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaaatgaatgcaattgttgttgttaataaaggaaatttattttcattgcaatagtgtgttggaattttttgtgtctctcaacaatttaaaggcaatgctaccaaatactaattgagtgtatgtaaacttctgacccactgggaatgtgatgaaagaaataaaagctgaaatgaatcattctctctactattattctgatatttcacattcttaaaataaagtggtgatcctaactgacctaagacagggaatttttactaggattaaatgtcaggaattgtgaaaaagtgagtttaaatgtatttggctaaggtgtatgtaaacttccgacttcaactgtatagggatcctctagctagagtcgacctc

SEQ ID NO: 125 - DNA, 1929 bp, synthetic construct; identical to SEQ ID NO: 121
  source 1..1929: mol_type = other DNA

SEQ ID NO: 126 - DNA, 1876 bp, synthetic construct; identical to SEQ ID NO: 122
  source 1..1876: mol_type = other DNA

SEQ ID NO: 127 - DNA, 1929 bp, synthetic construct; identical to SEQ ID NO: 123
  source 1..1929: mol_type = other DNA

SEQ ID NO: 128 - DNA, 1876 bp, synthetic construct; identical to SEQ ID NO: 124
  source 1..1876: mol_type = other DNA

(19) United States
(12) Patent Application Publication
(10) Pub. No.: US 2025/0179531 A1
(43) Pub. Date: Jun. 5, 2025

(54) COMPOSITIONS AND METHODS FOR HOMOLOGY-DIRECTED REPAIR GENE MODIFICATION

(71) Applicant: VOR BIOPHARMA INC., Cambridge, MA (US)

(72) Inventors: Ravindra Amunugama, Cambridge, MA (US); Tirtha Chakraborty, Cambridge, MA (US); Elizabeth Paik, Cambridge, MA (US); Abhinav Dhall, Cambridge, MA (US); John Lydeard, Cambridge, MA (US); Nipulkumar Patel, Cambridge, MA (US)

(73) Assignee: VOR BIOPHARMA INC., Cambridge, MA (US)

(21) Appl. No.: 18/840,740

(22) PCT Filed: Feb. 24, 2023
(86) PCT No.: PCT/US2023/063252

§ 371 Date: Aug. 22, 2024

Related U.S. Application Data
(60) Provisional application No. 63/314,279, filed on Feb. 25, 2022.

(51) Int. Cl. C12N 15/90; C12N 5/0783; C12N 9/22; C12N 15/11

(52) U.S. Cl. CPC C12N 15/907; C12N 5/0636; C12N 9/22; C12N 15/11; C12N 2310/20; C12N 2510/00; C12N 2800/80

(57) ABSTRACT

Provided herein are methods and compositions for genetically engineering a cell (e.g., a hematopoietic cell) using CRISPR/Cas systems and homology-directed repair, genetically engineered cells produced by such methods, and methods involving administering such genetically engineered cells to a subject, such as a subject having a genetic disease.

### RELATED APPLICATIONS

This application claims the benefit under 35 U.S.C. 119 (e) of U.S. Provisional Application No. 63/314,279 filed Feb. 25, 2022 which is incorporated by reference in its entirety.

### REFERENCE TO AN ELECTRONIC SEQUENCE LISTING

The contents of the electronic sequence listing (V029170035WO00-SEQ-CEW.xml; Size: 233,030 bytes; and Date of Creation: Feb. 22, 2023) is herein incorporated by reference in its entirety.

### SUMMARY

Some aspects of the present disclosure provide compositions and methods for genetic modification (or gene editing) of cells using homology-directed repair (HDR). In some embodiments, the compositions and methods for HDR-mediated gene editing provided herein can be applied to any cell type, but are particularly useful for editing mammalian cells, for example, human cells, and, in particular, for editing human hematopoietic cells, for example, human hematopoietic stem cells. In some embodiments, the compositions and methods for HDR-mediated gene editing provided herein can be used for the targeted correction of a genomic mutation, for example, a genomic mutation that is characteristic for, or causally associated with, a genetic disease or disorder. Accordingly, in some embodiments, the compositions and methods provided herein are useful to generate genetically modified cells or cell populations, in which such a genomic mutation has been corrected using HDR-mediated gene editing approaches provided herein. Some aspects of this disclosure provide therapeutic approaches, strategies, modalities, compositions, and methods based on HDR-mediated gene editing as described herein. For example, in some embodiments, the present disclosure provides genetically modified cells, e.g., cells obtained from a patient having a genetic disorder characterized by a genomic mutation, in which the respective mutation has been corrected, or in which the genomic DNA sequence in proximity to the mutation has been altered to a sequence that is not characteristic for the respective disease or disorder, using the presently provided HDR-mediated gene editing approaches, methods, and compositions. Accordingly, some aspects of the present disclosure provide methods and compositions, including genetically modified cells, e.g., human hematopoietic cells, for therapeutic purposes, for example, to treat a genetic disease or disorder. While the presently provided methods and compositions are suitable for use in the context of correcting a variety of mutations characteristic of various diseases or disorders, in some embodiments, methods and compositions that are particularly useful in the context of hematologic diseases or disorders, for example, Gaucher disease, or other enzyme deficiency diseases or lipid storage disorders, are provided. In some embodiments, methods and compositions described herein combine the sequence-specific nuclease activity of CRISPR/Cas systems with HDR, enabling targeted integration of sequences from a template polynucleotide at a target sequence specified by both the CRISPR/Cas system (e.g., a guide RNA (gRNA)) and by homology of portions of the template polynucleotide to the target sequence. In some embodiments, methods and compositions described herein are characterized by a high HDR-mediated editing efficiency in mammalian cells, e.g., in human hematopoietic cells, such as, for example, human hematopoietic stem cells. In some embodiments, methods and compositions described herein are characterized by a high HDR-mediated editing efficiency and a high rate of survival or high viability in the resulting edited cell populations, e.g., in populations of edited human hematopoietic cells, such as, for example, human hematopoietic stem cells.

Accordingly, in one aspect the disclosure is directed to a method comprising contacting a hematopoietic cell with a Clustered Regularly Interspaced Short Palindromic Repeats (CRISPR)-CRISPR associated (Cas) (CRISPR/Cas) system comprising a Cas nuclease and a guide RNA (gRNA) comprising a nucleotide sequence that hybridizes to a target DNA in the genome of the hematopoietic cell, and a template polynucleotide. In some embodiments, contacting also comprises contacting the hematopoietic cell with one or both of an expansion agent and a homology-directed repair (HDR) promoting agent.

In some embodiments, the CRISPR/Cas system creates a double-stranded break (DSB) in the target DNA in the genome of the hematopoietic cell. In some embodiments, the template polynucleotide is a single-stranded donor oligonucleotide (ssODN) or a double-stranded donor oligonucleotide (dsODN). In some embodiments, the template polynucleotide hybridizes to a genomic sequence flanking the DSB in the target DNA and integrates into the target DNA. In some embodiments, the template polynucleotide comprises a donor sequence, a first flanking sequence which is homologous to a genomic sequence upstream of the DSB in the target DNA and a second flanking sequence which is homologous to a genomic sequence downstream of the DSB in the target DNA. In some embodiments, the donor sequence of the template polynucleotide is integrated into the genome of the hematopoietic cell by homology-directed repair (HDR). In some embodiments, the template polynucleotide is a template for homology-directed repair (HDR) of a prior mutation in the target DNA. In some embodiments, the template polynucleotide is a template for homology-directed repair (HDR) insertion of a gene in the target DNA.

In some embodiments, contacting comprises contacting a population of hematopoietic cells. In some embodiments, a method described herein further comprises sorting the population of hematopoietic cells. In some embodiments, sorting comprises selecting for viable hematopoietic cells. In some embodiments, sorting comprises selecting for hematopoietic cells that integrated the donor sequence into their genome. In some embodiments, sorting comprises Fluorescence Activated Cell Sorting (FACS). In some embodiments, sorting comprises selecting for viable long term engrafting HSCs.

In some embodiments, the editing efficiency in the population of hematopoietic cells is at least 30, at least 40, at least 50, at least 60, at least 70, at least 80, at least 90, at least 95, or at least 99%. In some embodiments, the percent viability in the population of hematopoietic cells is at least 50, at least 60, at least 70, at least 80, at least 90, at least 95, or at least 99%. In some embodiments, the efficiency of HDR is 50% or higher. In some embodiments, the efficiency of HDR is 60% or higher. In some embodiments, the efficiency of HDR is 80% or higher.

In some embodiments, the expansion agent comprises at least one of StemRegenin (SR1), UM171, and IL-6. In some embodiments, the expansion agent comprises SR1 and UM171. In some embodiments, the HDR promoting agent comprises at least one of SCR7, NU7441, Rucaparib, and RS-1. In some embodiments, the HDR promoting agent comprises at least two of SCR7, NU7441, Rucaparib, and RS-1. In some embodiments, the HDR promoting agent comprises at least three of SCR7, NU7441, Rucaparib, and RS-1. In some embodiments, the HDR promoting agent comprises SCR7, NU7441, Rucaparib, and RS-1. In some embodiments, the SR1 is present at a concentration of 0.1-1.5, 0.3-1.5, 0.5-1.5, 0.7-1.5, 1-1.5, 1.2-1.5, 0.1-1, 0.3-1, 0.5-1, 0.7-1, 0.1-0.8, 0.3-0.8, 0.5-0.8, 0.7-0.8, 0.1-0.5, 0.3-0.5, or 0.1-0.3 μM. In some embodiments, the UM171 is present at a concentration of 1-100, 1-80, 1-60, 1-40, 1-20, 1-10, 20-100, 20-80, 20-60, 20-40, 30-100, 30-80, 30-60, 30-40, 50-100, 50-80, 50-60, or 80-100 nM. In some embodiments, the SCR7 is present at a concentration of 0.1-20, 0.1-15, 0.1-10, 0.1-8, 0.1-6, 0.1-5, 0.1-4, 0.1-3, 0.1-2, 0.1-1, 1-20, 1-15, 1-10, 1-8, 1-6, 1-5, 1-4, 1-3, 1-2, 3-20, 3-15, 3-10, 3-8, 3-6, 3-5, 3-4, 5-20, 5-15, 5-10, 5-8, 5-6, 8-20, 8-15, 8-10, 10-15, 10-20, or 15-20 μM. In some embodiments, the NU7441 is present at a concentration of 0.05-10, 0.05-8, 0.05-6, 0.05-5, 0.05-4, 0.05-3, 0.05-2, 0.05-1, 0.05-0.1, 0.1-20, 0.1-15, 0.1-10, 0.1-8, 0.1-6, 0.1-5, 0.1-4, 0.1-3, 0.1-2, 0.1-1, 1-20, 1-15, 1-10, 1-8, 1-6, 1-5, 1-4, 1-3, 1-2, 3-20, 3-15, 3-10, 3-8, 3-6, 3-5, 3-4, 5-20, 5-15, 5-10, 5-8, 5-6, 8-20, 8-15, 8-10, 10-15, 10-20, or 15-20 μM. In some embodiments, the RS-1 is present at a concentration of 0.1-50, 0.1-20, 0.1-15, 0.1-10, 0.1-8, 0.1-6, 0.1-5, 0.1-4, 0.1-3, 0.1-2, 0.1-1, 1-50, 1-20, 1-15, 1-10, 1-8, 1-6, 1-5, 1-4, 1-3, 1-2, 3-50 3-20, 3-15, 3-10, 3-8, 3-6, 3-5, 3-4, 5-50, 5-20, 5-15, 5-10, 5-8, 5-6, 8-50, 8-20, 8-15, 8-10, 10-50, 10-15, 10-20, 15-50, 15-20, or 20-50 μM. In some embodiments, Rucaparib is present at a concentration of 0.05-10, 0.05-8, 0.05-6, 0.05-5, 0.05-4, 0.05-3, 0.05-2, 0.05-1, 0.05-0.1, 0.1-20, 0.1-15, 0.1-10, 0.1-8, 0.1-6, 0.1-5, 0.1-4, 0.1-3, 0.1-2, 0.1-1, 1-20, 1-15, 1-10, 1-8, 1-6, 1-5, 1-4, 1-3, 1-2, 3-20, 3-15, 3-10, 3-8, 3-6, 3-5, 3-4, 5-20, 5-15, 5-10, 5-8, 5-6, 8-20, 8-15, 8-10, 10-15, 10-20, or 15-20 μM.

In some embodiments, the hematopoietic cell is a hematopoietic stem cell (HSC). In some embodiments, the hematopoietic cell is a CD34+ cell. In some embodiments, the hematopoietic cell is obtained from bone marrow, blood, umbilical cord, or peripheral blood mononuclear cells (PBMCs). In some embodiments, the hematopoietic cell is human.

In some embodiments, contacting also comprises contacting the hematopoietic cell with growth media. In some embodiments, the growth media is a Stromal Cell Growth Media (SCGM™), e.g., as available from Lonza Bioscience), or serum- and feeder-free media (SFFM). In some embodiments, the growth media comprises one or more cytokines. In some embodiments, the one or more cytokines are selected from one, two, or all of human stem cell factor (hSCF), Fms-like tyrosine kinase 3 ligand (FLT3-L), or thrombopoietin (TPO).

In some embodiments, the hematopoietic cell is capable of long-term engraftment into a human recipient. In some embodiments, the hematopoietic cell is capable of reconstituting the hematopoietic system in a human recipient after engraftment.

In some embodiments, the target DNA comprises a portion of a glucosylceramidase beta (GBA) gene. In some embodiments, the template polynucleotide comprises a first flanking sequence which is homologous to a first portion of the GBA gene and a second flanking sequence which is homologous to a second portion of the GBA gene.

In some embodiments, the target DNA comprises a portion of a C—C Motif Chemokine Receptor 5 (CCR5) gene. In some embodiments, the template polynucleotide comprises a first flanking sequence which is homologous to a first portion of the CCR5 gene and a second flanking sequence which is homologous to a second portion of the CCR5 gene.

In one aspect, the disclosure is directed to a method comprising contacting a hematopoietic cell with a Clustered Regularly Interspaced Short Palindromic Repeats (CRISPR)-CRISPR associated (Cas) (CRISPR/Cas) system comprising a Cas nuclease and a guide RNA (gRNA) comprising a nucleotide sequence that hybridizes to a target DNA in a glucosylceramidase beta (GBA) gene in the genome of the hematopoietic cell, wherein the CRISPR/Cas system creates a double-stranded break (DSB) in the GBA gene; and a template polynucleotide comprising a donor sequence, a first flanking sequence which is homologous to a first portion of the GBA gene and a second flanking sequence which is homologous to a second portion of the GBA gene.

In some embodiments, the first portion of the GBA gene comprises a portion of exon 9 or a sequence proximal thereto. In some embodiments, the second portion of the GBA gene comprises a portion of exon 9 or a sequence proximal thereto, wherein the first portion and second portion are not identical. In some embodiments, the first portion of the GBA gene comprises a portion of exon 10 or a sequence proximal thereto. In some embodiments, the second portion of the GBA gene comprises a portion of exon 10 or a sequence proximal thereto, wherein the first portion and second portion are not identical.

In some embodiments, the donor sequence comprises a sequence corresponding to the codon encoding N409 or L483 in a wildtype GBA gene. In some embodiments, the wildtype GBA gene comprises the sequence of SEQ ID NO: 47.

In some embodiments, the sequence corresponding to the codon encoding N409 in the wildtype GBA gene encodes an asparagine. In some embodiments, the template polynucleotide comprises the sequence of any one of SEQ ID NOs: 51-54.

In some embodiments, the sequence corresponding to the codon encoding N409 in the wildtype GBA gene encodes a serine. In some embodiments, the template polynucleotide comprises the sequence of SEQ ID NOs: 25-28.

In some embodiments, the sequence corresponding to the codon encoding L483 in the wildtype GBA gene encodes a leucine. In some embodiments, the template polynucleotide comprises the sequence of any one of SEQ ID NOs: 55-57.

In some embodiments, the sequence corresponding to the codon encoding L483 in the wildtype GBA gene encodes a proline. In some embodiments, the template polynucleotide comprises the sequence of SEQ ID NOs: 29-30.

In some embodiments, the first flanking sequence comprises a flanking sequence set forth in any one of SEQ ID NOs: 25-30 or 51-57 or a sequence with at least 80, at least 85, at least 90, at least 95, at least 96, at least 97, at least 98, or at least 99% identity to any thereof. In some embodiments, the second flanking sequence comprises a flanking sequence set forth in any one of SEQ ID NOs: 25-30 or 51-57 or a sequence with at least 80, at least 85, at least 90, at least 95, at least 96, at least 97, at least 98, or at least 99% identity to any thereof. In some embodiments, the donor sequence comprises a donor sequence selected from any one of SEQ ID NOs: 25-30 or 51-57 or a sequence with at least 80, at least 85, at least 90, at least 95, at least 96, at least 97, at least 98, or at least 99% identity to any thereof. In some embodiments, the template polynucleotide comprises the sequence of SEQ ID NOs: 25-30 or 51-57 or a sequence with at least 80, at least 85, at least 90, at least 95, at least 96, at least 97, at least 98, or at least 99% identity to any thereof.

In one aspect, the disclosure is directed to a method comprising contacting a hematopoietic cell with a Clustered Regularly Interspaced Short Palindromic Repeats (CRISPR)-CRISPR associated (Cas) (CRISPR/Cas) system comprising a Cas nuclease and a guide RNA (gRNA) comprising a nucleotide sequence that hybridizes to a target DNA in a CCR5 gene in the genome of the hematopoietic cell, wherein the CRISPR/Cas system creates a double-stranded break (DSB) in the CCR5 gene; and a template polynucleotide comprising a donor sequence, a first flanking sequence which is homologous to a first portion of the CCR5 gene and a second flanking sequence which is homologous to a second portion of the CCR5 gene. In some embodiments, the first portion of the CCR5 gene and second portion of the CCR5 gene are not identical.

In some embodiments, the first flanking sequence comprises a flanking sequence set forth in any one of SEQ ID NOs: 43-46 or a sequence with at least 80, at least 85, at least 90, at least 95, at least 96, at least 97, at least 98, or at least 99% identity to any thereof. In some embodiments, the second flanking sequence comprises a flanking sequence set forth in any one of SEQ ID NOs: 43-46 or a sequence with at least 80, at least 85, at least 90, at least 95, at least 96, at least 97, at least 98, or at least 99% identity to any thereof. In some embodiments, the donor sequence comprises a donor sequence selected from any one of SEQ ID NOs: 43-46 or a sequence with at least 80, at least 85, at least 90, at least 95, at least 96, at least 97, at least 98, or at least 99% identity to any thereof. In some embodiments, the template polynucleotide comprises the sequence of SEQ ID NOs: 43-46 or a sequence with at least 80, at least 85, at least 90, at least 95, at least 96, at least 97, at least 98, or at least 99% identity to any thereof.

In some embodiments, the donor sequence comprises a restriction site or a unique sequence tag. In some embodiments, the sequence comprising the restriction site or a unique sequence tag is an insertion relative to the target DNA. In some embodiments, the sequence comprising the restriction site or a unique sequence tag is not an insertion relative to the target DNA. In some embodiments, the sequence comprising the restriction site or a unique sequence tag does not alter an amino acid sequence encoded by the target DNA.

In some embodiments, the first flanking sequence, second flanking sequence, or both comprise a PAM site sequence or a sequence complementary to the PAM site sequence. In some embodiments, the restriction site is no more than 20, no more than 15, no more than 10, no more than 9, no more than 8, no more than 7, no more than 6, no more than 5, no more than 4, no more than 3, no more than 2, or no more than 1 nucleotides from the PAM site sequence or the sequence complementary to the PAM site sequence.

In some embodiments, the donor sequence comprises a second mutation relative to the target DNA. In some embodiments, the second mutation is a silent mutation. In some embodiments, the second mutation is situated in a codon that is contiguous with the HDR mutation or HDR insertion.

In some embodiments, wherein the ssODN comprises, from 5′ to 3′, the first flanking sequence, the donor sequence, and the second flanking sequence.

In some embodiments, the first flanking sequence is 50-200, 50-180, 50-160, 50-140, 50-120, 50-100, 50-80, 50-60, 70-200, 70-180, 70-160, 70-140, 70-120, 70-100, 70-80, 100-200, 100-180, 100-160, 100-140, 100-120, 120-200, 120-180, 120-160, 120-140, 150-200, 150-180, or 150-160 nucleotides in length. In some embodiments, the second flanking sequence is 50-200, 50-180, 50-160, 50-140, 50-120, 50-100, 50-80, 50-60, 70-200, 70-180, 70-160, 70-140, 70-120, 70-100, 70-80, 100-200, 100-180, 100-160, 100-140, 100-120, 120-200, 120-180, 120-160, 120-140, 150-200, 150-180, or 150-160 nucleotides in length. In some embodiments, the donor sequence is 1-100, 1-80, 1-60, 1-40, 1-20, 1-15, 1-10, 1-9, 1-8, 1-7, 1-6, 1-5, 1-4, 1-3, 1-2, 5-100, 5-80, 5-60, 5-40, 5-20, 5-15, 5-10, 5-9, 5-8, 5-7, 5-6, 10-100, 10-80, 10-60, 10-40, 10-20, 10-15, 20-100, 20-80, 20-60, 20-40, 60-100, or 60-80 nucleotides in length.

In some embodiments, the CRISPR/Cas system comprises a guide nucleic acid comprising a sequence chosen from any one of SEQ ID NOs: 3, 6, 9, 12, 15, 18, 21, 24, 33, 36, 39, and 42, or a sequence having no more than 1, no more than 2, no more than 3, no more than 4, or no more than 5 substitutions relative to any thereof.

In some embodiments, the donor sequence is integrated into the genome of the hematopoietic stem cell by homology-directed repair (HDR).

In some embodiments, a method described herein is a method of producing a genetically modified hematopoietic cell or population of genetically modified hematopoietic cells.

In one aspect, the disclosure is directed to a method comprising providing a genetically modified hematopoietic cell wherein the hematopoietic cell was genetically modified to comprise one, two, or three of: an endogenous glucosylceramidase beta (GBA) gene that encodes an asparagine at a position corresponding to position 409 of a wildtype GBA gene; an endogenous GBA gene that encodes a leucine at a position corresponding to position 409 of a wildtype GBA gene; or a heterologous copy of a GBA gene that encodes an asparagine at a position corresponding to position 409 of a wildtype GBA gene and a leucine at a position corresponding to position 409 of a wildtype GBA gene, and administering the genetically modified hematopoietic cell to a subject. In some embodiments, the method is a method of treating Gaucher disease in the subject. In some embodiments, the genetically modified hematopoietic cell is a genetically modified hematopoietic stem cell. In some embodiments, providing comprises genetically modifying the hematopoietic cell by contacting the cell with a Clustered Regularly Interspaced Short Palindromic Repeats (CRISPR)-CRISPR associated (Cas) (CRISPR/Cas) system comprising a Cas nuclease and a guide RNA (gRNA) comprising a nucleotide sequence that hybridizes to a target DNA in a glucosylceramidase beta (GBA) gene in the genome of the hematopoietic cell, wherein the CRISPR/Cas system creates a double-stranded break (DSB) in the GBA gene; and a template polynucleotide comprising a donor sequence, a first flanking sequence which is homologous to a first portion of the GBA gene and a second flanking sequence which is homologous to a second portion of the GBA gene. In some embodiments, the genetically modified hematopoietic cell administered to a subject was produced by a method described herein. In some embodiments, the genetically modified hematopoietic stem cell is autologous to the subject.

In one aspect, the disclosure is directed to a template polynucleotide comprising a nucleic acid single-strand that comprises, from 5′ to 3′: a first flanking sequence complementary to a first portion of a glucosylceramidase beta (GBA) gene, a donor sequence, and a second flanking sequence complementary to a second portion of the GBA gene. In some embodiments, the template polynucleotide is a single-strand donor oligonucleotide (ssODN) or a double-stranded oligonucleotide (dsODN) donor.

In some embodiments, the template polynucleotide is a template for homology-directed repair (HDR) of a mutation in the GBA gene. In some embodiments, the template polynucleotide is a template for homology-directed repair (HDR) insertion of a GBA gene or portion thereof. In some embodiments, the first portion of the GBA gene comprises a portion of exon 9 or a sequence proximal thereto. In some embodiments, the second portion of the GBA gene comprises a portion of exon 9 or a sequence proximal thereto, wherein the first portion and second portion are not identical. In some embodiments, the first portion of the GBA gene comprises a portion of exon 10 or a sequence proximal thereto. In some embodiments, the second portion of the GBA gene comprises a portion of exon 10 or a sequence proximal thereto, wherein the first portion and second portion are not identical. In some embodiments, the donor sequence comprises a sequence corresponding to the codon encoding N409 or L483 in a wildtype GBA gene. In some embodiments, the wildtype GBA gene comprises the sequence of SEQ ID NO: 47. In some embodiments, the sequence corresponding to the codon encoding N409 in the wildtype GBA gene encodes an asparagine. In some embodiments, the template polynucleotide comprises the sequence of any one of SEQ ID NOs: 51-54 or a sequence with at least 80, at least 85, at least 90, at least 95, at least 96, at least 97, at least 98, or at least 99% identity to any thereof. In some embodiments, the sequence corresponding to the codon encoding N409 in the wildtype GBA gene encodes a serine. In some embodiments, the donor sequence comprises the sequence of SEQ ID NOS: 25-28 or a sequence with at least 80, at least 85, at least 90, at least 95, at least 96, at least 97, at least 98, or at least 99% identity to any thereof. In some embodiments, the sequence corresponding to the codon encoding L483 in the wildtype GBA gene encodes a leucine. In some embodiments, the template polynucleotide comprises the sequence of any one of SEQ ID NOs: 55-57 or a sequence with at least 80, at least 85, at least 90, at least 95, at least 96, at least 97, at least 98, or at least 99% identity to any thereof. In some embodiments, the sequence corresponding to the codon encoding L483 in the wildtype GBA gene encodes a proline. In some embodiments, the template polynucleotide comprises the sequence of SEQ ID NOs: 29-30 or a sequence with at least 80, at least 85, at least 90, at least 95, at least 96, at least 97, at least 98, or at least 99% identity to any thereof. In some embodiments, the first flanking sequence comprises a flanking sequence as set forth in any one of SEQ ID NOs: 25-30 or 51-57 or a sequence with at least 80, at least 85, at least 90, at least 95, at least 96, at least 97, at least 98, or at least 99% identity to any thereof. In some embodiments, the second flanking sequence comprises a flanking sequence as set forth in any one of SEQ ID NOs: 25-30 or 51-57 or a sequence with at least 80, at least 85, at least 90, at least 95, at least 96, at least 97, at least 98, or at least 99% identity to any thereof. In some embodiments, the donor sequence comprises a donor sequence of any one of SEQ ID NOs: 25-30 or 51-57 or a sequence with at least 80, at least 85, at least 90, at least 95, at least 96, at least 97, at least 98, or at least 99% identity to any thereof.

In some embodiments, the donor sequence comprises a restriction site or a unique sequence tag. In some embodiments, the sequence comprising the restriction site or unique sequence tag is an insertion relative to a target site in the GBA gene. In some embodiments, the sequence comprising the restriction site or unique sequence tag is not an insertion relative to a target site in the GBA gene. In some embodiments, the sequence comprising the restriction site or unique sequence tag does not alter an amino acid sequence encoded by the target site. In some embodiments, the first flanking sequence, second flanking sequence, or both comprise a PAM site sequence or a sequence complementary to a PAM site sequence present in the GBA gene. In some embodiments, the restriction site or unique sequence tag is no more than 20, no more than 15, no more than 10, no more than 9, no more than 8, no more than 7, no more than 6, no more than 5, no more than 4, no more than 3, no more than 2, or no more than 1 nucleotides from the PAM site sequence or the sequence complementary to a PAM site sequence.

In some embodiments, the donor sequence comprises a second mutation relative to the target DNA. In some embodiments, the second mutation is a silent mutation. In some embodiments, the second mutation is situated in a codon that is contiguous with the HDR mutation or HDR insertion.

In one aspect, the disclosure is directed to a guide nucleic acid comprising a sequence complementary to a portion of the glucosylceramidase beta (GBA) gene, wherein the portion comprises a portion of exon 9 or exon 10 and a PAM site sequence.

In one aspect, the disclosure is directed to a guide nucleic acid comprising the sequence of any one of SEQ ID NOs: 3, 6, 9, 12, 15, 18, 21, 24, 33, 36, 39, or 42, or a sequence having no more than 1, no more than 2, no more than 3, no more than 4, or no more than 5 substitutions relative to any thereof.

In one aspect, the disclosure is directed to a mixture comprising: a template polynucleotide comprising a nucleic acid single-strand that comprises a donor sequence, a first flanking sequence and a second flanking sequence; a Clustered Regularly Interspaced Short Palindromic Repeats (CRISPR)-CRISPR associated (Cas) (CRISPR/Cas) system comprising a Cas nuclease and a guide RNA (gRNA) comprising a nucleotide sequence that hybridizes to a target DNA in the genome of the hematopoietic cell; and one or both of an expansion agent selected from at least one of StemRegenin 1 (SR1), and UM171, and a homology-directed repair (HDR) promoting agent selected from at least one of SCR7, NU7441, Rucaparib, and RS-1.

In one aspect, the disclosure is directed to a kit comprising: a template polynucleotide comprising a nucleic acid single-strand that comprises a donor sequence, a first flanking sequence and a second flanking sequence; a Clustered Regularly Interspaced Short Palindromic Repeats (CRISPR)-CRISPR associated (Cas) (CRISPR/Cas) system comprising a Cas nuclease and a guide RNA (gRNA) comprising a nucleotide sequence that hybridizes to a target DNA in the genome of the hematopoietic cell; and one or both of an expansion agent selected from at least one of StemRegenin 1 (SR1), and UM171, and a homology-directed repair (HDR) promoting agent selected from at least one of SCR7, NU7441, Rucaparib, and
RS-1. In some embodiments, the template polynucleotide is a template polynucleotide described herein.

In some embodiments, a kit comprises one or more containers comprising the components of the kit (e.g., the template polynucleotide, the CRISPR/Cas system, the expansion agent(s), and the HDR promoting agent(s)), e.g., separate containers for each component. In some embodiments, a kit comprises instructions for producing a genetically modified hematopoietic stem cell. In some embodiments, a kit comprises instructions to perform a method described herein (e.g., of genetically engineering a hematopoietic cell).

In some embodiments, a kit or mixture comprises expansion agents comprising at StemRegenin 1 (SR1) and UM171. In some embodiments, a kit or mixture comprises HDR promoting agents comprising at least two of SCR7, NU7441, Rucaparib, and RS-1. In some embodiments, a kit comprises HDR promoting agents comprising at least three of SCR7, NU7441, Rucaparib, and RS-1. In some embodiments, a kit or mixture comprises HDR promoting agents comprising SCR7, NU7441, Rucaparib, and RS-1. In some embodiments, the SR1 is present at a concentration of 0.1-1.5, 0.3-1.5, 0.5-1.5, 0.7-1.5, 1-1.5, 1.2-1.5, 0.1-1, 0.3-1, 0.5-1, 0.7-1, 0.1-0.8, 0.3-0.8, 0.5-0.8, 0.7-0.8, 0.1-0.5, 0.3-0.5, or 0.1-0.3 μM. In some embodiments, the UM171 is present at a concentration of 1-100, 1-80, 1-60, 1-40, 1-20, 1-10, 20-100, 20-80, 20-60, 20-40, 30-100, 30-80, 30-60, 30-40, 50-100, 50-80, 50-60, or 80-100 nM. In some embodiments, the SCR7 is present at a concentration of 0.1-20, 0.1-15, 0.1-10, 0.1-8, 0.1-6, 0.1-5, 0.1-4, 0.1-3, 0.1-2, 0.1-1, 1-20, 1-15, 1-10, 1-8, 1-6, 1-5, 1-4, 1-3, 1-2, 3-20, 3-15, 3-10, 3-8, 3-6, 3-5, 3-4, 5-20, 5-15, 5-10, 5-8, 5-6, 8-20, 8-15, 8-10, 10-15, 10-20, or 15-20 μM. In some embodiments, the NU7441 is present at a concentration of 0.05-10, 0.05-8, 0.05-6, 0.05-5, 0.05-4, 0.05-3, 0.05-2, 0.05-1, 0.05-0.1, 0.1-20, 0.1-15, 0.1-10, 0.1-8, 0.1-6, 0.1-5, 0.1-4, 0.1-3, 0.1-2, 0.1-1, 1-20, 1-15, 1-10, 1-8, 1-6, 1-5, 1-4, 1-3, 1-2, 3-20, 3-15, 3-10, 3-8, 3-6, 3-5, 3-4, 5-20, 5-15, 5-10, 5-8, 5-6, 8-20, 8-15, 8-10, 10-15, 10-20, or 15-20 μM. In some embodiments, the RS-1 is present at a concentration of 0.1-50, 0.1-20, 0.1-15, 0.1-10, 0.1-8, 0.1-6, 0.1-5, 0.1-4, 0.1-3, 0.1-2, 0.1-1, 1-50, 1-20, 1-15, 1-10, 1-8, 1-6, 1-5, 1-4, 1-3, 1-2, 3-50 3-20, 3-15, 3-10, 3-8, 3-6, 3-5, 3-4, 5-50, 5-20, 5-15, 5-10, 5-8, 5-6, 8-50, 8-20, 8-15, 8-10, 10-50, 10-15, 10-20, 15-50, 15-20, or 20-50 μM. In some embodiments, the Rucaparib is present at a concentration of 0.05-10, 0.05-8, 0.05-6, 0.05-5, 0.05-4, 0.05-3, 0.05-2, 0.05-1, 0.05-0.1, 0.1-20, 0.1-15, 0.1-10, 0.1-8, 0.1-6, 0.1-5, 0.1-4, 0.1-3, 0.1-2, 0.1-1, 1-20, 1-15, 1-10, 1-8, 1-6, 1-5, 1-4, 1-3, 1-2, 3-20, 3-15, 3-10, 3-8, 3-6, 3-5, 3-4, 5-20, 5-15, 5-10, 5-8, 5-6, 8-20, 8-15, 8-10, 10-15, 10-20, or 15-20 μM.

In some embodiments, a Cas nuclease (e.g., for use in a method, kit, or mixture described herein) is Cas9. In some embodiments, the Cas nuclease is Streptococcus pyogenes Cas9 (spCas9). In some embodiments, the Cas nuclease is Staphylococcus aureus Cas9 (saCas9). In some embodiments, the Cas nuclease is Cas12a. In some embodiments, the Cas nuclease is Cas12b. In some embodiments, the Cas nuclease is Cas13.

In some embodiments, the contacting comprises introducing the CRISPR/Cas system into the cell in the form of a pre-formed ribonucleoprotein (RNP) complex. In some embodiments, the pre-formed RNP complex is introduced into the cell via electroporation. In some embodiments, the contacting comprises introducing the template polynucleotide into the cell via electroporation. In some embodiments, the template polynucleotide and CRISPR/Cas system are electroporated into the cell simultaneously. In some embodiments, the CRISPR/Cas system is introduced into the hematopoietic cell within 0, 1, or 2 days after culturing the hematopoietic cell.

In some embodiments, a CRISPR/Cas system for use in a method, kit, or mixture described herein comprises a guide nucleic acid which comprises one or more nucleotide residues that are chemically modified. In some embodiments, the chemically modified nucleotide residues comprise 2′O-methyl moieties. In some embodiments, the chemically modified nucleotide residues comprise phosphorothioate moieties. In some embodiments, the chemically modified nucleotide residues comprise thioPACE moieties.

In some embodiments, a genetically modified hematopoietic stem cell has reduced or eliminated expression of a lineage-specific cell-surface antigen relative to a wildtype hematopoietic stem cell. In some embodiments, the lineage-specific cell-surface antigen is selected from the group consisting of CD33, CD19, CD123, CLL-1, CD30, CD5, CD6, CD7, CD38, CD45, and BCMA.

In one aspect, the disclosure is directed to a genetically modified hematopoietic stem cell, or descendant thereof, produced by a method described herein.

In one aspect, the disclosure is directed to a cell population comprising a plurality of cells obtained by or obtainable by a method described herein, or a plurality of genetically modified hematopoietic cells (e.g., hematopoietic stem cells) described herein.

In one aspect, the disclosure is directed to a pharmaceutical composition comprising a cell, or a descendant thereof, or cell population described herein.

### BRIEF DESCRIPTION OF THE DRAWINGS

FIG. 1 shows a diagram for the design of an exemplary template polynucleotide (e.g., a single-stranded donor oligonucleotide (ssODN)) which serves as a template sequence used in homology-directed repair (HDR)-mediated editing of a genomic locus targeted by CRISPR/Cas9. CRISPR/Cas9 cleavage and subsequent HDR using the sequence encoded by the ssODN introduces a Pvu1 restriction enzyme site located 3 nucleotides (nt) upstream of the PAM site.

FIGS. 2A and 2B show enzymatic characterization of HDR insertion. FIG. 2A shows gel electrophoresis analysis of Pvu1 restriction site cleavage products inserted by ssODNs-based HDR into the CCR5 gene in cells that were either not electroporated (No EP) or subjected to electroporation under conditions to promote gene editing via CRISPR/Cas9 (HDR-edited). FIG. 2B is a quantification of the cleavage of the Pvu1 restriction site in cells as a result of electroporation.

FIG. 3 shows the percentage of insertion of 6 nt Pvu1 restriction site in the CCR5 gene following electroporation of CD34+ cells with either ribonucleoprotein (RNP; a complex formed by a guide RNA (gRNA) targeting the CCR5 gene and Cas9), ssODN, or RNP+ssODN. Percentage of insertion was detected by sequencing and quantified using a software that interprets CRISPR/Cas9 editing outcomes called inference of CRISPR-edits (ICE).

FIGS. 4A-4C show HDR editing efficiencies based on the presence of HSC expansion molecules and DNA repair modulators as determined by detection of a 6 nt Pvu1 restriction site insertion in the CCR5 gene in CD34+ cells via ICE analysis of sequencing data. FIG. 4A shows the effect of DNA repair modulators with or without cell expansion compounds (SFT and ISU) on ssODN-based HDR efficiency. DNA repair modulators included SCR7 (a ligase IV inhibitor), NU7441 (a DNA-PK inhibitor), Rucaparib (a PARP inhibitor), and RS-1 (an HDR enhancer). All cells were cultured using differentiation and proliferation compounds (hSCF, FLT3-L, TPO; SFT). FIGS. 4B and 4C show the effect of addition of IL-6 to media on editing and HDR efficiency, respectively.

FIGS. 5A and 5B show editing of long term-human stem cells (LT-HSCs) by ssODN-based HDR. FIG. 5A is a diagram of the strategy for generating ssODN-based HDR edited LT-HSCs from CD34+ cells. FIG. 5B shows editing efficiency of ssODN-based HDR in LT-HSCs relative to control CD34+ cells 3 days post electroporation (EP) as determined by ICE analysis of sequencing data.

FIGS. 6A and 6B show the percent viability and cell counts following electroporation (post-EP) of Cas9 RNP and ssODN at the indicated concentrations on day 0 (Day-0 post-EP) and 3 days following electroporation (Day-3 post-EP). FIG. 6A shows the relationship between cell viability and ssODN concentration. FIG. 6B shows the relationship between cell count and ssODN concentration.

FIG. 7 shows outcomes of HDR-mediated editing of the CCR5 locus in T cells in response to optimized media conditions. Total editing and Pvu1 restriction site insertion percentages were determined by ICE analysis of sequencing data at 7 days (Day-7) and 10 days (Day-10) post-electroporation.

FIGS. 8A-8B shows quantification characterizing the LT-HSC gating strategy for analysis of HDR-edited cells. FIG. 8A shows quantifications of flow cytometry gating plots of HDR-edited cells. FIG. 8B shows quantifications of flow cytometry gating plots of control cells.

FIGS. 9A-9E show design and validation of sgRNAs for Cas9-based editing of the glucosylceramidase beta (GBA) gene. FIG. 9A shows the location of exemplary sgRNAs, SG1-SG4 encoding the 1226A>G mutation, relative to the target sequences in exon 9 of the GBA gene, to produce the N409S mutation (from top to bottom, SEQ ID NOs: 74-76). FIG. 9B shows the location of exemplary gRNAs, SG5-SG8 encoding the 1448T>C mutation, relative to the target sequences in exon 10 of the GBA gene, to produce the L483P mutation (from top to bottom, SEQ ID NOs: 77-79). FIG. 9C shows editing efficiency of the eight sgRNA candidates (SG1-8) which target either exon 9 (SG1-4) or exon 10 (SG5-8) of the GBA gene as determined by ICE analysis of sequencing data. R2 values reflect how well the indel distribution proposed by ICE fits the sequence of the edited samples. FIG. 9D shows SG1, SG4, SG6, and SG7 editing efficiency in the GBA gene as determined by ICE analysis of sequencing data. FIG. 9E shows an exemplary approach for using donor sequences comprising one or more genomic modifications encoding silent mutations in codons proximal to N409 in exon 9 of the GBA gene, which is useful, for example, to tag edited cells, for example, for identifying edited cells (from top to bottom, SEQ ID NOs: 80-82).

FIGS. 10A-10D show that Gaucher disease-related mutations in the GBA gene can be introduced in CD34+ cells using ssODN-based HDR. FIG. 10A is a diagram of the experimental design for editing CD34+ cells using ssODN-based HDR via CRISPR/Cas9. FIG. 10B shows HDR efficiency of ssODNs encoding silent mutations (SM; ssODN1-4 encoding N409S and ssODN5-6 encoding L483P) in contiguous codons that were electroporated either alone or in the presence of single guide ribonucleoproteins (sgRNP; sgRNPs comprised of Cas9 complexed with SG1, SG4, SG6, or SG7). FIG. 10C shows cell viability following electroporation with either RNPs and ssODNs, ssODNs alone, or mock electroporation and no electroporation controls. FIG. 10D shows cell count in response to electroporation with either sgRNPs+ssODNs, or ssODNs alone.

FIGS. 11A-11C show sgRNA design for re-editing of mutated GBA loci. FIG. 11A shows the location of an exemplary gRNAs, SG13, which produced the S409N mutation in the target sequence located in exon 9 of the GBA gene (from top to bottom, SEQ ID NOs: 83-85). FIG. 11B shows the location of an exemplary gRNA, SG14, which produced the P483L mutation in the target sequence located in exon 10 of the GBA gene (from top to bottom, SEQ ID NOs: 86-88). FIG. 11C shows sequences corresponding to gRNAs SG13 (SEQ ID NO: 62) and SG14 (SEQ ID NO: 63).

FIG. 12 shows a diagram of the strategy used to re-edit CD34+ cells comprising mutated GBA loci.

FIGS. 13A-13C show re-editing outcomes associated with correction of GBA mutation N409S in CD34+ cells. FIG. 13A shows insertion sequences of exemplary ssODNs (from top top to bottom, SEQ ID NOs: 89 and 90). FIG. 13B shows Sanger sequencing analysis of ssODN insertion sequence integration into mutated GBA loci encoding Gaucher mutation N409S. FIG. 13C shows next-generation sequencing (NGS) analysis of ssODN insertion sequence integration into mutated GBA loci encoding Gaucher mutation N409S.

FIGS. 14A-14C show an exemplary strategy for CCR5 editing in T cells. FIG. 14A shows design of an exemplary long ssODN comprising an EGFP reporter for CCR5 editing. FIG. 14B shows a diagram of an exemplary strategy for editing CCR5 using HDR. FIG. 14C shows flow cytometry analyses of reporter expression as a result of optimization of electroporation conditions.

FIGS. 15A-15B show an exemplary strategy for AAVS1 editing in T cells using uncapped, dsODN. FIG. 15A shows design of an exemplary uncapped, dsODN comprising an EGFP reporter for AAVS1 editing. FIG. 15B shows a diagram of an exemplary strategy for editing AAVS1 using an uncapped, dsODN via HDR. FIG. 15C shows flow cytometry analyses of reporter expression as a result of editing the AAVS1 and CCR5 loci in T cells.

FIGS. 16A-16B show an exemplary strategy for RAB11a editing in T cells using capped, dsODN. FIG. 16A shows design of an exemplary capped, dsODN comprising an EGFP reporter for RAB11a editing. FIG. 16B shows a diagram of an exemplary strategy for editing RAB11a using a capped, dsODN via HDR.

FIGS. 17A-17B show flow cytometry analyses of expression from edited AAVS1 and RAB11a loci generated via electroporation with Cas9 RNPs and capped, dsODNs in T cells. FIG. 17A shows flow cytometry expression analyses of reporter expression from edited AAVS1 and RAB11a loci. FIG. 17B shows expression analyses of engineered an RAB11a locus edited via electroporation in the presence of Cas9 RNPs, dsODN, and non-homologous end-joining (NHEJ) inhibitors.

FIG. 18 shows an exemplary recombinant adeno-associated virus (rAAV)-encoded donor template comprising an EGFP reporter for editing of the AAVS1 locus.

FIG. 19 shows an exemplary strategy for editing AAVS1 in T cells using rAAVs comprising a donor template designed for integration into AAVS1.

FIG. 20 shows flow cytometry analyses of reporter expression from edited AAVS1 generated via electroporation with Cas9 RNPs and rAAV-delivered donor templates.

FIGS. 21A-21C show an exemplary strategy for integration of CD33-targeted chimeric antigen receptors (CARs) in T cells. FIG. 21A shows design of exemplary sgRNAs, SG17 and SG18, relative to target sequences in exon 2 of the TRAC locus (from top to bottom, SEQ ID NOs: 91 and 92). FIG. 21B shows a schematic of exemplary capped, double-stranded CD33-CAR donor templates designed for integration at the TRAC locus. FIG. 21C shows a schematic of exemplary capped, double-stranded CD33-CAR donor templates integrated at the RAB11a and AAVS1 loci.

FIG. 22 shows an exemplary strategy for integration of capped, double-stranded CD33-CAR donor templates in T cells.

FIG. 23 shows flow cytometry analyses of reporter expression from CD33-CAR donor templates integrated at the TRAC locus in T cells.

FIG. 24 shows flow cytometry analyses of reporter expression from CD33-CAR donor templates integrated at the RAB11a locus in T cells.

### DETAILED DESCRIPTION

The disclosure is directed to methods and compositions for genetic modification of cells (e.g., hematopoietic cells, e.g., hematopoietic stem cells (HSCs)) using HDR. Without wishing to be bound by theory, breaks in target DNA can be sequence-specifically induced by CRISPR/Cas systems and then repaired with HDR using a template polynucleotide with further specificity to the site of the break. In addition, DNA repair can be directed to HDR pathways by the addition of one or more HDR-promoting agents. Some aspects of this disclosure are based, at least in part, on the surprising finding that hematopoietic cells, for example, hematopoietic stem cells, can be genetically engineered at high efficiency via HDR-mediated mechanisms, for example, by using template polynucleotides, for example, single-stranded or double-stranded template polynucleotides, together with CRISPR editing systems as provided herein. Accordingly, some aspects of the present disclosure provide compositions, strategies, methods, and modalities useful for generating genetically engineered cells (for example, genetically engineered hematopoietic cells). Some of the compositions, strategies, methods, and modalities useful for generating genetically engineered cells provided herein include, for example, template polynucleotides, CRISPR editing systems comprising such template polynucleotides, kits and genetic modification mixtures, and methods of using such polynucleotides, CRISPR editing systems, kits and mixtures. Some aspects of this disclosure provide genetically engineered cells, e.g., genetically engineered hematopoietic cells, and cell populations comprising such genetically engineered cells, generated by using the compositions, strategies, methods, and modalities provided herein. Some aspects of this disclosure provide methods of using genetically engineered cells or cell populations as provided herein, for example, in the context of methods of treating a subject in need thereof, for example, a subject having or diagnosed with a disease or disorder characterized by a mutation that can be corrected by using the compositions, strategies, methods, and modalities useful for generating genetically engineered cells (for example, genetically engineered hematopoietic cells) provided herein.

### Homology-Directed Repair (HDR) Using Template Polynucleotides

In some embodiments, the present disclosure provides genetically engineered cells and cell populations, and methods of producing genetically engineered cells and cell populations using HDR-mediated gene editing, e.g., CRISPR/Cas-based HDR-mediated gene editing. Without being bound by any particular theory, HDR is a process wherein damage to DNA (e.g., a break in the DNA) is repaired using a donor sequence with flanking sequences comprising homology to the site of DNA damage. In some embodiments, a CRISPR/Cas system is used to introduce a break in the DNA (e.g., a double-stranded break (DSB)). Without wishing to be bound by theory, by providing a donor sequence (e.g., via a template polynucleotide) in the presence of a DSB, it is thought that HDR can be promoted (e.g., relative to other DNA repair pathways, e.g., NHEJ). HDR can result in substitution or insertion mutations that replace endogenous or naturally occurring sequences with those of the donor sequence. For example, methods described herein can be used to introduce a mutation into a target DNA, e.g., to correct a disease-associated genetic mutation. As a further example, methods described herein can be used to introduce a mutation, e.g., to insert a nucleotide sequence (e.g., a nucleotide sequence correcting a mutation in a genomic sequence).

In some embodiments, the donor sequence is provided by, for example, a template polynucleotide. When the donor sequence differs at one or more positions relative to a target DNA, integration of the donor sequence by HDR results in a mutation. In some embodiments, the target DNA comprises a mutation relative to a reference sequence (e.g., a wild-type sequence, or a sequence dominant in a population of subjects, or a sequence not characteristic of, or causally associated with, a disease or disorder); such a mutation may be referred to herein as a prior mutation (as distinguished from a mutation introduced by a method described herein). In some embodiments, the prior mutation is characteristic of, or causally associated with, a disease, e.g., a mutation that is known to cause a genetic disease, or is a mutation that is known to convey increased risk of a genetic disease. In some embodiments, a method described herein alters a genomic sequence comprising a mutation characteristic for and/or causally associated with a disease or disorder, changing the genomic sequence to a sequence that is not characteristic for and/or causally associated with that disease or disorder. In some embodiments, such alteration comprises correcting a prior mutation. In some embodiments, such alteration comprises introducing a silent mutation, a restriction site, or a tag sequence.

In some embodiments, a donor sequence differs from a sequence in the target DNA in one or more nucleotides, and integration of the donor sequence into the target DNA produces a genetic modification in the target DNA. In some embodiments, the donor sequence differs from a target DNA in a manner that integration of the donor sequence corrects a prior mutation in the target DNA, e.g., integration of the donor sequence into a target DNA comprising a prior mutation results in a modification of the mutation within the target DNA, e.g., in a modification of the target DNA sequence to the wild-type sequence, to the dominant sequence in a population of subjects, or, where the prior mutation is characteristic of, or causally associated with, a disease or disorder, in a modification to a sequence that is not characteristic of, or causally associated with a disease or disorder. In some embodiments, the prior mutation is characteristic for, or causally associated with, a disease or disorder. In some embodiments, the prior mutation is not characteristic for, or not causally associated with, a disease or disorder. In some such embodiments, a template polynucleotide comprising the donor sequence is referred to as a template for HDR of the mutation. In some embodiments, the donor sequence comprises a gene or a portion thereof, e.g., a gene, or portion thereof, that (e.g., prior to any genetic modification described herein) is mutated or non-functional in the target DNA or the genome of a cell. In some such embodiments, a template polynucleotide comprising the donor sequence is referred to as a template for HDR insertion of a gene, or portion thereof, in the target DNA.

In some embodiments, a template polynucleotide is single-stranded, e.g., a single-strand donor oligonucleotide (ssODN). In some embodiments, a template polynucleotide is double-stranded, e.g., a plasmid or a double-stranded donor oligonucleotide (dsODN). In some embodiments, a template polynucleotide is a minicircle plasmid. In some embodiments, a template polynucleotide is a nanoplasmid. Those of ordinary skill in the art will recognize that a minicircle plasmid is a plasmid that contains double stranded donor template without conventional plasmid backbones. Minicircle plasmids may be processed via a single DSB leading to linearization, whereas larger plasmids might require two DSBs which flank the template polynucleotide to excise the donor. Those of ordinary skill in the art will also recognize that a nanoplasmid comprises a circular DNA molecule of 500 base pairs or less and can be generated using services provided by Aldevron®. As such, minicircle plasmids and nanoplasmids are, in some embodiments, cut in a host cell prior to HDR via, for example, an exogenous nuclease (e.g., Cas9) targeting gRNA cut sites engineered into the plasmid sequence. However, in some embodiments, minicircle plasmids and nanoplasmids comprising a template polynucleotide need not be cut by an exogenous prior to HDR.

As used herein, a template polynucleotide refers to a nucleic acid that is a template for HDR, e.g., HDR of a mutation in the target DNA. In some embodiments, a template polynucleotide is approximately 10, 20, 30, 40, 50, 60, 70, 80, 90, 100, 110, 120, 130, 140, 150, 160, 170, 180, 190, or 200 nucleotides long+/−1, 2, 3, 4, 5, 6, 7, 8, 9, or 10 nucleotides long. In some embodiments, a template polynucleotide is approximately 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, or 3500 nucleotides+/−10, 25, 50, or 75 nucleotides long.

In some embodiments, the donor sequence comprises a modification as compared to the target DNA, for example, a mutation, e.g., an insertion, deletion, or substitution as compared to the target DNA nucleotide sequence. In some embodiments, the donor sequence comprises a substitution of a single nucleotide as compared to the target DNA. Such donor sequences are useful, for example, to effect genetic modifications that correct a single nucleotide mutation in a target DNA sequence that is characteristic for, or causally associated with, a disease or disorder. In some embodiments, the donor sequence comprises a substitution of two or more nucleotides as compared to the target DNA. Such donor sequences are useful, for example, to effect genetic modifications that correct more complex mutations, e.g., affecting two or more nucleotides, in a target DNA sequence that are characteristic for, or causally associated with, a disease or disorder. In some embodiments, the donor sequence comprises one or more insertions (e.g., of one or more nucleotides) as compared to the target DNA. Such donor sequences are useful, for example, to effect genetic modifications that create insertion mutations in a target DNA sequence. In some embodiments, the donor sequence comprises one or more deletions (e.g., of one or more nucleotides) as compared to the target DNA. Such donor sequences are useful, for example, to effect genetic modifications that create deletion mutations in a target DNA sequence. In some embodiments, the donor sequence comprises two or more substitutions as compared to the target DNA, wherein, if integrated into the target DNA, at least one such substitution results in the correction of a mutation that is characteristic of, or causally associated with, a disease or mutation, and wherein at least one such substitution results in a silent mutation in the target DNA, e.g., a substitution of a wobble base within an amino acid-encoding codon of a target DNA. Such donor sequences are useful, for example, to effect genetic modifications that correct disease-associated mutations in a target DNA sequence, while at the same time creating a sequence tag, e.g., a non-naturally occurring sequence or a sequence that did was not previously present in the target DNA, which is useful for identification and/or tracking of the modified cells. In some embodiments, the donor sequence comprises a restriction site or a unique sequence tag, for example, a unique primer binding site. In some embodiments, the sequence comprising the restriction site or a unique sequence tag is an insertion relative to the target DNA, e.g., the target DNA does not comprise a restriction site or a unique sequence tag where the donor sequence comprises one. In some embodiments, the sequence comprising the restriction site or a unique sequence tag is not an insertion relative to the target DNA. For example, in some embodiments, the sequence comprising the restriction site or a unique sequence tag comprises a mutation (e.g., a substitution) as compared to the target DNA that, upon integration into the target DNA, produces a restriction site or a unique sequence tag. In some embodiments, the sequence comprising the restriction site or a unique sequence tag does not alter an amino acid sequence encoded by the target DNA. A restriction site or a unique sequence tag thus introduced may be used, e.g., to confirm the success of integration of the donor sequence (e.g., in an experiment where the modified target DNA is cleaved and fragments or sequences thereof are analyzed). In some embodiments, the restriction endonuclease site comprises a Pvu1 site, e.g., 5′-CGATCG-3′.

In some embodiments, the target DNA comprises a prior mutation and the donor sequence differs from the target DNA in a manner such that integration of the donor sequence corrects the prior mutation and produces one or more additional mutations (e.g., a second, third, fourth, or fifth mutation relative to the correction of the prior mutation (the first mutation)). In some embodiments, the one or more additional mutations comprise one or more silent mutations that do not alter the amino acid encoded by the nucleic acid sequence of the target DNA. In some embodiments, the one or more silent mutations are contiguous (i.e., directly adjacent) to the prior mutation or the codon containing the prior mutation. In some embodiments, silent mutations are used, e.g., as identifiers (e.g., “tags” or “bar codes”) of a given correction of a prior mutation or to facilitate confirmation of integration of the donor sequence (e.g., in an experiment where the modified target DNA sequences are analyzed).

In some embodiments, methods and compositions provided by the present disclosure are applied to a target DNA, e.g., in order to modify the target DNA. For example, in some embodiments, the target DNA comprises a nucleotide sequence that is characteristic for, or causally associated with, a disease or disorder. Where such a target DNA sequence is different from the wild-type DNA sequence, or from a dominant DNA sequence at this locus within a population of subjects not affected by the disease or disorder, the divergence between the target DNA and the wild-type or dominant DNA sequence is also sometimes referred to herein as a prior mutation.

As used herein, a target DNA refers to any nucleic acid in which a break (e.g., a double-stranded break (DSB)) is targeted (e.g., by a CRISPR/Cas system). In some embodiments, a DSB in a target DNA can be repaired by HDR. In some embodiments, the target DNA is a genomic nucleic acid sequence, e.g., in a cell, e.g., in a subject, e.g., a human subject. In some embodiments, the target DNA comprises a gene or a portion thereof (e.g., a coding portion thereof, e.g., an exon). In some embodiments, the target DNA comprises a non-coding portion of a gene, e.g., an intron, a UTR, or a promotor region. In some embodiments, the target DNA comprises a regulatory region, e.g., an enhancer or inhibitor binding sequence. In some embodiments, the target DNA encodes a gene product (e.g., an mRNA and/or protein) characteristic of, or causally associated with, a disease or disorder. In some embodiments, the target DNA encodes a gene product (e.g., an mRNA and/or protein) that is not characteristic of, or causally associated with, a disease or disorder. In some embodiments, the target DNA does not comprise a coding sequence. In some embodiments, the target DNA comprises an intronic sequence. In some embodiments, the target DNA comprises an expression regulatory sequence, e.g., a promoter or an enhancer. In some embodiments, the target DNA comprises a splice site. In some embodiments, the target DNA comprises a heterochromatic sequence. In some embodiments, the target DNA comprises a repetitive sequence, e.g., a nucleotide expansion disease-associated repetitive sequence.

In some embodiments, producing a genetic modification using HDR comprises contacting cells with a template polynucleotide, a CRISPR/Cas system, and one or more other agents (e.g., one or more HDR-promoting agents or expansion agents), e.g., contacting cells with a genetic modification mixture described herein. The disclosure provides, in part, methods and compositions that achieve unexpectedly high editing efficiencies utilizing HDR. In some embodiments, efficiency of HDR-mediated editing and/or efficiency of total/overall editing (HDR- and non-HDR-mediated) is determined by a method described herein (e.g., in Example 2). In some embodiments, the efficiency of HDR is at least 20, 30, 40, 50, 60, 70, 80, 90, 95, or 99% (e.g., 50%, 60%, 70%, 80%, 90% or higher). In some embodiments, contacting cells to produce a genetic modification using HDR comprises contacting cells with one or more HDR-promoting agents as described herein. Without wishing to be bound by theory, the disclosure is directed, in part, to the discovery that the presence of one or more HDR-promoting agents may result in unexpectedly and advantageously high efficiency of HDR. Accordingly, methods describing contacting a cell herein also contemplate contacting a population of cells to produce a population of genetically modified cells, e.g., an editing efficiency, percent viability, and/or HDR efficiency described herein.

In some embodiments, producing a genetic modification using HDR comprises contacting a cell with a genetic modification mixture. As used herein, a genetic modification mixture refers to a mixture comprising a plurality of components that may be used to genetically modify a target DNA, e.g., in a cell. In some embodiments, a genetic modification mixture comprises one, two, three, or all of a CRISPR/Cas system, a template polynucleotide, one or more HDR-promoting agents, and one or more expansion agents. In some embodiments, a genetic modification mixture promotes HDR and HDR-mediated genetic modification (e.g., relative to another DNA repair pathway or genetic modifications utilizing another DNA repair pathway).

In some embodiments, contacting a cell with the genetic modification mixture comprises adding the genetic modification mixture directly to media comprising the cell. In some embodiments, contacting a cell with the genetic modification mixture comprises adding media comprising the genetic modification mixture to the cell or adding the cell to media comprising the genetic modification mixture. In some embodiments, the media is a growth media, e.g., a growth media suited to a hematopoietic cells (e.g., hematopoietic stem cells (HSCs)). Examples of growth media include, but are not limited to, a Stromal cell Growth Media (SCGM™, e.g. as available from Lonza Bioscience) or serum- and feeder-free media (SFFM). In some embodiments, contacting a cell with the genetic modification mixture comprises electroporating the genetic modification mixture or one or more components of the mixture into the cell. In some embodiments, contacting a cell with the genetic modification mixture comprises solvating the mixture in a lipid-permeable buffer, e.g., to serve as a carrier for movement of mixture components across the cell membrane. Examples of lipid-permeable buffers include, but are not limited to, DMSO and lipofectamine.

In some embodiments, the genetic modification mixture comprises a template polynucleotide, e.g., a single-strand donor oligonucleotide (ssODN), a double-stranded donor oligonucleotide (dsODN), a minicircle plasmid, or a nanoplasmid, comprising a donor sequence, a first flanking sequence and a second flanking sequence. In some embodiments, the genetic modification mixture comprises a dsODN comprising “capped” or “closed-ends” in order to minimize the chances that the NHEJ pathway is used in the genetic modification process. In some embodiments, the dsODN comprising “capped” or “closed-ends” is a GenWand® double-stranded DNA. In some embodiments, the genetic modification mixture comprises a CRISPR/Cas system capable of producing a break, e.g., a double-stranded break, at a target site in the genome of the cell. In some embodiments, the genetic modification mixture comprises one or more other agents (e.g., an expansion agent and/or HDR-promoting agent) that promote genetic modification. In some embodiments, the template polynucleotide, e.g., ssODN, dsODN, minicircle plasmid, or nanoplasmid, and the CRISPR/Cas system of the genetic modification mixture is mixed with the one or more other agents that promote genetic modification.

HDR may be induced by a DNA damage event that is capable of being mutagenic if left unrepaired or unprocessed, e.g., a double-stranded break. In some embodiments, the DNA damage event is induced by a CRISPR/Cas system, e.g., comprising a Cas nuclease, e.g., Cas9. Examples of DNA damage capable of producing a mutation include, but are not limited to, DNA alkylation, base deamination, base depurination, incidence of abasic sites, single-stranded breaks, and double-stranded breaks. Once DNA is damaged, the damage is repaired in multiple steps wherein cellular nucleases degrade nucleotide sequences at and proximal to the sites of the damage on one strand of the DNA. As used in this context, sequence “proximal” to the sites of damage is defined as a sequence that is found anywhere 10, 20, 30, 40, 50, 60, 70, 80, 90, 100, 150, 200, 250, 300, 350, 400, 450, or 500 nucleotides in the 5′ or 3′ direction of site of damage. Processing by nucleases, in turn, generates single-stranded overhangs comprised of a stretch of nucleotides that are not participating in base pairing interactions with nucleotides on the cognate strand to which the strand bearing the overhang is hybridized. Strand invasion follows, wherein the overhangs transiently base pair with a donor sequence that is located in close physical proximity to the damaged DNA molecule. In this way, template polynucleotide homology to a target site provided by the flanking sequences directs template polynucleotide participation in HDR. Strand invasion is followed by cellular polymerase-dependent recombination wherein the donor sequence serves as the template to direct the repair of the damaged DNA. Recombination between the donor sequence and the damaged DNA can incorporate the sequence of the donor sequence into the damaged DNA molecule. Following recombination, the repair is completed by a cellular ligase enzyme.

In some embodiments, a template polynucleotide comprises a first flanking sequence and a second flanking sequence, also referred to herein as a first homology sequence and a second homology sequence. In some embodiments, the first flanking sequence and second flanking sequence direct the binding of the template polynucleotide to a target DNA sequence in the cell. In some embodiments, a first flanking sequence is at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, or at least 250 nucleotides long (and optionally no more than 1000, no more than 750, no more than 500, no more than 400, no more than 300, or no more than 250 nucleotides long). In some embodiments, a first flanking sequence comprises 25-300, 50-300, 75-300, 100-300, 125-300, 150-300, 175-300, 200-300, 225-300, 250-300, 275-300, 100-400, 125-400, 150-400, 175-400, 200-400, 225-400, 250-400, 275-400, 300-400, 325, 400, 350-400, 375-400, 200-500, 225-500, 250-500, 275-500, 300-500, 325-500, 350-500, 375-500, 400-500, 425-500, 450-500, or 475-500 nucleotides in length. In some embodiments, a first flanking sequence comprises 500-2000, 600-2000, 700-2000, 800-2000, 900-2000, 1000-2000, 1100-2000, 1200-2000, 1300-2000, 1400-2000, 1500-2000, 1600-2000, 1700-2000, 1800-2000, or 1900-2000 nucleotides in length. In some embodiments, the first flanking sequence has at least 50%, at least 60%, at least 70%, at least at least 80%, at least 90%, at least 91%, at least 92%, at least 93%, at least 94%, at least 95%, at least 96%, at least 97%, at least 98%, at least 99%, or at least 100% identity to a sequence upstream of a DSB in the target DNA (e.g., upstream of a site where a DSB is produced by a CRISPR/Cas system described herein), or a sequence complementary thereto. In some embodiments, the first flanking sequence has 100% identity to a sequence upstream of a DSB in the target DNA (e.g., upstream of a site where a DSB is produced by a CRISPR/Cas system described herein), or a sequence complementary thereto. As used in this context, sequence “upstream” and “downstream” refer to a region within 10, within 20, within 30, within 40, within 50, within 60, within 70, within 80, within 90, or within 100 nucleotides of a feature in the DNA (e.g., a DSB), with each term referring to a different direction from the target site, and, in the case where the target DNA is a gene or portion thereof upstream is toward the transcription start site for the gene and downstream is away from the transcription start site for the gene. In some embodiments, the first flanking sequence is a 5′ homology arm of a template polynucleotide and is 5′ of a donor sequence, e.g., in an ssODN, dsODN, minicircle plasmid, or nanoplasmid. In some embodiments, a second flanking sequence is at least 50, at least 60, at least 70, at least 80, at least 90, at least 100, at least 110, at least 120, at least 130, at least 140, at least 150, at least 160, at least 170, at least 180, at least 190, at least 200, at least 210, at least 220, at least 230, at least 240, or at least 250 nucleotides long (and optionally no more than 1000, no more than 750, no more than 500, no more than 400, no more than 300, or no more than 250 nucleotides long). In some embodiments, a second flanking sequence comprises 25-300, 50-300, 75-300, 100-300, 125-300, 150-300, 175-300, 200-300, 225-300, 250-300, 275-300, 100-400, 125-400, 150-400, 175-400, 200-400, 225-400, 250-400, 275-400, 300-400, 325, 400, 350-400, 375-400, 200-500, 225-500, 250-500, 275-500, 300-500, 325-500, 350-500, 375-500, 400-500, 425-500, 450-500, or 475-500 nucleotides in length. In some embodiments, a second flanking sequence comprises 500-2000, 600-2000, 700-2000, 800-2000, 900-2000, 1000-2000, 1100-2000, 1200-2000, 1300-2000, 1400-2000, 1500-2000, 1600-2000, 1700-2000, 1800-2000, or 1900-2000 nucleotides in length. In some embodiments, the second flanking sequence has at least 50%, at least 60%, at least 70%, at least 80%, at least 90%, at least 91%, at least 92%, at least 93%, at least 94%, at least 95%, at least 96%, at least 97%, at least 98%, at least 99%, or at least 100% identity to a sequence downstream of a target site (e.g., downstream of a DSB produced by a CRISPR/Cas system in the target site), or a sequence complementary thereto. In some embodiments, the second flanking sequence has 100% identity to a sequence downstream of a DSB in the target DNA (e.g., downstream of a site where a DSB is produced by a CRISPR/Cas system described herein), or a sequence complementary thereto. In some embodiments, the second flanking sequence is a 3′ homology arm of a template polynucleotide and is 3′ of a donor sequence, e.g., in an ssODN, dsODN, minicircle plasmid, or nanoplasmid. In some embodiments, the first flanking sequence and the second flanking sequence have identity or complementarity to different sequences within or proximal to the target DNA. For example, in some embodiments the first flanking sequence has identity or complementarity to a first target sequence within or proximal to a target DNA and the second flanking sequence has identity or complementarity to a second target sequence within or proximal to the target DNA. In some embodiments, the first target sequence and second target sequence are no more than 5, no more than 10, no more than 20, no more than 30, no more than 40, no more than 50, no more than 100, no more than 150, no more than 200, no more than 250, no more than 300, no more than 500, or no more than 1000 bases apart in the nucleic acid molecule comprising the target DNA. In some embodiments, the first flanking sequence has 100% identity to a sequence upstream of a DSB in the target DNA, or a sequence complementary thereto, and the second flanking sequence has 100% identity to a sequence downstream of a DSB in the target DNA, or a sequence complementary thereto.

In some embodiments, a flanking sequence comprises 500-2000, 600-2000, 700-2000, 800-2000, 900-2000, 1000-2000, 1100-2000, 1200-2000, 1300-2000, 1400-2000, 1500-2000, 1600-2000, 1700-2000, 1800-2000, or 1900-2000 consecutive nucleotides that are 100% identical to a target sequence within a target DNA. In some embodiments, a second flanking sequence comprises 500-2000, 600-2000, 700-2000, 800-2000, 900-2000, 1000-2000, 1100-2000, 1200-2000, 1300-2000, 1400-2000, 1500-2000, 1600-2000, 1700-2000, 1800-2000, or 1900-2000 nucleotides in length. In some embodiments, a flanking sequence (e.g., a 3′ homology arm or 5′ homology arm) comprises 25-300, 50-300, 75-300, 100-300, 125-300, 150-300, 175-300, 200-300, 225-300, 250-300, 275-300, 100-400, 125-400, 150-400, 175-400, 200-400, 225-400, 250-400, 275-400, 300-400, 325, 400, 350-400, 375-400, 200-500, 225-500, 250-500, 275-500, 300-500, 325-500, 350-500, 375-500, 400-500, 425-500, 450-500, or 475-500 consecutive nucleotides that are 100% identical to a target sequence within a target DNA. In some embodiments, a flanking sequence (e.g., a 3′ homology arm or 5′ homology arm) comprises 2-100, 10-100, 20-100, 30-100, 40-100, 50-100, 60-100, 70-100, 80-100, 90-100, 2-150, 2-200, 2-250, 10-150, 10-200, 10-250, 50-150, 50-200, 50-250, 100-150, 100-200, 100-250, 150-200, 150-200, or 200-250 consecutive nucleotides that are 100% identical to a target sequence within a target DNA. In some embodiments, a flanking sequence (e.g., a 3′ homology arm or 5′ homology arm) comprises at least 2, at least 10, at least 20, at least 30, at least 40, at least 50, at least 60, at least 70, at least 80, at least 90, or at least 100 consecutive nucleotides that are 100% identical to a target sequence within a target DNA (and optionally no more than 200, no more than 180, no more than 160, no more than 140, no more than 120, or no more than 100 consecutive nucleotides that are 100% identical to a target sequence within a target DNA). In some embodiments, a flanking sequence (e.g., a 3′ homology arm or a 5′ homology arm) comprises a nucleotide sequence that is 100% identical to a PAM sequence in the target DNA. In some embodiments, the nucleotide sequence identical to the PAM sequence is 2-3, 2-4, 2-5, 2-6, 3-4, 3-5, 3-6, 4-5, 4-6, or 5-6 nucleotides in length (e.g., 2, 3, 4, 5, or 6 nucleotides in length).

In some embodiments, a template polynucleotide comprises a donor sequence. In some embodiments, the donor sequence is integrated into a target DNA at the site of a DSB. In some embodiments, the donor sequence is homologous to the target DNA or a portion thereof, e.g., the sequence of the target DNA surrounding or adjacent to the DSB. In some embodiments, the donor sequence is contiguous with the first and second flanking sequences in a template polynucleotide. For example, in some embodiments a target DNA comprises a gene or a portion thereof, and the donor sequence is homologous to the target DNA or a portion thereof (e.g., in proximity to a DSB or a site targeted for a DSB by a CRISPR/Cas system as described herein). In some embodiments, the first and second flanking sequences guide binding of the template polynucleotide to a target DNA, facilitating interaction of the donor sequence with its homologous sequence in the target DNA and/or with cellular DNA repair (e.g., HDR) pathway components. In some embodiments, the donor sequence differs from a homologous sequence of the target DNA at 1, 2, 3, 4, 5, 6, 7, 8, 9, or 10 bases (e.g., 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 bases), or at a number of positions corresponding to up to 1, 5, 10, 15, or 20% of the length of the donor sequence. In some embodiments, a donor sequence is 1-100, 1-80, 1-60, 1-40, 1-20, 1-15, 1-10, 1-9, 1-8, 1-7, 1-6, 1-5, 1-4, 1-3, 1-2, 5-100, 5-80, 5-60, 5-40, 5-20, 5-15, 5-10, 5-9, 5-8, 5-7, 5-6, 10-100, 10-80, 10-60, 10-40, 10-20, 10-15, 20-100, 20-80, 20-60, 20-40, 60-100, or 60-80 nucleotides in length. In some embodiments, a donor sequence is no more than 100, no more than 90, no more than 80, no more than 70, no more than 60, no more than 50, no more than 45, no more than 40, no more than 35, no more than 30, no more than 25, no more than 20, no more than 15, no more than 14, no more than 13, no more than 12, no more than 11, no more than 10, no more than 9, no more than 8, no more than 7, no more than 6, no more than 5, no more than 4, no more than 3, no more than 2, or no more than 1 bases long. In some embodiments, a donor sequence is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 bases long. In some embodiments, a donor sequence differs from a homologous sequence of the target DNA at a position or positions corresponding to a prior mutation in the target DNA (e.g., characteristic of, or causally associated with, a disease or disorder, or risk of developing a disease or disorder), e.g., a prior point mutation. In some embodiments, the donor sequence comprises sequence corresponding to the wild-type, functional, and/or naturally-occurring sequence at a position or positions corresponding to a prior mutation in the target DNA. In some embodiments, the donor sequence comprises an artificial or heterologous sequence. In some embodiments, a donor sequence is 200-2000, 200-1900, 200-1800, 200-1700, 200-1600, 200-1500, 200-1400, 200-1300, 200-1200, 200-1100, 100-1000, 100-900, 100-800, 100-700, 100-600, 100-500, 100-400, 100-300, or 100-200 nucleotides in length. In some embodiments, a donor sequence is no more than 2000, no more than 1900, no more than 1800, no more than 1700, no more than 1600, no more than 1500, no more than 1400, no more than 1300, no more than 1200, no more than 1100, no more than 1000, no more than 900, no more than 800, no more than 700, no more than 600, no more than 500, no more than 400, no more than 300, or no more than 200 nucleotides in length.

A schematic of an exemplary template polynucleotide, such as an ssODN, a double-stranded dsODN, minicircle plasmid, or nanoplasmid is provided below:
[5′-homology arm]-[donor sequence]-[3′ homology arm]
   Each homology arm (e.g., a flanking sequence described herein) has homology to a sequence in the target DNA proximal to the sequence homologous to the donor sequence.

In some embodiments, a homology arm comprises a sequence homologous to a PAM sequence in the target DNA. In some embodiments, a CRISPR/Cas system for use in a method of the disclosure comprises a Cas nuclease that recognizes a PAM sequence in the target DNA and cuts the target DNA at a position near to the PAM sequence (e.g., 5′ or 3′ of the PAM sequence). Accordingly, in some embodiments a PAM homologous sequence is present in a 3′ homology arm or a 5′ homology arm of a template polynucleotide. In some embodiments, the PAM homologous sequence is positioned such that HDR of a DSB produced by a Cas nuclease promotes integration of a donor sequence. In some embodiments, the DSB is positioned in a target DNA sequence homologous to the donor sequence.

A schematic of an exemplary 3′ homology arm (e.g., where a CRISPR/Cas system (e.g., comprising Cas9) cuts a target DNA 5′ of a PAM sequence) is provided below:
[N]ₓ-[PAM]-[N]_{y}
   For example, an exemplary Cas nuclease, Cas9, cuts a target DNA 3-4 nucleotides 5′ of a PAM sequence. In some embodiments, x is 3-4, and y is the number of nucleotides in the remaining length of the homology arm (e.g., wherein the length of the homology arm is described herein). For example, for x=3, and a homology arm length of 100 nucleotides, y would be 100 minus 3 and minus the length of the PAM homologous sequence (e.g., where the PAM sequence is 3 nucleotides long, y would be 94 (100-3-3). In some embodiments, x is 2 and the homology arm is 50-60 nucleotides long. In some embodiments, x is 2 and the homology arm is 60-70 nucleotides long. In some embodiments, x is 2 and the homology arm is 70-80 nucleotides long. In some embodiments, x is 2 and the homology arm is 80-90 nucleotides long. In some embodiments, x is 2 and the homology arm is 90-100 nucleotides long. In some embodiments, x is 2 and the homology arm is 100-110 nucleotides long. In some embodiments, x is 2 and the homology arm is 110-120 nucleotides long. In some embodiments, x is 2 and the homology arm is 120-130 nucleotides long. In some embodiments, x is 2 and the homology arm is 130-140 nucleotides long. In some embodiments, x is 2 and the homology arm is 140-150 nucleotides long. In some embodiments, x is 2 and the homology arm is 150-160 nucleotides long. In some embodiments, x is 2 and the homology arm is 160-170 nucleotides long. In some embodiments, x is 2 and the homology arm is 170-180 nucleotides long. In some embodiments, x is 2 and the homology arm is 180-190 nucleotides long. In some embodiments, x is 2 and the homology arm is 190-200 nucleotides long. In some embodiments, x is 2 and the homology arm is 210-220 nucleotides long. In some embodiments, x is 2 and the homology arm is 220-230 nucleotides long. In some embodiments, x is 2 and the homology arm is 230-240 nucleotides long. In some embodiments, x is 2 and the homology arm is 240-250 nucleotides long. In some embodiments, x is 3 and the homology arm is 50-60 nucleotides long. In some embodiments, x is 3 and the homology arm is 60-70 nucleotides long. In some embodiments, x is 3 and the homology arm is 70-80 nucleotides long. In some embodiments, x is 3 and the homology arm is 80-90 nucleotides long. In some embodiments, x is 3 and the homology arm is 90-100 nucleotides long. In some embodiments, x is 3 and the homology arm is 100-110 nucleotides long. In some embodiments, x is 3 and the homology arm is 110-120 nucleotides long. In some embodiments, x is 3 and the homology arm is 120-130 nucleotides long. In some embodiments, x is 3 and the homology arm is 130-140 nucleotides long. In some embodiments, x is 3 and the homology arm is 140-150 nucleotides long. In some embodiments, x is 3 and the homology arm is 150-160 nucleotides long. In some embodiments, x is 3 and the homology arm is 160-170 nucleotides long. In some embodiments, x is 3 and the homology arm is 170-180 nucleotides long. In some embodiments, x is 3 and the homology arm is 180-190 nucleotides long. In some embodiments, x is 3 and the homology arm is 190-200 nucleotides long. In some embodiments, x is 3 and the homology arm is 210-220 nucleotides long. In some embodiments, x is 3 and the homology arm is 220-230 nucleotides long. In some embodiments, x is 3 and the homology arm is 230-240 nucleotides long. In some embodiments, x is 3 and the homology arm is 240-250 nucleotides long. In some embodiments, x is 4 and the homology arm is 50-60 nucleotides long. In some embodiments, x is 4 and the homology arm is 60-70 nucleotides long. In some embodiments, x is 4 and the homology arm is 70-80 nucleotides long. In some embodiments, x is 4 and the homology arm is 80-90 nucleotides long. In some embodiments, x is 4 and the homology arm is 90-100 nucleotides long. In some embodiments, x is 4 and the homology arm is 100-110 nucleotides long. In some embodiments, x is 4 and the homology arm is 110-120 nucleotides long. In some embodiments, x is 4 and the homology arm is 120-130 nucleotides long. In some embodiments, x is 4 and the homology arm is 130-140 nucleotides long. In some embodiments, x is 4 and the homology arm is 140-150 nucleotides long. In some embodiments, x is 4 and the homology arm is 150-160 nucleotides long. In some embodiments, x is 4 and the homology arm is 160-170 nucleotides long. In some embodiments, x is 4 and the homology arm is 170-180 nucleotides long. In some embodiments, x is 4 and the homology arm is 180-190 nucleotides long. In some embodiments, x is 4 and the homology arm is 190-200 nucleotides long. In some embodiments, x is 4 and the homology arm is 210-220 nucleotides long. In some embodiments, x is 4 and the homology arm is 220-230 nucleotides long. In some embodiments, x is 4 and the homology arm is 230-240 nucleotides long. In some embodiments, x is 4 and the homology arm is 240-250 nucleotides long.

A schematic of an exemplary 5′ homology arm (e.g., where a CRISPR/Cas system (e.g., comprising Cas12a) cuts a target DNA 3′ of a PAM sequence) is provided below:
[N]ₐ-[PAM]-[N]_{b}
   As a further example, another exemplary Cas nuclease, Cas12a, cuts a target DNA 18-19 nucleotides 3′ of a PAM sequence. In some embodiments, b is 18-19, and a is the number of nucleotides in the remaining length of the homology arm (e.g., wherein the length of the homology arm is described herein). For example, for b=18, and a homology arm length of 100 nucleotides, a would be 100 minus 18 and minus the length of the PAM homologous sequence (e.g., where the PAM sequence is 3 nucleotides long, a would be 79 (100-18-3). In some embodiments, b is 17 and the homology arm is 50-60 nucleotides long. In some embodiments, b is 17 and the homology arm is 60-70 nucleotides long. In some embodiments, b is 17 and the homology arm is 70-80 nucleotides long. In some embodiments, b is 17 and the homology arm is 80-90 nucleotides long. In some embodiments, b is 17 and the homology arm is 90-100 nucleotides long. In some embodiments, b is 17 and the homology arm is 100-110 nucleotides long. In some embodiments, b is 17 and the homology arm is 110-120 nucleotides long. In some embodiments, b is 17 and the homology arm is 120-130 nucleotides long. In some embodiments, b is 17 and the homology arm is 130-140 nucleotides long. In some embodiments, b is 17 and the homology arm is 140-150 nucleotides long. In some embodiments, b is 17 and the homology arm is 150-160 nucleotides long. In some embodiments, b is 17 and the homology arm is 160-170 nucleotides long. In some embodiments, b is 17 and the homology arm is 170-180 nucleotides long. In some embodiments, b is 17 and the homology arm is 180-190 nucleotides long. In some embodiments, b is 17 and the homology arm is 190-200 nucleotides long. In some embodiments, b is 17 and the homology arm is 210-220 nucleotides long. In some embodiments, b is 17 and the homology arm is 220-230 nucleotides long. In some embodiments, b is 17 and the homology arm is 230-240 nucleotides long. In some embodiments, b is 17 and the homology arm is 240-250 nucleotides long. In some embodiments, b is 18 and the homology arm is 50-60 nucleotides long. In some embodiments, b is 18 and the homology arm is 60-70 nucleotides long. In some embodiments, b is 18 and the homology arm is 70-80 nucleotides long. In some embodiments, b is 18 and the homology arm is 80-90 nucleotides long. In some embodiments, b is 18 and the homology arm is 90-100 nucleotides long. In some embodiments, b is 18 and the homology arm is 100-110 nucleotides long. In some embodiments, b is 18 and the homology arm is 110-120 nucleotides long. In some embodiments, b is 18 and the homology arm is 120-130 nucleotides long. In some embodiments, b is 18 and the homology arm is 130-140 nucleotides long. In some embodiments, b is 18 and the homology arm is 140-150 nucleotides long. In some embodiments, x is 3 and the homology arm is 150-160 nucleotides long. In some embodiments, b is 18 and the homology arm is 160-170 nucleotides long. In some embodiments, b is 18 and the homology arm is 170-180 nucleotides long. In some embodiments, b is 18 and the homology arm is 180-190 nucleotides long. In some embodiments, b is 18 and the homology arm is 190-200 nucleotides long. In some embodiments, b is 18 and the homology arm is 210-220 nucleotides long. In some embodiments, b is 18 and the homology arm is 220-230 nucleotides long. In some embodiments, b is 18 and the homology arm is 230-240 nucleotides long. In some embodiments, b is 18 and the homology arm is 240-250 nucleotides long. In some embodiments, b is 19 and the homology arm is 50-60 nucleotides long. In some embodiments, b is 19 and the homology arm is 60-70 nucleotides long. In some embodiments, b is 19 and the homology arm is 70-80 nucleotides long. In some embodiments, b is 19 and the homology arm is 80-90 nucleotides long. In some embodiments, b is 19 and the homology arm is 90-100 nucleotides long. In some embodiments, b is 19 and the homology arm is 100-110 nucleotides long. In some embodiments, b is 19 and the homology arm is 110-120 nucleotides long. In some embodiments, b is 19 and the homology arm is 120-130 nucleotides long. In some embodiments, b is 19 and the homology arm is 130-140 nucleotides long. In some embodiments, b is 19 and the homology arm is 140-150 nucleotides long. In some embodiments, b is 19 and the homology arm is 150-160 nucleotides long. In some embodiments, b is 19 and the homology arm is 160-170 nucleotides long. In some embodiments, b is 19 and the homology arm is 170-180 nucleotides long. In some embodiments, b is 19 and the homology arm is 180-190 nucleotides long. In some embodiments, b is 19 and the homology arm is 190-200 nucleotides long. In some embodiments, b is 19 and the homology arm is 210-220 nucleotides long. In some embodiments, b is 19 and the homology arm is 220-230 nucleotides long. In some embodiments, b is 19 and the homology arm is 230-240 nucleotides long. In some embodiments, b is 19 and the homology arm is 240-250 nucleotides long.

In some embodiments, the first and second flanking sequence of the template polynucleotide (e.g., ssODN, dsODN, minicircle plasmid, or nanoplasmid) comprise sequences complementarity to a first and second portion of the glucosylceramidase beta (GBA) gene. In some embodiments, the first portion of the GBA gene comprises a portion of exon 9 or a sequence proximal to exon 9 wherein “proximal is defined as a sequence that is found anywhere 10, 20, 30, 40, 50, 60, 70, 80, 90, or 100 nucleotides in the 5′ or 3′ direction of exon 9 of the GBA gene. In some embodiments, the second portion of the GBA gene comprises a portion of exon 9 or a sequence proximal to exon 9. In some embodiments, the first portion of the GBA gene comprises a portion of exon 10 or a sequence proximal to exon 10 wherein “proximal” is defined as a sequence that is found anywhere 10, 20, 30, 40, 50, 60, 70, 80, 90, or 100 nucleotides in the 5′ or 3′ direction of exon 10 of the GBA gene. In some embodiments, the second portion of the GBA gene comprises a portion of exon 10 or a sequence proximal to exon 10. In some embodiments, the first flanking sequence of the ssODN comprises a flanking sequence set forth in any of SEQ ID NO: 25-30. In some embodiments, the second flanking sequence of the ssODN comprises a flanking sequence set forth in any of of SEQ ID NOs: 25-30.

In some embodiments, the donor sequence of the template polynucleotide (e.g., ssODN, dsODN, minicircle plasmid, or nanoplasmid) comprises a homologous sequence to the sequence encoding N409 or L483 in a wildtype GBA gene as set forth in the nucleotide sequence provided in GenBank: NG_009783.1 or as set forth in the amino acid sequence provided in GenBank: AAC51820.1, or the sequence of a corresponding amino acid position in a homologous GBA gene. In some embodiments, the donor sequence of the template polynucleotide, e.g., ssODN, dsODN, minicircle plasmid, or nanoplasmid, comprises a sequence homologous to the codon encoding N409 in the wildtype GBA gene, or a corresponding position in a homologous GBA gene, and encodes an asparagine at said position. In some embodiments, the donor sequence of the template polynucleotide, e.g., ssODN, dsODN, minicircle plasmid, or nanoplasmid, comprises a sequence homologous to the codon encoding L483 in the wildtype GBA gene, or a corresponding position in a homologous GBA gene, and encodes a leucine at said position. In some embodiments, the donor sequence of the template polynucleotide, e.g., ssODN, dsODN, minicircle plasmid, or nanoplasmid, comprises a donor sequence set forth in any one of SEQ ID NOs: 25-30. For example, a template polynucleotide comprising the sequence of any one of SEQ ID NOs: 25-30 can be used, for example, to genetically engineer a cell (e.g., a hematopoietic cell) to comprise a mutation characteristic of, or causally associated with, a disease or disorder, or characteristic of a risk of developing a disease or disorder to produce a genetically engineered cell useful, e.g., as a model for the disease or disorder. Such a model could also be used to test additional template polynucleotides comprising donor sequences designed to correct the disease-associated mutation, and methods and compositions using said template polynucleotides. For example, a template polynucleotide comprising the sequence of any one of SEQ ID NOs: 89-90 can be used, for example, to genetically engineer a cell (e.g., a hematopoietic cell) to comprise a nucleotide that corrects a mutation (i.e., reverts the position to encode the nucleotide found in the wild-type version of the gene) that previously existed at that position. In some embodiments, a template polynucleotide comprising the sequence of any one of SEQ ID NOs: 89-90 can be used, for example, to correct a mutation in a GBA gene, wherein the mutation encodes a serine at amino acid position 409 instead of an asparagine or a proline at amino acid position 483 instead of a leucine.

In some embodiments, the donor sequence comprises a heterologous or exogenous gene sequence that does not naturally occur in the modified cell. In some embodiments, the donor sequence encodes a heterologous or exogenous gene sequence that disrupts an endogenous gene in the target cell resulting in knockout of that endogenous gene. In some embodiments, the donor sequence comprises a chimeric antigen receptor (CAR) that disrupts an endogenous gene in the target cell resulting in knockout of that endogenous gene. In some embodiments, the donor sequence comprises a CAR capable of binding to CD33. In some embodiments, the CAR comprises an antigen binding domain specific for CD33, a transmembrane domain, and an intracellular T cell signaling domain. In some embodiments, the transmembrane domain is a CDS transmembrane domain. In some embodiments, the antigen-binding domain of the CAR capable of binding to CD33 comprises a light chain variable region and/or a heavy chain variable region. In some embodiments, the heavy chain variable region comprises a CDR1 region, a CDR2 region, and a CDR3 region. In some embodiments, the light chain variable region of the anti-CD33 antigen binding domain may comprise a light chain CDR1 region, a light chain CDR2 region, and a light chain CDR3. In some embodiments, the anti-CD33 antigen binding domain may comprise any antigen binding portion of an anti-CD33 antibody. The antigen binding portion can be any portion that has at least one antigen binding site, such as Fab, F(ab′)2, dsFv, scFv, diabodies, and triabodies. In some embodiments, the antigen binding portion is a single-chain variable region fragment (scFv) antibody fragment. An scFv is a truncated Fab fragment including the variable (V) domain of an antibody heavy chain linked to a V domain of a light antibody chain via a synthetic peptide linker. In some embodiments, the light chain variable region and the heavy chain variable region of the anti-CD33 antigen binding domain can be joined to each other by a linker. In some embodiments, the antigen binding domain comprises one or more leader sequences (signal peptides). In some embodiments, the CAR construct comprises a hinge domain. In some embodiments, the hinge domain is a CDS hinge domain. In some embodiments, the CDS hinge domain is human. In some embodiments the CAR construct comprises an intracellular T cell signaling domain. In some embodiments, the intracellular T cell signaling domain comprises a 4-IBB intracellular T cell signaling sequence. In some embodiments, the intracellular T cell signaling domain comprises a CD3 zeta(s) intracellular T cell signaling sequence. Non-limiting examples of CD33-targeted CARs that may be used as donor sequences as described herein may be found in, for example, PCT/US2019/022309.

Recombinant Adeno-Associated Viruses (rAAVs)

Some aspects of the present disclosure relate to recombinant adeno-associated viruses (rAAVs) comprising template polynucleotides and genetic modification mixtures thereof. rAAV vectors typically comprise, at a minimum, a transgene including its regulatory sequences, and 5′ and 3′ AAV inverted terminal repeats (ITRs). In some embodiments, the 5′ and 3′ ITRs may be alternatively referred to as “first” and “second” ITRs, respectively. The rAAVs of the present disclosure may comprise a transgene comprising template polynucleotide in addition to expression control sequences (e.g., a promoter, an enhancer, a poly(A) signal, etc.), as described elsewhere in this disclosure.

In some embodiments, the rAAV vectors comprising a template polynucleotide of the present disclosure comprise at least, in order from 5′ to 3′, a first adeno-associated virus (AAV) inverted terminal repeat (ITR) sequence, a promoter operably linked to the sequence of the template polynucleotide, a polyadenylation signal, and a second AAV inverted terminal repeat (ITR) sequence. In some embodiments, the transgene comprising a template polynucleotide of the present disclosure comprises at least, in order from 5′ to 3′, a first adeno-associated virus (AAV) inverted terminal repeat (ITR) sequence, a first flanking sequence, an SFFV promoter, a Kozack sequence operably linked to a donor sequence, a beta-globin poly(A) signal, a second flanking sequence, and a second AAV ITR. In some embodiments, the transgene comprising a template polynucleotide of the present disclosure comprises the nucleic acid sequence set forth in SEQ ID NO: 120.

In some embodiments, the rAAV vector genome comprising a template polynucleotide is circular. In some embodiments, the rAAV vector genome comprising a template polynucleotide is linear. In some embodiments, the rAAV vector genome comprising a template polynucleotide is single-stranded. In some embodiments, the rAAV vector genome comprising a template polynucleotide is double-stranded. In some embodiments, the rAAV genome vector comprising a template polynucleotide is a self-complementary rAAV vector.

Inverted terminal repeat (ITR) sequences are about 145 bp in length. While the entire sequences encoding the ITRs are commonly used in engineering rAAVs, some degree of minor modification of these sequences is permissible. The ability to modify these ITR sequences is within the capabilities of one of ordinary skill in the pertinent the art. (See, e.g., texts such as Sambrook et al., Molecular Cloning. A Laboratory Manual, 2d ed., Cold Spring Harbor Laboratory, New York (1989); and K. Fisher et al., J Virol., 70:520 532 (1996)).

The rAAV particles comprising a template polynucleotide or particles within an rAAV preparation comprising a template polynucleotide disclosed herein, may be of any AAV serotype, including any derivative or pseudotype (e.g., 1, 2, 3, 4, 5, 6, 7, 8, 9, 10, 2/1, ⅖, 2/8, 2/9, 3/1, ⅗, ⅜, or 3/9). As used herein, the serotype of an rAAV refers to the serotype of the capsid proteins of the recombinant virus. Non-limiting examples of derivatives, pseudotypes, and/or other vector types include, but are not limited to, AAVrh.10, AAVrh.74, AAV2/1, AAV2/5, AAV2/6, AAV2/8, AAV2/9, AAV2-AAV3 hybrid, AAVhu.14, AAV3a/3b, AAVrh32.33, AAV-HSC15, AAV-HSC17, AAVhu.37, AAVrh.8, CHt-P6, AAV2.5, AAV6.2, AAV218, AAV-HSC15/17, AAVM41, AAV9.45, AAV6 (Y445F/Y731F), AAV2.5T, AAV-HAE1/2, AAV clone 32/83, AAVShHIO, AAV2 (Y->F), AAV8 (Y733F), AAV2.15, AAV2.4, AAVM41, and AAVr3.45. Such AAV serotypes and derivatives/pseudotypes, and methods of producing such derivatives/pseudotypes are known in the art (see, e.g., Mol. Ther. 2012 April; 20 (4): 699-708. doi: 10.1038/mt.2011.287. Epub 2012 Jan. 24. The AAV vector toolkit: poised at the clinical crossroads. Asokan A I, Schaffer D V, Samulski R J.). Methods for producing and using pseudotyped rAAV vectors are known in the art (see, e.g., Duan et al, J. Virol., 75:7662-7671, 2001; Halbert et al, J. Virol., 74:1524-1532, 2000; Zolotukhin et al, Methods, 28:158-167, 2002; and Auricchio et al., Hum. Molec. Genet., 10:3075-3081, 2001).

The components to be cultured in the host cell (e.g., 293T cell) to package a rAAV vector comprising a template polynucleotide in an AAV capsid may be provided to the host cell (e.g., 293T cell) in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Such a stable host cell will contain the required component(s) under the control of either an inducible promoter, tissue-specific, or a constitutive promoter. In some embodiments, the rAAV genome comprises a vector. In some embodiments, the rAAV genome comprises a plasmid. In some embodiments, the plasmid is pAV1.

The recombinant AAV vector comprising a template polynucleotide, rep sequences, cap sequences, and helper functions required for producing the rAAV comprising a template polynucleotide of this disclosure may be delivered to the packaging host cell (e.g., 293T cell) using any appropriate genetic element (e.g., a vector). The selected genetic element may be delivered by any suitable method including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions are well known and the selection of a suitable method is not a limitation on this disclosure. See, e.g., K. Fisher et al., J. Virol., 70:520-532 (1993) and U.S. Pat. No. 5,478,745.

In some embodiments, recombinant AAVs comprising a template polynucleotide may be produced using the triple transfection method (described in detail in U.S. Pat. No. 6,001,650). Typically, the recombinant AAVs are produced by transfecting a host cell (e.g., 293T cell) with an AAV vector (comprising a transgene flanked by ITR elements) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses, such as adenovirus, herpes virus (other than herpes simplex virus type-1), and vaccinia virus.

Purified rAAVs comprising a polynucleotide template and compositions thereof may be administered to a cell or subject to promote editing via a variety of methods known in the art. For instance, administration of an rAAV comprising a polynucleotide template to an isolated cell may be performed through electroporation or transfection. In other instances, administration of an rAAV comprising a polynucleotide template to a subject may be performed through infusion subcutaneously, intraocularly, intravitreally, parenterally, subcutaneously, intravenously, intracerebro-ventricularly, intramuscularly, intracranially, intrathecally, orally, intraperitoneally, or by oral or nasal inhalation, or by direct injection to one or more cells, tissues, or organs. rAAVs comprising a polynucleotide template described herein may be suitably formulated in composition comprising, for example, adjuvants such as preservatives, wetting agents, emulsifying agents, dispersing agents, pharmaceutically acceptable excipients, a liposome, a lipid, a lipid complex, a lipid nanoparticle, a microsphere, a microparticle, a nanosphere, a nanoparticle, or any combination thereof or in order to be properly formulated for administration to the cells, tissues, organs, or body of a subject in need thereof. Those of skill in the art will recognize doses/amounts of rAAVs suitable for administration to cells or subjects. Accordingly, those of ordinary skill in the art will recognize that doses/amounts of rAAVs for administration may be measured in units of multiplicity of infection (MOIs) (e.g., MOI=5, 10, 100) or vector genomes/kilogram of body weight (e.g., 1×10¹³ vg/kg, 5×10¹³ vg/kg, and 1×10¹⁴ vg/kg).

### Safe Harbor Loci

In some embodiments, a template polynucleotide directs insertion of a donor sequence into a non-homologous target DNA. In some embodiments, a template polynucleotide comprises a first flanking sequence, a second flanking sequence, and a donor sequence, wherein the first and second flanking sequences specify binding to a target DNA that is not homologous to the donor sequence. In some embodiments, the non-homologous target DNA is a safe harbor locus. Safe harbor loci are known in the art, and refer to sites in the genomic DNA of a cell (e.g., an HSC) in which mutations, e.g., insertion mutations, results in an approximately neutral biological outcome. A person of skill in the art can readily understand that what qualifies as an approximately neutral biological outcome may vary between cell types and the purpose of the genetically modified cell. In some embodiments, a safe harbor locus is a site where insertion does not decrease viability of the cell or disrupt the function or structure of a protein (e.g., an essential protein). In some embodiments, a safe harbor locus is a site where an inserted nucleic acid sequence is expressed at a detectable level in a cell (e.g., is not silenced within heterochromatin).

The disclosure is directed, in part, to methods of genetically modifying HSCs for the purpose of transplanting the modified HSCs into a subject. In some embodiments, a safe harbor loci in an HSC is a loci at which a mutation, e.g., an insertion, does not lead to any detrimental effects in the HSC that would impair the therapeutic effect of the HSC after administration to a subject. In some embodiments, a detrimental effect comprises one or more of a decrease in viability, a change (e.g., decrease) in growth rate or capacity to grow/divide, a change (e.g., decrease) in differentiation capacity or the distribution of lineages produced by cells descended from the HSC, or an alteration in cell surface protein expression (e.g., resulting in altered immune system reactivity to the HSC).

In some embodiments, a donor sequence for use in a template polynucleotide directing insertion of the donor sequence at a non-homologous (e.g., safe harbor) target DNA comprises a gene or a portion of a gene. In some embodiments, the donor sequence is greater than 100, 200, 300, 400, 500, 600, 700, 800, 900, 1000, 1500, 2000, 2500, 3000, 4000, or 5000 bases long. In other embodiments, the donor sequence is no more than 100 bases long (e.g., as described elsewhere herein).

In some embodiments, the safe harbor locus is in the C—C Motif Chemokine Receptor 5 (CCR5) gene. CCR5 is a protein that binds to chemokines. In exemplary embodiments, the genomic sequence of CCR5 is the sequence provided in GenBank: NG_012637.1. CCR5 consists of seven transmembrane domains and is expressed in various cell populations including macrophages, dendritic cells, memory cells in the immune system, endothelium, epithelium, vascular smooth muscle cells, fibroblasts, microglia, neurons, and astrocytes. In some embodiments, the template polynucleotide, e.g., ssODN dsODN, minicircle plasmid, or nanoplasmid, comprises a first flanking sequence complementary to a first portion of a CCR5 gene, a donor sequence, and a second flanking sequence complementary to a second portion of the CCR5 gene. In some embodiments, the template polynucleotide, e.g., ssODN, dsODN, minicircle plasmid, or nanoplasmid, comprises a first flanking sequence that is complementary to a sequence proximal to a sequence found in a CCR5 gene. In some embodiments, the template polynucleotide, e.g., ssODN, dsODN, minicircle plasmid, or nanoplasmid, comprises a second flanking sequence that is complementary to a sequence proximal to a second sequence found in a CCR5 gene. In some embodiments, the template polynucleotide, e.g., ssODN, dsODN, minicircle plasmid, or nanoplasmid, comprises a first flanking sequence that is complementary to a portion of a CCR5 gene, and a second flanking sequence that is complementary to a sequence proximal to a sequence found in a CCR5 gene. As used in this context, sequence “proximal” to a CCR5 gene is defined as a sequence that is found anywhere 10, 20, 30, 40, 50, 60, 70, 80, 90, or 100 nucleotides in the 5′ or 3′ direction of a CCR5 gene. In some embodiments, the first and second portions of a CCR5 gene are not identical sequences. Exemplary portions of a CCR5 gene include, but are not limited to, reverse complementary sequences to the flanking sequences set forth in any one of SEQ ID NOs: 43-46. In some embodiments, the first and/or second flanking sequences are chosen from flanking sequences set forth in any one of SEQ ID NOs: 43-46.

In some embodiments, the safe harbor locus is in the Adeno-Associated Virus Integration Site 1 (AAVS1) gene. AAVS1 is a genomic site in humans where adeno-associated virus (parvovirus) integrates. In exemplary embodiments, the genomic sequence of AAVS1 is the sequence provided at genomic location 19q13. In some embodiments, the template polynucleotide, e.g., ssODN, dsODN, minicircle plasmid, or nanoplasmid, comprises a first flanking sequence complementary to a first portion of a AAVS1 gene, a donor sequence, and a second flanking sequence complementary to a second portion of the AAVS1 gene. In some embodiments, the template polynucleotide, e.g., ssODN, dsODN, minicircle plasmid, or nanoplasmid, comprises a first flanking sequence that is complementary to a sequence proximal to a sequence found in a AAVS1 gene. In some embodiments, the template polynucleotide, e.g., ssODN, dsODN, minicircle plasmid, or nanoplasmid, comprises a second flanking sequence that is complementary to a sequence proximal to a second sequence found in a AAVS1 gene. In some embodiments, the template polynucleotide, e.g., ssODN, dsODN, minicircle plasmid, or nanoplasmid, comprises a first flanking sequence that is complementary to a portion of a AAVS1 gene, and a second flanking sequence that is complementary to a sequence proximal to a sequence found in a AAVS1 gene. As used in this context, sequence “proximal” to a AAVS1 gene is defined as a sequence that is found anywhere 10, 20, 30, 40, 50, 60, 70, 80, 90, or 100 nucleotides in the 5′ or 3′ direction of a AAVS1 gene. In some embodiments, the first and second portions of a AAVS1 gene are not identical sequences. Exemplary portions of a AAVS1 gene include, but are not limited to, reverse complementary sequences to the flanking sequences set forth in any one of SEQ ID NOs: 96, 112, 120, 123-124, and 127-128. In some embodiments, the first and/or second flanking sequences are chosen from flanking sequences set forth in any one of SEQ ID NOs: 96, 112, 120, 123-124, and 127-128. In some embodiments, the safe harbor locus is in the RAB11a gene. RAB11A encodes the Rab11a small GTPAse which regulates intracellular membrane trafficking including formation of transport vesicles to their fusion with membranes. In exemplary embodiments, the genomic sequence of RAB11a is the sequence provided in GenBank: NC_000015.10. In exemplary embodiments, the genomic sequence of RAB11a is the sequence provided at genomic location 15q22.31. In some embodiments, the template polynucleotide, e.g., ssODN, dsODN, minicircle plasmid, or nanoplasmid, comprises a first flanking sequence complementary to a first portion of a RAB11A gene, a donor sequence, and a second flanking sequence complementary to a second portion of the RAB11A gene. In some embodiments, the template polynucleotide, e.g., ssODN, dsODN, minicircle plasmid, or nanoplasmid, comprises a first flanking sequence that is complementary to a sequence proximal to a sequence found in a RAB11A gene. In some embodiments, the template polynucleotide, e.g., ssODN, dsODN, minicircle plasmid, or nanoplasmid, comprises a second flanking sequence that is complementary to a sequence proximal to a second sequence found in a RAB11A gene. In some embodiments, the template polynucleotide, e.g., ssODN, dsODN, minicircle plasmid, or nanoplasmid, comprises a first flanking sequence that is complementary to a portion of a AAVS1 gene, and a second flanking sequence that is complementary to a sequence proximal to a sequence found in a RAB11A gene. As used in this context, sequence “proximal” to a RAB11A gene is defined as a sequence that is found anywhere 10, 20, 30, 40, 50, 60, 70, 80, 90, or 100 nucleotides in the 5′ or 3′ direction of a RAB11A gene. In some embodiments, the first and second portions of a RAB11A gene are not identical sequences. Exemplary portions of a RAB11A gene include, but are not limited to, reverse complementary sequences to the flanking sequences set forth in any one of SEQ ID NOs: 102-104 and 111. In some embodiments, the first and/or second flanking sequences are chosen from flanking sequences set forth in any one of SEQ ID NOs: 102-104 and 111.

### Nucleic Acid Modification

In some embodiments, a template polynucleotide, e.g., ssODN, dsODN, minicircle plasmid, or nanoplasmid, provided herein comprises one or more nucleotides that are chemically modified. Nucleic acids comprising one or more nucleotides that are chemically modified are also referred to herein as modified nucleic acids. Chemical modifications of nucleotides have previously been described, and suitable chemical modifications include any modifications that are beneficial for nucleotides function and do not measurably increase any undesired characteristics, e.g., off-target effects, of a given gRNA. Suitable chemical modifications include, for example, those that make a nucleic acid less susceptible to endo- or exonuclease catalytic activity, and include, without limitation, phosphorothioate backbone modifications, 2′-O-Me-modifications (e.g., at one or both of the 3′ and 5′ termini), 2′F-modifications, replacement of the ribose sugar with the bicyclic nucleotide-cEt, 3′thioPACE (MSP) modifications, or any combination thereof. Additional suitable nucleic acid modifications will be apparent to the skilled artisan based on this disclosure, and such suitable nucleic acid modifications include, without limitation, those described, e.g., Eckstein, Antisense Nucleic Acid Drug Dev. 2000 Apr. 10 (2): 117-21, Rusckowski et al. Antisense Nucleic Acid Drug Dev. 2000 Oct. 10 (5): 333-45, Stein, Antisense Nucleic Acid Drug Dev. 2001 Oct. 11 (5): 317-25, Vorobjev et al. Antisense Nucleic Acid Drug Dev. 2001 Apr. 11 (2): 77-85, Duffy. BMC Bio. 2020 Sep. 2 (8): 112, and U.S. Pat. No. 5,684,143 each of which is incorporated herein by reference in its entirety. In some embodiments, a template polynucleotide comprises a modified nucleotide positioned within the template polynucleotide as described herein with regard to guide RNAs (e.g., with regard to proximity to a 3′ or 5′ end of the template polynucleotide.

### Genetic Modification Mixtures

The disclosure is directed, in part, to genetic modification mixtures. In some embodiments, producing a genetic modification using HDR comprises contacting cells (e.g., HSCs) with a genetic modification mixture comprising one or more other agents that promote genetic modification. In some embodiments, the one or more other agents comprise one or more expansion agents. In some embodiments, the one or more other agents comprise one or more HDR-promoting agents. In some embodiments, the one or more other agents comprise one or more expansion agents and one or more HDR-promoting agents. In some embodiments, producing a genetic modification using HDR comprises contacting HSCs with one or more HDR-promoting agents and/or one or more expansion agents.

As used herein, an HDR-promoting agent refers to a compound that increases the repair of DNA damage by the HDR pathway (e.g., relative to other DNA repair pathways and/or compared to otherwise similar conditions lacking the HDR-promoting agent). Examples of HDR-promoting agents include, but are not limited to: (a) SCR7 which is an inhibitor of DNA ligase IV that is responsible for the repair of DNA double-strand breaks via the non-homologous end joining repair pathway; (b) NU7441 which is an inhibitor of DNA-dependent protein kinase (DNA-PK), an enzyme involved in the non-homologous end joining DNA repair pathway; (c) Rucaparib which is a poly ADP ribose polymerase (PARP) inhibitor that plays a role in the repair of single-stranded breaks in DNA through the base excision repair and nonhomologous end-joining pathways such that inhibition of PARP with rucaparib causes accumulation of single-strand breaks which ultimately results in double-stranded breaks thereby enhancing homology-directed repair activity to promote genome integrity; and (d) RS-1 which is a stimulator of the human homologous recombination protein RAD51 that functions by stimulating binding of human RAD51 to single stranded DNA and enhances recombinogenic activity by stabilizing the active form of human RAD51 filaments without inhibiting human RAD51 ATPase activity.

In some embodiments, the genetic modification mixture comprises one or more HDR-promoting agents comprising SCR7. In some embodiments, the genetic modification mixture comprises one or more HDR-promoting agents comprising NU7441. In some embodiments, the genetic modification mixture comprises one or more HDR-promoting agents comprising rucaparib. In some embodiments, the genetic modification mixture comprises one or more HDR-promoting agents comprising RS-1. In some embodiments, contacting comprises culturing the cell (e.g., the HSCs) in media comprising the one or more HDR-promoting agents. In some embodiments, the cell is contacted with the one or more HDR-promoting agents prior to being contacted with a CRISPR/Cas system, e.g., Cas9, and/or prior to being contacted with a template polynucleotide. In some embodiments, a cell is contacted with a single HDR-promoting agent, e.g., a genetic modification mixture comprises a single HDR-promoting agent. In some embodiments, a cell is contacted with 2, 3, or 4 different HDR-promoting agent, e.g., the genetic modification mixture comprises 2, 3, or 4 different HDR-promoting agents. In some embodiments, a cell is contacted with the different HDR-promoting agents at the same time (e.g., by addition to culture media or by contact with a genetic modification mixture).

As used herein, an expansion agent refers to a compound that specifically promotes the proliferation, differentiation, and/or growth of CD34+ cells such as HSCs. In some embodiments, an expansion agent can be added to culture media. Examples of expansion agents include, but are not limited to: (a) human stem cell factor (hSCF) which is a protein that is critical for hematopoiesis and mast cell differentiation and also plays roles in survival and function of other cell types such as tumor and myeloid-derived suppressor cells wherein hSCF binding to receptor tyrosine kinases induces activation of AKT, ERK, JNK, and p38 pathways in target cells; (b) Fms-like tyrosine kinase 3 Ligand (FLT3-L) which is a hematopoietic cytokine that plays an important role as a co-stimulatory factor in the proliferation, differentiation, and survival of hematopoietic stem and progenitor cells and in the development of the immune system wherein FLT3-L exists as membrane-bound and soluble isoforms such that both isoforms are biologically active and signal through the class III tyrosine kinase receptor; (c) thrombopoietin (TPO) which is a key regulator of megakaryocytopoiesis and thrombopoiesis in vitro and in vivo wherein TPO stimulates the proliferation and maturation of megakaryocytes and has an important role in regulating the level of circulating platelets in vivo; promoting the survival, self-renewal, and expansion of hematopoietic stem cells and primitive multilineage progenitor cells; (d) interleukin 6 (IL-6) which is a pleiotropic growth factor with a wide range of biological activities in immune regulation, hematopoiesis, and oncogenesis such that IL-6 is produced by a variety of cell types including T cells, B cells, monocytes and macrophages, fibroblasts, hepatocytes, vascular endothelial cells, and various tumor cell lines. IL-6 signals through a cell surface type I cytokine receptor complex consisting of the ligand-binding IL-6a (CD126) and the signal-transducing gp130 subunits and the binding of IL-6 to its receptor system induces activation of JAK/STAT signaling pathway; (e) StemRegenin (SR1) which is an antagonist of the aryl hydrocarbon receptor and promotes ex vivo expansion of CD34+ human hematopoietic stem cells and the generation of CD34+ hematopoietic progenitor cells from non-human primate induced pluripotent stem cells such that SR1 has been shown to collaborate with UM729 in preventing differentiation of acute myeloid leukemia (AML) cells in culture and stimulating the proliferation and differentiation of CD34+ hematopoietic progenitor cells into dendritic cells; and (f) UM171 which is a pyrimidoindole small molecule that was discovered in a screen of compounds capable of promoting CD34+ cell expansion when used in combination with other cytokines in culture.

In some embodiments, the genetic modification mixture comprises one or more expansion agents comprising hSCF. In some embodiments, the genetic modification mixture comprises one or more expansion agents comprising FLT3-L. In some embodiments, the genetic modification mixture comprises one or more expansion agents comprising TPO. In some embodiments, the genetic modification mixture comprises one or more expansion agents comprising IL-6. In some embodiments, the genetic modification mixture comprises one or more expansion agents comprising SR1. In some embodiments, the genetic modification mixture comprises one or more expansion agents comprising UM171. In some embodiments, contacting comprises culturing the cell (e.g., the HSCs) in media comprising the one or more expansion agents. In some embodiments, the cell is contacted with the one or more expansion agents prior to being contacted with CRISPR/Cas system, e.g., Cas9, and/or prior to being contacted with a template polynucleotide or an rAAV comprising a template polynucleotide. In some embodiments, a cell is contacted with a single expansion agent, e.g., a genetic modification mixture comprises a single expansion agent. In some embodiments, a cell is contacted with 3, 4, or 5 different expansion agents, e.g., a genetic modification mixture comprises 2, 3, 4, or 5 different expansion agents. In some embodiments, a cell is contacted with the different expansion agents at the same time (e.g., by addition to culture media or by contact with a genetic modification mixture).

In some embodiments, a cell is contacted with 1, 2, 3, 4, or 5 expansion agents and 1, 2, 3, or 4 HDR-promoting agents, e.g., by addition to culture media or by contact with a genetic modification mixture comprising the aforementioned). In some embodiments, the cell is contacted with the one or more expansion agents and one or more HDR-promoting agents prior to being contacted with a CRISPR/Cas system, e.g., Cas9, and/or prior to being contacted with a template polynucleotide.

In some embodiments, producing a genetic modification using HDR comprises using a kit described herein. In some embodiments, a kit comprises a collection of agents that, when used in combination with each other, produce a result such as genetic modification of HSCs. In some embodiments, a kit comprises instructions for use, e.g., instructions for producing a genetically modified HSC. In some embodiments, the instructions comprise instructions for a method described herein. In some embodiments, a kit, e.g., for genetic modification of HSCs, comprises: (a) a template polynucleotide (e.g., a single-strand donor oligonucleotide (ssODN), double-stranded donor ODN (dsODN), minicircle plasmid, or nanoplasmid, comprising a donor sequence, a first flanking sequence and a second flanking sequence); and (b) a CRISPR/Cas system capable of producing a double-stranded break at a target site in the genome of a cell, e.g., an HSC. In some embodiments, a kit comprises (c) one or both of: one or more expansion agents described herein, and one or more HDR promoting agent described herein.

### Gaucher Disease Treatment

Gaucher disease (GD) is an autosomal recessive disorder caused by mutations in the glucosylceramidase beta (GBA) gene, also referred to as the beta-glucocerebrosidase or glucocerebrosidase gene. The GBA gene encodes glucocerebrosidase (GCase). GCase is a lyosomal enzyme which catalyzes the cleavage of a major glycolipid glucosylceramide (GlcCer) into glucose and ceramide. Gaucher disease results in a high degree of clinical heterogeneity among affected individuals. Over 30 mutations in the GBA gene have been associated with etiology of Gaucher disease. There are 3 types of Gaucher disease. Common to all three types of Gaucher disease are skeletal abnormalities such as weakened bones, enlarged liver and spleen, impaired motor coordination, and blood abnormalities. Type 1 is the most common occurring in about 90% of affected individuals. Type 2 usually occurs in children 3-6 months of age and can also result in brain damage, seizures, abnormal eye movements, and poor ability to suck and swallow. Type 2 is usually fatal within the first 2-4 years of life. Type 3 is also known as chronic neuronopathic Gaucher disease and results in symptoms including seizures, eye movement issues, cognitive irregularities, and respiratory problems. Treatment for Gaucher disease has previously included enzyme replacement therapies, small molecules which inhibit the biosynthesis of GBA substrates, blood transfusions, bone marrow transplant, and surgical intervention for spleen reduction/removal and joint replacement. The most common genetic mutations associated with type 1 Gaucher disease are missense mutations, N409S and L483P.

The disclosure is directed, in part, to a method of treating Gaucher disease. As used herein, “treatment” and “treating” refer to administering a therapeutic agent to a subject diagnosed with Gaucher disease, showing symptoms associated with Gaucher disease, or at risk of developing Gaucher disease. The therapeutic agent used in the treatment can be administered to either prevent a subject from developing Gaucher disease, slow the progression of Gaucher disease in a subject, lessen the severity of Gaucher disease in a subject, or lessen one or more symptoms of Gaucher disease in a subject. In some embodiments, the therapeutic agent is a genetically modified HSC, e.g., comprising a modification in the GBA gene (e.g., relative to a naturally occurring GBA gene). In some embodiments, a method of treating Gaucher disease comprises providing a hematopoietic cell (e.g., an HSC), e.g., comprising a genetic modification, e.g., produced by a method described herein. In some embodiments, a method of treating Gaucher disease comprises genetically engineering the GBA gene of an HSC. In some embodiments, a method of treating Gaucher disease comprises obtaining an HSC from a subject (e.g., a subject having or at risk of Gaucher disease), and genetically engineering the GBA gene of the HSC. In some embodiments, a method of treating Gaucher disease comprises administering a genetically engineered HSC to a subject wherein the HSC is autologous to the subject. In some embodiments, a method of treating Gaucher disease comprises administering a genetically engineered HSC to a subject wherein the HSC is allogenic to the subject.

In some embodiments, a method of treating Gaucher disease comprises administering to a subject a genetically modified HSC described herein. In some embodiments, the genetically modified HSC is produced by: contacting the HSC with: a template polynucleotide, e.g., a ssODN, dsODN, minicircle plasmid, or nanoplasmid, comprising a donor sequence, a first flanking sequence complementary to a first portion of a GBA gene and a second flanking sequence complementary to a second portion of the GBA gene or an rAAV thereof; and a CRISPR/Cas system capable of producing a double-stranded break at a target site in the genome of the hematopoietic stem cell. In some embodiments, the genetically modified HSC is produced by also contacting the HSC with one or more expansion agents and/or one or more HDR-promoting agents. In some embodiments, the template polynucleotide, e.g., ssODN, dsODN, minicircle plasmid, or nanoplasmid, comprises a first flanking sequence that is complementary to a first portion of exon 9 of the GBA gene. In some embodiments, the template polynucleotide, e.g., ssODN, dsODN, minicircle plasmid, or nanoplasmid, comprises a second flanking sequence that is complementary to a second portion of exon 9 of the GBA gene. In some embodiments, the template polynucleotide, e.g., ssODN, dsODN, minicircle plasmid, or nanoplasmid, comprises a first flanking sequence that is complementary to a sequence proximal to a sequence found in exon 9 of the GBA gene. In some embodiments, the template polynucleotide, e.g., ssODN, dsODN, minicircle plasmid, or nanoplasmid, comprises a second flanking sequence that is complementary to a sequence proximal to a second sequence found in exon 9 of the GBA gene. In some embodiments, the template polynucleotide, e.g., ssODN, dsODN, minicircle plasmid, or nanoplasmid, comprises a first flanking sequence that is complementary to a portion of exon 9 of the GBA gene, and a second flanking sequence that is complementary to a sequence proximal to a sequence found in exon 9 of the GBA gene. As used in this context, sequence “proximal” to an exon in the GBA gene is defined as a sequence that is found anywhere 10, 20, 30, 40, 50, 60, 70, 80, 90, or 100 nucleotides in the 5′ or 3′ direction of exon 9 of the GBA gene. In some embodiments, the first and second portions of exon 9 are not identical sequences. Exemplary portions of exon 9 include, but are not limited to, reverse complementary sequences to the flanking sequences set forth any one of SEQ ID NOs: 25-28. In some embodiments, the template polynucleotide, e.g., ssODN, dsODN, minicircle plasmid, or nanoplasmid, comprises a first flanking sequence that is complementary to a first portion of exon 10 of the GBA gene. In some embodiments, the template polynucleotide, e.g., ssODN, dsODN, minicircle plasmid, or nanoplasmid, comprises a second flanking sequence that is complementary to a second portion of exon 10 of the GBA gene. In some embodiments, the template polynucleotide, e.g., ssODN, dsODN, minicircle plasmid, or nanoplasmid, comprises a first flanking sequence that is complementary to a sequence proximal to a sequence found in exon 10 of the GBA gene. In some embodiments, the template polynucleotide, e.g., ssODN, dsODN, minicircle plasmid, or nanoplasmid, comprises a second flanking sequence that is complementary to a sequence proximal to a second sequence found in exon 10 of the GBA gene. In some embodiments, the template polynucleotide, e.g., ssODN, dsODN, minicircle plasmid, or nanoplasmid, comprises a first flanking sequence that is complementary to a portion of exon 10 of the GBA gene, and a second flanking sequence that is complementary to a sequence proximal to a sequence found in exon 10 of the GBA gene. As used in this context, sequence “proximal” to an exon in the GBA gene is defined as a sequence that is found anywhere 10, 20, 30, 40, 50, 60, 70, 80, 90, or 100 nucleotides in the 5′ or 3′ direction of exon 10 of the GBA gene. In some embodiments, the first and second portions of exon 10 are not identical sequences. Exemplary portions of exon 10 include, but are not limited to, reverse complementary sequences to the flanking sequences set forth in any one of SEQ ID NOs: 29 or 30. In some embodiments, the first and/or second flanking sequences are chosen from flanking sequences set forth in any one of SEQ ID NOs: 25-30.

In some embodiments, Gaucher disease is treated by a method that comprises generating a genetically modified hematopoietic stem cell. In some embodiments, a method of genetically modifying an HSC produces one or more substitutions; wherein the substitution corrects a naturally occurring mutation characteristic of, or causally associated with, Gaucher disease. In some embodiments, the hematopoietic stem cell is genetically modified to comprise one, two, or three of: (a) an endogenous glucosylceramidase beta (GBA) gene that encodes an asparagine at a position corresponding to position 409 of a wildtype GBA gene; (b) an endogenous GBA gene that encodes a leucine at a position corresponding to position 483 of a wildtype GBA gene; or (c) a heterologous copy of a GBA gene that encodes an asparagine at a position corresponding to position 409 of a wildtype GBA gene and a leucine at a position corresponding to position 483 of a wildtype GBA gene, and then administering the genetically modified hematopoietic stem cell to the subject. In some embodiments, the hematopoietic stem cell is genetically modified to comprise an endogenous glucosylceramidase beta (GBA) gene that encodes an asparagine at a position corresponding to position 409 of a wildtype GBA gene. In some embodiments, the hematopoietic stem cell is genetically modified to comprise an endogenous GBA gene that encodes a leucine at a position corresponding to position 483 of a wildtype GBA gene. In some embodiments, the hematopoietic stem cell is genetically modified to comprise a heterologous copy of a GBA gene that encodes an asparagine at a position corresponding to position 409 of a wildtype GBA gene and a leucine at a position corresponding to position 483 of a wildtype GBA gene. In some embodiments, the genetic modification is to a safe harbor locus.

### CRISPR/Cas Systems

The disclosure is directed, in part, to methods of producing a genetically modified cell, using a genome editing technology to produce a break in a cell's genomic DNA that can be resolved by homology directed repair (HDR), thereby genetically modifying the cell.

One exemplary suitable genome editing technology is “gene editing,” comprising the use of a RNA-guided nuclease, e.g., a CRISPR/Cas nuclease, to introduce targeted single- or double-stranded DNA breaks in the genome of a cell, which trigger cellular repair mechanisms, such as, for example, nonhomologous end joining (NHEJ), microhomology-mediated end joining (MMEJ, also sometimes referred to as “alternative NHEJ” or “alt-NHEJ”), or homology-directed repair (HDR) that typically result in an altered nucleic acid sequence (e.g., via nucleotide or nucleotide sequence insertion, deletion, inversion, or substitution) at or immediately proximal to the site of the nuclease cut. As used in this context, “proximal” to the site of a nuclease cut is defined as a sequence that is found anywhere 10, 20, 30, 40, 50, 60, 70, 80, 90, or 100 nucleotides in the 5′ or 3′ direction of a CRISPR/Cas nuclease cut site. See, e.g., Yeh et al. Nat. Cell. Biol. (2019) 21:1468-1478; e.g., Hsu et al. Cell (2014) 157:1262-1278; Jasin et al. DNA Repair (2016) 44:6-16; Sfeir et al. Trends Biochem. Sci. (2015) 40:701-714.

An RNA-guided nuclease in some embodiments is catalytically impaired, or partially catalytically impaired. Examples of suitable RNA-guided nucleases include CRISPR/Cas nucleases. For example, in some embodiments, a suitable RNA-guided nuclease for use in the methods of genetically engineering cells provided herein is a Cas9 nuclease, e.g., a SpCas9 or an SaCas9 nuclease. For another example, in some embodiments, a suitable RNA-guided nuclease for use in the methods of genetically engineering cells provided herein is a Cas12 nuclease, e.g., a Cas12a nuclease. Exemplary suitable Cas12 nucleases include, without limitation, AsCas12a, FnCas12a, other Cas12a orthologs, and Cas12a derivatives, such as the MAD7 system (MAD7TM, Inscripta, Inc.), or the Alt-R Cas12a (Cpf1) Ultra nuclease (Alt-R® Cas12a Ultra; Integrated DNA Technologies, Inc.). See, e.g., Gill et al. 2018 U.S. Pat. No. 9,982,279 and Gill et al. 2018 U.S. Pat. No. 10,011,849. In United States: Inscripta Inc.; Price et al. Biotechnol. Bioeng. (2020) 117 (6): 1805-1816.

In some embodiments, a genetically engineered cell (e.g., a genetically engineered hematopoietic cell, such as, for example, a genetically engineered hematopoietic stem or progenitor cell or a genetically engineered immune effector cell) described herein is generated by targeting an RNA-guided nuclease, e.g., a CRISPR/Cas nuclease, such as, for example, a Cas9 nuclease or a Cas12a nuclease, to a suitable target site in the genome of the cell, under conditions suitable for the RNA-guided nuclease to bind the target site and cut the genomic DNA of the cell. A suitable RNA-guided nuclease can be targeted to a specific target site within the genome by a suitable guide RNA (gRNA). Suitable gRNAs for targeting CRISPR/Cas nucleases according to aspects of this disclosure are provided herein and exemplary suitable gRNAs are described in more detail elsewhere herein.

In some embodiments, a GBA gRNA (i.e., a guide RNA complementary to a portion of the GBA gene) described herein is complexed with a CRISPR/Cas nuclease, e.g., a Cas9 nuclease. Various Cas9 nucleases are suitable for use with the gRNAs provided herein to effect genome editing according to aspects of this disclosure, e.g., to create a genomic modification in the GBA gene. Typically, the Cas nuclease and the gRNA are provided in a form and under conditions suitable for the formation of a Cas/gRNA complex, that targets a target site on the genome of the cell, e.g., a target site within the GBA gene. In some embodiments, a Cas nuclease is used that exhibits a desired PAM gene. Suitable target domains and corresponding gRNA targeting domain sequences are provided herein.

In some embodiments, a Cas/gRNA complex is formed, e.g., in vitro, and a target cell is contacted with the Cas/gRNA complex, e.g., via electroporation of the Cas/gRNA complex into the cell. In some embodiments, the cell is contacted with Cas protein and gRNA separately, and the Cas/gRNA complex is formed within the cell. In some embodiments, the cell is contacted with a nucleic acid, e.g., a DNA or RNA, encoding the Cas protein, and/or with a nucleic acid encoding the gRNA, or both.

In some embodiments, genetically engineered cells as provided herein are generated using a suitable genome editing technology, wherein the genome editing technology is characterized by the use of a Cas9 nuclease. In some embodiments, the Cas9 molecule is of, or derived from, Streptococcus pyogenes (SpCas9), Staphylococcus aureus (SaCas9), or Streptococcus thermophilus (stCas9). Additional suitable Cas9 molecules include those of, or derived from, Neisseria meningitidis (NmCas9), Acidovorax avenae, Actinobacillus pleuropneumoniae, Actinobacillus succinogenes, Actinobacillus suis, Actinomyces sp., Cycliphilus denitrificans, Aminomonas paucivorans, Bacillus cereus, Bacillus smithii, Bacillus thuringiensis, Bacteroides sp., Blastopirellula marina, Bradyrhizobium sp., Brevibacillus laterosporus, Campylobacter coli, Campylobacter jejuni (CjCas9), Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gamma proteobacterium, Gluconacetobacter diazotrophicus, Haemophilus parainfluenzae, Haemophilus sputorum, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus lugdunensis, Streptococcus sp., Subdoligranulum sp., Tistrella mobilis, Treponema sp., or Verminephrobacter eiseniae.

In some embodiments, catalytically impaired, or partially impaired, variants of such Cas9 nucleases are used. Additional suitable Cas9 nucleases, and nuclease variants, will be apparent to those of skill in the art based on the present disclosure. The disclosure is not limited in this respect.

In some embodiments, the Cas nuclease is a naturally occurring Cas molecule. In some embodiments, the Cas nuclease is an engineered, altered, or modified Cas molecule that differs, e.g., by at least one amino acid residue, from a reference sequence, e.g., the most similar naturally occurring Cas9 molecule or a sequence of Table 50 of PCT Publication No. WO2015/157070, which is herein incorporated by reference in its entirety.

In some embodiments, a Cas nuclease is used that belongs to class 2 type V of Cas nucleases. Class 2 type V Cas nucleases can be further categorized as type V-A, type VB, type V-C, and type V-U. See, e.g., Stella et al. Nature Structural & Molecular Biology (2017) 24:882-892. In some embodiments, the Cas nuclease is a type V-B Cas endonuclease, such as a C2c1. See, e.g., Shmakov et al. Mol Cell (2015) 60:385-397. In some embodiments, the Cas nuclease used in the methods of genome editing provided herein is a type V-A Cas endonuclease, such as a Cpf1 (Cas12a) nuclease. See, e.g., Strohkendl et al. Mol. Cell (2018) 71:1-9. In some embodiments, a Cas nuclease used in the methods of genome editing provided herein is a Cpf1 nuclease derived from Provetella spp. or Francisella spp., Acidaminococcus sp. (AsCpf1), Lachnospiraceae bacterium (LpCpf1), or Eubacterium rectale. In some embodiments, the Cas nuclease is MAD7TM (Inscripta).

Both naturally occurring and modified variants of CRISPR/Cas nucleases are suitable for use according to aspects of this disclosure. For example, dCas or nickase variants, Cas variants having altered PAM specificities, and Cas variants having improved nuclease activities are embraced by some embodiments of this disclosure.

Some features of some exemplary, non-limiting suitable Cas nucleases are described in more detail herein, without wishing to be bound to any particular theory.

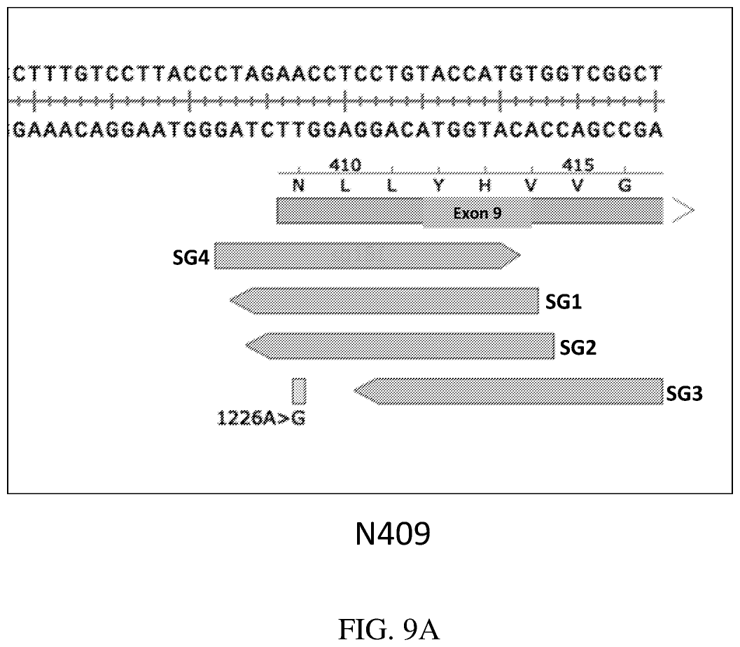

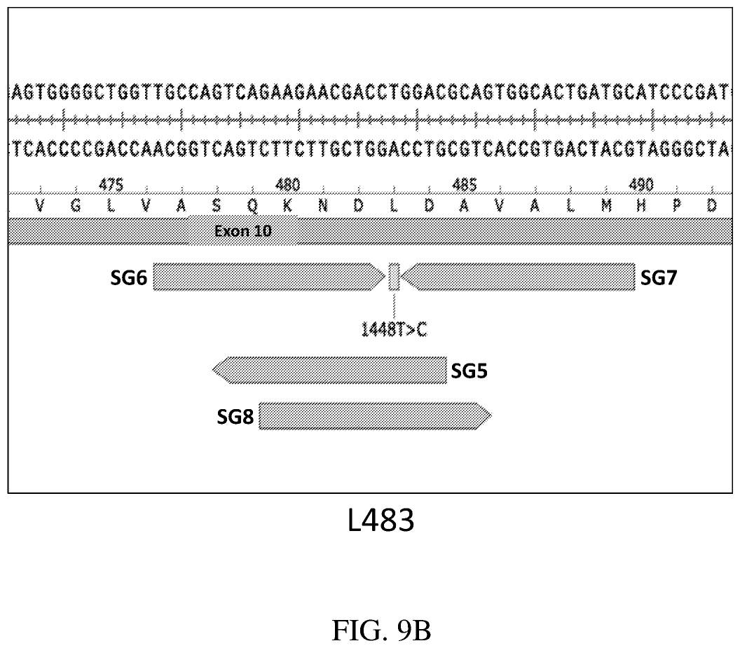

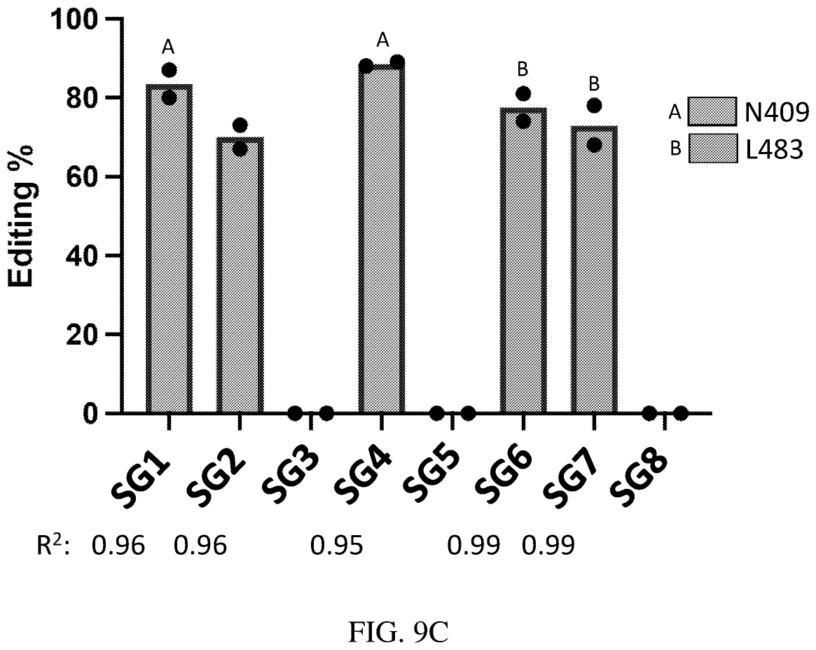

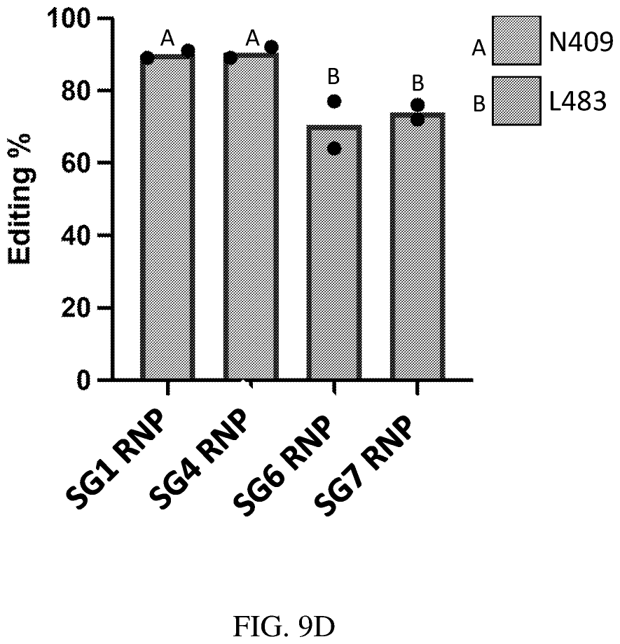

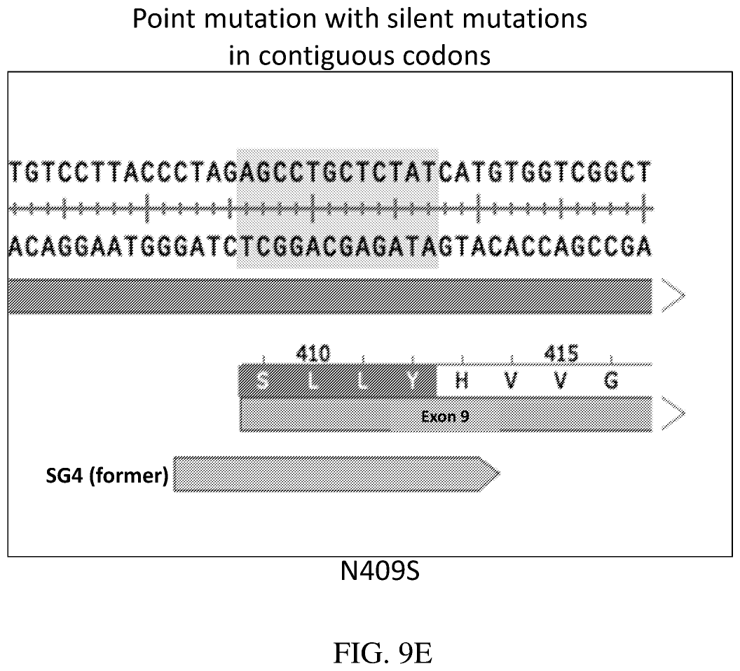

A naturally occurring Cas9 nuclease typically comprises two lobes: a recognition (REC) lobe and a nuclease (NUC) lobe; each of which further comprises domains described, e.g., in PCT Publication No. WO2015/157070, e.g., in FIGS. 9A-9B therein (which application is incorporated herein by reference in its entirety).

The REC lobe comprises the arginine-rich bridge helix (BH), the REC1 domain, and the REC2 domain. The REC lobe appears to be a Cas9-specific functional domain. The BH domain is a long alpha helix and arginine rich region and comprises amino acids 60-93 of the sequence of S. pyogenes Cas9. The REC1 domain is involved in recognition of the repeat: anti-repeat duplex, e.g., of a gRNA or a tracrRNA. The REC1 domain comprises two REC1 motifs at amino acids 94 to 179 and 308 to 717 of the sequence of S. pyogenes Cas9. These two REC1 domains, though separated by the REC2 domain in the linear primary structure, assemble in the tertiary structure to form the REC1 domain. The REC2 domain, or parts thereof, may also play a role in the recognition of the repeat: antirepeat duplex. The REC2 domain comprises amino acids 180-307 of the sequence of S. pyogenes Cas9.

The NUC lobe comprises the RuvC domain, the HNH domain, and the PAM interacting (PI) domain. The RuvC domain shares structural similarity to retroviral integrase superfamily members and cleaves a single strand, e.g., the non-complementary strand of the target nucleic acid molecule. The RuvC domain is assembled from the three split RuvC motifs (RuvC I, RuvCII, and RuvCIII, which are often commonly referred to in the art as RuvCI domain, or N-terminal RuvC domain, RuvCII domain, and RuvCIII domain) at amino acids 1-59, 718-769, and 909-1098, respectively, of the sequence of S. pyogenes Cas9. Similar to the REC1 domain, the three RuvC motifs are linearly separated by other domains in the primary structure, however in the tertiary structure, the three RuvC motifs assemble and form the RuvC domain. The HNH domain shares structural similarity with HNH endonucleases, and cleaves a single strand, e.g., the complementary strand of the target nucleic acid molecule. The HNH domain lies between the RuvC II-III motifs and comprises amino acids 775-908 of the sequence of S. pyogenes Cas9. The PI domain interacts with the PAM of the target nucleic acid molecule and comprises amino acids 1099-1368 of the sequence of S. pyogenes Cas9.

Crystal structures have been determined for naturally occurring bacterial Cas9 nucleases (see, e.g., Jinek et al., Science, 343 (6176): 1247997, 2014) and for S. pyogenes Cas9 with a guide RNA (e.g., a synthetic fusion of crRNA and tracrRNA) (Nishimasu et al., Cell (2014) 156:935-949; and Anders et al., Nature (2014) doi: 10.1038/nature13579).

In some embodiments, a Cas9 molecule described herein exhibits nuclease activity that results in the introduction of a double strand DNA break in or directly proximal to a target site, e.g., the binding site of a guide RNA to which the Cas9 molecule is complexed. As used in this context, “proximal” refers to a sequence that is found anywhere 10, 20, 30, 40, 50, 60, 70, 80, 90, or 100 nucleotides in the 5′ or 3′ direction of the guide RNA binding site. In some embodiments, the Cas9 molecule has been modified to inactivate one of the catalytic residues of the endonuclease. In some embodiments, the Cas9 molecule is a nickase and produces a single stranded break. See, e.g., Dabrowska et al. Frontiers in Neuroscience (2018) 12 (75). It has been shown that one or more mutations in the RuvC and HNH catalytic domains of the enzyme may improve Cas9 efficiency. See, e.g., Safari et al. Currently Pharma. Biotechnol. (2017) 18 (13): 1038-1054. In some embodiments, the Cas9 molecule is fused to a second domain, e.g., a domain that modifies DNA or chromatin, e.g., a deaminase or demethylase domain. In some such embodiments, the Cas9 molecule is modified to eliminate its endonuclease activity.

In some embodiments, a Cas nuclease or a Cas/gRNA complex described herein is administered together with a template for homology directed repair (HDR). In some embodiments, a Cas nuclease or a Cas/gRNA complex described herein is administered without a HDR template.

In some embodiments, a Cas9 nuclease is used that is modified to enhance specificity of the enzyme (e.g., reduce off-target effects, maintain robust on-target cleavage).

In some embodiments, the Cas9 molecule is an enhanced specificity Cas9 variant (e.g., eSpCas9). See, e.g., Slaymaker et al. Science (2016) 351 (6268): 84-88. In some embodiments, the Cas9 molecule is a high fidelity Cas9 variant (e.g., SpCas9-HF1). See, e.g., Kleinstiver et al. Nature (2016) 529:490-495.

Various Cas nucleases are known in the art and may be obtained from various sources and/or engineered/modified to modulate one or more activities or specificities of the enzymes. PAM sequence preferences and specificities of suitable Cas nucleases, e.g., suitable Cas9 nucleases, such as, for example, SpCas9 and SaCas9 are known in the art. In some embodiments, the Cas nuclease has been engineered/modified to recognize one or more PAM sequence. In some embodiments, the Cas nuclease has been engineered/modified to recognize one or more PAM sequence that is different than the PAM sequence the Cas nuclease recognizes without engineering/modification. In some embodiments, the Cas nuclease has been engineered/modified to reduce off-target activity of the enzyme.

In some embodiments, a Cas nuclease is used that is modified further to alter the specificity of the endonuclease activity (e.g., reduce off-target cleavage, decrease the endonuclease activity or lifetime in cells, increase homology-directed recombination and reduce non-homologous end joining). See, e.g., Komor et al. Cell (2017) 168:20-36. In some embodiments, a Cas nuclease is used that is modified to alter the PAM recognition or preference of the endonuclease. For example, SpCas9 recognizes the PAM sequence NGG, whereas some variants of SpCas9 comprising one or more modifications (e.g., VQR SpCas9, EQR SpCas9, VRER SpCas9) may recognize variant PAM sequences, e.g., NGA, NGAG, and/or NGCG. For another example, SaCas9 recognizes the PAM sequence NNGRRT, whereas some variants of SaCas9 comprising one or more modifications (e.g., KKH SaCas9) may recognize the PAM sequence NNNRRT. In another example, FnCas9 (Cas9 from Francisella novicida) recognizes the PAM sequence NNG, whereas a variant of the FnCas9 comprises one or more modifications (e.g., RHA FnCas9) may recognize the PAM sequence YG. In another example, the Cas12a nuclease comprising substitution mutations S542R and K607R recognizes the PAM sequence TYCV. In another example, a Cpf1 endonuclease comprising substitution mutations S542R, K607R, and N552R recognizes the PAM sequence TATV. See, e.g., Gao et al. Nat. Biotechnol. (2017) 35 (8): 789-792.

In some embodiments, more than one (e.g., 2, 3, or more) Cas9 molecules are used. In some embodiments, at least one of the Cas9 molecules is a Cas9 enzyme. In some embodiments, at least one of the Cas molecules is a Cpf1 enzyme. In some embodiments, at least one of the Cas9 molecule is derived from Streptococcus pyogenes. In some embodiments, at least one of the Cas9 molecule is derived from Streptococcus pyogenes and at least one Cas9 molecule is derived from an organism that is not Streptococcus pyogenes. Some aspects of this disclosure provide guide RNAs that are suitable to target an RNA-guided nuclease, e.g., as provided herein, to a suitable target site in the genome of a cell in order to effect a modification in the genome of the cell that results in a loss of expression of GBA, or expression of a variant form of GBA that is not recognized by an immunotherapeutic agent targeting GBA.

The terms “guide RNA” and “gRNA” are used interchangeably herein and refer to a nucleic acid, typically an RNA that is bound by an RNA-guided nuclease and promotes the specific targeting or homing of the RNA-guided nuclease to a target nucleic acid, e.g., a target site within the genome of a cell. A gRNA typically comprises at least two domains: a “binding domain,” also sometimes referred to as “gRNA scaffold” or “gRNA backbone” that mediates binding to an RNA-guided nuclease (also referred to as the “binding domain”), and a “targeting domain” that mediates the targeting of the gRNA-bound RNA guided nuclease to a target site. Some gRNAs comprise additional domains, e.g., complementarity domains, or stem-loop domains. The structures and sequences of naturally occurring gRNA binding domains and engineered variants thereof are well known to those of skill in the art. Some suitable gRNAs are unimolecular, comprising a single nucleic acid sequence, while other suitable gRNAs comprise two sequences (e.g., a crRNA and tracrRNAsequence).

Some exemplary suitable Cas9 gRNA scaffold sequences are provided herein, and additional suitable gRNA scaffold sequences will be apparent to the skilled artisan based on the present disclosure. Such additional suitable scaffold sequences include, without limitation, those recited in Jinek, et al. Science (2012) 337 (6096): 816-821, Ran, et al. Nature Protocols (2013) 8:2281-2308, PCT Publication No. WO2014/093694, and PCT Publication No. WO2013/176772.

For example, the binding domains of naturally occurring SpCas9 gRNA typically comprise two RNA molecules, the crRNA (partially) and the tracrRNA. Variants of SpCas9 gRNAs that comprise only a single RNA molecule including both crRNA and tracrRNA sequences, covalently bound to each other, e.g., via a tetraloop or via click chemistry type covalent linkage, have been engineered and are commonly referred to as “single guide RNA” or “sgRNA.” Suitable gRNAs for use with other Cas nucleases, for example, with Cas12a nucleases, typically comprise only a single RNA molecule, as the naturally occurring Cas12a guide RNA comprises a single RNA molecule. A suitable gRNA may thus be unimolecular (having a single RNA molecule), sometimes referred to herein as sgRNAs, or modular (comprising more than one, and typically two, separate RNA molecules).

A gRNA suitable for targeting a target site in the GBA gene may comprise a number of domains. In some embodiments, e.g., in some embodiments where a Cas9 nuclease is used, a unimolecular sgRNA, may comprise, from 5′ to 3′: a targeting domain corresponding to a target site sequence in the GBA gene; a first complementarity domain; a linking domain; a second complementarity domain (which is complementary to the first complementarity domain); a proximal domain; and optionally, a tail domain.

Each of these domains is now described in more detail.

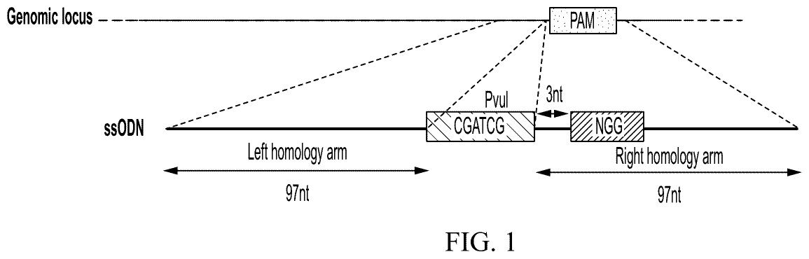

A gRNA as provided herein typically comprises a targeting domain that binds to a target site in the genome of a cell. The target site is typically a double-stranded DNA sequence comprising the PAM sequence and, on the same strand as, and directly adjacent to, the PAM sequence, the target domain. The targeting domain of the gRNA typically comprises an RNA sequence that corresponds to the target domain sequence in that it resembles the sequence of the target domain, sometimes with one or more mismatches, but typically comprises an RNA instead of a DNA sequence. The targeting domain of the gRNA thus base-pairs (in full or partial complementarity) with the sequence of the double-stranded target site that is complementary to the sequence of the target domain, and thus with the strand complementary to the strand that comprises the PAM sequence. It will be understood that the targeting domain of the gRNA typically does not include the PAM sequence. It will further be understood that the location of the PAM may be 5′ or 3′ of the target domain sequence, depending on the nuclease employed. For example, the PAM is typically 3′ of the target domain sequences for Cas9 nucleases, and 5′ of the target domain sequence for Cas12a nucleases. For an illustration of the location of the PAM and the mechanism of gRNA binding a target site, see, e.g., Figure 1 of Vanegas et al., Fungal Biol Biotechnol. 2019; 6:6, which is incorporated by reference herein. For additional illustration and description of the mechanism of gRNA targeting an RNA-guided nuclease to a target site, see Fu Y et al, Nat Biotechnol 2014 (doi: 10.1038/nbt.2808) and Sternberg S H et al., Nature 2014 (doi: 10.1038/nature13011), both incorporated herein by reference.

The targeting domain may comprise a nucleotide sequence that corresponds to the sequence of the target domain, i.e., the DNA sequence directly adjacent to the PAM sequence (e.g., 5′ of the PAM sequence for Cas9 nucleases, or 3′ of the PAM sequence for Cas12a nucleases). The targeting domain sequence typically comprises between 17 and 30 nucleotides and corresponds fully with the target domain sequence (i.e., without any mismatch nucleotides), or may comprise one or more, but typically not more than 4, mismatches. As the targeting domain is part of an RNA molecule, the gRNA, it will typically comprise ribonucleotides, while the DNA targeting domain will comprise deoxyribonucleotides.

An exemplary illustration of a Cas9 target site, comprising a 22 nucleotide target domain, and an NGG PAM sequence, as well as of a gRNA comprising a targeting domain that fully corresponds to the target domain (and thus base-pairs with full complementarity with the DNA strand complementary to the strand comprising the target domain and PAM) is provided below:

```
      [ target domain (DNA) ] [PAM]
5′-N-N-N-N-N-N-N-N-N-N-N-N-N-N-N-N-N-N-N-N-N-N-N-G-G-3′ (DNA)
3′-N-N-N-N-N-N-N-N-N-N-N-N-N-N-N-N-N-N-N-N-N-N-N-C-C-5′ (DNA)
      | | | | | | | | | | | | | | | | | | | | | |
5′-N-N-N-N-N-N-N-N-N-N-N-N-N-N-N-N-N-N-N-N-N-N-[gRNA scaffold]-3′ (RNA)
      [ targeting domain (RNA) ][binding domain]
```

An exemplary illustration of a Cas12a target site, comprising a 22 nucleotide target domain, and a TTN PAM sequence, as well as of a gRNA comprising a targeting domain that fully corresponds to the target domain (and thus base-pairs with full complementarity with the DNA strand complementary to the strand comprising the target domain and PAM) is provided below:

```
    [ PAM ][ target domain (DNA) ]
5′-T-T-N-N-N-N-N-N-N-N-N-N-N-N-N-N-N-N-N-N-N-N-N-N-N-3′ (DNA)
3′-A-A-N-N-N-N-N-N-N-N-N-N-N-N-N-N-N-N-N-N-N-N-N-N-N-5′
                  | | | | | | | | | | | | | | | | | | | | | |
5′-[gRNA scaffold]-N-N-N-N-N-N-N-N-N-N-N-N-N-N-N-N-N-N-N-N-N-N-3′ (RNA)
    [binding domain][ targeting domain (RNA) ]
```

In some embodiments, the Cas12a PAM sequence is 5′-T-T-T-V-3′.

While not wishing to be bound by theory, at least in some embodiments, it is believed that the length and complementarity of the targeting domain with the target sequence contributes to specificity of the interaction of the gRNA/Cas9 molecule complex with a target nucleic acid. In some embodiments, the targeting domain of a gRNA provided herein is 5 to 50 nucleotides in length. In some embodiments, the targeting domain is 15 to 25 nucleotides in length. In some embodiments, the targeting domain is 18 to 22 nucleotides in length. In some embodiments, the targeting domain is 19-21 nucleotides in length. In some embodiments, the targeting domain is 15 nucleotides in length. In some embodiments, the targeting domain is 16 nucleotides in length. In some embodiments, the targeting domain is 17 nucleotides in length. In some embodiments, the targeting domain is 18 nucleotides in length. In some embodiments, the targeting domain is 19 nucleotides in length. In some embodiments, the targeting domain is 20 nucleotides in length. In some embodiments, the targeting domain is 21 nucleotides in length. In some embodiments, the targeting domain is 22 nucleotides in length. In some embodiments, the targeting domain is 23 nucleotides in length. In some embodiments, the targeting domain is 24 nucleotides in length. In some embodiments, the targeting domain is 25 nucleotides in length. In some embodiments, the targeting domain fully corresponds, without mismatch, to a target domain sequence provided herein, or a part thereof. In some embodiments, the targeting domain of a gRNA provided herein comprises 1 mismatch relative to a target domain sequence provided herein. In some embodiments, the targeting domain comprises 2 mismatches relative to the target domain sequence. In some embodiments, the target domain comprises 3 mismatches relative to the target domain sequence.

In some embodiments, a targeting domain comprises a core domain and a secondary targeting domain, e.g., as described in PCT Publication No. WO2015/157070, which is incorporated by reference in its entirety. In some embodiments, the core domain comprises about 8 to about 13 nucleotides from the 3′ end of the targeting domain (e.g., the most 3′ 8 to 13 nucleotides of the targeting domain). In some embodiments, the secondary domain is positioned 5′ to the core domain. In some embodiments, the core domain corresponds fully with the target domain sequence, or a part thereof. In other embodiments, the core domain may comprise one or more nucleotides that are mismatched with the corresponding nucleotide of the target domain sequence.

In some embodiments, e.g., in some embodiments where a Cas9 gRNA is provided, the gRNA comprises a first complementarity domain and a second complementarity domain, wherein the first complementarity domain is complementary with the second complementarity domain, and, at least in some embodiments, has sufficient complementarity to the second complementarity domain to form a duplexed region under at least some physiological conditions. In some embodiments, the first complementarity domain is 5 to 30 nucleotides in length. In some embodiments, the first complementarity domain comprises 3 subdomains, which, in the 5′ to 3′ direction are: a 5′ subdomain, a central subdomain, and a 3′ subdomain. In some embodiments, the 5′ subdomain is 4 to 9, e.g., 4, 5, 6, 7, 8 or 9 nucleotides in length. In some embodiments, the central subdomain is 1, 2, or 3, e.g., 1, nucleotide in length. In some embodiments, the 3′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, or 25 nucleotides in length. The first complementarity domain can share homology with, or be derived from, a naturally occurring first complementarity domain. In an embodiment, it has at least 50% homology with a S. pyogenes, S. aureus or S. thermophilus, first complementarity domain.

The sequence and placement of the above-mentioned domains are described in more detail in PCT Publication No. WO2015/157070, which is herein incorporated by reference in its entirety, including p. 88-112 therein.

A linking domain may serve to link the first complementarity domain with the second complementarity domain of a unimolecular gRNA. The linking domain can link the first and second complementarity domains covalently or non-covalently. In some embodiments, the linkage is covalent. In some embodiments, the linking domain is, or comprises, a covalent bond interposed between the first complementarity domain and the second complementarity domain. In some embodiments, the linking domain comprises one or more, e.g., 2, 3, 4, 5, 6, 7, 8, 9, or 10 nucleotides. In some embodiments, the linking domain comprises at least one non-nucleotide bond, e.g., as disclosed in PCT Publication No. WO2018/126176, the entire contents of which are incorporated herein by reference.

In some embodiments, the second complementarity domain is complementary, at least in part, with the first complementarity domain, and in an embodiment, has sufficient complementarity to the second complementarity domain to form a duplexed region under at least some physiological conditions. In some embodiments, the second complementarity domain can include a sequence that lacks complementarity with the first complementarity domain, e.g., a sequence that loops out from the duplexed region. In some embodiments, the second complementarity domain is 5 to 27 nucleotides in length. In some embodiments, the second complementarity domain is longer than the first complementarity region. In an embodiment, the complementary domain is 5, 6, 7, 8, 9, 10, 11, 12, 13, 14, 15, 16, 17, 18, 19, 20, 21, 22, 23, 24, 25, 26, or 27 nucleotides in length. In some embodiments, the second complementarity domain comprises 3 subdomains, which, in the 5′ to 3′ direction are: a 5′ subdomain, a central subdomain, and a 3′ subdomain. In some embodiments, the 5′ subdomain is 3 to 25, e.g., 4 to 22, 4 to 18, or 4 to 10, or 3, 4, 5, 6, 7, 8, 9, 10, 11, 12, 13, 14, 15, 16, 17, 18, 19, 20, 21, 22, 23, 24, 25, 26, or 27 nucleotides in length. In some embodiments, the central subdomain is 1, 2, 3, 4 or 5, e.g., 3, nucleotides in length. In some embodiments, the 3′ subdomain is 4 to 9, e.g., 4, 5, 6, 7, 8 or 9 nucleotides in length. In some embodiments, the 5′ subdomain and the 3′ subdomain of the first complementarity domain, are respectively, complementary, e.g., fully complementary, with the 3′ subdomain and the 5′ subdomain of the second complementarity domain.

In some embodiments, the proximal domain is 5 to 20 nucleotides in length. In some embodiments, the proximal domain can share homology with or be derived from a naturally occurring proximal domain. In an embodiment, it has at least 50% homology with a proximal domain from S. pyogenes, S. aureus, or S. thermophilus.

A broad spectrum of tail domains are suitable for use in gRNAs. In some embodiments, the tail domain is 0 (absent), 1, 2, 3, 4, 5, 6, 7, 8, 9, or 10 nucleotides in length. In some embodiments, the tail domain nucleotides are from or share homology with a sequence from the 5′ end of a naturally occurring tail domain. In some embodiments, the tail domain includes sequences that are complementary to each other and which, under at least some physiological conditions, form a duplexed region. In some embodiments, the tail domain is absent or is 1 to 50 nucleotides in length. In some embodiments, the tail domain can share homology with or be derived from a naturally occurring proximal tail domain. In some embodiments, the tail domain has at least 50% homology/identity with a tail domain from S. pyogenes, S. aureus or S. thermophilus. In some embodiments, the tail domain includes nucleotides at the 3′ end that are related to the method of in vitro or in vivo transcription.

In some embodiments, a gRNA provided herein comprises: a first strand comprising, e.g., from 5′ to 3′: a targeting domain (which corresponds to a target domain in the GBA gene); and a first complementarity domain; and a second strand, comprising, e.g., from 5′ to 3′: optionally, a 5′ extension domain; a second complementarity domain; a proximal domain; and optionally, a tail domain.

In some embodiments, any of the nucleic acids (e.g., template polynucleotides or gRNAs) provided herein comprise one or more nucleotides that are chemically modified. Chemical modifications of gRNAs have previously been described, and suitable chemical modifications include any modifications that are beneficial for gRNA function and do not measurably increase any undesired characteristics, e.g., off-target effects, of a given gRNA. Suitable chemical modifications include, for example, those that make a gRNA less susceptible to endo- or exonuclease catalytic activity, and include, without limitation, phosphorothioate backbone modifications, 2′-O-Me-modifications (e.g., at one or both of the 3′ and 5′ termini), 2′F-modifications, replacement of the ribose sugar with the bicyclic nucleotide-cEt, 3′thioPACE (MSP) modifications, or any combination thereof. Additional suitable gRNA modifications will be apparent to the skilled artisan based on this disclosure, and such suitable gRNA modifications include, without limitation, those described, e.g., in Rahdar et al. PNAS (2015) 112 (51) E7110-E7117 and Hendel et al., Nat Biotechnol. (2015); 33 (9): 985-989, each of which is incorporated herein by reference in its entirety.

For example, in some embodiments a gRNA provided herein comprises one or more 2′-O modified nucleotide, e.g., a 2′-O-methyl nucleotide. In some embodiments, the gRNA comprises a 2′-O modified nucleotide, e.g., 2′-O-methyl nucleotide at the 5′ end of the gRNA. In some embodiments, the gRNA comprises a 2′-O modified nucleotide, e.g., 2′-Omethyl nucleotide at the 3′ end of the gRNA. In some embodiments, the gRNA comprises a 2′-O-modified nucleotide, e.g., a 2′-O-methyl nucleotide at both the 5′ and 3′ ends of the gRNA. In some embodiments, the gRNA is 2′-O-modified, e.g. 2′-O-methyl-modified at the nucleotide at the 5′ end of the gRNA, the second nucleotide from the 5′ end of the gRNA, and the third nucleotide from the 5′ end of the gRNA. In some embodiments, the gRNA is 2′-O-modified, e.g. 2′-O-methyl-modified at the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified, e.g. 2′-O-methyl-modified at the second nucleotide from the 3′ end of the gRNA, the third nucleotide from the 3′ end of the gRNA, and at the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified, e.g. 2′-O-methyl-modified, at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA. In some embodiments, the 2′-O-methyl nucleotide comprises a phosphate linkage to an adjacent nucleotide. In some embodiments, the 2′-O-methyl nucleotide comprises a phosphorothioate linkage to an adjacent nucleotide. In some embodiments, the 2′-O-methyl nucleotide comprises a thioPACE linkage to an adjacent nucleotide. In some embodiments, a gRNA provided herein comprises one or more 2′-O-modified and 3′phosphorous-modified nucleotide, e.g., a 2′-O-methyl 3′phosphorothioate nucleotide. In some embodiments, the gRNA comprises a 2′-O-modified and 3′phosphorous-modified, e.g., 2′-O-methyl 3′phosphorothioate nucleotide at the 5′ end of the gRNA. In some embodiments, the gRNA comprises a 2′-O-modified and 3′phosphorous modified, e.g., 2′-O-methyl 3′phosphorothioate nucleotide at the 3′ end of the gRNA. In some embodiments, the gRNA comprises a 2′-O-modified and 3′phosphorous-modified, e.g., 2′-O-methyl 3′phosphorothioate nucleotide at the 5′ and 3′ ends of the gRNA. In some embodiments, the gRNA comprises a backbone in which one or more non-bridging oxygen atoms has been replaced with a sulfur atom. In some embodiments, the gRNA is 2′-O modified and 3′phosphorous-modified, e.g. 2′-O-methyl 3′phosphorothioate-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g. 2′-O-methyl 3′phosphorothioate-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′phosphorothioate-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′phosphorothioate-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein comprises one or more 2′-O-modified and 3′-phosphorous-modified, e.g., 2′-O-methyl 3′thioPACE nucleotide. In some embodiments, the gRNA comprises a 2′-O-modified and 3′phosphorous-modified, e.g., 2′-O-methyl 3′thioPACE nucleotide at the 5′ end of the gRNA. In some embodiments, the gRNA comprises a 2′-O-modified and 3′phosphorous-modified, e.g., 2′-O-methyl 3′thioPACE nucleotide at the 3′ end of the gRNA. In some embodiments, the gRNA comprises a 2′-O modified and 3′phosphorous-modified, e.g., 2′-O-methyl 3′thioPACE nucleotide at the 5′ and 3′ ends of the gRNA. In some embodiments, the gRNA comprises a backbone in which one or more non-bridging oxygen atoms have been replaced with a sulfur atom and one or more non-bridging oxygen atoms have been replaced with an acetate group. In some embodiments, the gRNA is 2′-O-modified and 3′phosphorous-modified, e.g., 2′-O-methyl 3′ thioPACE-modified at the nucleotide at the 5′ end of the gRNA, the second nucleotide from the 5′ end of the gRNA, and the third nucleotide from the 5′ end of the gRNA. In some embodiments, the gRNA is 2′-O-modified and 3′phosphorous-modified, e.g., 2′-O-methyl 3′thioPACE-modified at the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA. In some embodiments, the gRNA is 2′-O-modified and 3′phosphorous-modified, e.g., 2′-O-methyl 3′thioPACE-modified at the second nucleotide from the 3′ end of the gRNA, the third nucleotide from the 3′ end of the gRNA, and the fourth nucleotide from the 3′ end of the gRNA. In some embodiments, the nucleotide at the 3′ end of the gRNA is not chemically modified. In some embodiments, the nucleotide at the 3′ end of the gRNA does not have a chemically modified sugar. In some embodiments, the gRNA is 2′-O-modified and 3′phosphorous-modified, e.g. 2′-O-methyl 3′thioPACE-modified at the nucleotide at the 5′ end of the gRNA, the second nucleotide from the 5′ end of the gRNA, the third nucleotide from the 5′ end of the gRNA, the second nucleotide from the 3′ end of the gRNA, the third nucleotide from the 3′ end of the gRNA, and the fourth nucleotide from the 3′ end of the gRNA.

In some embodiments, a gRNA provided herein comprises a chemically modified backbone. In some embodiments, the gRNA comprises a phosphorothioate linkage. In some embodiments, one or more non-bridging oxygen atoms have been replaced with a sulfur atom. In some embodiments, the nucleotide at the 5′ end of the gRNA, the second nucleotide from the 5′ end of the gRNA, and the third nucleotide from the 5′ end of the gRNA each comprise a phosphorothioate linkage. In some embodiments, the nucleotide at the 3′ end of the gRNA, the second nucleotide from the 3′ end of the gRNA, and the third nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a phosphorothioate linkage. In some embodiments, the second nucleotide from the 3′ end of the gRNA, the third nucleotide from the 3′ end of the gRNA, and at the fourth nucleotide from the 3′ end of the gRNA each comprise a phosphorothioate linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a phosphorothioate linkage.

In some embodiments, a gRNA provided herein comprises a thioPACE linkage. In some embodiments, the gRNA comprises a backbone in which one or more nonbridging oxygen atoms have been replaced with a sulfur atom and one or more non-bridging oxygen atoms have been replaced with an acetate group. In some embodiments, the nucleotide at the 5′ end of the gRNA, the second nucleotide from the 5′ end of the gRNA, and the third nucleotide from the 5′ end of the gRNA each comprise a thioPACE linkage. In some embodiments, the nucleotide at the 3′ end of the gRNA, the second nucleotide from the 3′ end of the gRNA, and the third nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end of the gRNA, the nucleotide at the 3′ end of the gRNA, the second nucleotide from the 3′ end of the gRNA, and the third nucleotide from the 3′ end of the gRNA each comprise a thioPACE linkage. In some embodiments, the second nucleotide from the 3′ end of the gRNA, the third nucleotide from the 3′ end of the gRNA, and at the fourth nucleotide from the 3′ end of the gRNA each comprise a thioPACE linkage. In some embodiments, the nucleotide at the 5′ end of the gRNA, the second nucleotide from the 5′ end of the gRNA, the third nucleotide from the 5′ end, the second nucleotide from the 3′ end of the gRNA, the third nucleotide from the 3′ end of the gRNA, and the fourth nucleotide from the 3′ end of the gRNA each comprise a thioPACE linkage.

In some embodiments, a gRNA described herein comprises one or more 2′-Omethyl-3′-phosphorothioate nucleotides, e.g., at least 1, at least 2, at least 3, at least 4, at least 5, or at least 6 2′-O-methyl-3′-phosphorothioate nucleotides. In some embodiments, a gRNA described herein comprises modified nucleotides (e.g., 2′-O-methyl-3′-phosphorothioate nucleotides) at one or more of the three terminal positions and the 5′ end and/or at one or more of the three terminal positions and the 3′ end. In some embodiments, the gRNA comprises one or more modified nucleotides, e.g., as described in PCT Publication Nos. WO2017/214460, WO2016/089433, and WO2016/164356, which are incorporated by reference their entirety.

The gRNAs provided herein can be delivered to a cell in any manner suitable. Various suitable methods for the delivery of CRISPR/Cas systems, e.g., comprising an RNP including a gRNA bound to an RNA-guided nuclease, have been described, and exemplary suitable methods include, without limitation, electroporation of RNP into a cell, electroporation of mRNA encoding a Cas nuclease and a gRNA into a cell, various protein or nucleic acid transfection methods, and delivery of encoding RNA or DNA via viral vectors, such as, for example, retroviral (e.g., lentiviral) vectors. Any suitable delivery method is embraced by this disclosure, and the disclosure is not limited in this respect.

The present disclosure provides a number of GBA target sites and corresponding gRNAs that are useful for targeting an RNA-guided nuclease to human GBA, and also a number of safe harbor loci (e.g., CCR5, RAB11a, and AAVS1) target sites and corresponding gRNAs that are useful for targeting an RNA-guided nuclease to a safe harbor locus (e.g., CCR5, RAB11a, and AAVS1). Table 1 below illustrates preferred target domains in the human endogenous GBA gene that can be bound by gRNAs described herein. The exemplary target sequences of human GBA shown in Table 1, in some embodiments, are for use with a Cas9 nuclease, e.g., SpCas9.

TABLE 1
Exemplary Cas9 target site sequences of human
GBA are provided, as are exemplary gRNA targeting
domain sequences useful for targeting such sites.

| Guide | |
|---|---|
| Name | Target Domain Sequence |
| SG1 | (SEQ ID NO: 1) ACATGGTACAGGAGGTTCTA |
| | (SEQ ID NO: 2) TAGAACCTCCTGTACCATGT |
| | (SEQ ID NO: 3) ACAUGGUACAGGAGGUUCUA |
| SG2 | (SEQ ID NO: 4) CACATGGTACAGGAGGTTCT |
| | (SEQ ID NO: 5) AGAACCTCCTGTACCATGTG |
| | (SEQ ID NO: 6) CACAUGGUACAGGAGGUUCU |
| SG3 | (SEQ ID NO: 7) AGCCGACCACATGGTACAGG |
| | (SEQ ID NO: 8) CCTGTACCATGTGGTCGGCT |
| | (SEQ ID NO: 9) AGCCGACCACAUGGUACAGG |
| SG4 | (SEQ ID NO: 10) CTAGAACCTCCTGTACCATG |
| | (SEQ ID NO: 11) CATGGTACAGGAGGTTCTAG |
| | (SEQ ID NO: 12) CUAGAACCUCCUGUACCAUG |
| SG5 | (SEQ ID NO: 13) GTCCAGGTCGTTCTTCTGAC |
| | (SEQ ID NO: 14) GTCAGAAGAACGACCTGGAC |
| | (SEQ ID NO: 15) GUCCAGGUCGUUCUUCUGAC |
| SG6 | (SEQ ID NO: 16) TGCCAGTCAGAAGAACGACC |
| | (SEQ ID NO: 17) GGTCGTTCTTCTGACTGGCA |
| | (SEQ ID NO: 18) UGCCAGUCAGAAGAACGACC |
| SG7 | (SEQ ID NO: 19) GCATCAGTGCCACTGCGTCC |
| | (SEQ ID NO: 20) GGACGCAGTGGCACTGATGC |
| | (SEQ ID NO: 21) GCAUCAGUGCCACUGCGUCC |
| SG8 | (SEQ ID NO: 22) GAAGAACGACCTGGACGCAG |
| | (SEQ ID NO: 23) CTGCGTCCAGGTCGTTCTTC |
| | (SEQ ID NO: 24) GAAGAACGACCUGGACGCAG |

For each target site, the first sequence represents the DNA target domain sequence, the second sequence represents the reverse complement thereof, and the third sequence represents an exemplary targeting domain sequence of a gRNA that can be used to target the respective target site.

TABLE 2
Exemplary template polynucleotide ssODNs for
HDR-editing of human GBA are provided.

| ssODN | |
|---|---|
| Name | Sequence |
| ssODN1 | (SEQ ID NO: 25) |
| | TGCCTCTCCCACATGTGACCCTTACCTACACTCTCTGGGGACCCCCAGTGTTGCG |
| | CCTTTGTCTCTTTGCCTTTGTCCTTACCCTAGAGCCTGCTCTATCATGTGGTCGG |
| | CTGGACCGACTGGAACCTTGCCCTGAACCCCGAAGGAGGACCCAATTGGGTGCGT |
| | AACTTTGTCGACAGTCCCATCATTGTAGACATCAC |
| ssODN2 | (SEQ ID NO: 26) |
| | TGCCTCTCCCACATGTGACCCTTACCTACACTCTCTGGGGACCCCCAGTGTTGCG |
| | CCTTTGTCTCTTTGCCTTTGTCCTTACCCTAGAGCCTCCTGTACCATGTGGTCGG |
| | CTGGACCGACTGGAACCTTGCCCTGAACCCCGAAGGAGGACCCAATTGGGTGCGT |
| | AACTTTGTCGACAGTCCCATCATTGTAGACATCAC |
| ssODN3 | (SEQ ID NO: 27) |
| | TGATGGGACTGTCGACAAAGTTACGCACCCAATTGGGTCCTCCTTCGGGGTTCAG |
| | GGCAAGGTTCCAGTCGGTCCAGCCGACCACGTGATAGAGCAGGCTCTAGGGTAAG |
| | GACAAAGGCAAAGAGACAAAGGCGCAACACTGGGGGTCCCCAGAGAGTGTAGGTA |
| | AGGGTCACATGTGGGAGAGGCAGCTGTGGGTAGGT |
| ssODN4 | (SEQ ID NO: 28) |
| | TGATGGGACTGTCGACAAAGTTACGCACCCAATTGGGTCCTCCTTCGGGGTTCAG |
| | GGCAAGGTTCCAGTCGGTCCAGCCGACCACATGGTACAGGAGGCTCTAGGGTAAG |
| | GACAAAGGCAAAGAGACAAAGGCGCAACACTGGGGGTCCCCAGAGAGTGTAGGTA |
| | AGGGTCACATGTGGGAGAGGCAGCTGTGGGTAGGT |
| ssODN5 | (SEQ ID NO: 29) |
| | GGGAGGTACCCCGAGGGACTCTGACCATCTGTTCCCACATTCAGCAAGTTCATTC |
| | CTGAGGGCTCCCAGAGAGTGGGGCTGGTTGCTAGCCAGAAAAATGATCCGGACGC |
| | AGTGGCACTGATGCATCCCGATGGCTCTGCTGTTGTGGTCGTGCTAAACCGGTGA |
| | GGGCAATGGTGAGGTCTGGGAAGTGGGCTGAAGAC |
| ssODN6 | (SEQ ID NO: 30) |
| | CAACGCTGTCTTCAGCCCACTTCCCAGACCTCACCATTGCCCTCACCGGTTTAGC |
| | ACGACCACAACAGCAGAGCCATCGGGATGCATGAGGGCGACGGCATCCGGGTCGT |
| | TCTTCTGACTGGCAACCAGCCCCACTCTCTGGGAGCCCTCAGGAATGAACTTGCT |
| | GAATGTGGGAACAGATGGTCAGAGTCCCTCGGGGT |

TABLE 3
Exemplary Cas9 target site sequences of human CCR5
are provided, as are exemplary gRNA targeting
domain sequences useful for targeting such sites.

| Guide | |
|---|---|
| Name | Target Domain Sequence |
| SG9 | (SEQ ID NO: 31) TGACATCAATTATTATACAT |
| | (SEQ ID NO: 32) ATGTATAATAATTGATGTCA |
| | (SEQ ID NO: 33) UGACAUCAAUUAUUAUACAU |
| SG10 | (SEQ ID NO: 34) TTTTGCAGTTTATCAGGATG |
| | (SEQ ID NO: 35) CATCCTGATAAACTGCAAAA |
| | (SEQ ID NO: 36) UUUUGCAGUUUAUCAGGAUG |
| SG11 | (SEQ ID NO: 37) GTAGAGCGGAGGCAGGAGGC |
| | (SEQ ID NO: 38) GCCTCCTGCCTCCGCTCTAC |
| | (SEQ ID NO: 39) GUAGAGCGGAGGCAGGAGGC |
| SG12 | (SEQ ID NO: 40) TTCACATTGATTTTTTGGCA |
| | (SEQ ID NO: 41) TGCCAAAAAATCAATGTGAA |
| | (SEQ ID NO: 42) UUCACAUUGAUUUUUUGGCA |

For each target site, the first sequence represents the DNA target domain sequence, the second sequence represents the reverse complement thereof, and the third sequence represents an exemplary targeting domain sequence of a gRNA that can be used to target the respective target site.

TABLE 4
Exemplary template polynucleotide ssODNs for
HDR-editing of human CCR5 are provided.

| ssODN | |
|---|---|
| Name | Sequence |
| ssODN7 | (SEQ ID NO: 43) |
| | TCTAGGACTTTATAAAAGATCACTTTTTATTTATGCACAGGGTGGAACAAGATGG |
| | ATTATCAAGTGTCAAGTCCAATCTATGACATCAATTATTATACGATCGCATCGGA |
| | GCCCTGCCAAAAAATCAATGTGAAGCAAATCGCAGCCCGCCTCCTGCCTCCGCTC |
| | TACTCACTGGTGTTCATCTTTGGTTTTGTGGGCAA |
| ssODN8 | (SEQ ID NO: 44) |
| | CCAGAAGGGGACAGTAAGAAGGAAAAACAGGTCAGAGATGGCCAGGTTGAGCAGG |
| | TAGATGTCAGTCATGCTCTTCAGCCTTTTGCAGTTTATCAGGCGATCGATGAGGA |
| | TGACCAGCATGTTGCCCACAAAACCAAAGATGAACACCAGTGAGTAGAGCGGAGG |
| | CAGGAGGCGGGCTGCGATTTGCTTCACATTGATTT |
| ssODN9 | (SEQ ID NO: 45) |
| | ATGCTCTTCAGCCTTTTGCAGTTTATCAGGATGAGGATGACCAGCATGTTGCCCA |
| | CAAAACCAAAGATGAACACCAGTGAGTAGAGCGGAGGCAGGACGATCGGGCGGGC |
| | TGCGATTTGCTTCACATTGATTTTTTGGCAGGGCTCCGATGTATAATAATTGATG |
| | TCATAGATTGGACTTGACACTTGATAATCCATCTT |
| ssODN10 | (SEQ ID NO: 46) |
| | GGATGACCAGCATGTTGCCCACAAAACCAAAGATGAACACCAGTGAGTAGAGCGG |
| | AGGCAGGAGGCGGGCTGCGATTTGCTTCACATTGATTTTTTGCGATCGGCAGGGC |
| | TCCGATGTATAATAATTGATGTCATAGATTGGACTTGACACTTGATAATCCATCT |
| | TGTTCCACCCTGTGCATAAATAAAAAGTGATCTTT |

A representative nucleotide sequence of the GBA gene is provided by GenBank:

NG_009783.1, shown below.
(SEQ ID NO: 47)
CCGTGTTATCCAGGATGGTCTCAATCTCCTGACCTCGTGATCTGCCCGCCTCGGCCTCCCAAAGTGCTGGGATTA
CAGGCATGAGTCACCGTGCCCGGGCAATTTTTGTATTTTTTAGTAGAGACAGGGTTTCACCCTTTTGGCCAGGCT
GGTCTTGAACTCCTGACCTCAAGTGATCCACCCGCCTCGGCCTCCCAAAAGGATTTATTTTTTGAAACCAGTTCC
ACAGCTCTCAGCTTGGTCCACTTATCTGTCCTCCCCAAGCTTCAGCTGTCACTTGTTAAACATGTATAATAATAG
TACTTCAGGCCGGGCACGGTGGCTTGCACCTGTAATCCCAGCACTGTGGGAAGCTGAGGTGGGTGGATCACCTGA
GGTCGGGAGTTCGAGACCAGCCTGGCTAACATGGTGAAACCCTATCTCTACTAAAAATACAAAAATTAGCCAGGT
GTGGTGGAGCGCGCCTGTAATTCCAGCTACTAACAGAGAGGCTAAGGCAGGAGAATCGCTTGAACCTGGAAAGCA
GGGGTTGCCGTGAGCCAAGATCATGCCACTGCACTCCAGCCTGGGTGACAGAGACACACTCCATCTCAAAAACAC
AAACAAACAAACAAAAAACATGTATAATAACAGTACTTCAGCCATAGGCATTGTACTCGAAGATGCTGAGAAAGG
AGACAGTGGCCGGGCAAGGCTGTTCACACCTATAATCCCAGCACTTTGGGAGGCCAAGGCAGGTGGATCACCTGA
GGTCAGGAGTTCAAGACCAGCCTAGCCAACATGGTGAAACCCCCATCTCTACTAAAAATTGAAAAATTAGCTGGG
CCTGGTGGTGGACGCCTGTAATCCCAGCTACTAGGGAGGCTGAGGCAGGAGAATCGCTTGAACCTGGGAGGCGGA
GGTTGCAGTGAGCTGAGATCGTACCACTGCACTCCGGCCTGGGCAACACAGTGAGACTCCATCTCAAAAAAATAA
GAAAGGAGATAGTACTGGGGAACGCTCAGCACTGTGCGCCAGGTGCTGAACAACACCACTGCAGTCCTTGTTGTG
GTGGATTGTACCATCTAGTTGCTGGCTAATATGGACAGAGATGCTGGCCCTTTGATTGGGGATGGAGCGTGGGAG
CTGTGAAAGCTCCTCTGGGCTTGAGTTCCCACAGGAGGGTGGGCGTGTCCACAGAACACTTCCACTCACTCCCTG
TCTCCCTTTCTCTCTTCTCCCCAGCTGACTTCAGGGACCTTTATACCAAAGTGCTTGAGGAAGAAGCTGCTTCTG
TTTCCTCTGCAGATACAGGTCAGGCATGTGGTTTGCGCCCCAGGGATGGGGATTGGGCATGGCTGCCCAGCCCCC
TCTCCACCCTACAATACCATTCTCTTATCTCTGTCTCTCTGCAGGGCTCTGCTCTGAAGCCTGCCTCTTCCGCCT
AGCCCGCTGCCCTTCCCCCAAGTTGCTACGTGCCCGGTCAGCCGAGAAACGGCGCCCTGTGCCCACCTTCCAAAA
AGTTCCCCTGCCCTCGGGCCCTGCACCTGCCCACTCCCTGGGGGACCTAAAGGGCAGCTGGCCAGGTCGGGGCCT
GGTCACTCGTTTCCTCCAGATATCCAGGAAAGCCCCAGACCCCAGTGGGACTGGAGCTCATGGACATAAGCAGGT
AGGAATTCGGGGAGCCAGGAAAGATGTTTGGGAAAGCGTGGAGCTTCAGATTGAGCCTTATTGATGATGCCCTTT
CTTGTGTCCCTGTCCAGGTGCCCCGGAGCCTGTGGGGCCGGCCTGGCCGAGAGAGCCTCCACCTTCGCAGCTGCG
GAGATCTGAGCTCTAGCTCTTCCCTGCGGCGTCTCCTGTCTGGCCGCAGGCTGGAGCGTGGTACCCGCCCCCACA
GCCTCAGCCTCAACGGGGGCAGCCGGGAGACTGGGCTCTGACCTAGGCTTCTTGTCACACTGAACACATCCAGCC
ACAGGCACCAGCTGGTTGGGACCAGCAGCCCCCAGCATCCTCTTGCACTGGCTGGCACAAAAAGAAACCTGCTGT
ATACCCCCCAAAGTGTCCCTTTCCCTCCTACCTCTGGGGTCTCTTGCTGCTTGCCTCTGCTGCTCTGGACTGGGA
GAGCTTCTGTCCTGTGCTGCATGGGTATTTAGACTGTGGGGGAGATGCCCCTTCTTATAGCACTGGAGGAGGAAA
ACAAATTCTTGTCCCCCTCAGAATGAGAGTGGCTCTTTCTGATTTGCAAGGGCACTATGGTCAGGGCAAAGGCAT
GGCCCAGGTGTTTAAGTACAGGGTGACGTGTGCCTATGCAATGGGGTGGTAAGGCAGGCACGAAGAGTCCAAAAA
ATCTAGGTGGCCTCTCAGCTCTGCCACCTCTAGCTGCATGACCTTGGGCAAGCTATGTAACCCCAATTGCCTGCT
CCATTAAAGACTGTGAAGGTAGAATGTTTGTAAAGCTCTTAACAGTATGTAAGCCTTCAATAAATTTCAGTTTTC
CCCTTGTTTTCTTGATCATTCTCTGTCACCAGTGAAATTTGTTCTAGTGTCTCTCATATTTAAGAAAACTCTTTC
AGGACTGGGTATGGTGGCTCACACCTATAATCCTAGCACTTTGGGAGGCCGAAGCAAGAGGATCGCCTGAGCCTA
GGAATTCAAGACCAGCCTGGGCAACATAGTGAGACCCTGTCTCTACAAAAAACAAAAAATTAGCCAGGCATGGTG
GGACACGCCTGTAGTCCCAACTACTCAGGTGGCTAAGGTGAGAGGATCACTTGAGCTTGGGAAGTCCAGGCTGCA
GTAAGCTGTGATTGAGCCACTGCACTACAGCCTGGGCAACAGAGCAAGACCATGTCTCAAAAAAAAAAAAAAAAG
AAAAAAGAAACTTTCAAGACACTCTTTCCAACCACTAATTGTAACTCTGCTCCTCCTTTTCACAGCAATAGGTTT
TCTTTTTCTTCCCTCCACTGTTAAACATCCATTCTCTCCTCACCCACCCCCATCAGACTCCTTCCCCTATCTTTC
CACAGCCACTGCTCTGACCAAACTTTCCAGTGACCACAGTGGTGTCAGACCCAGTGACCATTTCTCTGCCTGCAT
CTCACTTGACCTCGAGGCAGCAATTAATACCCATAATCAGCATCTTCTTGAATTTGTCCCTTTGAAAAGGGAAAT
ATTGGCTCTTCTACTTTGTCCTGCTGAACTGCTTAACATTGGAGGGCCCCAGGGCCCTCACCTAAGCCCTCTTTC
CTACCTCCACTCTTTCTATAGGTGGCCCTACTACTAAAGTCCATGGCTTTAAATACCATCTTTCTATGTGTTAAT
CCATAACTCCAGCCTTGACCTCCCATGAGCGCCATCCAACTCAGCATGTCTGCTTGGATGTCTAATGGGCATTTC
AGATTCAACATGGCCACAACTGAACTCTTGATTCCCACCCCAGCACCGGTTATTTTTCCACTGTTCCCATCTCAA
TGGCACCTCCATTACCCATTTGCACATTCCAAAAGCTCAGGAACCATGGTGACTTCTTTTCCCATATCCAACACA
ACCAATCCTATCCTGAATTCATCCACATCCCACCACCTCCCCAGCTACCTAGCTCCAGCCATCCTCTCTCCACAA
CCTCTGAATCAGTCTTTCACTTTTCCCAGCAATCCATTCTCCACTCAGCAAAATGATGATAAAGCACGTCACATC
AAGGCTCTGCCTCAATTTAATGGCTTCCCATTGTATTTAGAATCATCTCCAAACTCCCAGAGACTATGGTCAGCT
ACAATCTGGCCCACCTTCTGTTCCAGCCAAATTTCCTCACAGCACAAGGACGTTTGCACCTGCTGTTTTCCAAGC
ATGAAACCCTTGGCCCCTATATCTGGTGCTATCACCTAATATCAGGITTTAGCTCCATTCTCACCATTTCAGTGA
GCACCCAATCCCCATCGCAGTCATTCTATCACATAGCCATGTTTTTTTTTGTTTGTTTGTTTCATTTTGTCTTTT
TTTGAGACAGGGTCTTGCTTTGTTACCCAGGCTGGAGTGCAGTGGTGTGATTTGGGCTCACTGCAACCTTCCACC
TCCTGGGTTCAAGCAATTCTCCTGCCTCAGCCTCCCGAGTAGCTGGGATTACAGGCGCCCGTCCCCATGCCCGCC
CAGCTAAATTTTGTATTTTTAGAAGAGATAGGGTTTCACCATGTTGGCCAGGCGGGTCTCAAACTCCTGACCTCA
AGTAATCCGCCTGCCTCGGTCTCCCAAAGTGCTGGGATTACAGGTGTGACTCACCGCGCCTGGCCACATACCCAT
GGTTTCAGCATGTATCACTATCTAAAATTATTATTTTTGTTTATATATCTGTGTCGTCCCATAGAATGTTAAGGT
CCCAAGATCAGAAACTTGCTCATTGCAGTGGGTCTAACACTCAGTAGGTCCTCAACAAACATTCGTTAAGATACT
AAAGTGGCAGGGTGGGGCCCTGTAAACAGCTTCAGGACCCTGTGCTTGTAGGGGCAACGTGGTGCCCTCCAAGGA
AGACAGGGAGGTGGGAGGAGCACTGCCCAGAGATGGCGTCAGGCTGCAAGACTTCTTGAATAATTCAGCATCATA
ACAACCCAGCCTCAGGAAGGGATAGGGCACGGCCAGGACGAAACATTAGGAGGCGATGGACAATGGGATTCCCAC
GGGGCAGCTTCTGCGCACTGGACGTTCCCTAACCTGAGGCTCTCTAAAGAGGAAGGTTAGGAATCCTCTGAGCTT
CGGTGGGCTGGACTCACTGTGGGAATTCAATCGCCCCCATCCACCAACAGTGTGCTGGCGGGAAAACGCCGACAC
GCATGCGTAGTTCTCGCGCCGGCTCCTCTCTCTCTCTCTCTCTCTCTCGCTCGCTCTCTCGCTCTCTCGCTCTCT
CTCGCTCGCTCTCTCGCTCTCGCTCTCTCTCTCTCTCCGGCTCGCCAGCGACACTTGTTCGTTCAACTTGACCAA
TGAGACTTGAGGAAGGGCTCTGAGTCCCGCCTCTGCATGAGTGACCGTCTCTTTTCCAATCCAGGTCCCGCCCCG
ACTCCCCAGGGCTGCTTTTCTCGCGGCTGCGGGTGGTCGGGCTGCATCCTGCCTTCAGAGTCTTACTGCGCGGGG
CCCCAGTCTCCAGTCCCGCCCAGGCGCCTTTGCAGGCTGCGGTGGGATTTCGTTTTGCCTCCGGTTGGGGCTGCT
GTTTCTCTTCGCCGACGGTAGGCGTAATGAATATTTCGACCTTTGGATCTTAGCTGTCCCCTCCCTGCGTTCGCA
CTTAACCTTTTTCACCATTATTATTATTATTGTTATTATTATTATTTTTTGAGGGAGTCTCGCCCTGTCGCCCAG
GCTGGAGTGTAATGGCGCCTTCTTGGCTCACTGCAACCTCCGCCTCCCGGGTTCAGGCGATTCTCCGACCTCAGC
CTCCCAAGTACGTGGGATTACAGGCACCCGCCACCACGCACGGCTAATTTTTTGTATCTTTTAGTAGAGACGGGG
TTTCACCATGTTGGTCAGGCTGGTCTCCAATTCCTGACCTCGTGATCCGCCCGCCTCGGCCTGCCAAACAGCTGT
GATTATAGGCGTGAGCCACCGCGCCCGGCCAACCATCATTATTATTTTTAACGGTAAGGATGGTCAGATTTTACT
AATGAAGAAGAGATTATAAAATCTTCAAGTCTTTATATCCACTTGCTTTTTGAGGGGTGGAGTGGGAAGAAGGTT
ATGTAATTCATACGTTCTTCAGACATGTGACAAACATTCACGGAGCCCGGCGACGAGCGTCGGGGTTGGGATTCG
CACTGGAGCTGCAGATGGGTGCCAGGATGGACTGGTCCCTACCCTCCGCTTGAACCTAGGAGGCGGAGGTTGCAG
TGAACCGAGATCGTGCCACTGCACTCCAGCCTGGGTGACAGAGATACTCCGTCTCAAAAAAAAAAACAAAACAAA
AAACAAGCGGACTGGGCGCAGTGCCTCACCCTGTAATCCCAGCACTTTGCAAAGCCAAGGCGGGAGGATCCTTTG
AGTTTAGGAGTTTGAGACCAACCTGCGCAACACAGTAAGACCCCGTCTCTACAAAAAATACAGAAATTAGCCAGG
TGTGGTGGTGTGCGCCTATAGTCCCAGCTATTCTGGAGGCTGAGGTGGGAGGATTGCTTATTCTGGAGGCAGAGG
TTGCACTGAGCCGAAATCAAGCTACTACACTCCATCCAGGGCAACATACGGAGACCCTGTCTCAAACAAACAAAC
AAAAAATTGCTCAGTACCTGGCCAAAAAAGAAGAGGCTCACTATGCAGAGGGGAAGTGGAAGGAGATGTTTGGAC
TTCTAAACTCAATAGAGCAGGAGAGGCAAATGTAGAATGTGCTCAGGAAATATCTGTGAGATGAATGAACTTGAG
GGAAGTAAGGTACTAGATATTACCTGCCCTACCCAGAACAAATCCTGTGCAATGTTTCCTTGAAAAGTGAGAAGT
CTGGAAGGGGTGGCTACTGACATAGTGAAGCAACTAGTTCAATTCTACAACTTGACAGCTACCCCTGTGCCAGGC
TATCTACGAGGATACTTAGAATGCATAAGACATTCCTTCAAGGAACTCCAGGAACAGAGGCCTGACATGTTGCAA
TGTTTAGTGTCAAGCAGTGTACTAGAGACACATTATCACACTCAAACCTCACAACAATTCTGTGAGGTAGGAGTT
ATCACTCCCCTTTTATAGATGAAACAGAGGCTTAGAGTGATTGATTTATTGAAAGTCAAACAGCCAGTAAATGGT
GTAGCCAGGATTCCAAACTTGCTGTCTCACTGAGACTGTACTTAATTACTGGAGGGACCGGGTGTGGTGGCTCAT
TGCTATAATCCCAACACCTTGGGAGGCTGAGGCTGGTGGATCACCTGAGGTCAGGGGTTCGAGACCAGCCTGGCC
AACATGGTGAAACCCCATCTCTACTAAAAATACAAAAATTAGCTGGGCATGGTGGTGGGCTCCTGTAATCCCAGC
TACTCAGGAGGCTGAGGCAGGGCAATTGCTTGAGCCGAGATCACACTGCACTCCAGCCTGGGCAACAGGGCAAGA
CTCTGTCTCAAAACCAAAAAAAAAAAAATTACTGGAGGAACCTAGAAGAAGAAATGATCAATTTTGCTTGGAGTG
TATCTAGAAAGACTTCACTGAGATCATTTAAAGAACAAAAAGGATGGCTGGGGTCCAGCGCAGTGGCTCATGCCT
GTAATCCCAGCACTTTCGGATACCAAGGCAGCAGATCACCTGAGGTCCAGAGTTTCAGACCAGCCTGGCCAACAT
AGTGAAACCCCATCTCTACTAAAAATAAAAAAATTAGCTGAGCATGTTGGAGGGCACCTGTAATCCCAGCTACTT
GGGAGGCTGAGGCAGGAGAATCACTCGAACCCAGGAGGTGGAGGTTGCAGTGAGCCAAGATCACGCCACTGCACT
CCAGCCTGGGCAACAGAGTGAGACTCTGTCTCAAAAAACAACAACAACAAAAAATACAAACAAGAGACAAGTAGT
TCCCAGGTGCCTACCAAGTGGTCAGGCACTGCACTTACCTCACTGACTGCAGTAACCACCCTTTGAGGTTGTGGC
ATTGCCTCCATTTTCCAGGCAAGGAAATGGGCTGAGAGCTGGGATTAGTCAGGTCATGACTGTGTGTGCCACTCC
CGCTAAATCTCATTTGATGTGGTTCATGAGGCCACACCATGGACAGCTTCCTCCTTGTGTCCACTGAGGATATGG
CTTTGTACAACACTTTGGTTTTTGAACGACTTTACAAACCTCCCTGTCTTGTGAGGAAGGAAGAACAGTTATTAC
CATCTGCATCTGATGATGAAACAAGGGACGCTGCAGAGGAGCCGCACTGACCACTCCCTCCCTCCAGTCCTGTCA
TCCCACTGCCAGTGTCCCACCCTCTTGTGCCCTGCACTTCACTGGCTAATAACCCCCCTCACTTTTTCCTCTGTG
AAGCCATCCTGGATAATTCCCCACCCACGAATGGTCCCTCCTCATCTCAGAGAGCTCTCCATGCACACCTGTTAC
CGTTTCTGTCTTTATCTGTAAATATCTGTGTGTCTGACTTCCATGCCTCACACACCTCTATAGGGCAAAGACTGT
CTTAAACATCTTGGTAGTGTCAGTATTTTGCACAGTGAAGTTTTTTTTTTTAAATTATATCAGCTTTATTTGTAC
CTTTTTGACATTTCTATCAAAAAAGAAGTGTGCCTGCTGTGGTTCCCATCCTCTGGGATTTAGGAGCCTCTACCC
CATTCTCCATGCAAATCTGTGTTCTAGGCTCTTCCTAAAGTTGTCACCCATACATGCCCTCCAGAGTTTTATAGG
GCATATAATCTGTAACAGATGAGAGGAAGCCAATTGCCCTTTAGAAATATGGCTGTGATTGCCTCACTTCCTGTG
TCATGTGACGCTCCTAGTCATCACATGACCCATCCACATCGGGAAGCCGGAATTACTTGCAGGGCTAACCTAGTG
CCTATAGCTAAGGCAGGTACCTGCATCCTTGTTTTTGTTTAGTGGATCCTCTATCCTTCAGAGACTCTGGAACCC
CTGTGGTCTTCTCTTCATCTAATGACCCTGAGGGGATGGAGTTTTCAAGTCCTTCCAGAGAGGTAAGAGAGAGAG
CTCCCAATCAGCATTGTCACAGTGCTTCTGGAATCCTGGCACTGGAATTTAATGAATGACAGACTCTCTTTGAAT
CCAGGGCCATCATGGCTCTTTGAGCAAGGCACAGATGGAGGGAGGGGTCGAAGTTGAAATGGGTGGGAAGAGTGG
TGGGGAGCATCCTGATTTGGGGTGGGCAGAGAGTTGTCATCAGAAGGGTTGCAGGGAGAGCTGCACCCAGGTTTC
TGTGGGCCTTGTCCTAATGAATGTGGGAGACCGGGCCATGGGCACCCAAAGGCAGCTAAGCCCTGCCCAGGAGAG
TAGTTGAGGGGTGGAGAGGGGCTTGCTTTTCAGTCATTCCTCATTCTGTCCTCAGGAATGTCCCAAGCCTTTGAG
TAGGGTAAGCATCATGGCTGGCAGCCTCACAGGATTGCTTCTACTTCAGGCAGTGTCGTGGGCATCAGGTGAGTG
AGTCAAGGCAGTGGGGAGGTAGCACAGAGCCTCCCTTCTGCCTCATAGTCCTTTGGTAGCCTTCCAGTAAGCTGG
TGGTAGACTTTTAGTAGGTGCTCAATAAATCCTTTTGAGTGACTGAGACCAACTTTGGGGTGAGGATTTTGTTTT
TTTTCTTTTGAAACAGAGTCTTACTCTGTTGCCTGGGCTGGAGTGCAGTGGTGCAATTTTGGCTCATTCCAACCT
CTGCCTCCCAGATTCAAGCGATTCTCTTGCTTCAGCTTCCCAGGTAGCTGGGATTACAGGCGGCCACCACTACGC
CCAGCTAATTTTTGTATTTTTAGTAGAGACGGGGTTTCACCATGCTGGCAAGGCAGGTCTCAAACTCCTCACCTC
AGGTGATCCGCCCACCTCGGCCTCCTAAAGTGCTAGGATTACAGGTGTGAGCCCCTGCGCCCGGCCAAGGGGTGA
GGAATTTTGAAACCGTGTTCAGTCTCTCCTAGCAGATGTGTCCATTCTCCATGTCTTCATCAGACCTCACTCTGC
TTGTACTCCCTCCCTCCCAGGTGCCCGCCCCTGCATCCCTAAAAGCTTCGGCTACAGCTCGGTGGTGTGTGTCTG
CAATGCCACATACTGTGACTCCTTTGACCCCCCGACCTTTCCTGCCCTTGGTACCTTCAGCCGCTATGAGAGTAC
ACGCAGTGGGCGACGGATGGAGCTGAGTATGGGGCCCATCCAGGCTAATCACACGGGCACAGGTAACCATTACAC
CCCTCACCCCCTGGGCCAGGCTGGGTCCTCCTAGAGGTAAATGGTGTCAGTGATCACCATGGAGTTTCCCGCTGG
GTACTGATACCCTTATTCCCTGTGGATGTCCTCAGGCCTGCTACTGACCCTGCAGCCAGAACAGAAGTTCCAGAA
AGTGAAGGGATTTGGAGGGGCCATGACAGATGCTGCTGCTCTCAACATCCTTGCCCTGTCACCCCCTGCCCAAAA
TTTGCTACTTAAATCGTACTTCTCTGAAGAAGGTGAGGAGGAAGGGGACAAGATGACATAGAGCCATTGAAACTT
TTCGTTTTTCTTTTCTTTTTTTAAAATTTTTTTGAGGCAGAATCTCACTCTGCCCATTCTGTCGGCGAGACAGGA
GTGCAGTGGTGTGATCTCCCCTCACAGCAACCTCTGCCTCCCAGGCTATAGTGATTCTCCTGCCTCAGCCTCCTG
AGTAGCTGGAATTATAGGCGTGCGCCACTACCACCTGGCTAATTTTTGTATTTTTAGTAGAGACAGGGTTTCATC
ATGTTGACCAGGCTAGTCTTAAACTCCTGACCTCAAATGATATACCTGCCTTGGCCTCCCGAAGTGCTGGAATTA
CAAGTGTGAGCCACCGAGCCCAGCAGACACTTTTCTTTTTTCTTTTTTTTTTTTTGAGACAGAGTCTCGCACTGT
CACCCAGGCTGGAGTGCAGTGGCACAATCTCAGCTCACTGCAACCTCCACCTCCCGGGTTCAGGTGATTCTCCTG
TCTCAGCCTCTCGAGTACCTGGGATTACAGGTGCCTGCCACCACGCCCGGCTAATTTTTTGTATTTTTAGTAGAG
ACAGGGTTTCACTATGTTGGCCAGGATGATTGCGAACTCCTGACCTCGTGATCTGCCCACATCGGCCTCCCAAAG
TGCTGGGATTACATGCGTGAGCCACTGACACTTTTCTTTGCCCTTTCTTTGGACCCTGACTTCTGCCCATCCCTG
ACATTTGGTTCCTGTTTTAATGCCCTGTGAAATAAGATTTCACCGCCTATCATCTGCTAACTGCTACGGACTCAG
GCTCAGAAAGGCCTGCGCTTCACCCAGGTGCCAGCCTCCACAGGTTCCAACCCAGGAGCCCAAGTTCCCTTTGGC
CCTGACTCAGACACTATTAGGACTGGCAAGTGATAAGCAGAGTCCCATACTCTCCTATTGACTCGGACTACCATA
TCTTGATCATCCTTTTCTGTAGGAATCGGATATAACATCATCCGGGTACCCATGGCCAGCTGTGACTTCTCCATC
CGCACCTACACCTATGCAGACACCCCTGATGATTTCCAGTTGCACAACTTCAGCCTCCCAGAGGAAGATACCAAG
CTCAAGGTAGGCATTCTAGCTTTTTCAGGCCCTGAGGGCCCTGATGTCTGGGGGTTGAGAAACTGTAGGGTAGGT
CTGCTTGTACAGACATTTTGTCCCCTGCTGTTTTGTCCTGGGGGTGGGAGGGTGGAGGCTAATGGCTGAACCGGA
TGCACTGGTTGGGCTAGTATGTGTTCCAACTCTGGGTGCTTCTCTCTTCACTACCTTTGTCTCTAGATACCCCTG
ATTCACCGAGCCCTGCAGTTGGCCCAGCGTCCCGTTTCACTCCTTGCCAGCCCCTGGACATCACCCACTTGGCTC
AAGACCAATGGAGCGGTGAATGGGAAGGGGTCACTCAAGGGACAGCCCGGAGACATCTACCACCAGACCTGGGCC
AGATACTTTGTGAAGTAAGGGATCAGCAAGGATGTGGGATCAGGACTGGCCTCCCATTTAGCCATGCTGATCTGT
GTCCCAACCCTCAACCTAGITCCACTTCCAGATCTGCCTGTCCTCAGCTCACCTTTCTACCTTCTGGGCCTTTCA
GCCTTGGGCCTGTCAATCTTGCCCACTCCATCAGGCTTCCTGTTCTCTCGGTCTGGCCCACTTTCTTTTTATTTT
TCTTCTTTTTTTTTTTTTTGAGAAGGAGTCTCTCTCTCTGTCACCCAGGCTGGAGTGCTGTGGCGCCATCTTCAC
TCACTGTAACCTCTGCCTCCTGAGTTCAAGCAATTCTCCTGCCTCAGCCTTCCAAGTAGCIGGGATTATAGGCGC
CTGCCACCAGGCCCAGCTGATTTTTCTATTTTTAGTAGAGACGGGGTTTCGCCAGGCTGTTCTCGAACTCCTGAA
CTCAAGTGATCCACCTGCCTCGGCTTCCCAAAGTGCTGGGATTACAGGTGTGAGCCACCACACCCAGCTGGTCTG
GTCCACTTTCTTGGCCGGATCATTCATGACCTTTCTCTTGCCAGGTTCCTGGATGCCTATGCTGAGCACAAGTTA
CAGTTCTGGGCAGTGACAGCTGAAAATGAGCCTTCTGCTGGGCTGTTGAGTGGATACCCCTTCCAGTGCCTGGGC
TTCACCCCTGAACATCAGCGAGACTTCATTGCCCGTGACCTAGGTCCTACCCTCGCCAACAGTACTCACCACAAT
GTCCGCCTACTCATGCTGGATGACCAACGCTTGCTGCTGCCCCACTGGGCAAAGGTGGTAAGGCCTGGACCTCCA
TGGTGCTCCAGTGACCTTCAAATCCAGCATCCAAATGACTGGCTCCCAAACTTAGAGCGATTTCTCTACCCAACT
ATGGATTCCTAGAGCACCATTCCCCTGGACCTCCAGGGTGCCATGGATCCCACAGTTGTCGCTTGAAACCTTTCT
AGGGGCTGGGCGAGGTGGCTCACTCATGCAAACCCAGCACTTTGGGAAGCCGAGGCGGGTGATCACCTGAGGTCA
GGAGTTTAAGACCACCCTGGCCAACGTGTTGAAACCCTGTGTCTACTAAAATACAAAAAAAAAAAATTATCTGGG
CATGATGGTGGGTGTCTGTAATCCCAGCTACTCAGGAGGCTGAGAAGGGAGAATCAGTTGAACCCGGGAGATGGT
GGTTGCGGTGAGCCGAGATCGCGCCACTGCACTCCAGCCTGGGAGGCTGAGCGAGACTCCATCTCGAAACAAAAC
AAAACAAAACTATCTAGGCTGGGGGTGGTGGTTCATGTATGTATGTGTATATACATATATATGTGTTTATATGTA
TATATATATACACACACACACATACATACACACACATACACACACAAATTAGCTGGGTGTGGCACCCGTGTAGTC
CCAGCTACTCAGGAGGCTAATGTGGGAGGATCAGTTGACCCTAGGAAGTCAAGGCTGCAGTGAGTCGTGATTGCG
CCACTGTACTCCAGCCCGAGTGACAGAGTGACATCCTGTCTCAAAAACAAAAAAAAATCTCCCCAAACCTCTCTA
GTTGCATTCTTCCCGTCACCCAACTCCAGGATTCCTACAACAGGAACTAGAAGTTCCAGAAGCCTGTGTGCAAGG
TCCAGGATCAGTTGCTCTTCCTTTGCAGGTACTGACAGACCCAGAAGCAGCTAAATATGTTCATGGCATTGCTGT
ACATTGGTACCTGGACTTTCTGGCTCCAGCCAAAGCCACCCTAGGGGAGACACACCGCCTGTTCCCCAACACCAT
GCTCTTTGCCTCAGAGGCCTGTGTGGGCTCCAAGTTCTGGGAGCAGAGTGTGCGGCTAGGCTCCTGGGATCGAGG
GATGCAGTACAGCCACAGCATCATCACGGTAAGCCACCCCAGTCTCCCTTCCTGCAAAGCAGACCTCAGACCTCT
TACTAGTTTCACCAAAGACTGACAGAAGCCCTTCCTGTCCAGCTTTCCCCAGCTAGCCTGCCCTTTTGAGCAACT
CTGGGGAACCATGATTCCCTATCTTCCCTTTCCTTCACAGGTCTGCACACCTCATTGCCCCTTTTGCAACTACTG
AGGCACTTGCAGCTGCCTCAGACTTCTCAGCTCCCCTTGAGATGCCTGGATCTTCACACCCCCAACTCCTTAGCT
ACTAAGGAATGTGCCCCTCACAGGGCTGACCTACCCACAGCTGCCTCTCCCACATGTGACCCTTACCTACACTCT
CTGGGGACCCCCAGTGTTGCGCCTTTGTCTCTTTGCCTTTGTCCTTACCCTAGAACCTCCTGTACCATGTGGTCG
GCTGGACCGACTGGAACCTTGCCCTGAACCCCGAAGGAGGACCCAATTGGGTGCGTAACTTTGTCGACAGTCCCA
TCATTGTAGACATCACCAAGGACACGITTTACAAACAGCCCATGTTCTACCACCTTGGCCACTTCAGGTGAGTGG
AGGGCGGGCACCCCCATTCCATACCAGGCCTATCATCTCCTACATCGGATGGCTTACATCACTCTACACCACGAG
GGAGCAGGAAGGTGTTCAGGGTGGAACCTCGGAAGAGGCACACCCATCCCCTTTTGCACCATGGAGGCAGGAAGT
GACTAGGTAGCAACAGAAAACCCCAATGCCTGAGGCTGGACTGCGATGCAGAAAAGCAGGGTCAGTGCCCAGCAG
CATGGCTCCAGGCCTAGAGAGCCAGGGCAGAGCCTCTGCAGGAGTTATGGGGTGGGTCCGTGGGTGGGTGACTTC
TTAGATGAGGGTTTCATGGGAGGTACCCCGAGGGACTCTGACCATCTGTTCCCACATTCAGCAAGTTCATTCCTG
AGGGCTCCCAGAGAGTGGGGCTGGTTGCCAGTCAGAAGAACGACCTGGACGCAGTGGCACTGATGCATCCCGATG
GCTCTGCTGTTGTGGTCGTGCTAAACCGGTGAGGGCAATGGTGAGGTCTGGGAAGTGGGCTGAAGACAGCGTTGG
GGGCCTTGGCAGGATCACACTCTCAGCTTCTCCTCCCTGCTCCCTAGCTCCTCTAAGGATGTGCCTCTTACCATC
AAGGATCCTGCTGTGGGCTTCCTGGAGACAATCTCACCTGGCTACTCCATTCACACCTACCTGTGGCGTCGCCAG
TGATGGAGCAGATACTCAAGGAGGCACTGGGCTCAGCCTGGGCATTAAAGGGACAGAGTCAGCTCACACGCTGTC
TGTGACTAAAGAGGGCACAGCAGGGCCAGTGTGAGCTTACAGCGACGTAAGCCCAGGGGCAATGGTTTGGGTGAC
TCACTTTCCCCTCTAGGTGGTGCCAGGGGCTGGAGGCCCCTAGAAAAAGATCAGTAAGCCCCAGTGTCCCCCCAG
CCCCCATGCTTATGTGAACATGCGCTGTGTGCTGCTTGCTTTGGAAACTGGGCCTGGGTCCAGGCCTAGGGTGAG
CTCACTGTCCGTACAAACACAAGATCAGGGCTGAGGGTAAGGAAAAGAAGAGACTAGGAAAGCTGGGCCCAAAAC
TGGAGACTGTTTGTCTTTCCTGGAGATGCAGAACTGGGCCCGTGGAGCAGCAGTGTCAGCATCAGGGCGGAAGCC
TTAAAGCAGCAGCGGGTGTGCCCAGGCACCCAGATGATTCCTATGGCACCAGCCAGGAAAAATGGCAGCTCTTAA
AGGAGAAAATGTTTGAGCCCAGTCAGTGTGAGTGGCTTTATTCTGGGTGGCAGCACCCCGTGTCCGGCTGTACCA
ACAACGAGGAGGCACGGGGGCCTCTGGAATGCATGAGAGTAGAAAAACCAGTCTTGGGAGCGTGAGGACAAATCA
TTCCTCTTCATCCTCCTCAGCCATGCCCAGGGTCCGGGTGCCTGGGGCCCGAGCAGGCGTTGCCCGCTGGATGGA
GACAATGCCGCTGAGCAAGGCGTAGCCCACCATGGCTGCCAGTCCTGCCAGCACAGATAGGATCTGGTTCCGGCG
CCGGTATGGCTCCTCCTCAGTCTCTGGGCCTGCTGGTGTCTGGCGTTGCGGTGGTACCTCAGCTGAGGGTCAAGG
AAGGAAGGTGTGTTAGGAGAACTAGTTCTTGGATCCCTGCCCACTCTCCCCAGGGCTGCCCCTCCCATCTGCCCC
TTACCTCCATCCCAGGGGAAGTAGAGACTGAGAATGTGGGTACAATAGGCACAGAGGTTGTGCAGCCCACGCAGG
TGGACCTGCAGCTTCCCACTGGGCAGCTTTGCCTGCAGCAGCAGGGCCAAGTAGCTGAAGACGAAGGCGTCCAAG
GAGGCAGGGCTGGAGCAGAGAGAGAAGGGTGGGATGGAGGAGAACCACTGGGGTAGAAGGGGTAAAGATGGAGCT
GGAGGAAGAGTCAGCCTTGGGAGGTGGGCTCTGGGCAGCAGGCGGCCACCAGGGAAGGACAGGACACACAGTTCT
AGACCTGGTATGGGGAGAGATCCCCAGGTGGCGCCAGCCTGGCCCTGAATAGGGCTCTATCCCAGGGCTGCATAA
AGGGCACACTCAGTGCCCCACAGCTCTTCAGGCCCTTCCTGTGCCTGGCTGCCCTCCCACCCTACCCTTTTGTAC
CTCTGAGAAGGCTCTGGCCCCACGCACAGCCCCACTGTCACCAGGGCCAGTATCTGTCTCAGGGACCTCCTATCC
AGAGCCTGAGCCAGCCCCAGCCCCAGCCCCAGCTCCAGCTGCTCCATCTGAACCTGTATCTTCTTCCAAGCCACC
CATTACCCTCTTGGAGTCAGACTCACGCATCTCCAAAGAAGAACTTTTGAGAGCCCAGGCGCTGAGAGAGCAGGG
TCAGACACTCCCGAGCCTCTCGGTACAGCTGTAGGGGCGACACAGGTAGGCTTGCAGCTGCGGGAACAGTGCCAC
CTCCGCACCTAAGCACTCCCATTCCTGGCCAGCATCCTTGGGGCTCATCTCATACAATAGCCCCCGGTCTCAGAG
CTACCTCCTTCTCCAGCTCTTCCTCGTCCTCAGGCCTGTGCTCCCCAGTCAGCAGCTGTAGCCGTTCCATGTACT
GCCGCTGCATGCGGCCAGGCAGGAAGAAGTTGAGGGGAAAGGGCATAGCCTCTGCATACCACTTCCGGGTCACTT
CTACGTAGTTCTTGGTGTCTATCCAAAAAGTATGTACCTGGATTGGGTGGGCAGGAAGAAACAGGCAGGTCTGAG
CCAGTGCACCTGTCTGATTCAAGGTGGGCTTCTGACCTCCATGCTCTCCTGAGTCTCTGTGTGGGTCTGTGTGTT
CCCGTCCCCTCCCCGGCTGGCCATGGATGCTGGGAGGTCTGGGCACACTCACCAGCACCGGGATCAACTTCTCCT
CCAGGAGAGACATGAAGGCCAGGGTGTCTGCCCCTTGCTGAGCTGACAGATCATAATCAGCATTGTACTTCTGTG
GAGGAAATATCCATGGCGTGGACGCTGGGGAGCTGCAAGGGCACTTCACCAGGGAGGAAGGAGTCCTGTCTGGTA
CCCCCCTCACTGGCCTCTGAGTGCAGTGGAGGTACAGCAAGGAACTTTTCCTGCCAAGGCCCCCTTGCCTGGGCC
CAGCCAGTAGCCTGTTGCTGTTGGCAAAAAGCCTGGGCCTTGGAGCCCGCTGGCCGTCAAGGTCCTGGGCCCATT
GAGAAGAAGGAAGAAAGGTTGGGCCGCAAACTAGGAGCAGCTCCCAGAATTTCCATGGAAAGCTGGAACAA

A representative amino acid sequence of full length GBA is provided by GenBank:

```
AAC51820.1, shown below.
  (SEQ ID NO: 48)
MEFSSPSREECPKPLSRVSIMAGSLTGLLLLQAVSWASGARPCIPKSFGYSSVVCVCNATYCDSFDPPTFPALGT
FSRYESTRSGRRMELSMGPIQANHTGTGLLLTLQPEQKFQKVKGFGGAMTDAAALNILALSPPAQNLLLKSYFSE
EGIGYNIIRVPMASCDESIRTYTYADTPDDFQLHNFSLPEEDTKLKIPLIHRALQLAQRPVSLLASPWTSPTWLK
TNGAVNGKGSLKGQPGDIYHQTWARYFVKFLDAYAEHKLQFWAVTAENEPSAGLLSGYPFQCLGFTPEHQRDFIA
RDLGPTLANSTHHNVRLLMLDDQRLLLPHWAKVVLTDPEAAKYVHGIAVHWYLDFLAPAKATLGETHRLFPNTML
FASEACVGSKFWEQSVRLGSWDRGMQYSHSIITNLLYHVVGWTDWNLALNPEGGPNWVRNFVDSPIIVDITKDTF
YKQPMFYHLGHFSKFIPEGSQRVGLVASQKNDLDAVALMHPDGSAVVVVLNRSSKDVPLTIKDPAVGFLETISPG
NG_0126371.1, shown below.
  (SEQ ID NO: 49)
TTGAAAGAAGTGGGTAAAATGCTATCAAATAGCATCACATGGTATGGAGAAATCTTTTGTGAAGGGAAGAGTCGA
CCAAGGTGGCAAATTGCATTGTCATCTTATTTTAAGAAATTGCCACAGCCACCCCCAGCTTTAGCAACCACCACC
CTGATCAGTAAGCAGCCATCAACATCAAAACAAGACCGCCATCCTCTTCAGCAAAAACACTATGACTTGCTGAAG
GCTCAGATGATGGTTAGCATTTTTAGCAATACAATATTTTTAATTAAGGTATGCACATTGGTTTTTCTGACATAA
TACTATTGCATACTTAATAGACTACAGTATAGGATAAACACAACTTTTATATGCACTGGGAAACCAAAAAGGTTA
TTTTTGAGATATTTGCTTTACTGTGGTGGTCTGAAGCTGAACTCACAATCTCACCAAGGTGTGCCTGAACCTCTT
TAGCTAACTGGCCACTGCCACAGTCCACTCTGTGTTGGTCAAGATGCCCCAGAGTGGCAGGCACACTGTGTGGTC
ACATCCAAGGGCCTAGATATGGTGGGGGCTCCAAATGGATCTAGATATGTGAGATCTCTCTTTGATTTGACTTCT
TCCAACCCACCATTTTCTGGGTGCTGGGCTCATCTCACCCAGAAAGTAGGACCCAATGTGACAGTTCCTGCCCAG
TTCCCTCCTGTGGTAGCCACTTGACCCAGGGGCACTCTTGATCCTTGCAGCCTCACTTACACACCCTATCTCTAC
CCCTATTAACTCTCTCCAATCCCCACTCCCCCTGCTCAGCTTGTCTGCTGCCCAGTGGGGGCCCCACCCATGCTG
GCCTCTCCTTTTGCAAGTCCCCATTCCTCATATGGTTTCTTCAGAGCCCCTTTCTTTGGCTTTGAGGAGAGATGC
CCTCACTCGCTTCCCCACCAATCCTGCCCACTTCTACAATCCATTCATTATCCTAATTGCCTCCGTATACAGACT
GGAGTGAGAGGAGTTGATGTGATGGGTGTGGATACAGGGCTGGTGCTGTCATCTTCTAGTAAGCCCTGGGAGAGG
TGTCTGAGCCCAGGTGTCAGTGGTTTTCTTTGGAACTGTGAGTGCATAACACTTCTTTGCCTTCAGCCTTAGGCC
ATAGTTGCTAGTTCTGGGACAACCAGAAAAGCCCTACATAATCTCGTGTTATGTGCAGAGCTGAGTATAGAGCTC
CAGGTATGATCTGACTCACTTAAGATCACAGTGAGTCTATTGTATTGTTGAACTGTTAGCTTAGACATCTGTTAC
TGTACCTACATGGCACTAGCCTCACGCCTAGACACCGATCTGAAAGAAATCCCCTAAATGCATAGAGAAGACTTC
TCAGCTGAGCTAAGGGGCTCCCACCAGGTTTGAGCCTATCTAATGAATCCATGAGGTAGACAGCCTGCACATGTC
CACTTGGTTTGATGAATTGCACAAATCCCTATGGGGGATGTGGTTCATGGGCTGGGAAGTGGGTTACCCTGGGAA
AGGTCTACAGGACAGAGGCAGGGATGGAGACAACAGCATGGTGAGTTCCCAACCCACCCACGATGATAGGTGTCT
GAGGCAGAAGGTAAAGAGGCTGTCACCTGGTGGGTGTCATAAGACTCAAGTGTCATTGTTGAGGCACATGGGTAA
CAAAGCGTGGCACTGGATGGGGGTAGATTCTTCCTATTTCTGTGAGGATCAGGGGGACTCCCTGGCTCTCCTGCT
AAAGGTGGCTCTAGGGACAGGAAGAGTGTACTTCTTGACAGGGATGTCAGAGCACTGATGGTGACAATCAGTGTG
ACACTGCTCACATGACTGAACAACCGAGAAGAGCCCGACTGTCTACTGAACAACGGGAAGAGCCCGACTGTCAAT
GACGGAGCTCTGTTAAATATAGTTAAGGCTATTTTGTTGAATGAATGAAGCCAGACAGGAAAGAGGACAGTATCT
TTAATCCATTTATAGAAGTTAAAGACAGGCTTATTTAATCTCTATGAAGACAGAGTGGCCCTTACCTCTGGGTGG
AGCAAAAGGCACCTTCTGAAGTGATAGGGATGTTCCTTATCATCTTGATCCGGAGTGGTAGTTACATGCATGTGT
GCATATCAAAACTCACCAAGCTGTACCACTAAGTGTGTTCTTCCTCAATAAAAATAATAAAGAACTACACTTATA
AAGAATTTTTTAATAATATAGGAAAATGTCTACACTATAATCTTTAGCTAAAAAAAAAAAAAAAAGAAGCCGCCT
ACAGAATGGTATATGCATGAGAACAATTAATCGAAAAGTGCATGGGAAAAGTCAGGATTGAAACATCATGTTTTA
AAAGACATTGTTTTGATACTGTGAGAATGTACCTAAGTTTTTCCTTTTTTCTGTTTTTCCCAATTTTATACAATG
AGCATGTGTTGGTTTTATAATTAGACATTTTGTTTGTTTGGTTTGGTTTTGAGACACAGCTTGCTGTCACCCAGG
TTGGAGTGCAATGGCCCAATCTTGGTTCACTGCAACCTCCATCTCCTGGGTTCAAGAGATTCTCCCACTTCAGCC
TCCTGAGTAGCTGGGACTATAGGGGCGCACCACCACATCCAGCTAATTTTGTGTATTTTTAGTAGAGATGGGGTT
TCACCATGCTGGCCAGGTTGGTCTCAAACTCCTGACCTCAAGTTATCCACTCGCCTTGGCTTCCCAAAGTGCTGG
GATTATAGGCATGAGCCACCGCACTTGGCCTAGACATTTGTTTTTAAAAATAAAAGATTCATTTGCTCTTTTTAC
AGCCCGTCTCACTGTTGACTGATATTGACCAGGAGTCAACTCAGGCCCCAGGGATTTTCACAACAGCTGCTGTAT
GGCAGGGTTTCTGCTCACTGTGCTCATGTAGTTGGCCCTTGCACCCAAAGTGAATAATTAACATTCTCCCCATCC
TGTTGACGATGCTCTGAAAATATGGTCCAGAAATGGTGTGAGCAAGGAGACAGCAAAGCAATGCTTGGAACATAG
GTGCAGTGACTAGACATGGGGCAGCTGTTTAAAGACAAAAAGGCCCCAAAAAGGAGGGATGGCACGAAACACCCT
CCAATATGGGCATGGAGTCTAGAGTGACAAAGTGATCAAAAGTTCATTTCCTATGGGGTGTCCGAATGTACTTAA
TAATAAAAAGAGAACAAGAGCCATGCAAACTGAGAGGGACAAAGTAGAAAGAGTAGCAGACACCAAGCAACTAAG
TCACAGCATGATAAGCTGCTAGCTTGTTGTCATTATTGTATCCAGAACAACATTTCATTTAAATGCTGAAGAATT
TCCCATGGGTCCCCACTTTCTTGTGAATCCTTGGGCTGAACCCCCCTGTCCTGAGTGGTTACTAGAACACACCTC
TGGACCAGAAACACAAAAGTGGAGTAACGCACACTGCAAAGCTGTGCTTCCTTGTTTCAGCCTGTGAATCCTCAC
CTTGTTTCCCATCTAGCCTATATTTTTCAAACTAACTTGGCCATAGAATCATGTAGTATTTAGGGTGGAAGCTGC
CCCAGGTCTAGCACGTCATTTAACAGATGAGGAAATGGAAGCTTGGGCAGTGGAAGTATCTTGCCGAGGTCACAC
AGCAAGTCAGCAGCACAGCGTGTGTGACTCCGAGCCTGCTCCGCTAGCCCACATTGCCCTCTGGGGGTGAGTATG
TCTTCACATCCTCCAATACCCTAATGACAGACAAACAGAACATGGCAAAGCCTCAGCTCTGCATGGTGAAAGTAA
GAACCAGCAATTGCCACAAACAGAAATACAGTGTTGGTCCGGCAGCCTCCGGGGGTTCTGCACAAGTGGATTACC
AGTGAATACAAGGCTATCTATCTTTCGAAAAACCAAAGTTGTATTTATGCTATCTATTTTCTATAAAATTTTATA
TTAATTTATTTGTTACCTATTTTTGAACTCTTTCAAAAGCACACTTTATATTTCCCTGCTTAAACAGTCCCCCGA
GGGTGGGTGCCCAAAAGGCTCTACACTTGTTATCATTCCCTCTCCACCACAGGCATATTGAGTAAGTTTGTATTT
GGGTTTTTTTAAAACCTCCACTCTACAGTTAAGAAAACTAAGGCACAGAGCTTCAATAATTTGGTCAGAGCCAAG
TAGCAGTAATGAAGCTGGAGGTTAAACCCAGCAGCATGACTGCAGTTCTTAATCAATGCCTTTTGAATTGCACAT
ATGGGATGAACTAGAACATTTTCTCGATGATTCGCTGTCCTTGTTATGATTATGTTACTGAGCTCTGTTGTAGCA
CAGACATATGTCCCTATATGGGGGGGGGTGGGGGTGTCTTGATCGCTGGGCTATTTCTATACTGTTCTGGCTTT
TCCCAAGCAGTCATTTCTTTCTATTCTCCAAGCACCAGCAATTAGCTTTACCTTTTCAGCTTCTAGTTTGCTGAA
ACTAATCTGCTATAGACAGAGACTCCGGTGAACCAATTTTATTAGGATTTGATCAAATAAACTCTCTCTGACAAA
GGACTGCTGAAAGAGTAACTAAGAGTTTGATGTTTACTGAGTGCATAGTATGTGCTAGATGCTGGCCGTGGATGC
CTCATAGAATCCTCCCAACAACTCATGAAATGACTACTGTCATTCAGCCCAATACCCAGACGAGAAAGCTGAGGG
TAAGACAGGTTTCAAGCTTGGCAGTCTGACTACAGAGGCCACTGGCTTAGCCCCTGGGTTAGTCTGCCTCTGTAG
GATTGGGGGCACGTAATTTTGCTGTTTGGGGTCTCATTTGCCTTCTTAGAGATCACAAGCCAAAGCTTTTTATTC
TAGAGCCAAGGTCACGGAAGCCCAGAGGGCATCTTGTGGCTCGGGAGTAGCTCTCTGCTGTCTTCTCAGCTCTGC
TGACAATACTTGAGATTTTCAGATGTCACCAACCGCCAAGAGAGCTTGATATGACTGTATATAGTATAGTCATAA
AGAACCTGAACTTGACCATATACTTATGTCATGTGGAAAATTTCTCATAGCTTCAGATAGATTATATCTGGAGTG
AAGAATCCTGCCACCTATGTATCTGGCATAGTGTGAGTCCTCATAAATGCTTACTGGTTTGAAGGGCAACAAAAT
AGTGAACAGAGTGAAAATCCCCACTAAGATCCTGGGTCCAGAAAAAGATGGGAAACCTGTTTAGCTCACCCGTGA
GCCCATAGTTAAAACTCTTTAGACAACAGGTTGTTTCCGTTTACAGAGAACAATAATATTGGGTGGTGAGCATCT
GTGTGGGGGTTGGGGTGGGATAGGGGATACGGGGAGAGTGGAGAAAAAGGGGACACAGGGTTAATGTGAAGTCCA
GGATCCCCCTCTACATTTAAAGTTGGTTTAAGTTGGCTTTAATTAATAGCAACTCTTAAGATAATCAGAATTTTC
TTAACCTTTTAGCCTTACTGTTGAAAAGCCCTGTGATCTTGTACAAATCATTTGCTTCTTGGATAGTAATTTCTT
TTACTAAAATGTGGGCTTTTGACTAGATGAATGTAAATGTTCTTCTAGCTCTGATATCCTTTATTCTTTATATTT
TCTAACAGATTCTGTGTAGTGGGATGAGCAGAGAACAAAAACAAAATAATCCAGTGAGAAAAGCCCGTAAATAAA
CCTTCAGACCAGAGATCTATTCTCTAGCTTATTTTAAGCTCAACTTAAAAAGAAGAACTGTTCTCTGATTCTTTT
CGCCTTCAATACACTTAATGATTTAACTCCACCCTCCTTCAAAAGAAACAGCATTTCCTACTTTTATACTGTCTA
TATGATTGATTTGCACAGCTCATCTGGCCAGAAGAGCTGAGACATCCGTTCCCCTACAAGAAACTCTCCCCGGTA
AGTAACCTCTCAGCTGCTTGGCCTGTTAGTTAGCTTCTGAGATGAGTAAAAGACTTTACAGGAAACCCATAGAAG
ACATTTGGCAAACACCAAGTGCTCATACAATTATCTTAAAATATAATCTTTAAGATAAGGAAAGGGTCACAGTTT
GGAATGAGTTTCAGACGGTTATAACATCAAAGATACAAAACATGATTGTGAGTGAAAGACTTTAAAGGGAGCAAT
AGTATTTTAATAACTAACAATCCTTACCTCTCAAAAGAAAGATTTGCAGAGAGATGAGTCTTAGCTGAAATCTTG
AAATCTTATCTTCTGCTAAGGAGAACTAAACCCTCTCCAGTGAGATGCCTTCTGAATATGTGCCCACAAGAAGTT
GTGTCTAAGTCTGGTTCTCTTTTTTCTTTTTCCTCCAGACAAGAGGGAAGCCTAAAAATGGTCAAAATTAATATT
AAATTACAAACGCCAAATAAAATTTTCCTCTAATATATCAGTTTCATGGCACAGTTAGTATATAATTCTTTATGG
TTCAAAATTAAAAATGAGCTTTTCTAGGGGCTTCTCTCAGCTGCCTAGTCTAAGGTGCAGGGAGTTTGAGACTCA
CAGGGTTTAATAAGAGAAAATTCTCAGCTAGAGCAGCTGAACTTAAATAGACTAGGCAAGACAGCTGGTTATAAG
ACTAAACTACCCAGAATGCATGACATTCATCTGTGGTGGCAGACGAAACATTTTTTATTATATTATTTCTTGGGT
ATGTATGACAACTCTTAATTGTGGCAACTCAGAAACTACAAACACAAACTTCACAGAAAATGTGAGGATTTTACA
ATTGGCTGTTGTCATCTATGACCTTCCCTGGGACTTGGGCACCCGGCCATTTCACTCTGACTACATCATGTCACC
AAACATCTGATGGTCTTGCCTTTTAATTCTCTTTTCGAGGACTGAGAGGGAGGGTAGCATGGTAGTTAAGAGTGC
AGGCTTCCCGCATTCAAAATCGGTTGCTTACTAGCTGTGTGGCTTTGAGCAAGTTACTCACCCTCTCTGTGCTTC
AAGGTCCTTGTCTGCAAAATGTGAAAAATATTTCCTGCCTCATAAGGTTGCCCTAAGGATTAAATGAATGAATGG
GTATGATGCTTAGAACAGTGATTGGCATCCAGTATGTGCCCTCGAGGCCTCTTAATTATTACTGGCTTGCTCATA
GTGCATGTTCTTTGTGGGCTAACTCTAGCGTCAATAAAAATGTTAAGACTGAGTTGCAGCCGGGCATGGTGGCTC
ATGCCTGTAATCCCAGCATTCTAGGAGGCTGAGGCAGGAGGATCGCTTGAGCCCAGGAGTTCGAGACCAGCCTGG
GCAACATAGTGTGATCTTGTATCTATAAAAATAAACAAAATTAGCTTGGTGTGGTGGCGCCTGTAGTCCCCAGCC
ACTTGGAGGGGTGAGGTGAGAGGATTGCTTGAGCCCGGGATGGTCCAGGCTGCAGTGAGCCATGATCGTGCCACT
GCACTCCAGCCTGGGCGACAGAGTGAGACCCTGTCTCACAACAACAACAACAACAACAAAAAGGCTGAGCTGCAC
CATGCTTGACCCAGTTTCTTAAAATTGTTGTCAAAGCTTCATTCACTCCATGGTGCTATAGAGCACAAGATTTTA
TTTGGTGAGATGGTGCTTTCATGAATTCCCCCAACAGAGCCAAGCTCTCCATCTAGTGGACAGGGAAGCTAGCAG
CAAACCTTCCCTTCACTACAAAACTTCATTGCTTGGCCAAAAAGAGAGTTAATTCAATGTAGACATCTATGTAGG
CAATTAAAAACCTATTGATGTATAAAACAGTTTGCATTCATGGAGGGCAACTAAATACATTCTAGGACTTTATAA
AAGATCACTTTTTATTTATGCACAGGGTGGAACAAGATGGATTATCAAGTGTCAAGTCCAATCTATGACATCAAT
TATTATACATCGGAGCCCTGCCAAAAAATCAATGTGAAGCAAATCGCAGCCCGCCTCCTGCCTCCGCTCTACTCA
CTGGTGTTCATCTTTGGTTTTGTGGGCAACATGCTGGTCATCCTCATCCTGATAAACTGCAAAAGGCTGAAGAGC
ATGACTGACATCTACCTGCTCAACCTGGCCATCTCTGACCTGTTTTTCCTTCTTACTGTCCCCTTCTGGGCTCAC
TATGCTGCCGCCCAGTGGGACTTTGGAAATACAATGTGTCAACTCTTGACAGGGCTCTATTTTATAGGCTTCTTC
TCTGGAATCTTCTTCATCATCCTCCTGACAATCGATAGGTACCTGGCTGTCGTCCATGCTGTGTTTGCTTTAAAA
GCCAGGACGGTCACCTTTGGGGTGGTGACAAGTGTGATCACTTGGGTGGTGGCTGTGTTTGCGTCTCTCCCAGGA
ATCATCTTTACCAGATCTCAAAAAGAAGGTCTTCATTACACCTGCAGCTCTCATTTTCCATACAGTCAGTATCAA
TTCTGGAAGAATTTCCAGACATTAAAGATAGTCATCTTGGGGCTGGTCCTGCCGCTGCTTGTCATGGTCATCTGC
TACTCGGGAATCCTAAAAACTCTGCTTCGGTGTCGAAATGAGAAGAAGAGGCACAGGGCTGTGAGGCTTATCTTC
ACCATCATGATTGTTTATTTTCTCTTCTGGGCTCCCTACAACATTGTCCTTCTCCTGAACACCTTCCAGGAATTC
TTTGGCCTGAATAATTGCAGTAGCTCTAACAGGTTGGACCAAGCTATGCAGGTGACAGAGACTCTTGGGATGACG
CACTGCTGCATCAACCCCATCATCTATGCCTTTGTCGGGGAGAAGITCAGAAACTACCTCTTAGTCTTCTTCCAA
AAGCACATTGCCAAACGCTTCTGCAAATGCTGTTCTATTTTCCAGCAAGAGGCTCCCGAGCGAGCAAGCTCAGTT
TACACCCGATCCACTGGGGAGCAGGAAATATCTGTGGGCTTGTGACACGGACTCAAGTGGGCTGGTGACCCAGTC
AGAGTTGTGCACATGGCTTAGTTTTCATACACAGCCTGGGCTGGGGGTGGGGTGGGAGAGGTCTTTTTTAAAAGG
AAGTTACTGTTATAGAGGGTCTAAGATTCATCCATTTATTTGGCATCTGTTTAAAGTAGATTAGATCTTTTAAGC
CCATCAATTATAGAAAGCCAAATCAAAATATGTTGATGAAAAATAGCAACCTTTTTATCTCCCCTTCACATGCAT
CAAGTTATTGACAAACTCTCCCTTCACTCCGAAAGTTCCTTATGTATATTTAAAAGAAAGCCTCAGAGAATTGCT
GATTCTTGAGTTTAGTGATCTGAACAGAAATACCAAAATTATTTCAGAAATGTACAACTTTTTACCTAGTACAAG
GCAACATATAGGTTGTAAATGTGTTTAAAACAGGTCTTTGTCTTGCTATGGGGAGAAAAGACATGAATATGATTA
GTAAAGAAATGACACTTTTCATGTGTGATTTCCCCTCCAAGGTATGGTTAATAAGTTTCACTGACTTAGAACCAG
GCGAGAGACTTGTGGCCTGGGAGAGCTGGGGAAGCTTCTTAAATGAGAAGGAATTTGAGTTGGATCATCTATTGC
TGGCAAAGACAGAAGCCTCACTGCAAGCACTGCATGGGCAAGCTTGGCTGTAGAAGGAGACAGAGCTGGTTGGGA
AGACATGGGGAGGAAGGACAAGGCTAGATCATGAAGAACCTTGACGGCATTGCTCCGTCTAAGTCATGAGCTGAG
CAGGGAGATCCTGGTTGGTGTTGCAGAAGGTTTACTCTGTGGCCAAAGGAGGGTCAGGAAGGATGAGCATTTAGG
GCAAGGAGACCACCAACAGCCCTCAGGTCAGGGTGAGGATGGCCTCTGCTAAGCTCAAGGCGTGAGGATGGGAAG
GAGGGAGGTATTCGTAAGGATGGGAAGGAGGGAGGTATTCGTGCAGCATATGAGGATGCAGAGTCAGCAGAACTG
GGGTGGATTTGGGTTGGAAGTGAGGGTCAGAGAGGAGTCAGAGAGAATCCCTAGTCTTCAAGCAGATTGGAGAAA
CCCTTGAAAAGACATCAAGCACAGAAGGAGGAGGAGGAGGTTTAGGTCAAGAAGAAGATGGATTGGTGTAAAAGG
ATGGGTCTGGTTTGCAGAGCTTGAACACAGTCTCACCCAGACTCCAGGCTGTCTTTCACTGAATGCTTCTGACTT
CATAGATTTCCTTCCCATCCCAGCTGAAATACTGAGGGGTCTCCAGGAGGAGACTAGATTTATGAATACACGAGG
TATGAGGTCTAGGAACATACTTCAGCTCACACATGAGATCTAGGTGAGGATTGATTACCTAGTAGTCATTTCATG
GGTTGTTGGGAGGATTCTATGAGGCAACCACAGGCAGCATTTAGCACATACTACACATTCAATAAGCATCAAACT
CTTAGTTACTCATTCAGGGATAGCACTGAGCAAAGCATTGAGCAAAGGGGTCCCATAGAGGTGAGGGAAGCCTGA
AAAACTAAGATGCTGCCTGCCCAGTGCACACAAGTGTAGGTATCATTTTCTGCATTTAACCGTCAATAGGCAAAG
GGGGGAAGGGACATATTCATTTGGAAATAAGCTGCCTTGAGCCTTAAAACCCACAAAAGTACAATTTACCAGCCT
CCGTATTTCAGACTGAATGGGGGTGGGGGGGGCGCCTTAGGTACTTATTCCAGATGCCTTCTCCAGACAAACCAG
AAGCAACAGAAAAAATCGTCTCTCCCTCCCTTTGAAATGAATATACCCCTTAGTGTTTGGGTATATTCATTTCAA
AGGGAGAGAGAGAGGTTTTTTTCTGTTCTGTCTCATATGATTGTGCACATACTTGAGACTGTTTTGAATTTGGGG
GATGGCTAAAACCATCATAGTACAGGTAAGGTGAGGGAATAGTAAGTGGTGAGAACTACTCAGGGAATGAAGGTG
TCAGAATAATAAGAGGTGCTACTGACTTTCTCAGCCTCTGAATATGAACGGTGAGCATTGTGGCTGTCAGCAGGA
AGCAACGAAGGGAAATGTCTTTCCTTTTGCTCTTAAGTTGTGGAGAGTGCAACAGTAGCATAGGACCCTACCCTC
TGGGCCAAGTCAAAGACATTCTGACATCTTAGTATTTGCATATTCTTATGTATGTGAAAGTTACAAATTGCTTGA
AAGAAAATATGCATCTAATAAAAAACACCTTCTAAAATAATTCATTATATTCTTGCTCTTTCAGTCAAGTGTACA
TTTAGAGAATAGCACATAAAACTGCCAGAGCATTTTATAAGCAGCTGTTTTCTTCCTTAGTGTGTGTGCATGTGT
GTGTGATGTATACAAAGAGAGAGATAATTGTATTTTTGTATTTTCTTTTAAATAATTTTTAAAATTGACCCTTTT
CCTGAGACAAATTGCCAGAATAGTTTGTATTTAGAGATGGTACCTCTAAGAGTAAGGTTGCTGGTTGCTGAGCAA
TTGACTTGAAAACTTTTAAAATTCAAATTTTAATTCCACTACTCAAAAGAATTGCCATGTTTTAAAAAAGAGAAT
TGGTGCCATAAGTTAGTTGTCTATGTTTGAAAATGAAGAAGATATGCAACGTCATGGCCTGGTCACTTACCCGCA
GCCCTGAGTTGTAGGCACATCATATGTGAGAATGAGGATGCTTTTCTTTCATTTAAAATCCCTCCCCAAAACTTG
GCTCTAATTGCAGTCATGACAATCATGTACATTTGGATTTATGTGCACGAGTCTCTTACCCTGAGAGAGGACAGG
TGCTACAGGTGGAGGGGACCCGTCTGGGTCACGTTCACATTTTGAACATGCTGGTTTTCAGTCACTGCACACTCA
TCTCCCAGCACAGGTCATGGGCAGCAGATGCAAAAGCTGCCCGTGGTCCTATTTGGAGGTGCATGAAATGAGCAG
AAGACAGAACAGCTTGATCTGACTAGAAGGGCAGCTTGTCCCTACCAAGACTTGAAGGATTGCCTTTCATCTGTT
AGGGTAAAAGGTAGAATGAACCAAGGAAGGGCAGGAGGGGGCTGGGGTTAGGGTAGAAGGAAGGGGCCATGGAGA
AGGGAGATCCATCCCATAGGAGGAAGGCAGTGCGGCAGGGAGGTTTGAAGGTATCAGCTTTTGTGGCTGACATAC
ATGCAGTCATGTCAATTGCTCGTTTTTCCTTTTCCATCTTATTAAATGTCTTCCAACGTTAGCACGAAGAAAAGC
TATTTGCAGTGTTGCCAGCCTTTCCAGAGCCCGTCCCCATTACCTCCCCAGGCCCATGCCTTTACTCCTTGGAGT
TTCAACTCACGACCTTCAGGATCTGACTTTATTCACCAACTCTGGGGTGAACGTACCTTCTGTCTCCACCCAGAG
GTCTCTATCAAAGAGGAGATTGCATGCCATGGATAAAGTCAAAGTAGAGGTGACTGTCCTTAGGAAGAGTAATGT
GAAAATTCATAAACTGGGATTCTGTTTACATTTTGTACTCCAGGGGTTCTTAGTTTAAATCGCTCTGAATAAATT
AAGATGCAATGGCATTTCAACTGTTATGATTAAATTTACAAATCATTTATTTTCTATCACGGGGAGAGATAGAGC
TCCAAATGCAAACATAACTGCTCAAGTGTTAACACTTATAATGAAAACATAAGAATTACCACCAACTACCCTGGG
GGCTAGAAGCAGAAATGTGAACCAGAAAACAAATCATGAACTTTCCTTTTTTTTTTTGAGATGGAGTCTCGCTCT
GTTGCCCAGGCTGGAGTGCAATGGTGCGATCTCGGCTCACTGCAACCACTGCCTCCCGGGTTCAAGCAATTCTCC
TGCCTCAGCCTCCTGAGTAGCTGGGACTACAGGCATGCACCACCACGCCTGGGTAATTTTTTGTATTTTTAGTAG
AGACAGGGTTTCACCGTATTAGCCAGGATGCTCTCGATCTCCTGACCTCGTGATCTGCCCGCCTCGGCCTCCCAC
CGAAGTGCTGGGATTACAGGCATGAGCCACTGTGCCCGGCCAACAAATCATGAACTTTCTAACTGCAGTTCCTTG
TAGCTTGTTAACACATCCACTTACTTATTGTCAGAGTACGTGGAGATTTTCCACAACCCTCGGGGATAAGGCTGA
ACAGAAGAGGCAAAAACGTGAAAACATTTCGATAGCTCCTATACTTTGAAATAAAATTCACTGTAAAAGTTGCTT
GTATTTTTCCAAAAC
```

A representative nucleotide sequence of the CCR5 gene is provided by GenBank: NG_012637.1, shown below. (SEQ ID NO: 49)

### Cells

The disclosure is directed, in part, to methods and compositions for genetically modifying cells, to genetically modified cells produced thereby, and for methods of using said modified cells (e.g., to treat a subject in need thereof). In some embodiments, a cell is a hematopoietic cell. In some embodiments, a hematopoietic cell is a hematopoietic stem cell (HSC) or hematopoietic progenitor cell (HPC). In some embodiments, the cell is a T cell. In some embodiments, a method or composition described herein is used to genetically modify a hematopoietic cell (e.g., an HSC or HPC), e.g., to modify a gene in its genome, e.g., the GBA gene. In some embodiments, a method or composition described herein is used to genetically modify a T cell, e.g., to modify a gene in its genome, e.g., the TRAC gene.

Accordingly, the disclosure is directed, in part, to genetically modified hematopoietic cells and genetically modified T cells and uses thereof. It will be understood that such a cell can be created by contacting the cell with a CRISPR/Cas system (e.g., a Cas nuclease and/or gRNA) and a template polynucleotide or an rAAV encoding the template polynucleotide, or the cell can be the daughter cell of a cell that was contacted with the CRISPR/Cas system and a template polynucleotide. In some embodiments, a cell described herein (e.g., a genetically engineered HSC or HPC) is capable of populating the HSC or HPC niche and/or of reconstituting the hematopoietic system of a subject. In some embodiments, a cell described herein (e.g., an HSC or HPC) is capable of one or more of (e.g., all of): engrafting in a human subject, producing myeloid lineage cells, and producing and lymphoid lineage cells. In some preferred embodiments, a genetically engineered hematopoietic cell provided herein, or its progeny, can differentiate into all blood cell lineages, preferably without any differentiation bias as compared to a hematopoietic cell of the same cell type, but not comprising the respective HDR-mediated genomic modification. In some embodiments, the cells, e.g., HSCs, contacted with the genetic modification mixture are autologous to a subject, e.g., a subject to be treated for a genetic disease, e.g., Gaucher disease. In some embodiments, the HSCs contacted with the genetic modification mixture are derived from a subject with a genetic disease or at risk of developing a genetic disease (e.g., Gaucher disease).

In some embodiments, a genetically engineered hematopoietic cell or T cell of the disclosure comprises a genetic modification proximal to a PAM sequence, e.g., a PAM sequence in a target DNA. In some embodiments, the genetic modification comprises integration of a donor sequence. In some embodiments, the integration of a donor sequence results in an insertion mutation or a substitution mutation. In some embodiments, a donor sequence is inserted 5′ of a PAM sequence, e.g., of a Cas9 PAM sequence. In some embodiments, a donor sequence is inserted 5′ of a PAM sequence. In some embodiments, a donor sequence is inserted 1, 2, 3, 4, 5, 6, 7, 8, 9, 10, 11, 12, 13, 14, 15, 16, 17, 18, 19, or 20 nucleotides 5′ of a PAM sequence. In some embodiments, a donor sequence is inserted 1-10, 1-8, 1-6, 1-4, 2-10, 2-8, 2-6, 2-4, 4-10, 4-8, 4-6, 6-10, 6-8, 8-10, 10-20, 15-20, 16-20, 17-20, 18-20, 19-20, 16-19, 17-19, 18-19, 16-18, or 17-18 nucleotides 5′ of a PAM sequence, e.g., 2, 3, or 4 nucleotides 5′ of a PAM sequence. In some embodiments, a donor sequence is inserted 1, 2, 3, 4, 5, 6, 7, 8, 9, 10, 11, 12, 13, 14, 15, 16, 17, 18, 19, or 20 nucleotides 3′ of a PAM sequence. In some embodiments, a donor sequence is inserted 1-10, 1-8, 1-6, 1-4, 2-10, 2-8, 2-6, 2-4, 4-10, 4-8, 4-6, 6-10, 6-8, 8-10, 10-20, 15-20, 16-20, 17-20, 18-20, 19-20, 16-19, 17-19, 18-19, 16-18, or 17-18 nucleotides 3′ of a PAM sequence, e.g., 17, 18, or 19 nucleotides 3′ of a PAM sequence.

In some embodiments, a genetically engineered hematopoietic cell or genetically engineered T cell comprises a genetic modification corresponding to integration of a donor sequence (e.g., from a template polynucleotide described herein) into a target DNA in the hematopoietic cell. In some embodiments, the genetic modification corresponds to a position or positions where the donor sequence differs from the sequence of the target DNA. In some embodiments, integration of the donor sequence results in modification at 1, 2, 3, 4, 5, 6, 7, 8, 9, or 10 bases (e.g., 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 bases) in the target DNA. In some embodiments, integration of the donor sequence results in an insertion of 1, 2, 3, 4, 5, 6, 7, 8, 9, or 10 bases (e.g., 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 bases) in the target DNA. In some embodiments, integration of the donor sequence results in substitution of 1, 2, 3, 4, 5, 6, 7, 8, 9, or 10 bases (e.g., 1-10, 1-9, 1-8, 1-7, 1-6, 1-5, 1-4, 1-3, 1-2, 2-10, 2-9, 2-8, 2-7, 2-6, 2-5, 2-4, 2-3, 3-10, 3-9, 3-8, 3-7, 3-6, 3-5, 3-4, 4-10, 4-9, 4-8, 4-7, 4-6, 4-5, 5-10, 5-9, 5-8, 5-7, 5-6, 6-10, 6-9, 6-8, 6-7, 7-10, 7-9, 7-8, 8-10, 8-9, or 9-10 bases) in the target DNA. In some embodiments, integration of the donor sequence results in modification at a number of positions in the target DNA corresponding to up to 1, 5, 10, 15, 20, 25, 30, 35, 40, 45, 50, 55, 60, 65, 70, 75, 80, 85, 90, 95, or 100% of the length of the donor sequence. In some embodiments, integration of the donor sequence results in insertion of a number of bases in the target DNA corresponding to up to 1, 5, 10, 15, 20, 25, 30, 35, 40, 45, 50, 55, 60, 65, 70, 75, 80, 85, 90, 95, or 100% of the length of the donor sequence. In some embodiments, a donor sequence is 200-2000, 200-1900, 200-1800, 200-1700, 200-1600, 200-1500, 200-1400, 200-1300, 200-1200, 200-1100, 100-1000, 100-900, 100-800, 100-700, 100-600, 100-500, 100-400, 100-300, or 100-200 nucleotides in length. In some embodiments, a donor sequence is no more than 2000, no more than 1900, no more than 1800, no more than 1700, no more than 1600, no more than 1500, no more than 1400, no more than 1300, no more than 1200, no more than 1100, no more than 1000, no more than 900, no more than 800, no more than 700, no more than 600, no more than 500, no more than 400, no more than 300, or no more than 200 nucleotides in length. In some embodiments, the donor sequence is 1-100, 1-80, 1-60, 1-40, 1-20, 1-15, 1-10, 1-9, 1-8, 1-7, 1-6, 1-5, 1-4, 1-3, 1-2, 5-100, 5-80, 5-60, 5-40, 5-20, 5-15, 5-10, 5-9, 5-8, 5-7, 5-6, 10-100, 10-80, 10-60, 10-40, 10-20, 10-15, 20-100, 20-80, 20-60, 20-40, 60-100, or 60-80 nucleotides in length. In some embodiments, a donor sequence is no more than 100, no more than 90, no more than 80, no more than 70, no more than 60, no more than 50, no more than 45, no more than 40, no more than 35, no more than 30, no more than 25, no more than 20, no more than 15, no more than 14, no more than 13, no more than 12, no more than 11, no more than 10, no more than 9, no more than 8, no more than 7, no more than 6, no more than 5, no more than 4, no more than 3, no more than 2, or no more than 1 bases long. In some embodiments, a donor sequence is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100 bases long. In some embodiments, integration of the donor sequence into the genetically engineered hematopoietic cell corrected a prior mutation in the target DNA (e.g., a disease-associated mutation (a mutation characteristic of, or causally associated with, a disease or disorder, or risk of developing a disease or disorder) or a non-disease-associated mutation), e.g., as described herein. In some embodiments, the donor sequence integrated comprises a sequence corresponding to the wild-type, functional, and/or naturally-occurring sequence at a position or positions corresponding to a prior mutation in the target DNA. In some embodiments, the donor sequence comprises an artificial or heterologous sequence. In some embodiments, integration of the donor sequence produces a restriction nuclease site or a unique sequence tag in the target DNA of the genetically engineered hematopoietic cell. In some embodiments, integration of the donor sequence into the target DNA of the genetically engineered hematopoietic cell produces one or more silent mutations along with a non-silent mutation (e.g., correction of a prior mutation, e.g., in a coding sequence). In some embodiments, the one or more silent mutations are contiguous with another mutation described herein (e.g., contiguous with correction of a prior mutation). For example, in some embodiments a genetically engineered hematopoietic cell comprises a genetic modification corresponding to correction of a prior mutation which substitutes a single prior mutation, e.g., a single nucleotide point mutation, for the corresponding base(s) present in a wild-type cell, and one or more silent mutations contiguous with the prior mutation. Accordingly, the disclosure provides a genetically engineered hematopoietic cell comprising a genetic modification corresponding to integration of a donor sequence as described herein, e.g., a donor sequence described herein. In some embodiments, integration of the donor sequence into a genetically engineered T cell results in the expression of a chimeric antigen receptor (CAR). In some embodiments, the CAR binds to at least one antigen present on a lineage-specific cell-surface antigen. In some embodiments, the lineage-specific cell surface antigen is CD33.

It will be understood that, upon engrafting donor cells into a recipient host organism, the relative levels of the engrafted donor cells (and descendants thereof) and the host cells, e.g., in a given niche (e.g., bone marrow), are important for physiological and/or therapeutic outcomes for the host organism. The level of engrafted donor cells or descendants thereof relative to host cells in a given tissue or niche is referred to herein as chimerism. In some embodiments, a cell described herein (e.g., an HSC or HPC) is capable of engrafting in a human subject and does not exhibit any difference in chimerism as compared to a hematopoietic cell of the same cell type, but not comprising a genomic modification that results in expression of a variant form (e.g., a wild-type form or having wild-type functionality) of a gene or a loss of expression of a gene. In some embodiments, a cell described herein (e.g., an HSC or HPC) is capable of engrafting in a human subject exhibits no more than a 1, no more than a 2, no more than a 5, no more than a 10, no more than a 15, no more than a 20, no more than a 25, no more than a 30, no more than a 35, no more than a 40, no more than a 45, or no more than a 50% difference in chimerism as compared to a hematopoietic cell of the same cell type, but not comprising a genomic modification that results in expression of a variant form (e.g., a wild-type form or having wild-type functionality) of a gene or a loss of expression of a gene.

In some embodiments, a genetically engineered cell provided herein comprises only one genomic modification, e.g., a genomic modification that results in expression of a variant form (e.g., a wild-type form or having wild-type functionality) of a gene or a loss of expression of a gene. In some embodiments, the genomic modification is a modification to the GBA gene. It will be understood that the gene editing methods provided herein may result in genomic modifications in one or both alleles of a target gene. In some embodiments, genetically engineered cells comprising a genomic modification in both alleles of a given genetic locus are preferred.

In some embodiments, a genetically engineered cell provided herein comprises two or more genomic modifications, e.g., one or more genomic modifications in addition to a genomic modification that results in expression of a variant form (e.g., a wild-type form or having wild-type functionality) of a gene or a loss of expression of a gene. For example, in some embodiments a genetically engineered cell comprises a modification to the GBA gene and one or more additional genomic modifications, e.g., modification to a second gene or one or more silent mutations proximal to (e.g., contiguous with) the modification to the GBA gene. As a further example, in some embodiments a genetically engineered cell comprises a modification that eliminates expression from an endogenous gene (e.g., an endogenous GBA gene) and a second modification that inserts an exogenous copy of the gene, e.g., at the site of the endogenous gene or at another site in the genome.

In some embodiments, a genetically engineered cell provided herein comprises a genomic modification that results in expression of a variant form (e.g., a wild-type form or having wild-type functionality) of a GBA gene. In some embodiments, the modification corrects a prior mutation in the GBA gene that produces a less functional or non-functional glucosylceramidase beta enzyme. In some embodiments, the modification results in a GBA gene encoding a glucosylceramidase beta that has wild-type glucosylceramidase beta activity, or at least 20, at least 30, at least 40, at least 50, at least 60, at least 70, at least 80, at least 90, at least 95, or at least 99% of the activity of a wild-type glucosylceramidase beta. In some embodiments, the modification inserts an exogenous (e.g., wild-type) copy of the GBA gene into the genome of the cell, e.g., at the site of the endogenous GBA gene or at another target DNA (e.g., a safe harbor locus).

Some aspects of this disclosure provide genetically engineered immune effector cells comprising a modification in their genome that results in expression of a variant gene (e.g., a GBA gene encoding a glucosylceramidase beta that has wild-type glucosylceramidase beta activity, or at least 20, at least 30, at least 40, at least 50, at least 60, at least 70, at least 80, at least 90, at least 95, or at least 99% of the activity of a wild-type glucosylceramidase beta). In some embodiments, the immune effector cell is a lymphocyte. In some embodiments, the immune effector cell is a T-lymphocyte. In some embodiments, the T-lymphocyte is an alpha/beta T-lymphocyte. In some embodiments, the T-lymphocyte is a gamma/delta T-lymphocyte. In some embodiments, the immune effector cell is a natural killer T (NKT cell). In some embodiments, the immune effector cell is a natural killer (NK) cell. In some embodiments, the immune effector cell expresses a chimeric antigen receptor (CAR). In some embodiments, the immune effector cell does not express a CAR and/or does not express any transgenic protein except as provided by a genetic modification described herein (e.g., except as modified using a method using HDR described herein), e.g., except for glucosylceramidase beta.

In some embodiments, the genetically engineered cells provided herein are hematopoietic cells, e.g., hematopoietic stem cells, hematopoietic progenitor cells (HPCs), hematopoietic stem or progenitor cells. Hematopoietic stem cells (HSCs) are cells characterized by pluripotency, self-renewal properties, and/or the ability to generate and/or reconstitute all lineages of the hematopoietic system, including both myeloid and lymphoid progenitor cells that further give rise to myeloid cells (e.g., monocytes, macrophages, neutrophils, basophils, dendritic cells, erythrocytes, platelets, etc.) and lymphoid cells (e.g., T cells, B cells, NK cells), respectively. HSCs are characterized by the expression of one or more cell surface markers, e.g., CD34 (e.g., CD34+), which can be used for the identification and/or isolation of HSCs, and absence of cell surface markers associated with commitment to a cell lineage. In some embodiments, a genetically engineered cell (e.g., genetically engineered HSC) described herein does not express one or more cell-surface markers typically associated with HSC identification or isolation, expresses a reduced amount of the cell-surface markers, or expresses a variant cell-surface marker not recognized by an immunotherapeutic agent targeting the cell-surface marker, but nevertheless is capable of self-renewal and can generate and/or reconstitute all lineages of the hematopoietic system.

In some embodiments, a population of genetically engineered cells described herein comprises a plurality of genetically engineered hematopoietic stem cells. In some embodiments, a population of genetically engineered cells described herein comprises a plurality of genetically engineered hematopoietic progenitor cells. In some embodiments, a population of genetically engineered cells described herein comprises a plurality of genetically engineered hematopoietic stem cells and a plurality of genetically engineered hematopoietic progenitor cells. In some embodiments, a population of genetically engineered cells described herein comprises a plurality of genetically engineered T cells.

In some embodiments, the genetically engineered HSCs are obtained from a subject, such as a human subject. Methods of obtaining HSCs are described, e.g., in PCT Publication No. US2016/057339, which is herein incorporated by reference in its entirety. In some embodiments, the HSCs are peripheral blood HSCs. In some embodiments, the mammalian subject is a non-human primate, a rodent (e.g., mouse or rat), a bovine, a porcine, an equine, or a domestic animal. In some embodiments, the HSCs are obtained from a human subject, such as a human subject having a hematopoietic malignancy. In some embodiments, the HSCs are obtained from a healthy donor. In some embodiments, the HSCs are obtained from the subject to whom the immune cells expressing the chimeric receptors will be subsequently administered. HSCs that are administered to the same subject from which the cells were obtained are referred to as autologous cells, whereas HSCs that are obtained from a subject who is not the subject to whom the cells will be administered are referred to as allogeneic cells.

In some embodiments, a population of genetically engineered cells is a heterogeneous population of cells, e.g. heterogeneous population of genetically engineered cells containing different mutations, e.g., different mutations in a target gene or differently positioned exogenous copies of a target gene (e.g. the GBA gene). In some embodiments, at least 40%, at least 50%, at least 60%, at least 70%, at least 75%, at least 80%, at least 85%, at least 90%, or at least 95% of copies of a target gene (e.g., GBA) in the population of genetically engineered cells comprise a mutation effected by a genome editing approach described herein. In some embodiments, at least 40%, at least 50%, at least 60%, at least 70%, at least 75%, at least 80%, at least 85%, at least 90%, or at least 95% of target loci (e.g., a safe harbor locus) in the population of genetically engineered cells comprise a mutation (e.g., an insertion comprising an exogenous copy of a gene) effected by a genome editing approach described herein. By way of example, a population of genetically engineered cells can comprise a plurality of different GBA mutations and each mutation of the plurality may contribute to the percent of copies of GBA in the population of cells that have a mutation.

In some embodiments, the expression of a target gene, e.g., the GBA gene, in the genetically engineered hematopoietic cell is compared to the expression of the target gene in a reference hematopoietic cell (e.g., a wild-type counterpart, a counterpart comprising a disease-associated mutation (a mutation characteristic of, or causally associated with, a disease or disorder, or risk of developing a disease or disorder), or a mock genetically engineered hematopoietic cell (e.g., a hematopoietic cell that is contacted with Cas9 and a scrambled gRNA that does not effectively localize Cas9 to the target gene or a hematopoietic cell that is contacted with a targeting gRNA in the absence of Cas9)). In some embodiments, the genetic engineering results in a reduction in the expression level of a target gene (e.g., the endogenous copy of GBA) by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of the target gene (e.g., GBA) in a reference hematopoietic cell (e.g., a wild-type counterpart, a counterpart comprising a disease-associated mutation (a mutation characteristic of, or causally associated with, a disease or disorder, or risk of developing a disease or disorder), or a mock genetically engineered hematopoietic cell). In some embodiments, the genetic engineering results in an increase in the expression level of a target gene (e.g., the endogenous copy of the target gene or the overall level of expression of the target gene in the cell) by at least 50%, at least 60%, at least 70%, at least 80%, at least 90%, at least 91%, at least 92%, at least 93%, at least 94%, at least 95%, at least 96%, at least 97%, at least 98%, or at least 99% as compared to the expression of the target gene in a reference hematopoietic cell (e.g., a wild-type counterpart, a counterpart comprising a disease-associated mutation (a mutation characteristic of, or causally associated with, a disease or disorder, or risk of developing a disease or disorder), or a mock genetically engineered hematopoietic cell). For example, in some embodiments, the genetically engineered hematopoietic cell expresses less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, or less than 1% of the level of a target gene (e.g., an endogenous copy of a target gene, e.g., GBA) as compared to a reference hematopoietic cell (e.g., a wild-type counterpart, a counterpart comprising a disease-associated mutation (a mutation characteristic of, or causally associated with, a disease or disorder, or risk of developing a disease or disorder), or a mock genetically engineered hematopoietic cell). As a further example, in some embodiments, the genetically engineered hematopoietic cell expresses 5% more, 10% more, 20% more, 30% more, 40% more, 50% more, 75% more, 100% more, 125% more, 150% more, 200% more, 300% more, 400% more, 500% more, or 1000% more of a target gene (e.g., GBA) than the level of the target gene in a reference hematopoietic cell (e.g., containing a wild-type counterpart, a counterpart comprising a disease-associated mutation (a mutation characteristic of, or causally associated with, a disease or disorder, or risk of developing a disease or disorder), or a mock genetically engineered hematopoietic cell).

In some embodiments, a method of genetically engineering cells described herein comprises a step of providing a wild-type cell, e.g., a wild-type hematopoietic stem or progenitor cell. In some embodiments, the wile-type cell is an un-edited cell comprising (e.g., expressing) two functional copies of a gene encoding GBA. In some embodiments, the cell comprises a GBA gene sequence as provided in GenBank: NG_009783.1. In some embodiments, the cell comprises a GBA gene sequence encoding a GBA protein that is encoded in the sequence provided by GenBank: AAC51820.1.

In some embodiments, a cell (e.g., a hematopoietic cell, e.g., a hematopoietic stem cell) described herein is deficient for a lineage-specific cell-surface antigen. In some embodiments, a cell has reduced or eliminated expression of a lineage-specific cell-surface antigen relative to a wildtype hematopoietic stem cell. Cells having reduced or eliminated expression of a lineage-specific cell-surface antigen may be resistant or immune to targeting by immunotherapeutic agents which specifically bind to the lineage-specific cell-surface antigen. In some embodiments, a genetically modified cell produced by a method described herein comprises a genetic modification directed toward a genetic disease (e.g., a modification correcting a prior mutation as described herein) and also has reduced or eliminated expression of a lineage-specific cell-surface antigen relative to a wildtype (e.g., as a result of a different genetic modification). Without wishing to be bound by theory, such a multiply modified cell may advantageously be administered to a subject to treat a genetic disease and enable co-administration of an immunotherapeutic agent that might otherwise target the modified cell (e.g., and reduce its effectiveness). Lineage-specific cell surface antigens are known for a variety of cell types. In some embodiments, a lineage-specific cell-surface antigen is chosen from: BCMA, CD19, CD20, CD30, ROR1, B7H6, B7H3, CD23, CD33, CD38, CD45, C-type lectin like molecule-1, CS1, IL-5, L1-CAM, PSCA, PSMA, CD138, CD133, CD70, CD5, CD6, CD7, CD13, NKG2D, NKG2D ligand, CLEC12A, CD11, CD123, CD56, CD30, CD14, CD66b, CD41, CD61, CD62, CD235a, CD146, CD326, LMP2, CD22, CD52, CD10, CD3/TCR, CD79/BCR, and CD26. In some embodiments, a lineage-specific cell-surface antigen is chosen from: CD33, CD19, CD123, CLL-1, CD30, CD5, CD6, CD7, CD38, CD45, and BCMA. In some embodiments, a lineage-specific cell-surface antigen is chosen from: CD7, CD13, CD19, CD22, CD25, CD32, CD33, CD38, CD44, CD45, CD47, CD56, 96, CD117, CD123, CD135, CD174, CLL-1, folate receptor b, IL1RAP, MUC1, NKG2D/NKG2DL, TIM-3, and WT1. See also examples of lineage-specific cell-surface antigens from BD Biosciences Human CD Marker Chart, https://www.bdbiosciences.com/content/dam/bdb/campaigns/reagent-education/BD_Reagents_CDMarkerHuman_Poster.pdf (incorporated by reference in its entirety).

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985); Transcription and Translation (B. D. Hames & S. J. Higgins, eds. (1984); Immobilized Cells and Enzymes (IRL Press, (1986); and B. Perbal, A practical Guide To Molecular Cloning (1984).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

### Example 1: CRISPR/Cas9-Based and HDR-Mediated Modification of the Human CCR5 Locus

### HDR Editing System

Single-stranded template polynucleotides (e.g., ssODNs) to direct HDR-mediated editing of genomic CCR5 sequences using CRISPR/Cas9 were designed after identifying Cas9 PAM sequences within the CCR5 genomic sequence.

Suitable PAM sequences were identified, and Cas9 sgRNAs were designed, comprising the following targeting domains:

TABLE 5
Cas9 target domain sequences human CCR5 and
corresponding gRNA targeting domain sequences.

| Guide Name | Target Domain Sequence |
|---|---|
| SG9 | (SEQ ID NO: 31) TGACATCAATTATTATACAT |
| | (SEQ ID NO: 32) ATGTATAATAATTGATGTCA |
| | (SEQ ID NO: 33) UGACAUCAAUUAUUAUACAU |
| SG10 | (SEQ ID NO: 34) TTTTGCAGTTTATCAGGATG |
| | (SEQ ID NO: 35) CATCCTGATAAACTGCAAAA |
| | (SEQ ID NO: 36) UUUUGCAGUUUAUCAGGAUG |
| SG11 | (SEQ ID NO: 37) GTAGAGCGGAGGCAGGAGGC |
| | (SEQ ID NO: 38) GCCTCCTGCCTCCGCTCTAC |
| | (SEQ ID NO: 39) GUAGAGCGGAGGCAGGAGGC |
| SG12 | (SEQ ID NO: 40) TTCACATTGATTTTTTGGCA |
| | (SEQ ID NO: 41) TGCCAAAAAATCAATGTGAA |
| | (SEQ ID NO: 42) UUCACAUUGAUUUUUUGGCA |

For each target site, the first sequence represents the DNA target domain sequence, the second sequence represents the reverse complement thereof, and the third sequence represents the targeting domain sequence of a CCR5 gRNA.

Guide RNAs comprising the above targeting domains and Cas9 sgRNA scaffold sequences were synthesized.

Template polynucleotides, here ssODNs, were designed comprising the following structure:
[5′ homology arm]-[donor sequence]-[3′homology arm].

The 5′ and 3′ homology arms comprised a sequence of 97 nucleotides each, which was 100% homologous to the genomic DNA sequence directly adjacent (in either 5′ or 3′ direction) to the Cas9 cut site for each gRNA. Without wishing to be bound by theory, it is believed that Cas9 cuts between the third and fourth nucleotide 5′ of the PAM sequence. Accordingly, the 3′ homology arm comprised the following structure: N3-[PAM]-N91, i.e., three nucleotides homologous to the three genomic nucleotides directly 3′ of the Cas9 cut site, a nucleotide sequence homologous to the genomic PAM sequence targeted by the respective gRNA (NGG), and a sequence of 91 nucleotides with 100% homology to the genomic sequence directly 3′ to the PAM sequence. The donor sequence comprised a sequence of 6 nucleotides. A schematic of the ssODN structure (with an exemplary donor sequence comprising a sequence of 6 nucleotide that includes a Pvu1 restriction endonuclease recognition site) is depicted in FIG. 1.

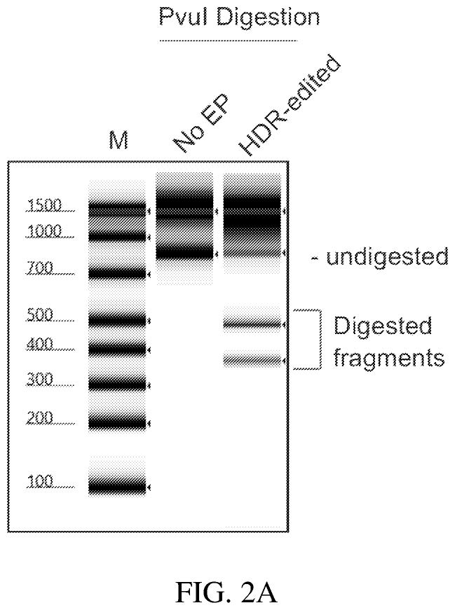

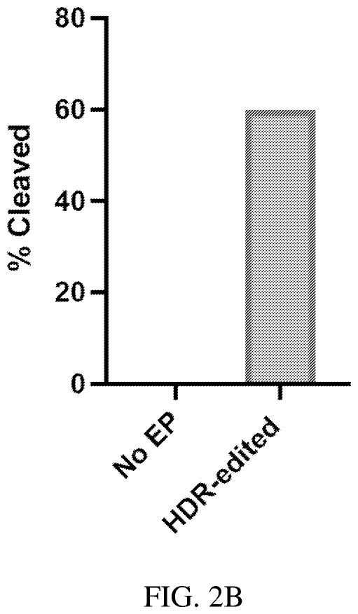

A number of ssODNs was designed to comprise a donor sequence that could be recognized by a restriction endonuclease, here Pvu1, in order to allow for identification of edited cells using a Pvu1 restriction digest (FIG. 2). The sequences of these ssODNs are provided below, with the donor sequence in bold and the respective PAM sequence underlined:

```
CCR5 ssODN7 (used with sgRNA SG9)
  (SEQ ID NO: 43)
TCTAGGACTTTATAAAAGATCACTTTTTATTTATGCACAGGGTGGAACAAGATGGATTATCAAGTGTCAAGTCCA
ATCTATGACATCAATTATTATACGATCGCATCGGAGCCCTGCCAAAAAATCAATGTGAAGCAAATCGCAGCCCGC
CTCCTGCCTCCGCTCTACTCACTGGTGTTCATCTTTGGTTTTGTGGGCAA
CCR5 ssODN28 (used with sgRNA SG10)
  (SEQ ID NO: 44)
CCAGAAGGGGACAGTAAGAAGGAAAAACAGGTCAGAGATGGCCAGGTTGAGCAGGTAGATGTCAGTCATGCTCTT
CAGCCTTTTGCAGTTTATCAGGCGATCGATGAGGATGACCAGCATGTTGCCCACAAAACCAAAGATGAACACCAG
TGAGTAGAGCGGAGGCAGGAGGCGGGCTGCGATTTGCTTCACATTGATTT
CCR5 ssODN9 (used with sgRNA SG11)
  (SEQ ID NO: 45)
ATGCTCTTCAGCCTTTTGCAGTTTATCAGGATGAGGATGACCAGCATGTTGCCCACAAAACCAAAGATGAACACC
AGTGAGTAGAGCGGAGGCAGGACGATCGGGCGGGCTGCGATTTGCTTCACATTGATTTTTTGGCAGGGCTCCGAT
GTATAATAATTGATGTCATAGATTGGACTTGACACTTGATAATCCATCTT
CCR5 ssODN10 (used with sgRNA SG12)
(SEQ ID NO: 46)
GGATGACCAGCATGTTGCCCACAAAACCAAAGATGAACACCAGTGAGTAGAGCGGAGGCAGGAGGCGGGCTGCGA
TTTGCTTCACATTGATTTTTTGCGATCGGCAGGGCTCCGATGTATAATAATTGATGTCATAGATTGGACTTGACA
CTTGATAATCCATCTTGTTCCACCCTGTGCATAAATAAAAAGTGATCTTT
```

### Enzymatic Characterization of HDR-Mediated Editing

HDR-mediated editing of CD34+ cells was carried out by electroporation of CD34-positive cells obtained from PBMCs in the presence of an RNP comprising a Cas9 nuclease and a single guide RNA (sgRNA), either SG9, SG, 10, SG11, or SG12, to direct Cas9 cleavage of the C—C Motif Chemokine Receptor 5 (CCR5) gene, and the respective ssODN (ssODN7, ssODN8, sSODN9, or ssODN10, respectively), to direct HDR-mediated genomic integration of the Pvu1 restriction site comprised in the donor sequence into the Cas9 target site. To screen cells for successful editing of the CCR5 locus, genomic DNA was isolated from both non-electroporated and electroporated cells and then digested using Pvu1. Digested DNA samples were separated via gel electrophoresis to detect Pvu1 restriction bands. Confirmation of HDR-mediated editing was determined via the presence of an electrophoretic mobility shift resulting in bands at ˜500 bp and ˜380 bp as seen in 60% of the CCR5 substrate isolated from electroporated cells (FIGS. 2A-2B).

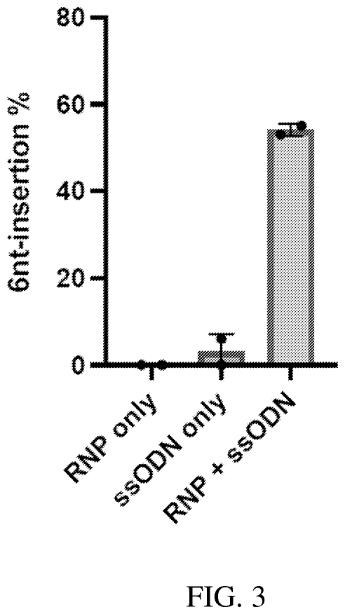

The presence of the Pvu1 restriction site in the CCR5 locus was also detected by sequencing. Sequencing data was analyzed via inference of CRISPR/Cas9 (ICE) software to quantify the percent of insertion of the 6 nucleotide Pvu1 site. While electroporation with ribonucleoprotein (RNP; the complex of sgRNA and Cas9) alone or ssODN alone resulted in no significant detection of the Pvu1 insert, electroporation of CD34+ cells with RNP and ssODN resulted in insertion of the Pvu1 in ˜55% of sequenced CCR5 products due to HDR-mediated editing (FIG. 3). The data demonstrate that highly efficient HDR-mediated gene editing was achieved by combining the ssODN design parameters above with CRISPR/Cas system induced DSBs.

### Example 2: Optimization of HDR-Mediated Editing

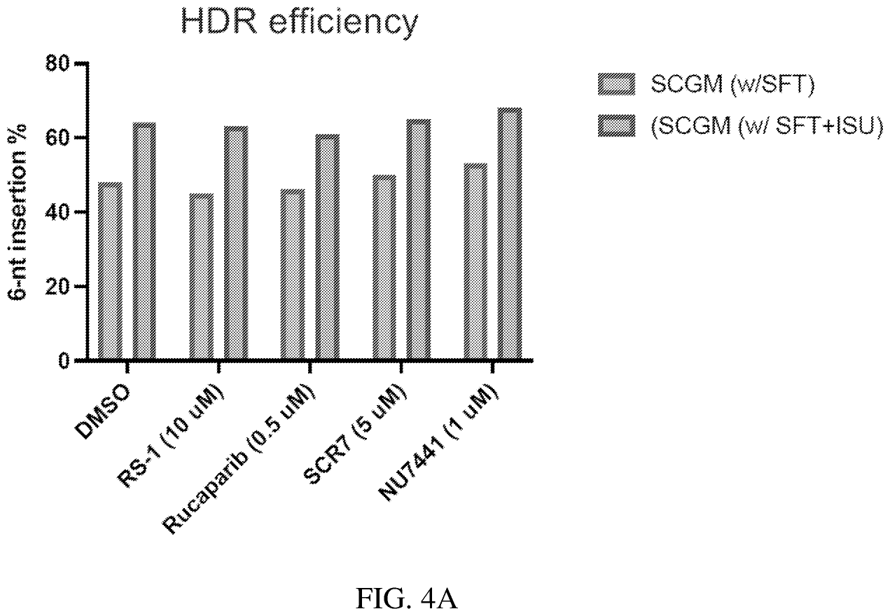

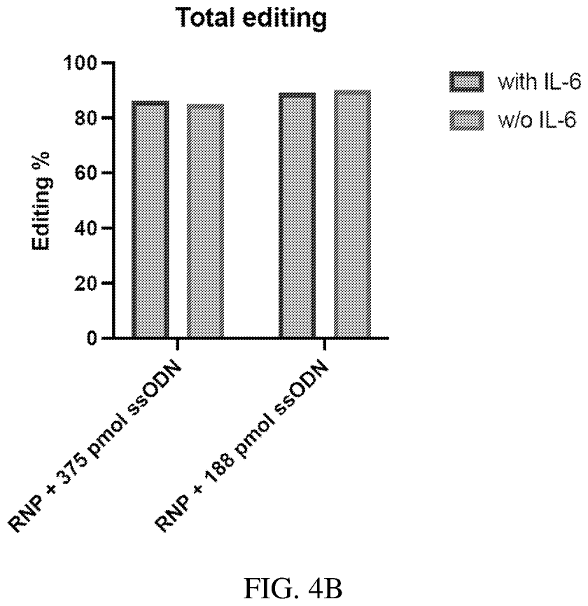

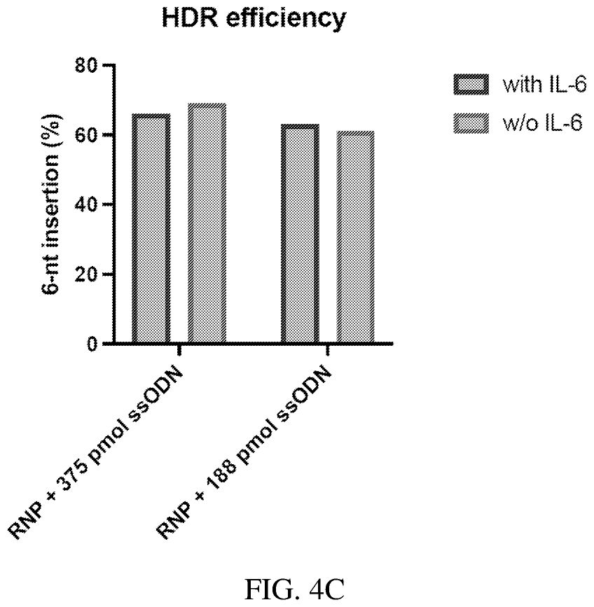

To determine conditions that would enhance HDR-mediated editing efficiency, the role of media conditions was assessed. CD34+ cells were cultured in stromal cell growth media (SCGM) supplemented with human stem cell factor (hSCF), Fms-like tyrosine kinase 3 Ligand (FLT3-L), and thrombopoietin (TPO) to promote cell differentiation and proliferation. Using ICE analysis of sequencing data to detect Pvu1 restriction site insertion into the CCR5 gene of CD34+ cells, cells were electroporated with DNA repair modulators to skew repair pathway utilization toward HDR following Cas9 cleavage in combination with cell expansion compounds Interleukin-6 (IL6), StemRegenin 1 (SR1), and UM171 in addition to RNP and ssODN. DNA repair modulators investigated included SCR7 (a ligase IV inhibitor), NU7441 (a DNA-PK inhibitor), Rucaparib (a PARP inhibitor), and RS-1 (an HDR enhancer). While addition of DNA repair modulators provided no significant advantage for promoting HDR, addition of IL6, SR1, and UM171 improved HDR-mediated editing efficiency in CD34+ cells (FIG. 4A). Analyses of the effect of IL6 addition to CD34+ cell media indicated that the presence or absence of IL6 did not significantly affect overall editing efficiency (FIG. 4B) or HDR (FIG. 4C). The data demonstrate that HDR-promoting agents (SR1 and UM171, in the presence or in the absence of IL-6) significantly increase the efficiency of HDR in CD34+ cells.

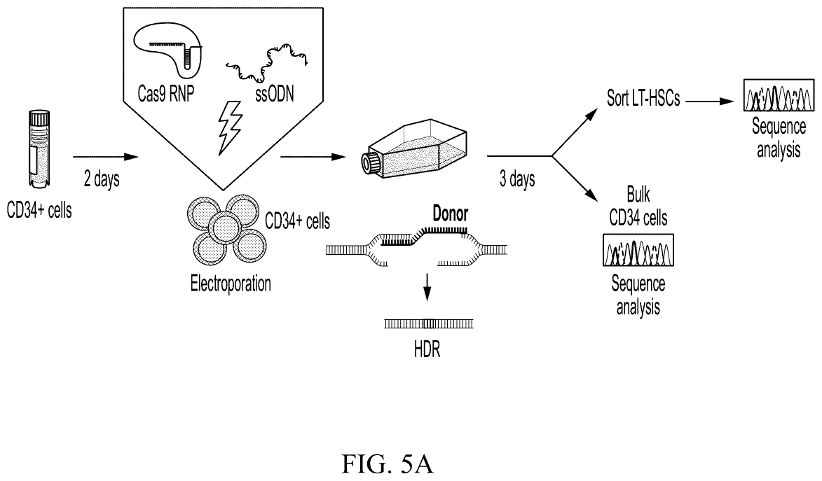

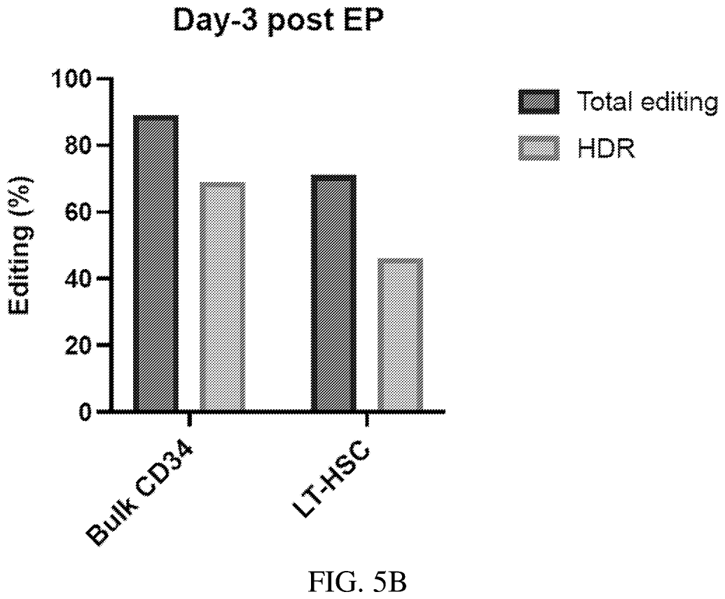

To determine if HDR-mediated editing could successfully generate long term-engrafting human stem cells (LT-HSCs) from CD34+ cells, CD34+ cells were cultured in serum-free expansion media (SFEM) supplemented with hSCF, FLT3-L, TPO, SR1, and UM171. CD34+ cells were electroporated with RNP and ssODN and then grown for 3 days. After 3 days, cells were sorted by flow cytometry using standard LT-HSC markers, and used for sequencing analysis (FIG. 5A). The results showed that while bulk CD34+ cells exhibited higher total editing and HDR-mediated editing efficiencies at the CCR5 locus relative to LT-HSCs, both populations exhibited editing efficiencies of ˜50% or greater (FIG. 5B). These data demonstrate that HDR-mediated editing utilizing HDR-promoting agents can generate stable genetic modifications in LT-HSCs at high frequencies.

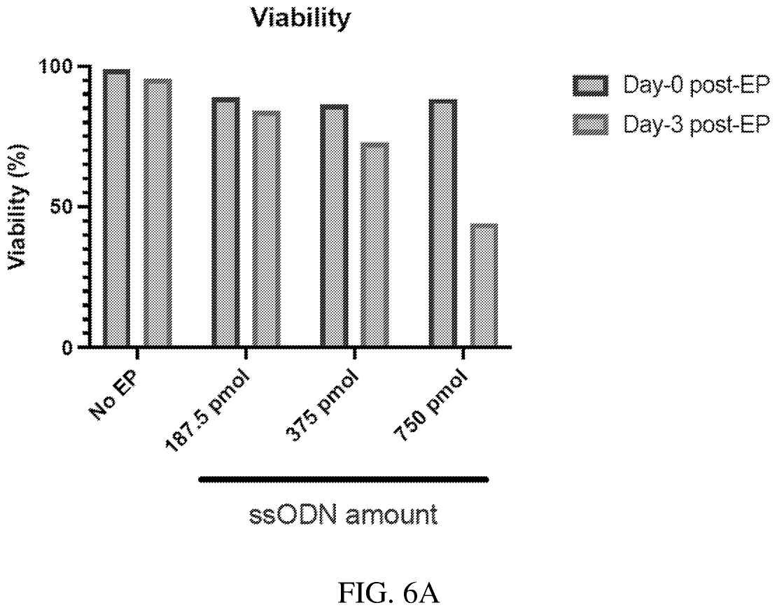

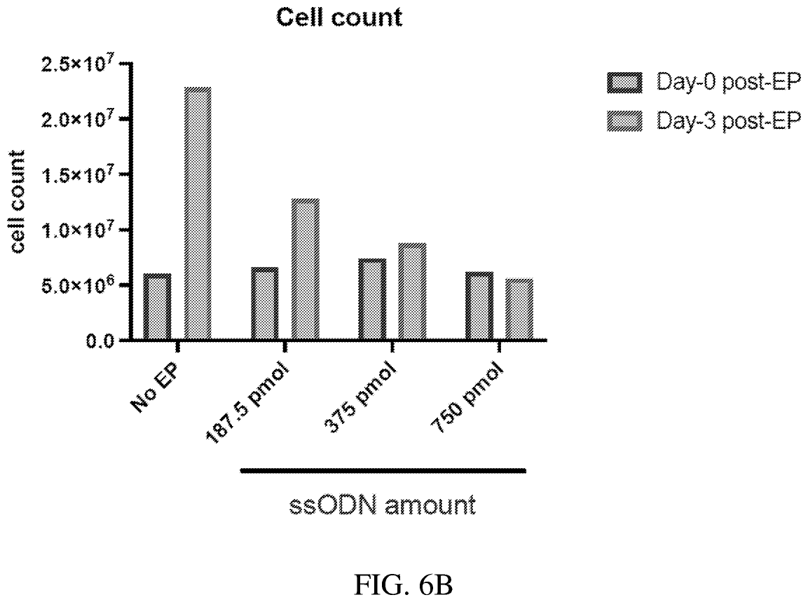

To determine if ssODN concentration affected cell vitality, CD34+ cells were electroporated with RNP and varying concentrations of ssODN and cell viability and cell count analyses were performed on 0 and 3 days following electroporation. While ssODN concentration had no effect on cell viability on day 0 post-electroporation, cell viability analyses performed 3 days post-electroporation indicated that increased ssODN concentrations are associated with decreased viability (FIG. 6A). Similarly, while cell counts taken on day 0 post-electroporation appeared unaffected by ssODN concentration, cell counts on day 3 post-electroporation were reduced with increasing concentrations of ssODN (FIG. 6B). This data demonstrates that use of lower concentrations of template polynucleotides (e.g., ssODNs, dsODNs, minicircle plasmids, or nanoplasmids) provides a viability advantage in methods of genetically engineering cells via HDR-mediated gene editing. Together with the data related to the use of HDR-promoting agents, which was tested at various ssODN concentrations (see, e.g., FIG. 4B and FIG. 4C) the results provided herein demonstrate that HDR-mediated gene editing in CD34+ HSCs can be achieved at high efficiencies with minimal loss of viability when lower concentrations of ssODNs are used in combination with HDR-promoting agents, e.g., SR1, and UM171, and/or one or more expansion agents.

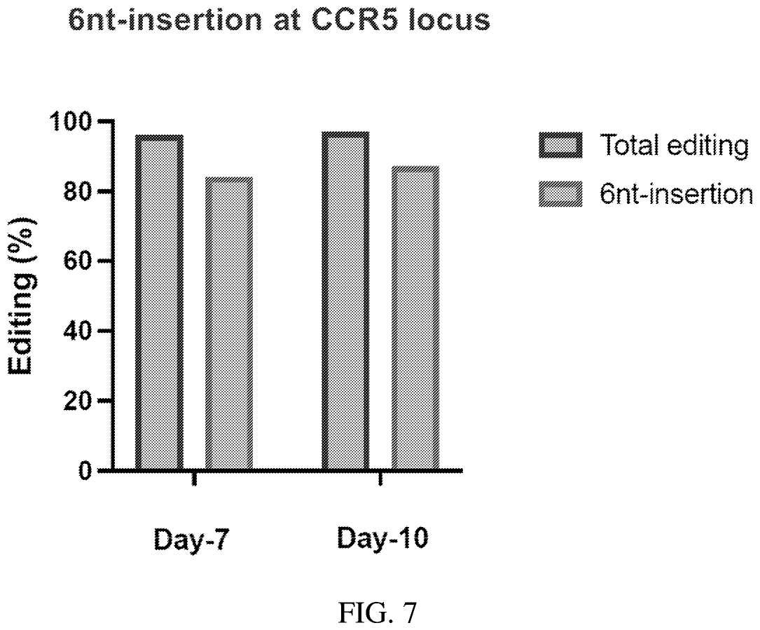

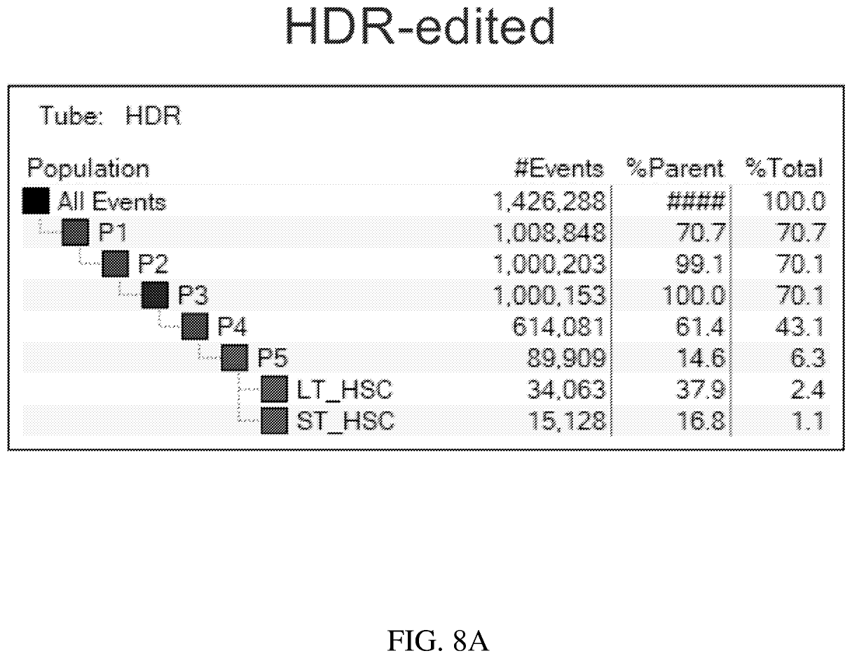

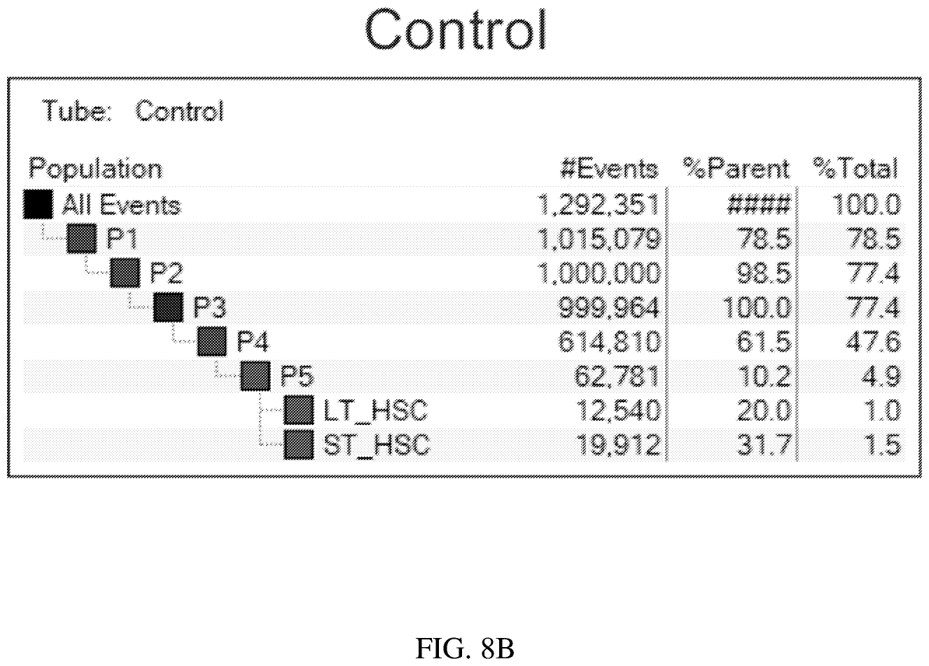

After optimizing media conditions, HDR-mediated editing rates in the CCR5 locus were determined by ICE analysis of sequencing data in T cells. The results indicated that at 7- and 10-days post-electroporation, T cells exhibited near 100% total editing efficiency and greater than 80% HDR efficiency (FIG. 7). This data shows that use of a genetic modification mixture of the disclosure can achieve high HDR efficiency in modifying a population of exemplary cells (here CD34+ HSCs), while maintaining viability and differentiation capacity.

### Example 3: CRISPR/Cas9-Based and HDR-Mediated Modification of the Human GBA Locus

### HDR Editing System

Single-stranded template polynucleotides (e.g., ssODNs) to direct HDR-mediated editing of genomic GBA sequences using CRISPR/Cas9 were designed after identifying Cas9 PAM sequences within the GBA genomic sequence proximal to two known genomic mutations that are causally associated with Gaucher disease in humans.

The first mutation frequently observed in Gaucher patients is 1226A>G, resulting in the AAC codon comprising the nucleotide at position 1226 to be changed into an AGC codon, and thus in a substitution of asparagine at position 409 of the GBA protein with serine (N409S), which is a mutation characteristic for, and causally associated with, Gaucher disease.

A second mutation frequently observed in Gaucher patients is 1448T>C, resulting in the CTG codon comprising the nucleotide at position 1448 to be changed into an CCG codon, and thus in a substitution of leucine at position 483 of the GBA protein with proline (L483P), which is also a mutation characteristic for, and causally associated with, Gaucher disease.

The 1226 and 1448 positions are illustrated below in their genomic context. Positions 1226 and 1448 are in bold and underlined:

(SEQ ID NO: 50)
ACTTTCTGGCTCCAGCCAAAGCCACCCTAGGGGAGACACACCGCCTGTTCCCCAACACCATGCTCTTTGCCTCAG
AGGCCTGTGTGGGCTCCAAGTTCTGGGAGCAGAGTGTGCGGCTAGGCTCCTGGGATCGAGGGATGCAGTACAGCC
ACAGCATCATCACGGTAAGCCACCCCAGTCTCCCTTCCTGCAAAGCAGACCTCAGACCTCTTACTAGTTTCACCA
AAGACTGACAGAAGCCCTTCCTGTCCAGCTTTCCCCAGCTAGCCTGCCCTTTTGAGCAACTCTGGGGAACCATGA
TTCCCTATCTTCCCTTTCCTTCACAGGTCTGCACACCTCATTGCCCCTTTTGCAACTACTGAGGCACTTGCAGCT
GCCTCAGACTTCTCAGCTCCCCTTGAGATGCCTGGATCTTCACACCCCCAACTCCTTAGCTACTAAGGAATGTGC
CCCTCACAGGGCTGACCTACCCACAGCTGCCTCTCCCACATGTGACCCTTACCTACACTCTCTGGGGACCCCCAG
TGTTGCGCCTTTGTCTCTTTGCCTTTGTCCTTACCCTAGAACCTCCTGTACCATGTGGTCGGCTGGACCGACTGG
AACCTTGCCCTGAACCCCGAAGGAGGACCCAATTGGGTGCGTAACTTTGTCGACAGTCCCATCATTGTAGACATC
ACCAAGGACACGTTTTACAAACAGCCCATGTTCTACCACCTTGGCCACTTCAGGTGAGTGGAGGGCGGGCACCCC
CATTCCATACCAGGCCTATCATCTCCTACATCGGATGGCTTACATCACTCTACACCACGAGGGAGCAGGAAGGTG
TTCAGGGTGGAACCTCGGAAGAGGCACACCCATCCCCTTTTGCACCATGGAGGCAGGAAGTGACTAGGTAGCAAC
AGAAAACCCCAATGCCTGAGGCTGGACTGCGATGCAGAAAAGCAGGGTCAGTGCCCAGCAGCATGGCTCCAGGCC
TAGAGAGCCAGGGCAGAGCCTCTGCAGGAGTTATGGGGTGGGTCCGTGGGTGGGTGACTTCTTAGATGAGGGTTT
CATGGGAGGTACCCCGAGGGACTCTGACCATCTGTTCCCACATTCAGCAAGTTCATTCCTGAGGGCTCCCAGAGA
GTGGGGCTGGTTGCCAGTCAGAAGAACGACCTGGACGCAGTGGCACTGATGCATCCCGATGGCTCTGCTGTTGTG
GTCGTGCTAAACCGGTGAGGGCAATGGTGAGGTCTGGGAAGTGGGCTGAAGACAGCGTTGGGGGCCTTGGCAGGA
TCACACTCTCAGCTTCTCCTCCCTGCTCCCTAGCTCCTCTAAGGATGTGCCTCTTACCATCAAGGATCCTGCTGT
GGGCTTCCTGGAGACAATCTCACCTGGCTACTCCATTCACACCTACCTGTGGCGTCGCCAGTGATGGAGCAGATA
CTCAAGGAGGCACTGGGCTCAGCCTGGGCATTAAAGGGACAGAGTCAGCTCACACGCTGTCTGTGACTAAAGAGG
GCACAGCAGGGCCAGTGTGAGCTTACAGCGACGTAAGCCCAGGGGCAATGGTTTGGGTGACTCACTTTCCCCTCT
AGGTGGTGCCAGGGGCTGGAGGCCCCTAGAAAAAGATCAGTAAGCCCCAGTGTCCCCCCAGCCCCCATGCTTAT

Suitable PAM sequences proximal to positions 1226 and 1448 in the human genomic GBA sequence were identified, and Cas9 sgRNAs were designed as follows: four sgRNAs (SG1-4) targeting sequences proximal to position 1226 in the human genomic GBA sequence were designed (FIG. 9A) and four sgRNAs (SG5-8) targeting sequences proximal to position 1448 in the human genomic GBA sequence were designed (FIG. 9B), comprising the following targeting domains:

TABLE 6
Cas9 sgRNAs targeting sequences proximal to
positions 1226 and 1448 of human GBA.

| Guide Name | Target Domain Sequence |
|---|---|
| SG1 | (SEQ ID NO: 1) ACATGGTACAGGAGGTTCTA |
| (1226) | (SEQ ID NO: 2) TAGAACCTCCTGTACCATGT |
| | (SEQ ID NO: 3) ACAUGGUACAGGAGGUUCUA |
| SG2 | (SEQ ID NO: 4) CACATGGTACAGGAGGTTCT |
| (1226) | (SEQ ID NO: 5) AGAACCTCCTGTACCATGTG |
| | (SEQ ID NO: 6) CACAUGGUACAGGAGGUUCU |
| SG3 | (SEQ ID NO: 7) AGCCGACCACATGGTACAGG |
| (1226) | (SEQ ID NO: 8) CCTGTACCATGTGGTCGGCT |
| | (SEQ ID NO: 9) AGCCGACCACAUGGUACAGG |
| SG4 | (SEQ ID NO: 10) CTAGAACCTCCTGTACCATG |
| (1226) | (SEQ ID NO: 11) CATGGTACAGGAGGTTCTAG |
| | (SEQ ID NO: 12) CUAGAACCUCCUGUACCAUG |
| SG5 | (SEQ ID NO: 13) GTCCAGGTCGTTCTTCTGAC |
| (1448) | (SEQ ID NO: 14) GTCAGAAGAACGACCTGGAC |
| | (SEQ ID NO: 15) GUCCAGGUCGUUCUUCUGAC |
| SG6 | (SEQ ID NO: 16) TGCCAGTCAGAAGAACGACC |
| (1448) | (SEQ ID NO: 17) GGTCGTTCTTCTGACTGGCA |
| | (SEQ ID NO: 18) UGCCAGUCAGAAGAACGACC |
| SG7 | (SEQ ID NO: 19) GCATCAGTGCCACTGCGTCC |
| (1448) | (SEQ ID NO: 20) GGACGCAGTGGCACTGATGC |
| | (SEQ ID NO: 21) GCAUCAGUGCCACUGCGUCC |
| SG8 | (SEQ ID NO: 22) AAGAACGACCTGGACGCAG |
| (1448) | (SEQ ID NO: 23) CTGCGTCCAGGTCGTTCTTC |
| | (SEQ ID NO: 24) GAAGAACGACCUGGACGCAG |

For each sgRNA, the first sequence represents the DNA target domain sequence, the second sequence represents the reverse complement thereof, and the third sequence represents an exemplary targeting domain sequence of an sgRNA that was used to target the respective target site.

Guide RNAs comprising the above targeting domains and Cas9 sgRNA scaffold sequences were synthesized.

Template nucleic acids, here ssODNs, were designed comprising the following structure:
[5′ homology arm]-[donor sequence]-[3′homology arm].

The 5′ and 3′ homology arms comprised a sequence of about 80-100 nucleotides each, which was 100% homologous to the genomic DNA sequence directly adjacent (in either 5′ or 3′ direction) to the Cas9 cut site for each gRNA.

First, a number of ssODNs were designed that included a donor sequence comprising a mutated 1226 or 1448 position, resulting in a 1226G or a 1448C after HDR-mediated integration of the ssODN into the genome, respectively. These ssODNs were used to edit wild-type CD34+ HSCs (CD34+ cells from a human subject not affected with Gaucher disease, and not carrying either one of these two mutations). Editing such wild-type cells with these ssODNs resulted in the creation of CD34+ HSCs carrying the respective Gaucher mutation (either 1226 A>G or 1448 T>C, as compared to the wild-type sequence, respectively), which are useful for modeling Gaucher disease, and also for rescue experiments, e.g., for evaluating gene editing strategies correcting these mutations. Some of the ssODNs designed for this purpose also included a number of silent mutations, e.g., nucleotide substitutions that did not result in any change in the encoded GBA amino acid sequence, which served as sequence tags to facilitate identification of edited cells and quantification of editing efficiencies and persistence of edited cells in cell populations over time.

The sequences of these ssODNs are provided below, with the respective 1226 or 1448 Gaucher mutation in bold and underline, the respective PAM sequence underlined, and any silent mutations in bold:

```
GBA ssODN1 (1226A>G and silent mutations, used with gRNA SG4)
(SEQ ID NO: 25)
TGCCTCTCCCACATGTGACCCTTACCTACACTCTCTGGGGACCCCCAGTGTTGCGCCTTTGTCTCTTTGCCTTTG
TCCTTACCCTAGAGCCTGCTCTATCATGTGGTCGGCTGGACCGACTGGAACCTTGCCCTGAACCCCGAAGGAGGA
CCCAATTGGGTGCGTAACTTTGTCGACAGTCCCATCATTGTAGACATCAC
GBA ssODN2 (1226A>G, used with gRNA SG4)
  (SEQ ID NO: 26)
TGCCTCTCCCACATGTGACCCTTACCTACACTCTCTGGGGACCCCCAGTGTTGCGCCTTTGTCTCTTTGCCTTTG
TCCTTACCCTAGAGCCTCCTGTACCATGTGGTCGGCTGGACCGACTGGAACCTTGCCCTGAACCCCGAAGGAGGA
CCCAATTGGGTGCGTAACTTTGTCGACAGTCCCATCATTGTAGACATCAC
GBA ssODN3 (1226A>G and silent mutations, used with gRNA SG1)
  (SEQ ID NO: 27)
TGATGGGACTGTCGACAAAGTTACGCACCCAATTGGGTCCTCCTTCGGGGTTCAGGGCAAGGTTCCAGTCGGTCC
AGCCGACCACGTGATAGAGCAGGCTCTAGGGTAAGGACAAAGGCAAAGAGACAAAGGCGCAACACTGGGGGTCCC
CAGAGAGTGTAGGTAAGGGTCACATGTGGGAGAGGCAGCTGTGGGTAGGT
GBA ssODN4 (1226A>G, used with gRNA SG1)
(SEQ ID NO: 28)
TGATGGGACTGTCGACAAAGTTACGCACCCAATTGGGTCCTCCTTCGGGGTTCAGGGCAAGGTTCCAGTCGGTCC
AGCCGACCACATGGTACAGGAGGCTCTAGGGTAAGGACAAAGGCAAAGAGACAAAGGCGCAACACTGGGGGTCCC
CAGAGAGTGTAGGTAAGGGTCACATGTGGGAGAGGCAGCTGTGGGTAGGT
GBA ssODN5 (1448T>C and silent mutations, used with gRNA SG6)
  (SEQ ID NO: 29)
GGGAGGTACCCCGAGGGACTCTGACCATCTGTTCCCACATTCAGCAAGTTCATTCCTGAGGGCTCCCAGAGAGTG
GGGCTGGTTGCTAGCCAGAAAAATGATCCGGACGCAGTGGCACTGATGCATCCCGATGGCTCTGCTGTTGTGGTC
GTGCTAAACCGGTGAGGGCAATGGTGAGGTCTGGGAAGTGGGCTGAAGAC
GBA ssODN6 (1448 T>C and silent mutations, used with gRNA SG7)
  (SEQ ID NO: 30)
CAACGCTGTCTTCAGCCCACTTCCCAGACCTCACCATTGCCCTCACCGGTTTAGCACGACCACAACAGCAGAGCC
ATCGGGATGCATGAGGGCGACGGCATCCGGGTCGTTCTTCTGACTGGCAACCAGCCCCACTCTCTGGGAGCCCTC
AGGAATGAACTTGCTGAATGTGGGAACAGATGGTCAGAGTCCCTCGGGGT
```

CD34+ cells were electroporated with RNP comprising the designed sgRNAs and with the designed ssODNs. Cells were screened by ICE analysis of sequencing data. SG1, SG2 and SG4 yielded high editing efficiency of exon 9 of the GBA gene while SG6 and SG7 yielded high editing efficiency of exon 10 of the GBA gene (FIGS. 9C-9D).

This data shows that HDR-mediated editing can induce mutations at the positions in the GBA gene that are implicated in Gaucher disease with high efficiency, indicating that HDR-mediated editing can be employed to modify genomic DNA at these positions, and thus demonstrating that Gaucher disease mutations can be addressed by HDR-mediated gene editing at clinically relevant efficiencies in CD34+ HSCs. Genetic modification of the Gaucher disease loci in cells was confirmed by detection of the presence of sequences comprising silent mutations that were introduced into the GBA genomic sequence via HDR-mediated integration of the ssODN donor sequences (FIG. 9E).

### HDR-Mediated Editing in Models of Gaucher Disease

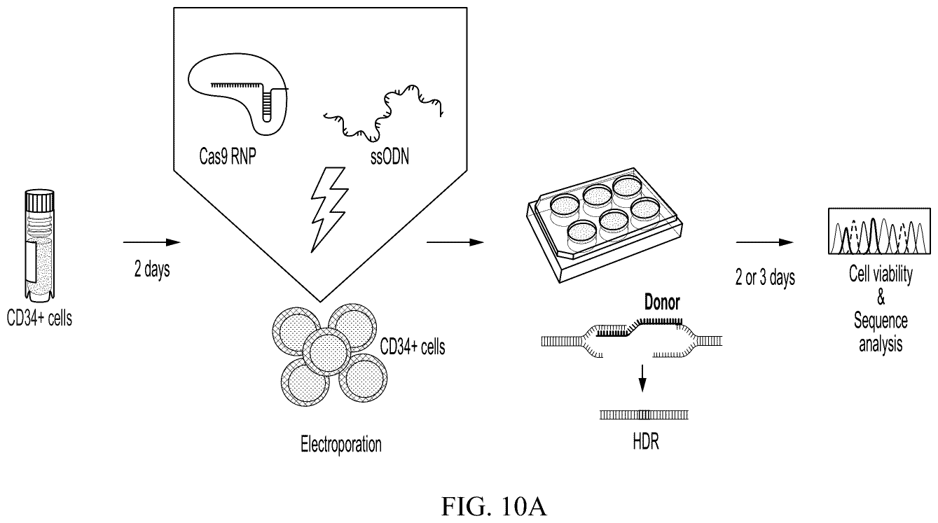

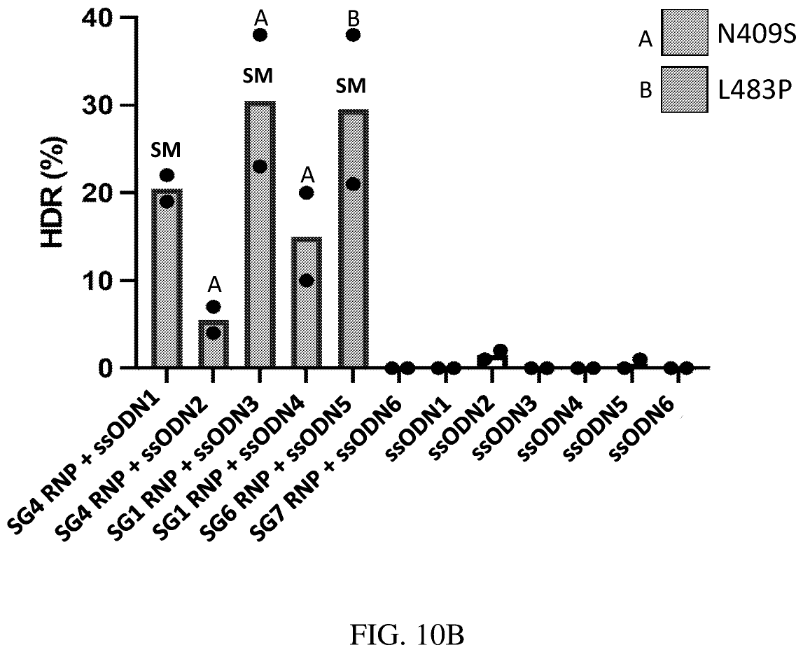

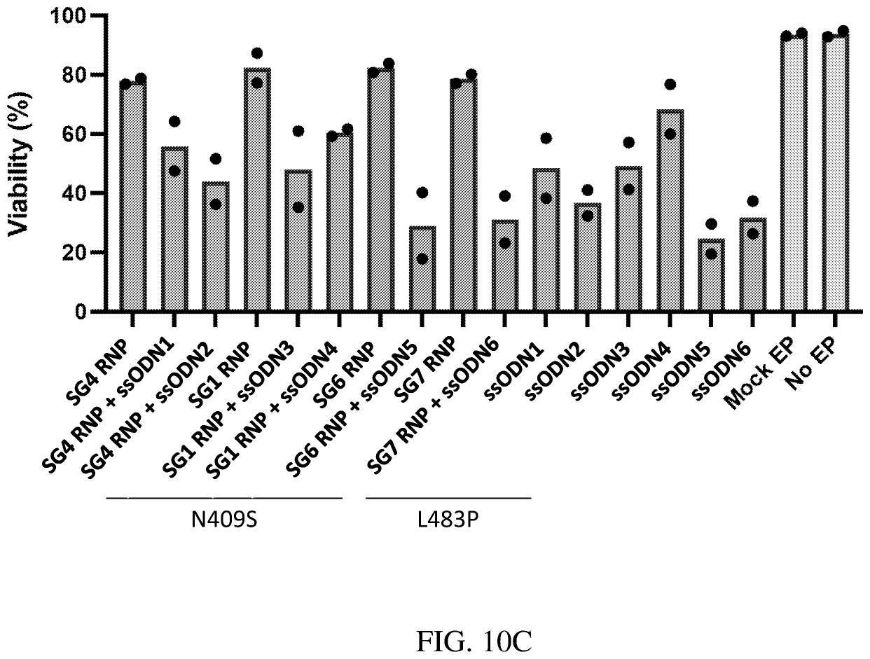

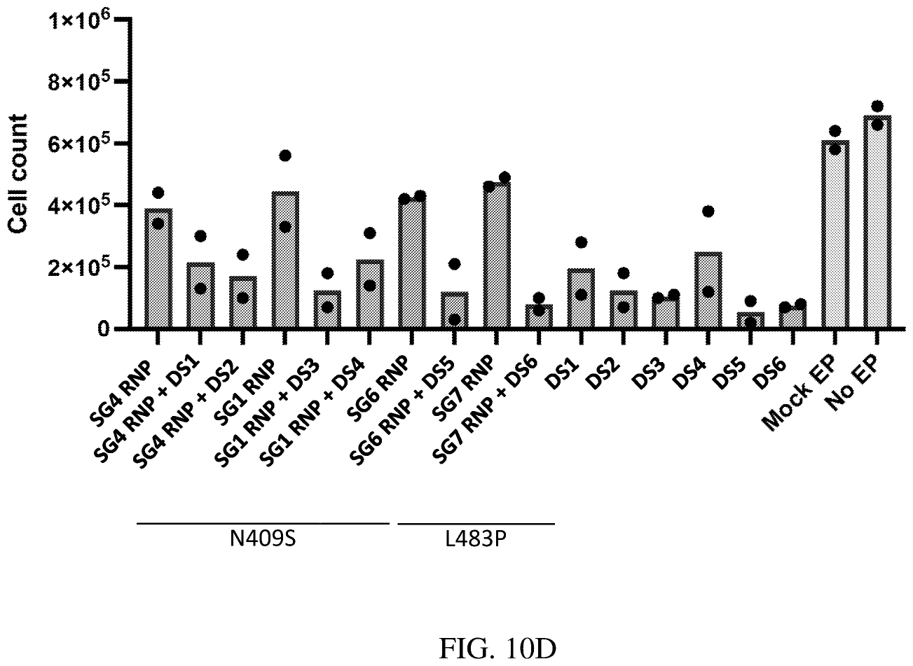

To optimize HDR-mediated editing of the GBA gene loci associated with Gaucher disease, ssODN candidates were screened for their ability to promote high editing efficiencies. Four ssODNs (ssODN1-4) comprising a sequence with a modification to generate the N409S mutation in exon 9 of the GBA gene and two ssODNs (ssODN5 and ssODN6) comprising a sequence with a modification to generate the L483P mutation in exon 10 of the GBA gene were screened. CD34+ cells were electroporated with different combinations of RNPs and ssODNs that corresponded to their respective target sequences, as described above, grown for 2-3 days, and then analyzed for cell viability and by sequencing (FIG. 10A). ICE analysis of sequencing data revealed that SG4+ssODN1, SG1+ssODN3, and SG6+ssODN5 yielded the highest HDR-mediated editing efficiencies at the GBA locus (FIG. 10B). While electroporation of CD34+ cells with SGs alone resulted in higher cell viability (FIG. 10C) and cell counts (FIG. 10D) as opposed to electroporation with ssODNs alone, electroporation of CD34+ cells with SGs combined with ssODNs resulted in sufficient levels of cell viability and cell counts (FIGS. 10C-10D) for clinical applications.

### Example 4: Correction of GBA N409S Mutation in HSCs

CD34+ HSCs are obtained from a Gaucher disease patient having a 1226 A>G mutation in the GBA gene, resulting in an N409S mutation in the GBA protein, which is the cause for Gaucher disease in the patient. The HSCs are contacted with a Cas9 RNP comprising a Cas9 nuclease and a guide RNA, as described above, in the presence of an ssODN comprising a GBA sequence characterized by an A nucleotide at position 1226. The following combinations of guide RNA and ssODN are used:

```
GBA ssODN11 (1226G>A, used with gRNA SG4)
  (SEQ ID NO: 51)
TGCCTCTCCCACATGTGACCCTTACCTACACTCTCTGGGGACCCCCAGTGTTGCGCCTTTGTCTCTTTGCCTTTG
TCCTTACCCTAGAACCTCCTGTACCATGTGGTCGGCTGGACCGACTGGAACCTTGCCCTGAACCCCGAAGGAGGA
CCCAATTGGGTGCGTAACTTTGTCGACAGTCCCATCATTGTAGACATCAC
GBA sSODN12 (1226G>A and silent mutations, used with gRNA SG4)
  (SEQ ID NO: 52)
TGCCTCTCCCACATGTGACCCTTACCTACACTCTCTGGGGACCCCCAGTGTTGCGCCTTTGTCTCTTTGCCTTTG
TCCTTACCCTAGAACCTGCTCTATCATGTGGTCGGCTGGACCGACTGGAACCTTGCCCTGAACCCCGAAGGAGGA
CCCAATTGGGTGCGTAACTTTGTCGACAGTCCCATCATTGTAGACATCAC
GBA ssODN13 (1226G>A, used with gRNA SG1)
  (SEQ ID NO: 53)
TGATGGGACTGTCGACAAAGTTACGCACCCAATTGGGTCCTCCTTCGGGGTTCAGGGCAAGGTTCCAGTCGGTCC
AGCCGACCACATGGTACAGGAGGTTCTAGGGTAAGGACAAAGGCAAAGAGACAAAGGCGCAACACTGGGGGTCCC
CAGAGAGTGTAGGTAAGGGTCACATGTGGGAGAGGCAGCTGTGGGTAGGT
GBA ssODN14 (1226G>A and silent mutations, used with gRNA SG1)
(SEQ ID NO: 54)
TGATGGGACTGTCGACAAAGTTACGCACCCAATTGGGTCCTCCTTCGGGGTTCAGGGCAAGGTTCCAGTCGGTCC
AGCCGACCACGTGATAGAGCAGGTTCTAGGGTAAGGACAAAGGCAAAGAGACAAAGGCGCAACACTGGGGGTCCC
CAGAGAGTGTAGGTAAGGGTCACATGTGGGAGAGGCAGCTGTGGGTAGGT
```

HDR-mediated introduction of the ssODN sequences into the GBA locus results in correction of the 1226A>G mutation, and the creation of a GBA sequence comprising an A nucleotide at position 1226, and thus encoding a GBA protein variant that is not associated with Gaucher disease. Glucocerebrosidase activity is measured and confirmed in edited HSCs, or in their in-vitro differentiated progeny cells, using standard assays (see, e.g., Lecourt et al., PLOS One. 2013 Jul. 25; 8 (7): e69293 A prospective study of bone marrow hematopoietic and mesenchymal stem cells in type 1 Gaucher disease patients). HDR-mediated editing is also confirmed via sequencing, detecting the 1226A nucleotide or, where an ssODN including silent mutations is used, detecting one or more of the silent mutations. The efficiency of HDR-mediated editing is measured by sequencing and confirmed to be suitable for clinical use, e.g., for re-administration to the patient the CD34+ cells were derived from.

### Example 5: Treatment of a Gaucher Disease Patient with a GBA N409S Mutation

For an autologous cell therapy of Gaucher disease in a patient, CD34+ HSCs are isolated from a Gaucher disease patient carrying a 1226A>G mutation using standard peripheral blood stem cell mobilization techniques. A Gaucher disease patient is administered an i.v. dose of granulocyte colony-stimulating factor (G-CSF) of 10 mg/kg per day and peripheral blood is obtained via standard apheresis procedures. CD34+ positive HSCs are enriched for using immunomagnetic beads. A minimum of 2×10⁶ CD34+ cells/kg body weight of the patient are collected using standard procedures (see, e.g., Park et al., Bone Marrow Transplantation (2003) 32:889).

Freshly isolated peripheral blood-derived CD34+ cells are seeded at 1×10⁶ cells/ml in serum-free CellGro SCGM Medium in the presence of cell culture grade Stem Cell Factor (SCF) 300 ng/ml, FLT3-L 300 ng/ml, Thrombopoietin (TPO) 100 ng/ml and IL-3 60 ng/ml. Following 24 hour of pre-stimulation, CD34+ HSCs are electroporated with RNP containing Cas9 and sgRNA in the presence of an ssODN, as described above. After electroporation, HSCs are cultured in the presence of HDR promoting agents SR1 and UM171, either in the presence or in the absence of IL-6. Successful editing is detected via sequence analysis or by detecting a GBA protein comprising a 409N residue, e.g., by immunochemical detection, and an editing efficiency of at least 40% is confirmed before re-administration of the edited HSCs to the patient.

HDR-edited CD34+ cells are re-infused to the patient via standard procedures. See, e.g., Somaraju et al., Cochrane Database Syst Rev. 2017 October; 2017 (10): CD006974, Hematopoietic stem cell transplantation for Gaucher disease).

The patient is monitored after HSC transplant, and in particular symptoms of Gaucher disease (fatigue, anemia, pain, etc.) are assessed. A marked increase in quality of life and a significant decrease in the severity of Gaucher disease symptoms is expected after the HSC transplant.

### Example 6: Correction of GBA L483P Mutation in HSCs

CD34+ HSCs are obtained from a Gaucher disease patient having a 1448 T>C mutation in the GBA gene, resulting in an L483P mutation in the GBA protein, which is the cause for Gaucher disease in the patient. The HSCs are contacted with a Cas9 RNP comprising a Cas9 nuclease and a guide RNA, as described above, in the presence of an ssODN comprising a GBA sequence characterized by a T nucleotide at position 1448. The following combinations of guide RNA and ssODN are used:

```
GBA ssODN15 (1448C>T, used with gRNA SG6)
  (SEQ ID NO: 55)
GGGAGGTACCCCGAGGGACTCTGACCATCTGTTCCCACATTCAGCAAGTTCATTCCTGAGGGCTCCCAGAGAGTG
GGGCTGGTTGCCAGTCAGAAGAACGACCTGGACGCAGTGGCACTGATGCATCCCGATGGCTCTGCTGTTGTGGTC
GTGCTAAACCGGTGAGGGCAATGGTGAGGTCTGGGAAGTGGGCTGAAGAC
GBA sSODN16 (1448C>T and silent mutations, used with gRNA SG6)
  (SEQ ID NO: 56)
GGGAGGTACCCCGAGGGACTCTGACCATCTGTTCCCACATTCAGCAAGTTCATTCCTGAGGGCTCCCAGAGAGTG
GGGCTGGTTGCTAGCCAGAAAAATGATCTGGACGCAGTGGCACTGATGCATCCCGATGGCTCTGCTGTTGTGGTC
GTGCTAAACCGGTGAGGGCAATGGTGAGGTCTGGGAAGTGGGCTGAAGAC
GBA ssODN17 (1448 T>C and silent mutations, used with gRNA SG7)
(SEQ ID NO: 57)
CAACGCTGTCTTCAGCCCACTTCCCAGACCTCACCATTGCCCTCACCGGTTTAGCACGACCACAACAGCAGAGCC
ATCGGGATGCATGAGGGCGACGGCATCCGGGTCGTTCTTCTGACTGGCAACCAGCCCCACTCTCTGGGAGCCCTC
AGGAATGAACTTGCTGAATGTGGGAACAGATGGTCAGAGTCCCTCGGGGT
```

HDR-mediated introduction of the ssODN sequences into the GBA locus results in correction of the 1448T>C mutation, and the creation of a GBA sequence comprising an T nucleotide at position 1448, and thus encoding a GBA protein variant that is not associated with Gaucher disease. Glucocerebrosidase activity is measured and confirmed in edited HSCs, or in their in-vitro differentiated progeny cells, using standard assays (see, e.g., Lecourt et al., PLOS One. 2013 Jul. 25; 8 (7): e69293 A prospective study of bone marrow hematopoietic and mesenchymal stem cells in type 1 Gaucher disease patients). HDR-mediated editing is also confirmed via sequencing, detecting the 1148T nucleotide or, where an ssODN including silent mutations is used, detecting one or more of the silent mutations. The efficiency of HDR-mediated editing is measured by sequencing and confirmed to be suitable for clinical use, e.g., for re-administration to the patient the CD34+ cells were derived from.

### Example 7: Treatment of a Gaucher Disease Patient with a GBA L483P Mutation

For an autologous cell therapy of Gaucher disease in a patient, CD34+ HSCs are isolated from a Gaucher disease patient using standard peripheral blood stem cell mobilization techniques. A Gaucher disease patient is administered an i.v. dose of granulocyte colony-stimulating factor (G-CSF) of 10 mg/kg per day and peripheral blood is obtained via standard apheresis procedures. CD34+ positive HSCs are enriched for using immunomagnetic beads. A minimum of 2×10⁶ CD34+ cells/kg body weight of the patient are collected using standard procedures (see, e.g., Park et al., Bone Marrow Transplantation (2003) 32:889).

Freshly isolated peripheral blood-derived CD34+ cells are seeded at 1×10⁶ cells/ml in serum-free CellGro SCGM Medium in the presence of cell culture grade Stem Cell Factor (SCF) 300 ng/ml, FLT3-L 300 ng/ml, Thrombopoietin (TPO) 100 ng/ml and IL-3 60 ng/ml. Following 24 hour of pre-stimulation, CD34+ HSCs are electroporated with RNP containing Cas9 and sgRNA in the presence of an ssODN, as described above. After electroporation, HSCs are cultured in the presence of HDR promoting agents SR1 and UM171, either in the presence or in the absence of IL-6. Successful editing is detected via sequence analysis or by detecting a GBA protein comprising a 483L residue, e.g., by immunochemical detection, and an editing efficiency of at least 40% is confirmed before re-administration of the edited HSCs to the patient.

HDR-edited CD34+ cells are re-infused to the patient via standard procedures. See, e.g., Somaraju et al., Cochrane Database Syst Rev. 2017 October; 2017 (10): CD006974, Hematopoietic stem cell transplantation for Gaucher disease).

The patient is monitored after HSC transplant, and in particular symptoms of Gaucher disease (fatigue, anemia, pain, etc.) are assessed. A marked increase in quality of life and a significant decrease in the severity of Gaucher disease symptoms is expected after the HSC transplant.

### Example 8: Re-Editing of Engineered GBA Locus in CD34+ Cells

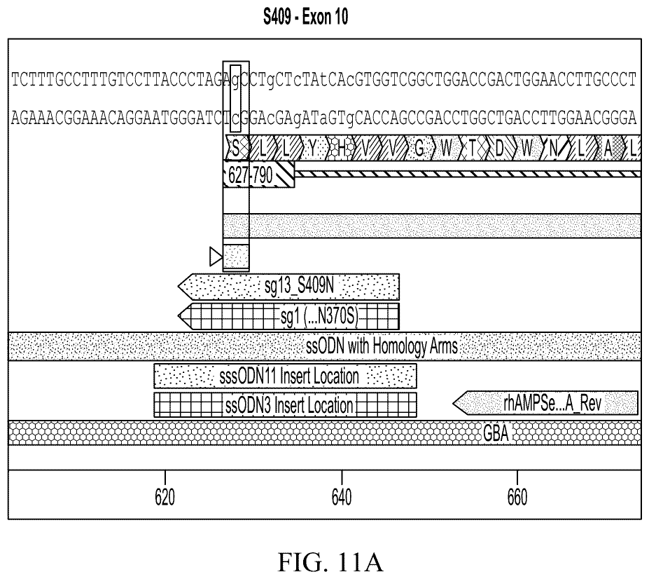

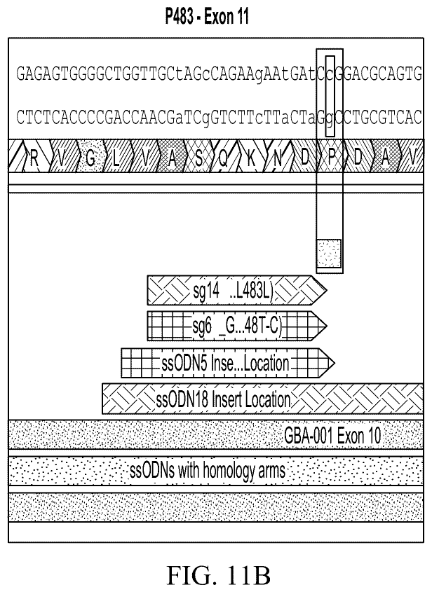

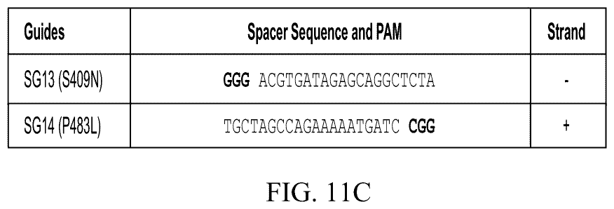

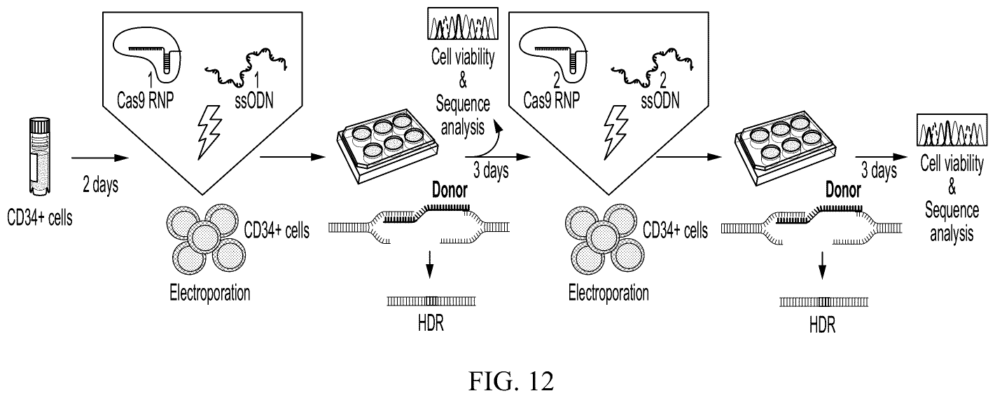

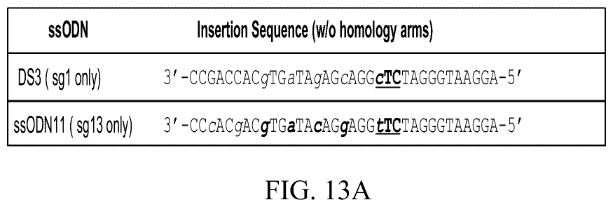

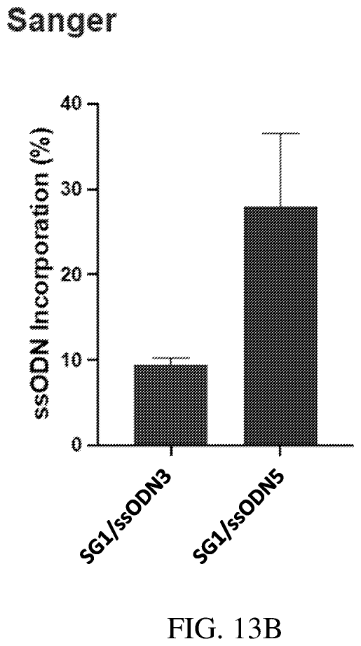

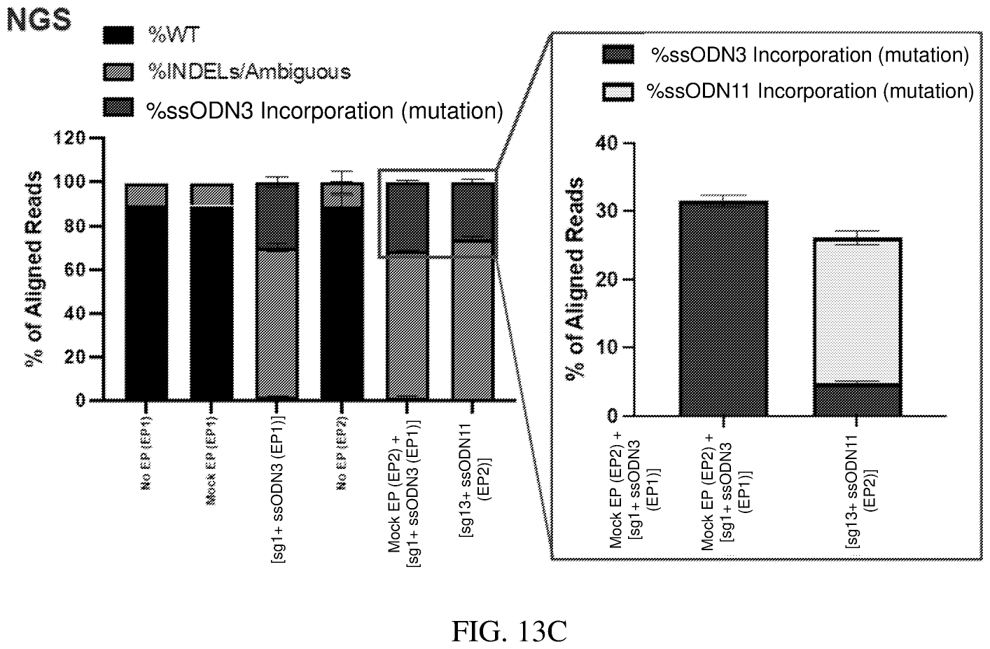

sgRNAs comprising the Gaucher Disease-associated N409S or L483P mutations in the GBA locus were designed to hybridize to target sequences in exons 9 and 10 of GBA, respectively (FIGS. 11A-11C; Table 7). A cell population comprising 1×10⁶ cells were thawed and cultured for two days prior to electroporation with either 3 μg Cas9/sgRNA RNP and 37 pmol of ssODN (see Table 7) or 15 μg Cas9/sgRNA RNP and 187.5 pmol ssODN. Integration of Gaucher Disease-associated mutations in GBA were confirmed by sequencing analysis. Three days-post editing, cells were subjected to a subsequent round of electroporation. Here, a sample of 2×10⁵ cells were electroporated with 3 μg Cas9/sgRNA and 37.5 pmol ssODN encoding a corrective mutation in GBA (see Table 7). After 3 days, cells were subjected to cell viability and sequencing analyses (FIG. 12). Sequencing analyses showed that mutation of GBA in the first round of editing occurred with approximately 30% efficiency. Within the population of edited cells comprising the Gaucher Disease-associated GBA mutation, approximately 81.7% editing efficiency was achieved in the subsequent round of electroporation leading to integration of the corrective mutation in GBA (FIGS. 13A-13C). These results indicated that Gaucher Disease-associated mutation N409S in GBA could be introduced and corrected in CD34+ cells using ssODN-based HDR.

TABLE 7

| | | | | | ssODN | |
|---|---|---|---|---|---|---|
| | | | | | Sequence | |
| | | | | | (w/o | |
| Edit | | Guide | | | homology | ssODN Sequence |
| Type | Guides | Sequence | Strand | ssODN | arms) | (w/ homology arms) |
| Mutation | SG19 | GGG | − | ssODN3 | CCGACCA | TGATGGGACTGTCGACAAAGTTA |
| Creation | | ACATGG | | | CgTGaTAg | CGCACCCAATTGGGTCCTCCTTC |
| (N409S) | | TACAGG | | | AGcAGGcT | GGGGTTCAGGGCAAGGTTCCAGT |
| | | AGGTTC | | | CTAGGGT | CGGTCCAGCCGACCACGTGATAG |
| | | TA (SEQ | | | AAGGA | AGCAGGCTCTAGGGTAAGGACA |
| | | ID NO: | | | (SEQ ID | AAGGCAAAGAGACAAAGGCGCA |
| | | 58) | | | NO: 64) | ACACTGGGGGTCCCCAGAGAGTG |
| | | | | | | TAGGTAAGGGTCACATGTGGGAG |
| | | | | | | AGGCAGCTGTGGGTAGGT (SEQ |
| | | | | | | ID NO: 27) |
| | | | | ssODN4 | CCGACCA | TGATGGGACTGTCGACAAAGTTA |
| | | | | | TGGTACA | CGCACCCAATTGGGTCCTCCTTC |
| | | | | | GGAGGcT | GGGGTTCAGGGCAAGGTTCCAGT |
| | | | | | CTAGGGT | CGGTCCAGCCGACCACATGGTAC |
| | | | | | AAGGA | AGGAGGCTCTAGGGTAAGGACA |
| | | | | | (SEQ ID | AAGGCAAAGAGACAAAGGCGCA |
| | | | | | NO: 65) | ACACTGGGGGTCCCCAGAGAGTG |
| | | | | | | TAGGTAAGGGTCACATGTGGGAG |
| | | | | | | AGGCAGCTGTGGGTAGGT (SEQ |
| | | | | | | ID NO: 28) |
| | | | | | | TGCCTCTCCCACATGTGACCCTTA |
| | | | | | TCCTTACC | CCTACACTCTCTGGGGACCCCCA |
| | sg4 | CTAGAA | + | sSODN1 | CTAGAgC | GTGTTGCGCCTTTGTCTCTTTGCC |
| | | CCTCCT | | | CTgCTcTAt | TTTGTCCTTACCCTAGAGCCTGCT |
| | | GTACCA | | | CATGTGGTC | CTATCATGTGGTCGGCTGGACCG |
| | | TG TGG | | | (SEQ ID | ACTGGAACCTTGCCCTGAACCCC |
| | | (SEQ ID | | | NO: 66) | GAAGGAGGACCCAATTGGGTGC |
| | | NO: 59) | | | | GTAACTTTGTCGACAGTCCCATC |
| | | | | | | ATTGTAGACATCAC (SEQ ID |
| | | | | | | NO: 25) |
| | | | | ssODN2 | TCCTTACC | TGCCTCTCCCACATGTGACCCTTA |
| | | | | | CTAGAgC | CCTACACTCTCTGGGGACCCCCA |
| | | | | | CTCCTGT | GTGTTGCGCCTTTGTCTCTTTGCC |
| | | | | | ACCATGT | TTTGTCCTTACCCTAGAGCCTCCT |
| | | | | | GGTC | GTACCATGTGGTCGGCTGGACCG |
| | | | | | (SEQ ID | ACTGGAACCTTGCCCTGAACCCC |
| | | | | | NO: 67) | GAAGGAGGACCCAATTGGGTGC |
| | | | | | | GTAACTTTGTCGACAGTCCCATC |
| | | | | | | ATTGTAGACATCAC (SEQ ID |
| | | | | | | NO: 26) |
| Mutation | sg6 | TGCCAG | + | ssODN5 | TgGTtGCtA | GGGAGGTACCCCGAGGGACTCTG |
| Creation | | TCAGAA | | | GcCAGAAg | ACCATCTGTTCCCACATTCAGCA |
| (L483P) | | GAACGA | | | AAtGAtCc | AGTTCATTCCTGAGGGCTCCCAG |
| | | CC TGG | | | GGACGCA | AGAGTGGGGCTGGTTGCTAGCCA |
| | | (SEQ ID | | | G (SEQ ID | GAAAAATGATCCGGACGCAGTG |
| | | NO: 60) | | | NO: 68) | GCACTGATGCATCCCGATGGCTC |
| | | | | | | TGCTGTTGTGGTCGTGCTAAACC |
| | | | | | | GGTGAGGGCAATGGTGAGGTCTG |
| | | | | | | GGAAGTGGGCTGAAGAC (SEQ |
| | | | | | | ID NO: 29) |
| | sg7 | AGG | − | ssODN6 | GATGCAT | CAACGCTGTCTTCAGCCCACTTC |
| | | GCATCA | | | gAGgGCgA | CCAGACCTCACCATTGCCCTCAC |
| | | GTGCCA | | | CgGCaTCC | CGGTTTAGCACGACCACAACAGC |
| | | CTGCGT | | | gGGTCGT | AGAGCCATCGGGATGCATGAGG |
| | | CC (SEQ | | | TC (SEQ ID | GCGACGGCATCCGGGTCGTTCTT |
| | | ID NO: | | | NO: 69) | CTGACTGGCAACCAGCCCCACTC |
| | | 61) | | | | TCTGGGAGCCCTCAGGAATGAAC |
| | | | | | | TTGCTGAATGTGGGAACAGATGG |
| | | | | | | TCAGAGTCCCTCGGGGT (SEQ |
| | | | | | | ID NO: 30) |
| Correct | sg13 | GGG | − | ssODN11 | CCcACgAC | TGATGGGACTGTCGACAAAGTTA |
| Mutation | | ACGTGA | | | gTGaTAcA | CGCACCCAATTGGGTCCTCCTTC |
| (S409N) | | TAGAGC | | | GgAGGtTC | GGGGTTCAGGGCAAGGTTCCAGT |
| | | AGGCTC | | | TAGGGTA | CGGTCCAGCCCACGACGTGATAC |
| | | TA (SEQ | | | AG (SEQ ID | AGGAGGTTCTAGGGTAAGGACA |
| | | ID NO: | | | NO: 70) | AAGGCAAAGAGACAAAGGCGCA |
| | | 62) | | | | ACACTGGGGGTCCCCAGAGAGTG |
| | | | | | | TAGGTAAGGGTCACATGTGGGAG |
| | | | | | | AGGCAGCTGTGGGTAGGT (SEQ |
| | | | | | | ID NO: 72) |
| Correct | sg14 | TGCTAG | + | ssODN12 | TcGTgGCt | GGGAGGTACCCCGAGGGACTCTG |
| Mutation | | CCAGAA | | | AGcCAGA | ACCATCTGTTCCCACATTCAGCA |
| (P483L) | | AAATGA | | | AgAAtGAc | AGTTCATTCCTGAGGGCTCCCAG |
| | | TC CGG | | | CtGGA | AGAGTGGGGCTCGTGGCTAGCCA |
| | | (SEQ ID | | | (SEQ ID | GAAGAATGACCTGGACGCAGTG |
| | | NO: 63) | | | NO: 71) | GCACTGATGCATCCCGATGGCTC |
| | | | | | | TGCTGTTGTGGTCGTGCTAAACC |
| | | | | | | GGTGAGGGCAATGGTGAGGTCTG |
| | | | | | | GGAAGTGGGCTGAAGAC (SEQ |
| | | | | | | ID NO: 73) |

Table Key:
Mutation codon, HDR Guide PAM, Silent mutations, Match silent mutations, Corrections, SNP

### Example 9: T Cell Engineering Using HDR

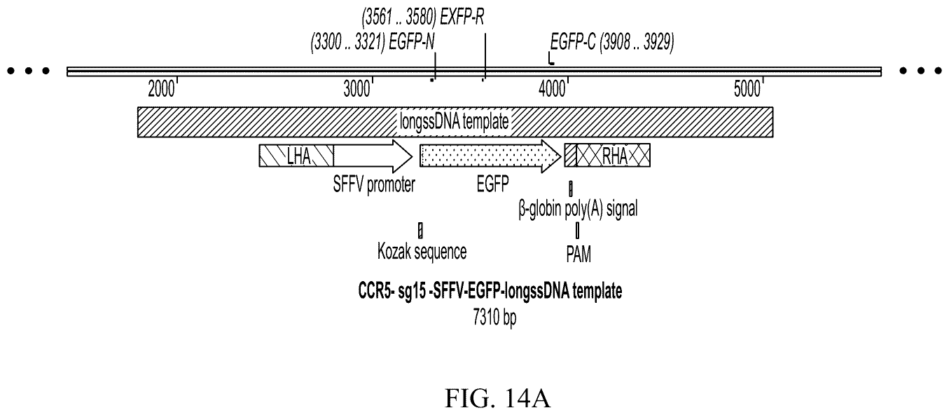

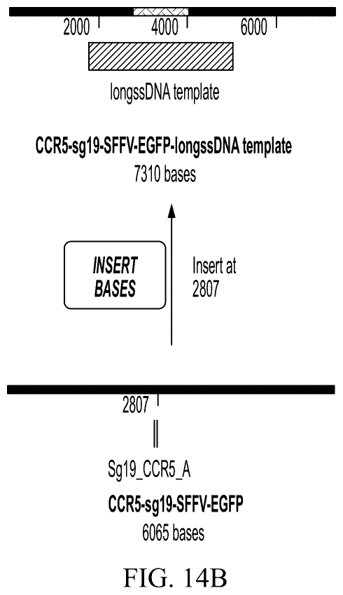

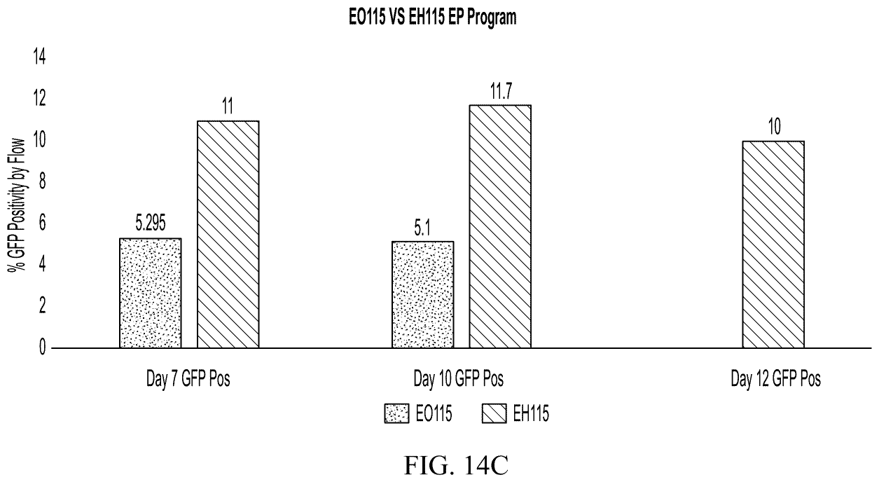

T cells were engineered using donor template-based HDR. Long, ssODNs encoding an EGFP reporter and homology arms corresponding to the CCR5 locus were designed to direct integration of the reporter at the CCR5 locus in T cells (FIGS. 14A-14B). T cells were electroporated with dsODN and Cas9 RNP in the presence of 0.6 μL of poly(glutamic acid) (PGA) at a concentration of 50 mg/mL. Flow cytometry analyses were used to confirm EGFP reporter expression in T cells post-electroporation. Electroporation program optimization was also confirmed using flow cytometry analyses of EGFP expression (FIG. 14C).

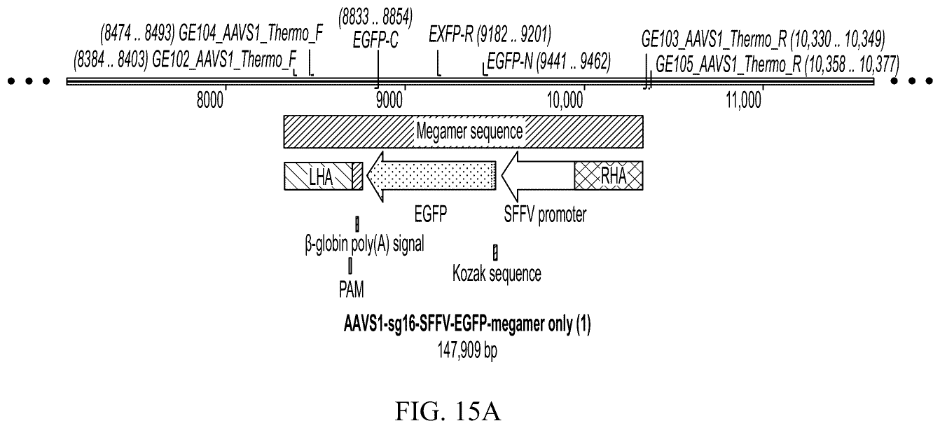

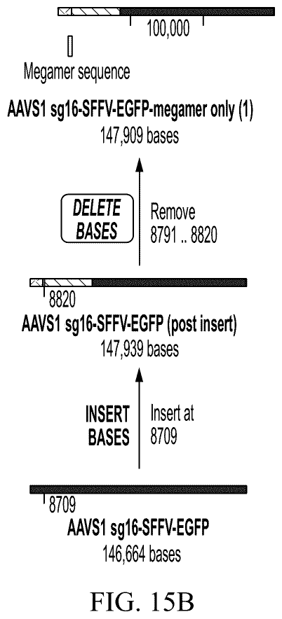

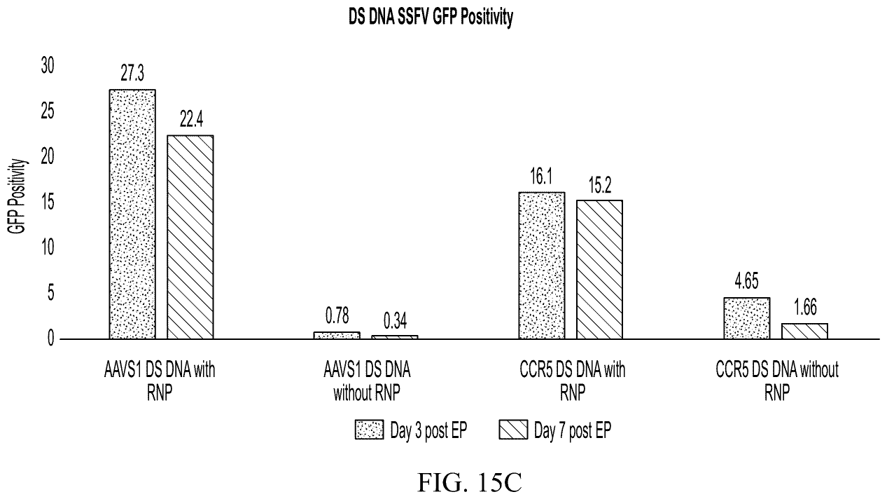

Two respective uncapped, dsODNs were designed comprising an EGFP reporter and homology arms to direct integration at either the AAVS1 or CCR5 loci in T cells (FIGS. 15A-15B; see Table 8). T cells were electroporated with dsODN and Cas9 RNP as described herein. Flow cytometry analyses were used to confirm EGFP expression in electroporated T cells. Approximately 26% of cells electroporated with un-capped, AAVS1 dsODN and 15% of cells electroporated with un-capped, CCR5 dsODN were EGFP-positive (FIG. 15C).

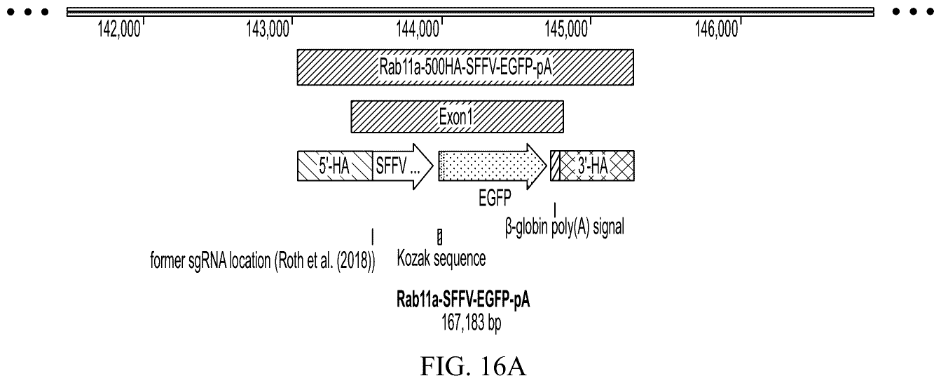

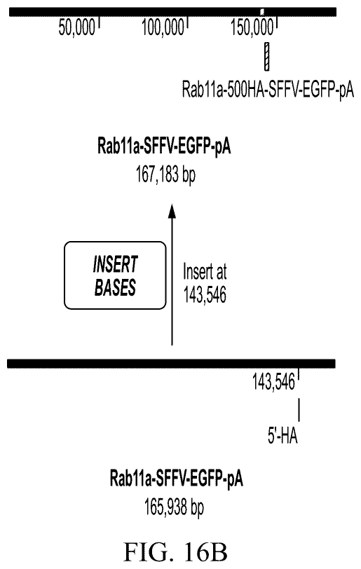

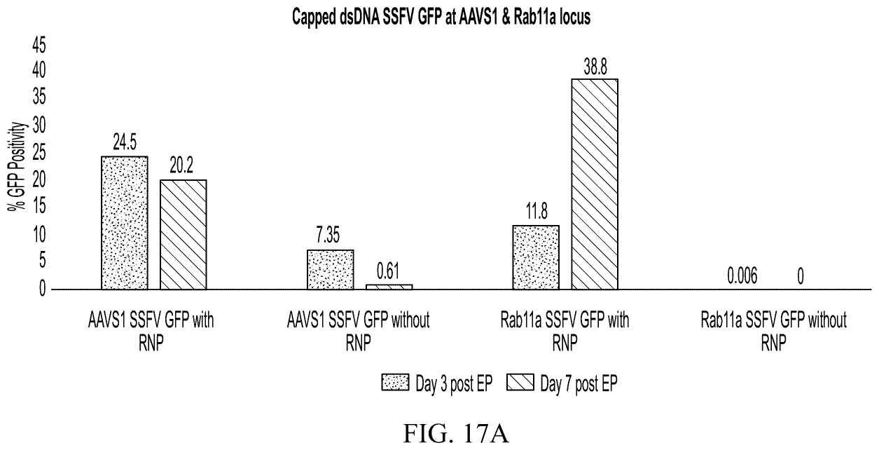

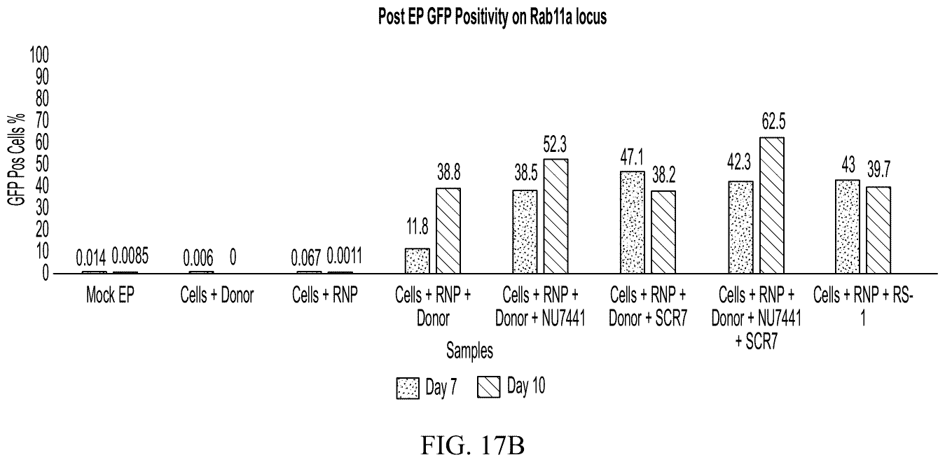

Two respective capped, dsODNs were designed comprising an EGFP reporter and homology arms to direct integration at either the AAVS1 or RAB11a loci in T cells (FIG. 16A-16B). T cells were electroporated with dsODN and Cas9 RNP as described herein. Flow cytometry analyses were used to confirm EGFP expression in electroporated T cells. Approximately 20% of cells electroporated with capped, AAVS1 dsODN and 38% of cells electroporated with capped, RAB11a dsODN were GFP positive (FIG. 17A). Further analyses indicated that T cells subjected to electroporation with 3 μg Cas9, 3 μg RAB11a sgRNA, and 1 μg capped dsODN followed by addition of NHEJ modulators (NU7441 at 1 μM, SCR7 at 5 μM, and/or SR1 at 5 μM) to the media for 24 hours post-electroporation resulted in approximately 10% increase in GFP expression but also reduced T cell viability (FIG. 17B).

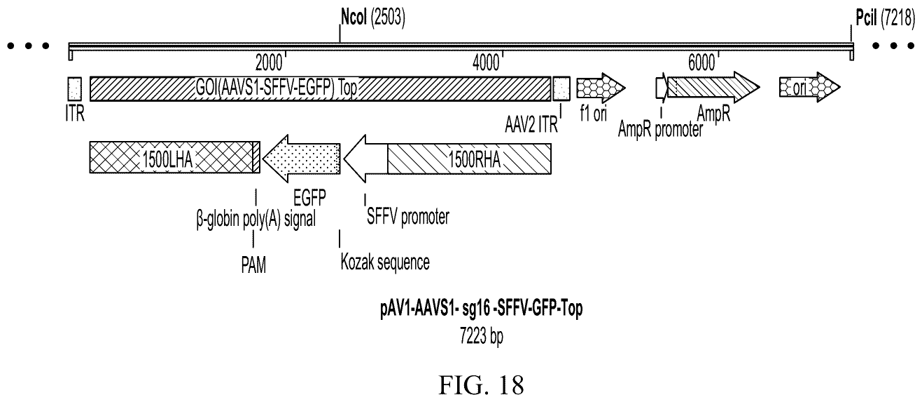

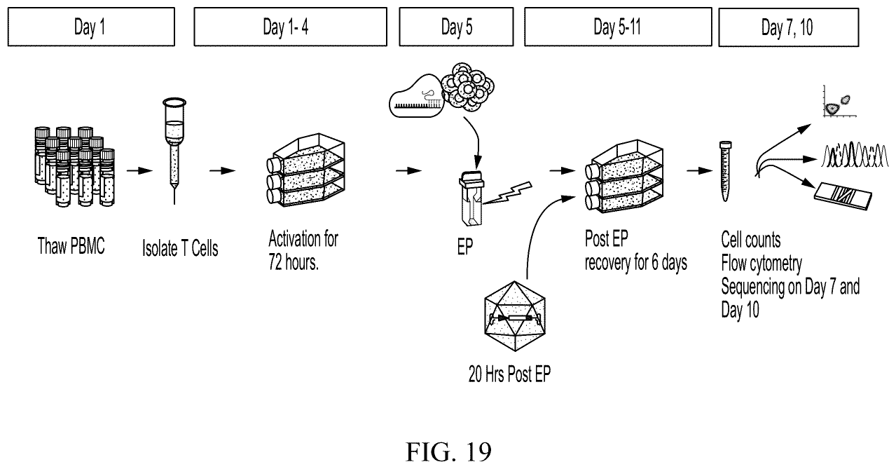

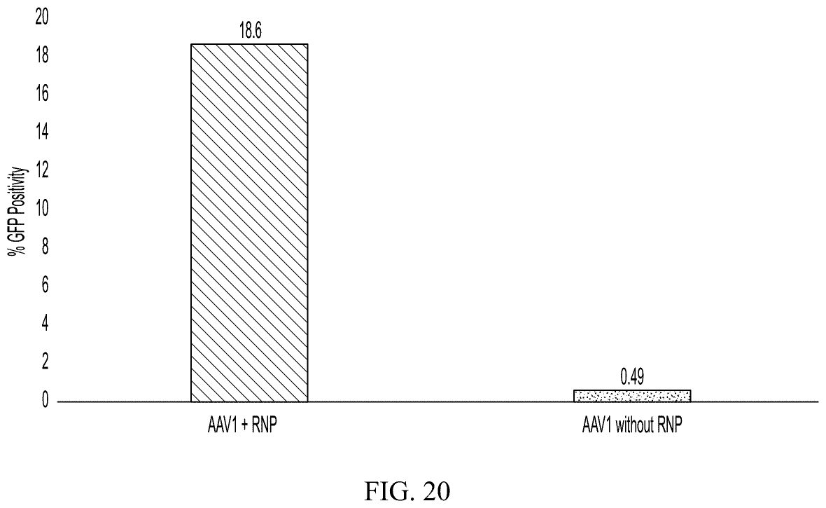

Recombinant adeno-associated viral (rAAV) vectors comprising dsODNs encoding a GFP reporter were designed to direct integration at the AAVS1 locus (FIG. 18). Donor T cells were thawed and isolated using the PAN T cell method. Cells were activated for three days via incubation with CD3 and CD28 antibodies. Subsequently, cells were cultured in X Vivo 15 with 5% FBS, 0.2 mM Glutamax, 10 mM N-acetyl cysteine, 200 u/mL IL-2, 5 ng/ml IL-7, and 5 ng/ml IL-15 for 24 hours prior to electroporation with Cas9 RNPs. At 20 hours post-electroporation, cells were contacted with rAAV particles of serotype AAV1 comprising AAVS1 dsODNs at a multiplicity of infection (MOI) of infection at 2×104 and allowed to recover for 6 days. On days 7 and 10 following electroporation, cells were subjected to cell counts, flow cytometry, and sequencing analyses (FIG. 19). Flow cytometry analyses showed that 18% of electroporated T cells exhibited GFP expression (FIG. 20).

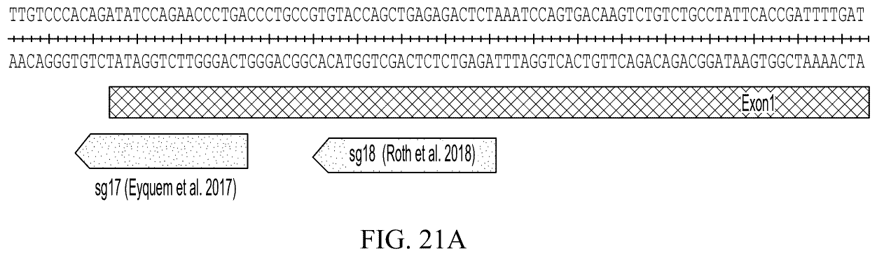

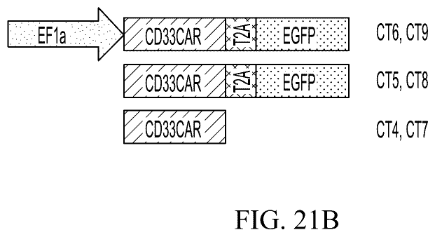

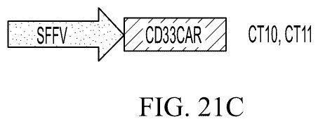

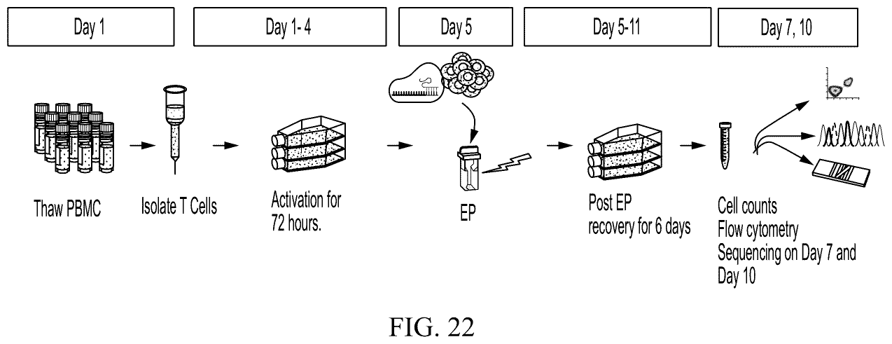

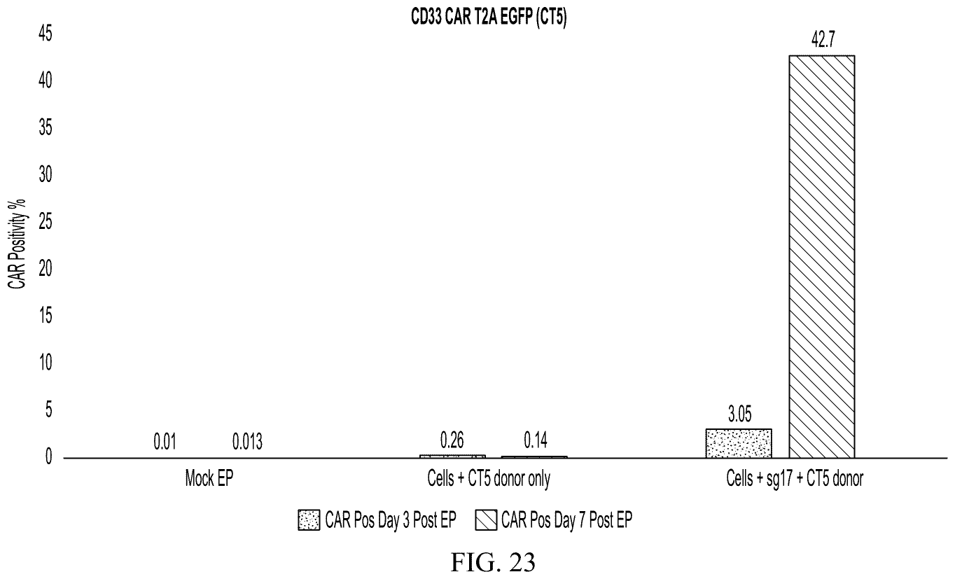

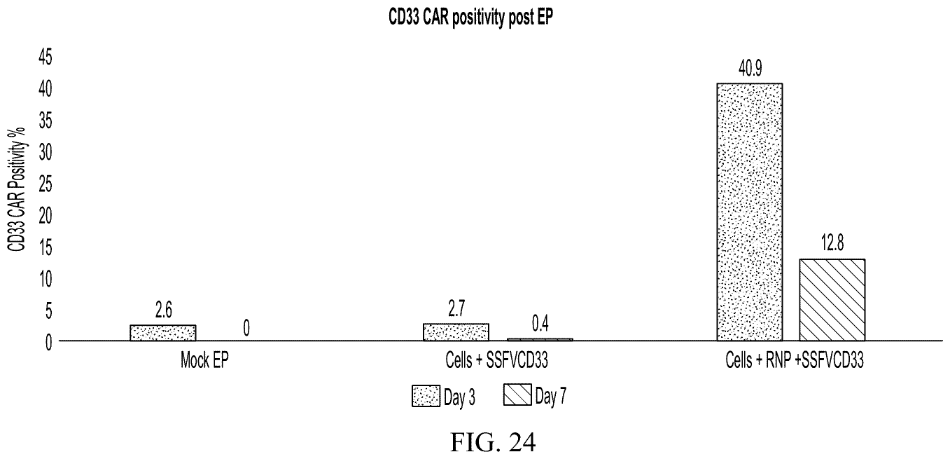

Capped, dsODNs encoding a CD33-targeted chimeric antigen receptor (CAR) insert flanked by 500 bp-long homology arms were designed. The homology arms were designed in order to direct integration that disrupted TCR-a by knockout of TRAC the gene is targeted by Cas9 RNPs comprising sgRNA against target sites on exon 1 of TRAC (FIGS. 21A-21C). Donor T cells were thawed and isolated using the PAN T cell method. Cells were activated for three days via incubation with CD3 and CD28 antibodies. Subsequently, cells were cultured in X Vivo 15 with 5% FBS, 0.2 mM Glutamax, 10 mM N-acetyl cysteine, 200 u/mL IL-2, 5 ng/mL IL-7, and 5 ng/ml IL-15 for 24 hours prior to electroporation with Cas9 RNPs and 1 μg of CD33-CAR dsODNs. Cells were allowed to recover for 6 days. On days 7 and 10 following electroporation, cells were subjected to cell counts, flow cytometry, and sequencing analyses (FIG. 22). Flow cytometry analyses indicated that electroporation with CT5 dsODN resulted in approximately 40% of cells expressing GFP (FIG. 23). When a similar approach was used to integrate a CD33-targeted CAR via knockout of the RAB11a locus using a capped dsDNA template polynucleotide, flow cytometry analyses indicated approximately 40% CAR positivity at the RAB11a locus at day 3 post-electroporation with the CAR construct (FIG. 24).

TABLE 8

| | | | SEQ |
|---|---|---|---|
| | | | ID NO |
| Long | SG9 | tgacatcaattattatacat | 31 |
| SSDNA | (CCR5) | | |
| | Long | GCCCGGGATGGTCCAGGCTGCAGTGAGCCATG | 93 |
| | SSDNA | ATCGTGCCACTGCACTCCAGCCTGGGCGACAGA | |
| | | GTGAGACCCTGTCTCACAACAACAACAACAAC | |
| | | AACAAAAAGGCTGAGCTGCACCATGCTTGACCC | |
| | | AGTTTCTTAAAATTGTTGTCAAAGCTTCATTCAC | |
| | | TCCATGGTGCTATAGAGCACAAGATTTTATTTG | |
| | | GTGAGATGGTGCTTTCATGAATTCCCCCAACAG | |
| | | AGCCAAGCTCTCCATCTAGTGGACAGGGAAGCT | |
| | | AGCAGCAAACCTTCCCTTCACTACAAAACTTCA | |
| | | TTGCTTGGCCAAAAAGAGAGTTAATTCAATGTA | |
| | | GACATCTATGTAGGCAATTAAAAACCTATTGAT | |
| | | GTATAAAACAGTTTGCATTCATGGAGGGCAACT | |
| | | AAATACATTCTAGGACTTTATAAAAGATCACTT | |
| | | TTTATTTATGCACAGGGTGGAACAAGATGGATT | |
| | | ATCAAGTGTCAAGTCCAATCTATGACATCAATT | |
| | | ATTATAGTAACGCCATTTTGCAAGGCATGGAAA | |
| | | AATACCAAACCAAGAATAGAGAAGTTCAGATC | |
| | | AAGGGCGGGTACATGAAAATAGCTAACGTTGG | |
| | | GCCAAACAGGATATCTGCGGTGAGCAGTTTCGG | |
| | | CCCCGGCCCGGGGCCAAGAACAGATGGTCACC | |
| | | GCAGTTTCGGCCCCGGCCCGAGGCCAAGAACA | |
| | | GATGGTCCCCAGATATGGCCCAACCCTCAGCAG | |
| | | TTTCTTAAGACCCATCAGATGTTTCCAGGCTCCC | |
| | | CCAAGGACCTGAAATGACCCTGCGCCTTATTTG | |
| | | AATTAACCAATCAGCCTGCTTCTCGCTTCTGTTC | |
| | | GCGCGCTTCTGCTTCCCGAGCTCTATAAAAGAG | |
| | | CTCACAACCCCTCACTCGGCGCGCCAGTCCTCC | |
| | | GACAGACTGAGTCGCCCGGGCCGCGGCCGCGG | |
| | | GCTAGCGGATCCCCACCGGTCGCCACCATGGTG | |
| | | AGCAAGGGCGAGGAGCTGTTCACCGGGGTGGT | |
| | | GCCCATCCTGGTCGAGCTGGACGGCGACGTAAA | |
| | | CGGCCACAAGTTCAGCGTGTCCGGCGAGGGCG | |
| | | AGGGCGATGCCACCTACGGCAAGCTGACCCTG | |
| | | AAGTTCATCTGCACCACCGGCAAGCTGCCCGTG | |
| | | CCCTGGCCCACCCTCGTGACCACCCTGACCTAC | |
| | | GGCGTGCAGTGCTTCAGCCGCTACCCCGACCAC | |
| | | ATGAAGCAGCACGACTTCTTCAAGTCCGCCATG | |
| | | CCCGAAGGCTACGTCCAGGAGCGCACCATCTTC | |
| | | TTCAAGGACGACGGCAACTACAAGACCCGCGC | |
| | | CGAGGTGAAGTTCGAGGGCGACACCCTGGTGA | |
| | | ACCGCATCGAGCTGAAGGGCATCGACTTCAAG | |
| | | GAGGACGGCAACATCCTGGGGCACAAGCTGGA | |
| | | GTACAACTACAACAGCCACAACGTCTATATCAT | |
| | | GGCCGACAAGCAGAAGAACGGCATCAAGGTGA | |
| | | ACTTCAAGATCCGCCACAACATCGAGGACGGC | |
| | | AGCGTGCAGCTCGCCGACCACTACCAGCAGAA | |
| | | CACCCCCATCGGCGACGGCCCCGTGCTGCTGCC | |
| | | CGACAACCACTACCTGAGCACCCAGTCCGCCCT | |
| | | GAGCAAAGACCCCAACGAGAAGCGCGATCACA | |
| | | TGGTCCTGCTGGAGTTCGTGACCGCCGCCGGGA | |
| | | TCACTCTCGGCATGGACGAGCTGTACAAGTAAA | |
| | | ATGAATGCAATTGTTGTTGTTAATAAAGGAAAT | |
| | | TTATTTTCATTGCAATAGTGTGTTGGAATTTTTT | |
| | | GTGTCTCTCACATCGGAGCCCTGCCAAAAAATC | |
| | | AATGTGAAGCAAATCGCAGCCCGCCTCCTGCCT | |
| | | CCGCTCTACTCACTGGTGTTCATCTTTGGTTTTG | |
| | | TGGGCAACATGCTGGTCATCCTCATCCTGATAA | |
| | | ACTGCAAAAGGCTGAAGAGCATGACTGACATC | |
| | | TACCTGCTCAACCTGGCCATCTCTGACCTGTTTT | |
| | | TCCTTCTTACTGTCCCCTTCTGGGCTCACTATGC | |
| | | TGCCGCCCAGTGGGACTTTGGAAATACAATGTG | |
| | | TCAACTCTTGACAGGGCTCTATTTTATAGGCTTC | |
| | | TTCTCTGGAATCTTCTTCATCATCCTCCTGACAA | |
| | | TCGATAGGTACCTGGCTGTCGTCCATGCTGTGT | |
| | | TTGCTTTAAAAGCCAGGACGGTCACCTTTGGGG | |
| | | TGGTGACAAGTGTGATCACTTGGGTGGTGGCTG | |
| | | TGTTTGCGTCTCTCCCAGGAATCATCTTTACCAG | |
| | | ATCTCAAAAAGAAGGTCTTCATTACACCTGCAG | |
| | | CTCTCATTTT | |
| Uncapped | SG15 | tgacatcaattattatacat | 31 |
| dsDNA | (CCR5) | | |
| | sg16 | GCCAGTAGCCAGCCCCGTCC | 94 |
| | (AAVS1-T) | | |
| | dsDNA | TTGACCCAGTTTCTTAAAATTGTTGTCAAAGCTT | 95 |
| | Donor | CATTCACTCCATGGTGCTATAGAGCACAAGATT | |
| | (CCR5) | TTATTTGGTGAGATGGTGCTTTCATGAATTCCCC | |
| | | CAACAGAGCCAAGCTCTCCATCTAGTGGACAGG | |
| | | GAAGCTAGCAGCAAACCTTCCCTTCACTACAAA | |
| | | ACTTCATTGCTTGGCCAAAAAGAGAGTTAATTC | |
| | | AATGTAGACATCTATGTAGGCAATTAAAAACCT | |
| | | ATTGATGTATAAAACAGTTTGCATTCATGGAGG | |
| | | GCAACTAAATACATTCTAGGACTTTATAAAAGA | |
| | | TCACTTTTTATTTATGCACAGGGTGGAACAAGA | |
| | | TGGATTATCAAGTGTCAAGTCCAATCTATGACA | |
| | | TCAATTATTATAgtaacgccattttgcaaggcatggaaaaataccaa | |
| | | accaagaatagagaagttcagatcaaggggggtacatgaaaatagctaacg | |
| | | ttgggccaaacaggatatctgcggtgagcagtttcggccccggcccggggcc | |
| | | aagaacagatggtcaccgcagtttcggccccggcccgaggccaagaacaga | |
| | | tggtccccagatatggcccaaccctcagcagtttcttaagacccatcagatgttt | |
| | | ccaggctcccccaaggacctgaaatgaccctgcgccttatttgaattaaccaat | |
| | | cagcctgcttctcgcttctgttcgcgcgcttctgcttcccgagctctataaaagag | |
| | | ctcacaacccctcactcggcgcgccagtcctccgacagactgagtcgcccgg | |
| | | gccgcggccgcgggctagcggatccccaccggtcgccaccatggtgagca | |
| | | agggcgaggagctgttcaccggggtggtgcccatcctggtcgagctggacg | |
| | | gcgacgtaaacggccacaagttcagcgtgtccggcgagggcgagggcgat | |
| | | gccacctacggcaagctgaccctgaagttcatctgcaccaccggcaagctgc | |
| | | ccgtgccctggcccaccctcgtgaccaccctgacctacggcgtgcagtgcttc | |
| | | agccgctaccccgaccacatgaagcagcacgacttcttcaagtccgccatgc | |
| | | ccgaaggctacgtccaggagcgcaccatcttcttcaaggacgacggcaacta | |
| | | caagacccgcgccgaggtgaagttcgagggcgacaccctggtgaaccgcat | |
| | | cgagctgaagggcatcgacttcaaggaggacggcaacatcctggggcacaa | |
| | | gctggagtacaactacaacagccacaacgtctatatcatggccgacaagcag | |
| | | aagaacggcatcaaggtgaacttcaagatccgccacaacatcgaggacggc | |
| | | agcgtgcagctcgccgaccactaccagcagaacacccccatcggcgacggc | |
| | | cccgtgctgctgcccgacaaccactacctgagcacccagtccgccctgagca | |
| | | aagaccccaacgagaagcgcgatcacatggtcctgctggagttcgtgaccgc | |
| | | cgccgggatcactctcggcatggacgagctgtacaagTAAaatgaatgca | |
| | | attgttgttgttaataaaggaaatttattttcattgcaatagtgtgttggaattttttgt | |
| | | gtctctcaCATCGGAGCCCTGCCAAAAAATCAATGT | |
| | | GAAGCAAATCGCAGCCCGCCTCCTGCCTCCGCT | |
| | | CTACTCACTGGTGTTCATCTTTGGTTTTGTGGGC | |
| | | AACATGCTGGTCATCCTCATCCTGATAAACTGC | |
| | | AAAAGGCTGAAGAGCATGACTGACATCTACCT | |
| | | GCTCAACCTGGCCATCTCTGACCTGTTTTTCCTT | |
| | | CTTACTGTCCCCTTCTGGGCTCACTATGCTGCCG | |
| | | CCCAGTGGGACTTTGGAAATACAATGTGTCAAC | |
| | | TCTTGACAGGGCTCTATTTTATAGGCTTCTTCTC | |
| | | TGGAATCTTCTTCATCATCCTCCTGACAATCGAT | |
| | | AGGTACCTGGCTGTCGTCCATGCTGTGTTTGCTT | |
| | | TAAAAGCCAGGACG | |
| | dsDNA | ATGCAGGGGAACGGGGATGCAGGGGAACGGGG | 96 |
| | Donor | CTCAGTCTGAAGAGCAGAGCCAGGAACCCCTGT | |
| | (AAVS1-T) | AGGGAAGGGGCAGGAGAGCCAGGGGCATGAG | |
| | | ATGGTGGACGAGGAAGGGGGACAGGGAAGCCT | |
| | | GAGCGCCTCTCCTGGGCTTGCCAAGGACTCAAA | |
| | | CCCAGAAGCCCAGAGCAGGGCCTTAGGGAAGC | |
| | | GGGACCCTGCTCTGGGCGGAGGAATATGTCCCA | |
| | | GATAGCACTGGGGACTCTTTAAGGAAAGAAGG | |
| | | ATGGAGAAAGAGAAAGGGAGTAGAGGCGGCCA | |
| | | CGACCTGGTGAACACCTAGGACGCACCATTCTC | |
| | | ACAAAGGGAGTTTTCCACACGGACACCCCCCTC | |
| | | CTCACCACAGCCCTGCCAGGATGAGAGACACA | |
| | | AAAAATTCCAACACACTATTGCAATGAAAATAA | |
| | | ATTTCCTTTATTAACAACAACAATTGCATTCATT | |
| | | TTACTTGTACAGCTCGTCCATGCCGAGAGTGAT | |
| | | CCCGGCGGCGGTCACGAACTCCAGCAGGACCA | |
| | | TGTGATCGCGCTTCTCGTTGGGGTCTTTGCTCAG | |
| | | GGCGGACTGGGTGCTCAGGTAGTGGTTGTCGGG | |
| | | CAGCAGCACGGGGCCGTCGCCGATGGGGGTGT | |
| | | TCTGCTGGTAGTGGTCGGCGAGCTGCACGCTGC | |
| | | CGTCCTCGATGTTGTGGCGGATCTTGAAGTTCA | |
| | | CCTTGATGCCGTTCTTCTGCTTGTCGGCCATGAT | |
| | | ATAGACGTTGTGGCTGTTGTAGTTGTACTCCAG | |
| | | CTTGTGCCCCAGGATGTTGCCGTCCTCCTTGAA | |
| | | GTCGATGCCCTTCAGCTCGATGCGGTTCACCAG | |
| | | GGTGTCGCCCTCGAACTTCACCTCGGCGCGGGT | |
| | | CTTGTAGTTGCCGTCGTCCTTGAAGAAGATGGT | |
| | | GCGCTCCTGGACGTAGCCTTCGGGCATGGCGGA | |
| | | CTTGAAGAAGTCGTGCTGCTTCATGTGGTCGGG | |
| | | GTAGCGGCTGAAGCACTGCACGCCGTAGGTCA | |
| | | GGGTGGTCACGAGGGTGGGCCAGGGCACGGGC | |
| | | AGCTTGCCGGTGGTGCAGATGAACTTCAGGGTC | |
| | | AGCTTGCCGTAGGTGGCATCGCCCTCGCCCTCG | |
| | | CCGGACACGCTGAACTTGTGGCCGTTTACGTCG | |
| | | CCGTCCAGCTCGACCAGGATGGGCACCACCCCG | |
| | | GTGAACAGCTCCTCGCCCTTGCTCACCATGGTG | |
| | | GCGACCGGTGGGGATCCGCTAGCCCGCGGCCG | |
| | | CGGCCCGGGCGACTCAGTCTGTCGGAGGACTGG | |
| | | CGCGCCGAGTGAGGGGTTGTGAGCTCTTTTATA | |
| | | GAGCTCGGGAAGCAGAAGCGCGCGAACAGAAG | |
| | | CGAGAAGCAGGCTGATTGGTTAATTCAAATAAG | |
| | | GCGCAGGGTCATTTCAGGTCCTTGGGGGAGCCT | |
| | | GGAAACATCTGATGGGTCTTAAGAAACTGCTGA | |
| | | GGGTTGGGCCATATCTGGGGACCATCTGTTCTT | |
| | | GGCCTCGGGCCGGGGCCGAAACTGCGGTGACC | |
| | | ATCTGTTCTTGGCCCCGGGCCGGGGCCGAAACT | |
| | | GCTCACCGCAGATATCCTGTTTGGCCCAACGTT | |
| | | AGCTATTTTCATGTACCCGCCCTTGATCTGAACT | |
| | | TCTCTATTCTTGGTTTGGTATTTTTCCATGCCTT | |
| | | GCAAAATGGCGTTACCGGGGCTGGCTACTGGCC | |
| | | TTATCTCACAGGTAAAACTGACGCACGGAGGA | |
| | | ACAATATAAATTGGGGACTAGAAAGGTGAAGA | |
| | | GCCAAAGTTAGAACTCAGGACCAACTTATTCTG | |
| | | ATTTTGTTTTTCCAAACTGCTTCTCCTCTTGGGA | |
| | | AGTGTAAGGAAGCTGCAGCACCAGGATCAGTG | |
| | | AAACGCACCAGACAGCCGCGTCAGAGCAGCTC | |
| | | AGGTTCTGGGAGAGGGTAGCGCAGGGTGGCCA | |
| | | CTGAGAACCGGGCAGGTCACGCATCCCCCCCTT | |
| | | CCCTCCCACCCCCTGCCAAGCTCTCCCTCCCAG | |
| | | GATCCTCTCTGGCTCCATCGTAAGCAAACCTTA | |
| | | GAGGTTCTGGCAAGGAGAGAGATGGCTCCAGG | |
| | | A | |
| Capped | Sg9 | tgacatcaattattatacat | 31 |
| dsDNA | (CCR5) | | |
| | sg16 | GCCAGTAGCCAGCCCCGTCC | 94 |
| | (AAVS1-T) | | |
| | sg17 | TCAGGGTTCTGGATATCTGT | 97 |
| | (TRAC) | | |
| | sg18 | AGAGTCTCTCAGCTGGTACA | 98 |
| | (TRAC) | | |
| | g1 | AGCGGTTGCAGAGACCCCAT | 99 |
| | (CD5) | | |
| | G2 | CATACCAGCTGAGCCGTCCG | 100 |
| | (CD5) | | |
| | g3 | CATAGCTGATGGTACCCCCC | 101 |
| | (CD5) | | |
| CT1 | Rab11a- | GGTAGCTAGGAGTTCCAGGACTCAGTTTCCCCT | 102 |
| | 300HA- | TTGAGCCTCCTTTAGCGACTAAAGCTTGAAGCC | |
| | GFP | CCACGCATCTCGACTCTCGCGCACACCGCCCTT | |
| | | GTTGGGCTCAGGGGGGGGCGCCGCCCCCGGA | |
| | | AGTACTTCCCCTTAAAGGCTGGGGCCTGCCGGA | |
| | | AATGGCGCAGCGGCAGGGAGGGGCTCTTCACC | |
| | | CAGTCCGGCAGTTGAAGCTCGGCGCTCGGGTTA | |
| | | CCCCTGCAGCGACGCCCCCTGGTCCCACAGATA | |
| | | CCACTGCTGCTCCCGCCCTTTCGCTCCTCGGCCG | |
| | | CGCAATGGGCGGATCGGGTGGGACTAGTGGCA | |
| | | GCAAGGGCGAGGAGCTGTTCACCGGGGTGGTG | |
| | | CCCATCCTGGTCGAGCTGGACGGCGACGTAAAC | |
| | | GGCCACAAGTTCAGCGTGCGCGGCGAGGGCGA | |
| | | GGGCGATGCCACCAACGGCAAGCTGACCCTGA | |
| | | AGTTCATCTGCACCACCGGCAAGCTGCCCGTGC | |
| | | CCTGGCCCACCCTCGTGACCACCCTGACCTACG | |
| | | GCGTGCAGTGCTTCAGCCGCTACCCCGACCACA | |
| | | TGAAGCGCCACGACTTCTTCAAGTCCGCCATGC | |
| | | CCGAAGGCTACGTCCAGGAGCGCACCATCAGCT | |
| | | TCAAGGACGACGGCACCTACAAGACCCGCGCC | |
| | | GAGGTGAAGTTCGAGGGCGACACCCTGGTGAA | |
| | | CCGCATCGAGCTGAAGGGCATCGACTTCAAGG | |
| | | AGGACGGCAACATCCTGGGGCACAAGCTGGAG | |
| | | TACAACTTCAACAGCCACAACGTCTATATCACC | |
| | | GCCGACAAGCAGAAGAACGGCATCAAGGCCAA | |
| | | CTTCAAGATCCGCCACAACGTGGAGGACGGCA | |
| | | GCGTGCAGCTCGCCGACCACTACCAGCAGAAC | |
| | | ACCCCCATCGGCGACGGCCCCGTGCTGCTGCCC | |
| | | GACAACCACTACCTGAGCACCCAGTCCGTGCTG | |
| | | AGCAAAGACCCCAACGAGAAGCGCGATCACAT | |
| | | GGTCCTGCTGGAGTTCGTGACCGCCGCCGGGAT | |
| | | CACTGGAACCGGTGCTGGAAGTGGTACACGCG | |
| | | ACGACGAGTACGACTACCTCTTTAAAGGTGAGG | |
| | | CCATGGGCTCTCGCACTCTACACAGTCCTCGTT | |
| | | CGGGGACCCGGGCCACTCCCGGTGGACCCTCGT | |
| | | GCCGGCCACCCCTGCACTGATATAGGCCTCCCT | |
| | | CAGCCCTTCCTTTTTGTGCGGTTCCGTCTCCTAC | |
| | | CCAGCTCAGCCTCTTCTCCCCCGCTCAGACAGG | |
| | | GGTCCCCATCACATGCCGCTCTCTGAGCGACCT | |
| | | CTCCATAGGCCTTCGCTGGCCTCAGAGCCCCTC | |
| | | CCTGCGTGTCCTTCCCCTGGCGGACTGCCTTCTC | |
| | | CCACATCGT | |
| CT2 | Rab11a- | gagtccagagtgctaaccattacaccatGGAACCGCCACGCAT | 103 |
| | 500HA- | GTGTAGCTGCCTTCGGCTGTCTAATCCTCAGAG | |
| | EGFP- | AACCCCGCCCCCATCCACAAACCCACCACTCAC | |
| | pA | AGGCGGTCCCGCCTGGTTCCAGCGAGCCGCTTC | |
| | | CGGCACGGTAGCTCGAGAAATGAGCAAGCGGC | |
| | | CACTAAGACTATGGTAGCTAGGAGTTCCAGGAC | |
| | | TCAGTTTCCCCTTTGAGCCTCCTTTAGCGACTAA | |
| | | AGCTTGAAGCCCCACGCATCTCGACTCTCGCGC | |
| | | ACACCGCCCTTGTTGGGCTCAGGGGCGGGGCGC | |
| | | CGCCCCCGGAAGTACTTCCCCTTAAAGGCTGGG | |
| | | GCCTGCCGGAAATGGCGCAGCGGCAGGGAGGG | |
| | | GCTCTTCACCCAGTCCGGCAGTTGAAGCTCGGC | |
| | | GCTCGGGTTACCCCTGCAGCGACGCCCCCTGGT | |
| | | CCCACAGATACCACTGCTGCTCCCGCCCTTTCG | |
| | | CTCCTCGGCCGCGCAATGGGCACCCGCGAGCCA | |
| | | CCATGGTGAGCAAGGGCGAGGAGCTGTTCACC | |
| | | GGGGTGGTGCCCATCCTGGTCGAGCTGGACGGC | |
| | | GACGTAAACGGCCACAAGTTCAGCGTGTCCGGC | |
| | | GAGGGCGAGGGCGATGCCACCTACGGCAAGCT | |
| | | GACCCTGAAGTTCATCTGCACCACCGGCAAGCT | |
| | | GCCCGTGCCCTGGCCCACCCTCGTGACCACCCT | |
| | | GACCTACGGCGTGCAGTGCTTCAGCCGCTACCC | |
| | | CGACCACATGAAGCAGCACGACTTCTTCAAGTC | |
| | | CGCCATGCCCGAAGGCTACGTCCAGGAGCGCA | |
| | | CCATCTTCTTCAAGGACGACGGCAACTACAAGA | |
| | | CCCGCGCCGAGGTGAAGTTCGAGGGCGACACC | |
| | | CTGGTGAACCGCATCGAGCTGAAGGGCATCGA | |
| | | CTTCAAGGAGGACGGCAACATCCTGGGGCACA | |
| | | AGCTGGAGTACAACTACAACAGCCACAACGTCT | |
| | | ATATCATGGCCGACAAGCAGAAGAACGGCATC | |
| | | AAGGTGAACTTCAAGATCCGCCACAACATCGA | |
| | | GGACGGCAGCGTGCAGCTCGCCGACCACTACC | |
| | | AGCAGAACACCCCCATCGGCGACGGCCCCGTG | |
| | | CTGCTGCCCGACAACCACTACCTGAGCACCCAG | |
| | | TCCGCCCTGAGCAAAGACCCCAACGAGAAGCG | |
| | | CGATCACATGGTCCTGCTGGAGTTCGTGACCGC | |
| | | CGCCGGGATCACTCTCGGCATGGACGAGCTGTA | |
| | | CAAGTAAAATGAATGCAATTGTTGTTGTTAATA | |
| | | AAGGAAATTTATTTTCATTGCAATAGTGTGTTG | |
| | | GAATTTTTTGTGTCTCTCACGACGAGTACGACT | |
| | | ACCTCTTTAAAGGTGAGGCCATGGGCTCTCGCA | |
| | | CTCTACACAGTCCTCGTTCGGGGACCCGGGCCA | |
| | | CTCCCGGTGGACCCTCGTGCCGGCCACCCCTGC | |
| | | ACTGATATAGGCCTCCCTCAGCCCTTCCTTTTTG | |
| | | TGCGGTTCCGTCTCCTACCCAGCTCAGCCTCTTC | |
| | | TCCCCCGCTCAGACAGGGGTCCCCATCACATGC | |
| | | CGCTCTCTGAGCGACCTCTCCATAGGCCTTCGC | |
| | | TGGCCTCAGAGCCCCTCCCTGCGTGTCCTTCCCC | |
| | | TGGCGGACTGCCTTCTCCCACATCGTCGAATTC | |
| | | CTTTCCCCGGGTTCTACGGCCCCGCCGCTCCTCC | |
| | | CACCATCTCTCTTTTCGGGTGTAGCGCCCCCTCC | |
| | | CCCTCGGCGTACACCCTTCCCAGCTCGCGTCCT | |
| | | CTCCCGAAGCCCCTCTGACGGGTTCTTCGCTTCC | |
| | | CTCTTGGCCTTGCCTTCGGTGCAGACTCCCATTA | |
| | | CAGGTCTTTTTCTTATC | |
| CT3 | Rab11a- | gagtccagagtgctaaccattacaccatGGAACCGCCACGCAT | 104 |
| | 500HA- | GTGTAGCTGCCTTCGGCTGTCTAATCCTCAGAG | |
| | SFFV- | AACCCCGCCCCCATCCACAAACCCACCACTCAC | |
| | EGFP- | AGGCGGTCCCGCCTGGTTCCAGCGAGCCGCTTC | |
| | pA | CGGCACGGTAGCTCGAGAAATGAGCAAGCGGC | |
| | | CACTAAGACTATGGTAGCTAGGAGTTCCAGGAC | |
| | | TCAGTTTCCCCTTTGAGCCTCCTTTAGCGACTAA | |
| | | AGCTTGAAGCCCCACGCATCTCGACTCTCGCGC | |
| | | ACACCGCCCTTGTTGGGCTCAGGGGGGGGCGC | |
| | | CGCCCCCGGAAGTACTTCCCCTTAAAGGCTGGG | |
| | | GCCTGCCGGAAATGGCGCAGCGGCAGGGAGGG | |
| | | GCTCTTCACCCAGTCCGGCAGTTGAAGCTCGGC | |
| | | GCTCGGGTTACCCCTGCAGCGACGCCCCCTGGT | |
| | | CCCACAGATACCACTGCTGCTCCCGCCCTTTCG | |
| | | CTCCTCGGCCGCGCAATGGGCACCCGCGAGTAA | |
| | | CGCCATTTTGCAAGGCATGGAAAAATACCAAAC | |
| | | CAAGAATAGAGAAGTTCAGATCAAGGGCGGGT | |
| | | ACATGAAAATAGCTAACGTTGGGCCAAACAGG | |
| | | ATATCTGCGGTGAGCAGTTTCGGCCCCGGCCCG | |
| | | GGGCCAAGAACAGATGGTCACCGCAGTTTCGG | |
| | | CCCCGGCCCGAGGCCAAGAACAGATGGTCCCC | |
| | | AGATATGGCCCAACCCTCAGCAGTTTCTTAAGA | |
| | | CCCATCAGATGTTTCCAGGCTCCCCCAAGGACC | |
| | | TGAAATGACCCTGCGCCTTATTTGAATTAACCA | |
| | | ATCAGCCTGCTTCTCGCTTCTGTTCGCGCGCTTC | |
| | | TGCTTCCCGAGCTCTATAAAAGAGCTCACAACC | |
| | | CCTCACTCGGCGCGCCAGTCCTCCGACAGACTG | |
| | | AGTCGCCCGGGCCGCGGCCGCGGGCTAGCGGA | |
| | | TCCCCACCGGTCGCCACCATGGTGAGCAAGGGC | |
| | | GAGGAGCTGTTCACCGGGGTGGTGCCCATCCTG | |
| | | GTCGAGCTGGACGGCGACGTAAACGGCCACAA | |
| | | GTTCAGCGTGTCCGGCGAGGGCGAGGGCGATG | |
| | | CCACCTACGGCAAGCTGACCCTGAAGTTCATCT | |
| | | GCACCACCGGCAAGCTGCCCGTGCCCTGGCCCA | |
| | | CCCTCGTGACCACCCTGACCTACGGCGTGCAGT | |
| | | GCTTCAGCCGCTACCCCGACCACATGAAGCAGC | |
| | | ACGACTTCTTCAAGTCCGCCATGCCCGAAGGCT | |
| | | ACGTCCAGGAGCGCACCATCTTCTTCAAGGACG | |
| | | ACGGCAACTACAAGACCCGCGCCGAGGTGAAG | |
| | | TTCGAGGGCGACACCCTGGTGAACCGCATCGAG | |
| | | CTGAAGGGCATCGACTTCAAGGAGGACGGCAA | |
| | | CATCCTGGGGCACAAGCTGGAGTACAACTACA | |
| | | ACAGCCACAACGTCTATATCATGGCCGACAAGC | |
| | | AGAAGAACGGCATCAAGGTGAACTTCAAGATC | |
| | | CGCCACAACATCGAGGACGGCAGCGTGCAGCT | |
| | | CGCCGACCACTACCAGCAGAACACCCCCATCGG | |
| | | CGACGGCCCCGTGCTGCTGCCCGACAACCACTA | |
| | | CCTGAGCACCCAGTCCGCCCTGAGCAAAGACCC | |
| | | CAACGAGAAGCGCGATCACATGGTCCTGCTGG | |
| | | AGTTCGTGACCGCCGCCGGGATCACTCTCGGCA | |
| | | TGGACGAGCTGTACAAGTAAAATGAATGCAATT | |
| | | GTTGTTGTTAATAAAGGAAATTTATTTTCATTGC | |
| | | AATAGTGTGTTGGAATTTTTTGTGTCTCTCACGA | |
| | | CGAGTACGACTACCTCTTTAAAGGTGAGGCCAT | |
| | | GGGCTCTCGCACTCTACACAGTCCTCGTTCGGG | |
| | | GACCCGGGCCACTCCCGGTGGACCCTCGTGCCG | |
| | | GCCACCCCTGCACTGATATAGGCCTCCCTCAGC | |
| | | CCTTCCTTTTTGTGCGGTTCCGTCTCCTACCCAG | |
| | | CTCAGCCTCTTCTCCCCCGCTCAGACAGGGGTC | |
| | | CCCATCACATGCCGCTCTCTGAGCGACCTCTCC | |
| | | ATAGGCCTTCGCTGGCCTCAGAGCCCCTCCCTG | |
| | | CGTGTCCTTCCCCTGGCGGACTGCCTTCTCCCAC | |
| | | ATCGTCGAATTCCTTTCCCCGGGTTCTACGGCCC | |
| | | CGCCGCTCCTCCCACCATCTCTCTTTTCGGGTGT | |
| | | AGCGCCCCCTCCCCCTCGGCGTACACCCTTCCC | |
| | | AGCTCGCGTCCTCTCCCGAAGCCCCTCTGACGG | |
| | | GTTCTTCGCTTCCCTCTTGGCCTTGCCTTCGGTG | |
| | | CAGACTCCCATTACAGGTCTTTTTCTTATC | |
| CT4 | 500bpHA- | ATGTGATAGATTTCCCAACTTAATGCCAACATA | 105 |
| | SG17- | CCATAAACCTCCCATTCTGCTAATGCCCAGCCT | |
| | TRAC- | AAGTTGGGGAGACCACTCCAGATTCCAAGATGT | |
| | CD33CAR | ACAGTTTGCTTTGCTGGGCCTTTTTCCCATGCCT | |
| | | GCCTTTACTCTGCCAGAGTTATATTGCTGGGGTT | |
| | | TTGAAGAAGATCCTATTAAATAAAAGAATAAG | |
| | | CAGTATTATTAAGTAGCCCTGCATTTCAGGTTTC | |
| | | CTTGAGTGGCAGGCCAGGCCTGGCCGTGAACGT | |
| | | TCACTGAAATCATGGCCTCTTGGCCAAGATTGA | |
| | | TAGCTTGTGCCTGTCCCTGAGTCCCAGTCCATC | |
| | | ACGAGCAGCTGGTTTCTAAGATGCTATTTCCCG | |
| | | TATAAAGCATGAGACCGTGACTTGCCAGCCCCA | |
| | | CAGAGCCCCGCCCTTGTCCATCACTGGCATCTG | |
| | | GACTCCAGCCTGGGTTGGGGCAAAGAGGGAAA | |
| | | TGAGATCATGTCCTAACCCTGATCCTCTTGTCCC | |
| | | ACAGtgaataattgagccaccatggctctgcccgtcacagctctgctgctg | |
| | | cctctggccctgctgctgcacgccgccagacctcaggtgcagctcgtgcaga | |
| | | gcggcgctgaggtgaagaaacctggcagcagcgtgaaggtgagctgcaag | |
| | | gcctccggctacaccttcaccgactacaacatgcactgggtgaggcaagccc | |
| | | ctggccagggactggagtggatcggctacatctacccttacaacggcggcac | |
| | | aggctacaaccagaagttcaagtccaaggccaccatcaccgccgatgagtcc | |
| | | accaataccgcctacatggagctcagcagcctgaggtccgaggacacagcc | |
| | | gtctactactgcgccaggggcaggcccgctatggactactggggccagggc | |
| | | accctggtgacagtgagctctggtggcggcggatccggcggcggcggcag | |
| | | cggcggcggcggctccgacattcagatgacccagagccctagcagcctgag | |
| | | cgcttccgtgggagacagggtgaccatcacatgcagggcctccgagagcgt | |
| | | ggacaattacggcatcagcttcatgaactggttccagcagaagcccggcaag | |
| | | gcccccaaactgctgatctatgccgccagcaatcagggctccggcgtgccta | |
| | | gcaggttttccggcagcggcagcggcaccgactttaccctgaccatctccagc | |
| | | ctgcagcctgacgatttcgccacctactactgccagcagagcaaggaggtgc | |
| | | cttggacctttggacagggcacaaaggtggagatcaagtccggagccgccg | |
| | | ccatcgaagtgatgtacccccctccctacctggataacgagaagagcaacgg | |
| | | caccatcatccacgtgaagggaaagcacctgtgtcccagccccctgtttcccg | |
| | | gccctagcaagcccttctgggtgctggtggtggtcggcggagtgctggcctg | |
| | | ctacagcctcctggtgaccgtggccttcatcatcttctgggtgaggagcaagag | |
| | | gtccaggctgctgcacagcgactacatgaatatgacccccagaaggcccggc | |
| | | cccaccagaaagcactatcagccctacgccccccccagggactttgccgcct | |
| | | acaggagcagggtgaagttcagcagatccgccgatgcccctgcttaccagca | |
| | | gggccagaaccagctgtataacgagctgaacctgggcaggagggaggaata | |
| | | cgacgtgctggataagaggaggggaagggaccccgagatgggcggaaag | |
| | | cccaggaggaagaacccccaggagggcctgtacaatgagctgcagaaaga | |
| | | caagatggccgaggcctacagcgagatcggcatgaagggcgagaggagga | |
| | | ggggcaagggccatgacggcctgtaccaaggcctgtccaccgccaccaag | |
| | | gatacctacgacgccctgcacatgcaggccctgcctcccaggggatcctgat | |
| | | aaACCAGCTGAGAGACTCTAAATCCAGTGACAA | |
| | | GTCTGTCTGCCTATTCACCGATTTTGATTCTCAA | |
| | | ACAAATGTGTCACAAAGTAAGGATTCTGATGTG | |
| | | TATATCACAGACAAAACTGTGCTAGACATGAGG | |
| | | TCTATGGACTTCAAGAGCAACAGTGCTGTGGCC | |
| | | TGGAGCAACAAATCTGACTTTGCATGTGCAAAC | |
| | | GCCTTCAACAACAGCATTATTCCAGAAGACACC | |
| | | TTCTTCCCCAGCCCAGGTAAGGGCAGCTTTGGT | |
| | | GCCTTCGCAGGCTGTTTCCTTGCTTCAGGAATG | |
| | | GCCAGGTTCTGCCCAGAGCTCTGGTCAATGATG | |
| | | TCTAAAACTCCTCTGATTGGTGGTCTCGGCCTTA | |
| | | TCCATTGCCACCAAAACCCTCTTTTTACTAAGA | |
| | | AACAGTGAGCCTTGTTCTGGCAGTCCAGAGAAT | |
| | | GACACGGGAAAAAAGCAGATGAAGAGAAGGTG | |
| | | GCAGGAGAGGGCACGTGGCCCAGCCTCAGTCT | |
| | | CTCCAAC | |
| CT5 | 500bpHA- | ATGTGATAGATTTCCCAACTTAATGCCAACATA | 106 |
| | SG17- | CCATAAACCTCCCATTCTGCTAATGCCCAGCCT | |
| | TRAC- | AAGTTGGGGAGACCACTCCAGATTCCAAGATGT | |
| | CD33CAR- | ACAGTTTGCTTTGCTGGGCCTTTTTCCCATGCCT | |
| | T2A- | GCCTTTACTCTGCCAGAGTTATATTGCTGGGGTT | |
| | EFGP | TTGAAGAAGATCCTATTAAATAAAAGAATAAG | |
| | | CAGTATTATTAAGTAGCCCTGCATTTCAGGTTTC | |
| | | CTTGAGTGGCAGGCCAGGCCTGGCCGTGAACGT | |
| | | TCACTGAAATCATGGCCTCTTGGCCAAGATTGA | |
| | | TAGCTTGTGCCTGTCCCTGAGTCCCAGTCCATC | |
| | | ACGAGCAGCTGGTTTCTAAGATGCTATTTCCCG | |
| | | TATAAAGCATGAGACCGTGACTTGCCAGCCCCA | |
| | | CAGAGCCCCGCCCTTGTCCATCACTGGCATCTG | |
| | | GACTCCAGCCTGGGTTGGGGCAAAGAGGGAAA | |
| | | TGAGATCATGTCCTAACCCTGATCCTCTTGTCCC | |
| | | ACAGtgaataattgagccaccatggctctgcccgtcacagctctgctgctg | |
| | | cctctggccctgctgctgcacgccgccagacctcaggtgcagctcgtgcaga | |
| | | gcggcgctgaggtgaagaaacctggcagcagcgtgaaggtgagctgcaag | |
| | | gcctccggctacaccttcaccgactacaacatgcactgggtgaggcaagccc | |
| | | ctggccagggactggagtggatcggctacatctacccttacaacggcggcac | |
| | | aggctacaaccagaagttcaagtccaaggccaccatcaccgccgatgagtcc | |
| | | accaataccgcctacatggagctcagcagcctgaggtccgaggacacagcc | |
| | | gtctactactgcgccaggggcaggcccgctatggactactggggccagggc | |
| | | accctggtgacagtgagctctggtggcggcggatccggcggcggcggcag | |
| | | cggcggcggcggctccgacattcagatgacccagagccctagcagcctgag | |
| | | cgcttccgtgggagacagggtgaccatcacatgcagggcctccgagagcgt | |
| | | ggacaattacggcatcagcttcatgaactggttccagcagaagcccggcaag | |
| | | gcccccaaactgctgatctatgccgccagcaatcagggctccggcgtgccta | |
| | | gcaggttttccggcagcggcagcggcaccgactttaccctgaccatctccagc | |
| | | ctgcagcctgacgatttcgccacctactactgccagcagagcaaggaggtgc | |
| | | cttggacctttggacagggcacaaaggtggagatcaagtccggagccgccg | |
| | | ccatcgaagtgatgtacccccctccctacctggataacgagaagagcaacgg | |
| | | caccatcatccacgtgaagggaaagcacctgtgtcccagccccctgtttcccg | |
| | | gccctagcaagcccttctgggtgctggtggtggtcggcggagtgctggcctg | |
| | | ctacagcctcctggtgaccgtggccttcatcatcttctgggtgaggagcaagag | |
| | | gtccaggctgctgcacagcgactacatgaatatgacccccagaaggcccggc | |
| | | cccaccagaaagcactatcagccctacgccccccccagggactttgccgcct | |
| | | acaggagcagggtgaagttcagcagatccgccgatgcccctgcttaccagca | |
| | | gggccagaaccagctgtataacgagctgaacctgggcaggagggaggaata | |
| | | cgacgtgctggataagaggaggggaagggaccccgagatgggcggaaag | |
| | | cccaggaggaagaacccccaggagggcctgtacaatgagctgcagaaaga | |
| | | caagatggccgaggcctacagcgagatcggcatgaagggcgagaggagga | |
| | | ggggcaagggccatgacggcctgtaccaaggcctgtccaccgccaccaag | |
| | | gatacctacgacgccctgcacatgcaggccctgcctcccaggggatccGG | |
| | | TGGCAGCGGTgaaggaaggggctctttgcttacttgtggagatgttga | |
| | | ggaaaatccaggacccgtgagcaagggcgaggagctgttcaccggggtggt | |
| | | gcccatcctggtcgagctggacggcgacgtaaacggccacaagttcagcgtg | |
| | | tccggcgagggcgagggcgatgccacctacggcaagctgaccctgaagttc | |
| | | atctgcaccaccggcaagctgcccgtgccctggcccaccctcgtgaccaccc | |
| | | tgacctacggcgtgcagtgcttcagccgctaccccgaccacatgaagcagca | |
| | | cgacttcttcaagtccgccatgcccgaaggctacgtccaggagcgcaccatct | |
| | | tcttcaaggacgacggcaactacaagacccgcgccgaggtgaagttcgagg | |
| | | gcgacaccctggtgaaccgcatcgagctgaagggcatcgacttcaaggagg | |
| | | acggcaacatcctggggcacaagctggagtacaactacaacagccacaacg | |
| | | tctatatcatggccgacaagcagaagaacggcatcaaggtgaacttcaagatc | |
| | | cgccacaacatcgaggacggcagcgtgcagctcgccgaccactaccagca | |
| | | gaacacccccatcggcgacggccccgtgctgctgcccgacaaccactacct | |
| | | gagcacccagtccgccctgagcaaagaccccaacgagaagcgcgatcacat | |
| | | ggtcctgctggagttcgtgaccgccgccgggatcactctcggcatggacgag | |
| | | ctgtacaagtgataaACCAGCTGAGAGACTCTAAATCCA | |
| | | GTGACAAGTCTGTCTGCCTATTCACCGATTTTG | |
| | | ATTCTCAAACAAATGTGTCACAAAGTAAGGATT | |
| | | CTGATGTGTATATCACAGACAAAACTGTGCTAG | |
| | | ACATGAGGTCTATGGACTTCAAGAGCAACAGTG | |
| | | CTGTGGCCTGGAGCAACAAATCTGACTTTGCAT | |
| | | GTGCAAACGCCTTCAACAACAGCATTATTCCAG | |
| | | AAGACACCTTCTTCCCCAGCCCAGGTAAGGGCA | |
| | | GCTTTGGTGCCTTCGCAGGCTGTTTCCTTGCTTC | |
| | | AGGAATGGCCAGGTTCTGCCCAGAGCTCTGGTC | |
| | | AATGATGTCTAAAACTCCTCTGATTGGTGGTCT | |
| | | CGGCCTTATCCATTGCCACCAAAACCCTCTTTTT | |
| | | ACTAAGAAACAGTGAGCCTTGTTCTGGCAGTCC | |
| | | AGAGAATGACACGGGAAAAAAGCAGATGAAGA | |
| | | GAAGGTGGCAGGAGAGGGCACGTGGCCCAGCC | |
| | | TCAGTCTCTCCAAC | |
| CT6 | 500bpHA- | ATGTGATAGATTTCCCAACTTAATGCCAACATA | 107 |
| | SG17- | CCATAAACCTCCCATTCTGCTAATGCCCAGCCT | |
| | TRAC- | AAGTTGGGGAGACCACTCCAGATTCCAAGATGT | |
| | EF1a- | ACAGTTTGCTTTGCTGGGCCTTTTTCCCATGCCT | |
| | CD33CAR- | GCCTTTACTCTGCCAGAGTTATATTGCTGGGGTT | |
| | T2A- | TTGAAGAAGATCCTATTAAATAAAAGAATAAG | |
| | EFGP | CAGTATTATTAAGTAGCCCTGCATTTCAGGTTTC | |
| | | CTTGAGTGGCAGGCCAGGCCTGGCCGTGAACGT | |
| | | TCACTGAAATCATGGCCTCTTGGCCAAGATTGA | |
| | | TAGCTTGTGCCTGTCCCTGAGTCCCAGTCCATC | |
| | | ACGAGCAGCTGGTTTCTAAGATGCTATTTCCCG | |
| | | TATAAAGCATGAGACCGTGACTTGCCAGCCCCA | |
| | | CAGAGCCCCGCCCTTGTCCATCACTGGCATCTG | |
| | | GACTCCAGCCTGGGTTGGGGCAAAGAGGGAAA | |
| | | TGAGATCATGTCCTAACCCTGATCCTCTTGTCCC | |
| | | ACAGtgaataattgaggctccggtgcccgtcagtgggcagagcgcacatc | |
| | | gcccacagtccccgagaagttggggggaggggtcggcaattgaaccggtgc | |
| | | ctagagaaggtggcgcggggtaaactgggaaagtgatgtcgtgtactggctc | |
| | | cgcctttttcccgaggggggggagaaccgtatataagtgcagtagtcgccgt | |
| | | gaacgttctttttcgcaacgggtttgccgccagaacacaggtaagtgccgtgtg | |
| | | tggttcccgcgggcctggcctctttacgggttatggcccttgcgtgccttgaatt | |
| | | acttccacctggctgcagtacgtgattcttgatcccgagcttcgggttggaagtg | |
| | | ggtgggagagttcgaggccttgcgcttaaggagccccttcgcctcgtgcttga | |
| | | gttgaggcctggcctgggcgctggggccgccgcgtgcgaatctggtggcac | |
| | | cttcgcgcctgtctcgctgctttcgataagtctctagccatttaaaatttttgatgac | |
| | | ctgctgcgacgctttttttctggcaagatagtcttgtaaatgcgggccaagatctg | |
| | | cacactggtatttcggtttttggggccgcgggcggcgacggggcccgtgcgt | |
| | | cccagcgcacatgttcggcgaggcggggcctgcgagcgcggccaccgaga | |
| | | atcggacgggggtagtctcaagctggccggcctgctctggtgcctggcctcg | |
| | | cgccgccgtgtatcgccccgccctgggcggcaaggctggcccggtcggcac | |
| | | cagttgcgtgagcggaaagatggccgcttcccggccctgctgcagggagctc | |
| | | aaaatggaggacgcggcgctcgggagagcggggggtgagtcacccacac | |
| | | aaaggaaaagggcctttccgtcctcagccgtcgcttcatgtgactccactgagt | |
| | | accgggcgccgtccaggcacctcgattagttctcgtgcttttggagtacgtcgt | |
| | | ctttaggttggggggaggggttttatgcgatggagtttccccacactgagtggg | |
| | | tggagactgaagttaggccagcttggcacttgatgtaattctccttggaatttgcc | |
| | | ctttttgagtttggatcttggttcattctcaagcctcagacagtggttcaaagtttttt | |
| | | tcttccatttcaggtgtcgtgagctagcctcgaggccaccatggctctgcccgtc | |
| | | acagctctgctgctgcctctggccctgctgctgcacgccgccagacctcaggt | |
| | | gcagctcgtgcagagcggcgctgaggtgaagaaacctggcagcagcgtga | |
| | | aggtgagctgcaaggcctccggctacaccttcaccgactacaacatgcactg | |
| | | ggtgaggcaagcccctggccagggactggagtggatcggctacatctaccct | |
| | | tacaacggcggcacaggctacaaccagaagttcaagtccaaggccaccatca | |
| | | ccgccgatgagtccaccaataccgcctacatggagctcagcagcctgaggtc | |
| | | cgaggacacagccgtctactactgcgccaggggcaggcccgctatggacta | |
| | | ctggggccagggcaccctggtgacagtgagctctggtggcggcggatccgg | |
| | | cggcggcggcagcggcggcggcggctccgacattcagatgacccagagcc | |
| | | ctagcagcctgagcgcttccgtgggagacagggtgaccatcacatgcaggg | |
| | | cctccgagagcgtggacaattacggcatcagcttcatgaactggttccagcag | |
| | | aagcccggcaaggcccccaaactgctgatctatgccgccagcaatcagggct | |
| | | ccggcgtgcctagcaggttttccggcagcggcagcggcaccgactttaccct | |
| | | gaccatctccagcctgcagcctgacgatttcgccacctactactgccagcaga | |
| | | gcaaggaggtgccttggacctttggacagggcacaaaggtggagatcaagtc | |
| | | cggagccgccgccatcgaagtgatgtacccccctccctacctggataacgag | |
| | | aagagcaacggcaccatcatccacgtgaagggaaagcacctgtgtcccagc | |
| | | cccctgtttcccggccctagcaagcccttctgggtgctggtggtggtcggcgg | |
| | | agtgctggcctgctacagcctcctggtgaccgtggccttcatcatcttctgggtg | |
| | | aggagcaagaggtccaggctgctgcacagcgactacatgaatatgaccccca | |
| | | gaaggcccggccccaccagaaagcactatcagccctacgccccccccagg | |
| | | gactttgccgcctacaggagcagggtgaagttcagcagatccgccgatgccc | |
| | | ctgcttaccagcagggccagaaccagctgtataacgagctgaacctgggcag | |
| | | gagggaggaatacgacgtgctggataagaggaggggaagggaccccgag | |
| | | atgggcggaaagcccaggaggaagaacccccaggagggcctgtacaatga | |
| | | gctgcagaaagacaagatggccgaggcctacagcgagatcggcatgaagg | |
| | | gcgagaggaggaggggcaagggccatgacggcctgtaccaaggcctgtcc | |
| | | accgccaccaaggatacctacgacgccctgcacatgcaggccctgcctccca | |
| | | ggggatccGGTGGCAGCGGTgaaggaaggggctctttgcttacttg | |
| | | tggagatgttgaggaaaatccaggacccgtgagcaagggcgaggagctgttc | |
| | | accggggtggtgcccatcctggtcgagctggacggcgacgtaaacggccac | |
| | | aagttcagcgtgtccggcgagggcgagggcgatgccacctacggcaagctg | |
| | | accctgaagttcatctgcaccaccggcaagctgcccgtgccctggcccaccct | |
| | | cgtgaccaccctgacctacggcgtgcagtgcttcagccgctaccccgaccac | |
| | | atgaagcagcacgacttcttcaagtccgccatgcccgaaggctacgtccagg | |
| | | agcgcaccatcttcttcaaggacgacggcaactacaagacccgcgccgaggt | |
| | | gaagttcgagggcgacaccctggtgaaccgcatcgagctgaagggcatcga | |
| | | cttcaaggaggacggcaacatcctggggcacaagctggagtacaactacaac | |
| | | agccacaacgtctatatcatggccgacaagcagaagaacggcatcaaggtga | |
| | | acttcaagatccgccacaacatcgaggacggcagcgtgcagctcgccgacc | |
| | | actaccagcagaacacccccatcggcgacggccccgtgctgctgcccgaca | |
| | | accactacctgagcacccagtccgccctgagcaaagaccccaacgagaagc | |
| | | gcgatcacatggtcctgctggagttcgtgaccgccgccgggatcactctcggc | |
| | | atggacgagctgtacaagtgataaACCAGCTGAGAGACTCTA | |
| | | AATCCAGTGACAAGTCTGTCTGCCTATTCACCG | |
| | | ATTTTGATTCTCAAACAAATGTGTCACAAAGTA | |
| | | AGGATTCTGATGTGTATATCACAGACAAAACTG | |
| | | TGCTAGACATGAGGTCTATGGACTTCAAGAGCA | |
| | | ACAGTGCTGTGGCCTGGAGCAACAAATCTGACT | |
| | | TTGCATGTGCAAACGCCTTCAACAACAGCATTA | |
| | | TTCCAGAAGACACCTTCTTCCCCAGCCCAGGTA | |
| | | AGGGCAGCTTTGGTGCCTTCGCAGGCTGTTTCC | |
| | | TTGCTTCAGGAATGGCCAGGTTCTGCCCAGAGC | |
| | | TCTGGTCAATGATGTCTAAAACTCCTCTGATTG | |
| | | GTGGTCTCGGCCTTATCCATTGCCACCAAAACC | |
| | | CTCTTTTTACTAAGAAACAGTGAGCCTTGTTCTG | |
| | | GCAGTCCAGAGAATGACACGGGAAAAAAGCAG | |
| | | ATGAAGAGAAGGTGGCAGGAGAGGGCACGTGG | |
| | | CCCAGCCTCAGTCTCTCCAAC | |
| CT7 | 500bpHA- | ACATACCATAAACCTCCCATTCTGCTAATGCCC | 108 |
| | SG18- | AGCCTAAGTTGGGGAGACCACTCCAGATTCCAA | |
| | TRAC- | GATGTACAGTTTGCTTTGCTGGGCCTTTTTCCCA | |
| | CD33CAR | TGCCTGCCTTTACTCTGCCAGAGTTATATTGCTG | |
| | | GGGTTTTGAAGAAGATCCTATTAAATAAAAGAA | |
| | | TAAGCAGTATTATTAAGTAGCCCTGCATTTCAG | |
| | | GTTTCCTTGAGTGGCAGGCCAGGCCTGGCCGTG | |
| | | AACGTTCACTGAAATCATGGCCTCTTGGCCAAG | |
| | | ATTGATAGCTTGTGCCTGTCCCTGAGTCCCAGT | |
| | | CCATCACGAGCAGCTGGTTTCTAAGATGCTATT | |
| | | TCCCGTATAAAGCATGAGACCGTGACTTGCCAG | |
| | | CCCCACAGAGCCCCGCCCTTGTCCATCACTGGC | |
| | | ATCTGGACTCCAGCCTGGGTTGGGGCAAAGAG | |
| | | GGAAATGAGATCATGTCCTAACCCTGATCCTCT | |
| | | TGTCCCACAGATATCCAGAACCCTGACCCTGCC | |
| | | GTGTtgaataattgagccaccatggctctgcccgtcacagctctgctgctgc | |
| | | ctctggccctgctgctgcacgccgccagacctcaggtgcagctcgtgcagag | |
| | | cggcgctgaggtgaagaaacctggcagcagcgtgaaggtgagctgcaagg | |
| | | cctccggctacaccttcaccgactacaacatgcactgggtgaggcaagcccct | |
| | | ggccagggactggagtggatcggctacatctacccttacaacggcggcacag | |
| | | gctacaaccagaagttcaagtccaaggccaccatcaccgccgatgagtccac | |
| | | caataccgcctacatggagctcagcagcctgaggtccgaggacacagccgtc | |
| | | tactactgcgccaggggcaggcccgctatggactactggggccagggcacc | |
| | | ctggtgacagtgagctctggtggcggcggatccggcggcggcggcagcgg | |
| | | cggcggcggctccgacattcagatgacccagagccctagcagcctgagcgc | |
| | | ttccgtgggagacagggtgaccatcacatgcagggcctccgagagcgtgga | |
| | | caattacggcatcagcttcatgaactggttccagcagaagcccggcaaggcc | |
| | | cccaaactgctgatctatgccgccagcaatcagggctccggcgtgcctagca | |
| | | ggttttccggcagcggcagcggcaccgactttaccctgaccatctccagcctg | |
| | | cagcctgacgatttcgccacctactactgccagcagagcaaggaggtgccttg | |
| | | gacctttggacagggcacaaaggtggagatcaagtccggagccgccgccat | |
| | | cgaagtgatgtacccccctccctacctggataacgagaagagcaacggcacc | |
| | | atcatccacgtgaagggaaagcacctgtgtcccagccccctgtttcccggccc | |
| | | tagcaagcccttctgggtgctggtggtggtcggcggagtgctggcctgctaca | |
| | | gcctcctggtgaccgtggccttcatcatcttctgggtgaggagcaagaggtcc | |
| | | aggctgctgcacagcgactacatgaatatgacccccagaaggcccggcccc | |
| | | accagaaagcactatcagccctacgccccccccagggactttgccgcctaca | |
| | | ggagcagggtgaagttcagcagatccgccgatgcccctgcttaccagcagg | |
| | | gccagaaccagctgtataacgagctgaacctgggcaggagggaggaatacg | |
| | | acgtgctggataagaggaggggaagggaccccgagatgggcggaaagcc | |
| | | caggaggaagaacccccaggagggcctgtacaatgagctgcagaaagaca | |
| | | agatggccgaggcctacagcgagatcggcatgaagggcgagaggaggag | |
| | | gggcaagggccatgacggcctgtaccaaggcctgtccaccgccaccaagga | |
| | | tacctacgacgccctgcacatgcaggccctgcctcccaggggatcctgataa | |
| | | ACCAGCTGAGAGACTCTAAATCCAGTGACAAGT | |
| | | CTGTCTGCCTATTCACCGATTTTGATTCTCAAAC | |
| | | AAATGTGTCACAAAGTAAGGATTCTGATGTGTA | |
| | | TATCACAGACAAAACTGTGCTAGACATGAGGTC | |
| | | TATGGACTTCAAGAGCAACAGTGCTGTGGCCTG | |
| | | GAGCAACAAATCTGACTTTGCATGTGCAAACGC | |
| | | CTTCAACAACAGCATTATTCCAGAAGACACCTT | |
| | | CTTCCCCAGCCCAGGTAAGGGCAGCTTTGGTGC | |
| | | CTTCGCAGGCTGTTTCCTTGCTTCAGGAATGGC | |
| | | CAGGTTCTGCCCAGAGCTCTGGTCAATGATGTC | |
| | | TAAAACTCCTCTGATTGGTGGTCTCGGCCTTATC | |
| | | CATTGCCACCAAAACCCTCTTTTTACTAAGAAA | |
| | | CAGTGAGCCTTGTTCTGGCAGTCCAGAGAATGA | |
| | | CACGGGAAAAAAGCAGATGAAGAGAAGGTGGC | |
| | | AGGAGAGGGCACGTGGCCCAGCCTCAGTCTCTC | |
| | | CAAC | |
| CT8 | 500bpHA- | ACATACCATAAACCTCCCATTCTGCTAATGCCC | 109 |
| | SG18- | AGCCTAAGTTGGGGAGACCACTCCAGATTCCAA | |
| | TRAC- | GATGTACAGTTTGCTTTGCTGGGCCTTTTTCCCA | |
| | CD33CAR- | TGCCTGCCTTTACTCTGCCAGAGTTATATTGCTG | |
| | T2A- | GGGTTTTGAAGAAGATCCTATTAAATAAAAGAA | |
| | EGFP | TAAGCAGTATTATTAAGTAGCCCTGCATTTCAG | |
| | | GTTTCCTTGAGTGGCAGGCCAGGCCTGGCCGTG | |
| | | AACGTTCACTGAAATCATGGCCTCTTGGCCAAG | |
| | | ATTGATAGCTTGTGCCTGTCCCTGAGTCCCAGT | |
| | | CCATCACGAGCAGCTGGTTTCTAAGATGCTATT | |
| | | TCCCGTATAAAGCATGAGACCGTGACTTGCCAG | |
| | | CCCCACAGAGCCCCGCCCTTGTCCATCACTGGC | |
| | | ATCTGGACTCCAGCCTGGGTTGGGGCAAAGAG | |
| | | GGAAATGAGATCATGTCCTAACCCTGATCCTCT | |
| | | TGTCCCACAGATATCCAGAACCCTGACCCTGCC | |
| | | GTGTtgaataattgagccaccatggctctgcccgtcacagctctgctgctgc | |
| | | ctctggccctgctgctgcacgccgccagacctcaggtgcagctcgtgcagag | |
| | | cggcgctgaggtgaagaaacctggcagcagcgtgaaggtgagctgcaagg | |
| | | cctccggctacaccttcaccgactacaacatgcactgggtgaggcaagcccct | |
| | | ggccagggactggagtggatcggctacatctacccttacaacggcggcacag | |
| | | gctacaaccagaagttcaagtccaaggccaccatcaccgccgatgagtccac | |
| | | caataccgcctacatggagctcagcagcctgaggtccgaggacacagccgtc | |
| | | tactactgcgccaggggcaggcccgctatggactactggggccagggcacc | |
| | | ctggtgacagtgagctctggtggcggcggatccggcggcggcggcagcgg | |
| | | cggcggcggctccgacattcagatgacccagagccctagcagcctgagcgc | |
| | | ttccgtgggagacagggtgaccatcacatgcagggcctccgagagcgtgga | |
| | | caattacggcatcagcttcatgaactggttccagcagaagcccggcaaggcc | |
| | | cccaaactgctgatctatgccgccagcaatcagggctccggcgtgcctagca | |
| | | ggttttccggcagcggcagcggcaccgactttaccctgaccatctccagcctg | |
| | | cagcctgacgatttcgccacctactactgccagcagagcaaggaggtgccttg | |
| | | gacctttggacagggcacaaaggtggagatcaagtccggagccgccgccat | |
| | | cgaagtgatgtacccccctccctacctggataacgagaagagcaacggcacc | |
| | | atcatccacgtgaagggaaagcacctgtgtcccagccccctgtttcccggccc | |
| | | tagcaagcccttctgggtgctggtggtggtcggcggagtgctggcctgctaca | |
| | | gcctcctggtgaccgtggccttcatcatcttctgggtgaggagcaagaggtcc | |
| | | aggctgctgcacagcgactacatgaatatgacccccagaaggcccggcccc | |
| | | accagaaagcactatcagccctacgccccccccagggactttgccgcctaca | |
| | | ggagcagggtgaagttcagcagatccgccgatgcccctgcttaccagcagg | |
| | | gccagaaccagctgtataacgagctgaacctgggcaggagggaggaatacg | |
| | | acgtgctggataagaggaggggaagggaccccgagatgggcggaaagcc | |
| | | caggaggaagaacccccaggagggcctgtacaatgagctgcagaaagaca | |
| | | agatggccgaggcctacagcgagatcggcatgaagggcgagaggaggag | |
| | | gggcaagggccatgacggcctgtaccaaggcctgtccaccgccaccaagga | |
| | | tacctacgacgccctgcacatgcaggccctgcctcccaggggatccGGTG | |
| | | GCAGCGGTgaaggaaggggctctttgcttacttgtggagatgttgagga | |
| | | aaatccaggacccgtgagcaagggcgaggagctgttcaccggggtggtgcc | |
| | | catcctggtcgagctggacggcgacgtaaacggccacaagttcagcgtgtcc | |
| | | ggcgagggcgagggcgatgccacctacggcaagctgaccctgaagttcatc | |
| | | tgcaccaccggcaagctgcccgtgccctggcccaccctcgtgaccaccctga | |
| | | cctacggcgtgcagtgcttcagccgctaccccgaccacatgaagcagcacga | |
| | | cttcttcaagtccgccatgcccgaaggctacgtccaggagcgcaccatcttctt | |
| | | caaggacgacggcaactacaagacccgcgccgaggtgaagttcgagggcg | |
| | | acaccctggtgaaccgcatcgagctgaagggcatcgacttcaaggaggacg | |
| | | gcaacatcctggggcacaagctggagtacaactacaacagccacaacgtcta | |
| | | tatcatggccgacaagcagaagaacggcatcaaggtgaacttcaagatccgc | |
| | | cacaacatcgaggacggcagcgtgcagctcgccgaccactaccagcagaac | |
| | | acccccatcggcgacggccccgtgctgctgcccgacaaccactacctgagc | |
| | | acccagtccgccctgagcaaagaccccaacgagaagcgcgatcacatggtc | |
| | | ctgctggagttcgtgaccgccgccgggatcactctcggcatggacgagctgta | |
| | | caagtgataaACCAGCTGAGAGACTCTAAATCCAGT | |
| | | GACAAGTCTGTCTGCCTATTCACCGATTTTGATT | |
| | | CTCAAACAAATGTGTCACAAAGTAAGGATTCTG | |
| | | ATGTGTATATCACAGACAAAACTGTGCTAGACA | |
| | | TGAGGTCTATGGACTTCAAGAGCAACAGTGCTG | |
| | | TGGCCTGGAGCAACAAATCTGACTTTGCATGTG | |
| | | CAAACGCCTTCAACAACAGCATTATTCCAGAAG | |
| | | ACACCTTCTTCCCCAGCCCAGGTAAGGGCAGCT | |
| | | TTGGTGCCTTCGCAGGCTGTTTCCTTGCTTCAGG | |
| | | AATGGCCAGGTTCTGCCCAGAGCTCTGGTCAAT | |
| | | GATGTCTAAAACTCCTCTGATTGGTGGTCTCGG | |
| | | CCTTATCCATTGCCACCAAAACCCTCTTTTTACT | |
| | | AAGAAACAGTGAGCCTTGTTCTGGCAGTCCAGA | |
| | | GAATGACACGGGAAAAAAGCAGATGAAGAGAA | |
| | | GGTGGCAGGAGAGGGCACGTGGCCCAGCCTCA | |
| | | GTCTCTCCAAC | |
| CT9 | 500bpHA- | ACATACCATAAACCTCCCATTCTGCTAATGCCC | 110 |
| | SG18- | AGCCTAAGTTGGGGAGACCACTCCAGATTCCAA | |
| | TRAC- | GATGTACAGTTTGCTTTGCTGGGCCTTTTTCCCA | |
| | EF1a- | TGCCTGCCTTTACTCTGCCAGAGTTATATTGCTG | |
| | CD33CAR- | GGGTTTTGAAGAAGATCCTATTAAATAAAAGAA | |
| | T2A- | TAAGCAGTATTATTAAGTAGCCCTGCATTTCAG | |
| | EGFP | GTTTCCTTGAGTGGCAGGCCAGGCCTGGCCGTG | |
| | | AACGTTCACTGAAATCATGGCCTCTTGGCCAAG | |
| | | ATTGATAGCTTGTGCCTGTCCCTGAGTCCCAGT | |
| | | CCATCACGAGCAGCTGGTTTCTAAGATGCTATT | |
| | | TCCCGTATAAAGCATGAGACCGTGACTTGCCAG | |
| | | CCCCACAGAGCCCCGCCCTTGTCCATCACTGGC | |
| | | ATCTGGACTCCAGCCTGGGTTGGGGCAAAGAG | |
| | | GGAAATGAGATCATGTCCTAACCCTGATCCTCT | |
| | | TGTCCCACAGATATCCAGAACCCTGACCCTGCC | |
| | | GTGTtgaataattgaggctccggtgcccgtcagtgggcagagcgcacatc | |
| | | gcccacagtccccgagaagttggggggaggggtcggcaattgaaccggtgc | |
| | | ctagagaaggtggcgcggggtaaactgggaaagtgatgtcgtgtactggctc | |
| | | cgcctttttcccgaggggggggagaaccgtatataagtgcagtagtcgccgt | |
| | | gaacgttctttttcgcaacgggtttgccgccagaacacaggtaagtgccgtgtg | |
| | | tggttcccgcgggcctggcctctttacgggttatggcccttgcgtgccttgaatt | |
| | | acttccacctggctgcagtacgtgattcttgatcccgagcttcgggttggaagtg | |
| | | ggtgggagagttcgaggccttgcgcttaaggagccccttcgcctcgtgcttga | |
| | | gttgaggcctggcctgggcgctggggccgccgcgtgcgaatctggtggcac | |
| | | cttcgcgcctgtctcgctgctttcgataagtctctagccatttaaaatttttgatgac | |
| | | ctgctgcgacgctttttttctggcaagatagtcttgtaaatgcgggccaagatctg | |
| | | cacactggtatttcggtttttggggccgcgggcggcgacggggcccgtgcgt | |
| | | cccagcgcacatgttcggcgaggcggggcctgcgagcgcggccaccgaga | |
| | | atcggacgggggtagtctcaagctggccggcctgctctggtgcctggcctcg | |
| | | cgccgccgtgtatcgccccgccctgggcggcaaggctggcccggtcggcac | |
| | | cagttgcgtgagcggaaagatggccgcttcccggccctgctgcagggagctc | |
| | | aaaatggaggacgcggcgctcgggagagcgggcgggtgagtcacccacac | |
| | | aaaggaaaagggcctttccgtcctcagccgtcgcttcatgtgactccactgagt | |
| | | accgggcgccgtccaggcacctcgattagttctcgtgcttttggagtacgtcgt | |
| | | ctttaggttggggggaggggttttatgcgatggagtttccccacactgagtggg | |
| | | tggagactgaagttaggccagcttggcacttgatgtaattctccttggaatttgcc | |
| | | ctttttgagtttggatcttggttcattctcaagcctcagacagtggttcaaagtttttt | |
| | | tcttccatttcaggtgtcgtgagctagcctcgaggccaccatggctctgcccgtc | |
| | | acagctctgctgctgcctctggccctgctgctgcacgccgccagacctcaggt | |
| | | gcagctcgtgcagagcggcgctgaggtgaagaaacctggcagcagcgtga | |
| | | aggtgagctgcaaggcctccggctacaccttcaccgactacaacatgcactg | |
| | | ggtgaggcaagcccctggccagggactggagtggatcggctacatctaccct | |
| | | tacaacggcggcacaggctacaaccagaagttcaagtccaaggccaccatca | |
| | | ccgccgatgagtccaccaataccgcctacatggagctcagcagcctgaggtc | |
| | | cgaggacacagccgtctactactgcgccaggggcaggcccgctatggacta | |
| | | ctggggccagggcaccctggtgacagtgagctctggtggcggcggatccgg | |
| | | cggcggcggcagcggcggcggcggctccgacattcagatgacccagagcc | |
| | | ctagcagcctgagcgcttccgtgggagacagggtgaccatcacatgcaggg | |
| | | cctccgagagcgtggacaattacggcatcagcttcatgaactggttccagcag | |
| | | aagcccggcaaggcccccaaactgctgatctatgccgccagcaatcagggct | |
| | | ccggcgtgcctagcaggttttccggcagcggcagcggcaccgactttaccct | |
| | | gaccatctccagcctgcagcctgacgatttcgccacctactactgccagcaga | |
| | | gcaaggaggtgccttggacctttggacagggcacaaaggtggagatcaagtc | |
| | | cggagccgccgccatcgaagtgatgtacccccctccctacctggataacgag | |
| | | aagagcaacggcaccatcatccacgtgaagggaaagcacctgtgtcccagc | |
| | | cccctgtttcccggccctagcaagcccttctgggtgctggtggtggtcggcgg | |
| | | agtgctggcctgctacagcctcctggtgaccgtggccttcatcatcttctgggtg | |
| | | aggagcaagaggtccaggctgctgcacagcgactacatgaatatgaccccca | |
| | | gaaggcccggccccaccagaaagcactatcagccctacgccccccccagg | |
| | | gactttgccgcctacaggagcagggtgaagttcagcagatccgccgatgccc | |
| | | ctgcttaccagcagggccagaaccagctgtataacgagctgaacctgggcag | |
| | | gagggaggaatacgacgtgctggataagaggaggggaagggaccccgag | |
| | | atgggcggaaagcccaggaggaagaacccccaggagggcctgtacaatga | |
| | | gctgcagaaagacaagatggccgaggcctacagcgagatcggcatgaagg | |
| | | gcgagaggaggaggggcaagggccatgacggcctgtaccaaggcctgtcc | |
| | | accgccaccaaggatacctacgacgccctgcacatgcaggccctgcctccca | |
| | | ggggatccGGTGGCAGCGGTgaaggaaggggctctttgcttacttg | |
| | | tggagatgttgaggaaaatccaggacccgtgagcaagggcgaggagctgttc | |
| | | accggggtggtgcccatcctggtcgagctggacggcgacgtaaacggccac | |
| | | aagttcagcgtgtccggcgagggcgagggcgatgccacctacggcaagctg | |
| | | accctgaagttcatctgcaccaccggcaagctgcccgtgccctggcccaccct | |
| | | cgtgaccaccctgacctacggcgtgcagtgcttcagccgctaccccgaccac | |
| | | atgaagcagcacgacttcttcaagtccgccatgcccgaaggctacgtccagg | |
| | | agcgcaccatcttcttcaaggacgacggcaactacaagacccgcgccgaggt | |
| | | gaagttcgagggcgacaccctggtgaaccgcatcgagctgaagggcatcga | |
| | | cttcaaggaggacggcaacatcctggggcacaagctggagtacaactacaac | |
| | | agccacaacgtctatatcatggccgacaagcagaagaacggcatcaaggtga | |
| | | acttcaagatccgccacaacatcgaggacggcagcgtgcagctcgccgacc | |
| | | actaccagcagaacacccccatcggcgacggccccgtgctgctgcccgaca | |
| | | accactacctgagcacccagtccgccctgagcaaagaccccaacgagaagc | |
| | | gcgatcacatggtcctgctggagttcgtgaccgccgccgggatcactctcggc | |
| | | atggacgagctgtacaagtgataaACCAGCTGAGAGACTCTA | |
| | | AATCCAGTGACAAGTCTGTCTGCCTATTCACCG | |
| | | ATTTTGATTCTCAAACAAATGTGTCACAAAGTA | |
| | | AGGATTCTGATGTGTATATCACAGACAAAACTG | |
| | | TGCTAGACATGAGGTCTATGGACTTCAAGAGCA | |
| | | ACAGTGCTGTGGCCTGGAGCAACAAATCTGACT | |
| | | TTGCATGTGCAAACGCCTTCAACAACAGCATTA | |
| | | TTCCAGAAGACACCTTCTTCCCCAGCCCAGGTA | |
| | | AGGGCAGCTTTGGTGCCTTCGCAGGCTGTTTCC | |
| | | TTGCTTCAGGAATGGCCAGGTTCTGCCCAGAGC | |
| | | TCTGGTCAATGATGTCTAAAACTCCTCTGATTG | |
| | | GTGGTCTCGGCCTTATCCATTGCCACCAAAACC | |
| | | CTCTTTTTACTAAGAAACAGTGAGCCTTGTTCTG | |
| | | GCAGTCCAGAGAATGACACGGGAAAAAAGCAG | |
| | | ATGAAGAGAAGGTGGCAGGAGAGGGCACGTGG | |
| | | CCCAGCCTCAGTCTCTCCAAC | |
| CT10 | 500bpHA- | gagtccagagtgctaaccattacaccatGGAACCGCCACGCAT | 111 |
| | Rab11a- | GTGTAGCTGCCTTCGGCTGTCTAATCCTCAGAG | |
| | SFFV- | AACCCCGCCCCCATCCACAAACCCACCACTCAC | |
| | CD33CAR | AGGCGGTCCCGCCTGGTTCCAGCGAGCCGCTTC | |
| | | CGGCACGGTAGCTCGAGAAATGAGCAAGCGGC | |
| | | CACTAAGACTATGGTAGCTAGGAGTTCCAGGAC | |
| | | TCAGTTTCCCCTTTGAGCCTCCTTTAGCGACTAA | |
| | | AGCTTGAAGCCCCACGCATCTCGACTCTCGCGC | |
| | | ACACCGCCCTTGTTGGGCTCAGGGGCGGGGCGC | |
| | | CGCCCCCGGAAGTACTTCCCCTTAAAGGCTGGG | |
| | | GCCTGCCGGAAATGGCGCAGCGGCAGGGAGGG | |
| | | GCTCTTCACCCAGTCCGGCAGTTGAAGCTCGGC | |
| | | GCTCGGGTTACCCCTGCAGCGACGCCCCCTGGT | |
| | | CCCACAGATACCACTGCTGCTCCCGCCCTTTCG | |
| | | CTCCTCGGCCGCGCAATGGGCACCCGCGAGTAA | |
| | | CGCCATTTTGCAAGGCATGGAAAAATACCAAAC | |
| | | CAAGAATAGAGAAGTTCAGATCAAGGGCGGGT | |
| | | ACATGAAAATAGCTAACGTTGGGCCAAACAGG | |
| | | ATATCTGCGGTGAGCAGTTTCGGCCCCGGCCCG | |
| | | GGGCCAAGAACAGATGGTCACCGCAGTTTCGG | |
| | | CCCCGGCCCGAGGCCAAGAACAGATGGTCCCC | |
| | | AGATATGGCCCAACCCTCAGCAGTTTCTTAAGA | |
| | | CCCATCAGATGTTTCCAGGCTCCCCCAAGGACC | |
| | | TGAAATGACCCTGCGCCTTATTTGAATTAACCA | |
| | | ATCAGCCTGCTTCTCGCTTCTGTTCGCGCGCTTC | |
| | | TGCTTCCCGAGCTCTATAAAAGAGCTCACAACC | |
| | | CCTCACTCGGCGCGCCAGTCCTCCGACAGACTG | |
| | | AGTCGCCCGGGCCGCGGCCGCGGGCTAGCGGA | |
| | | TCCCCACCGGTCGCCACCatggctctgcccgtcacagctctgc | |
| | | tgctgcctctggccctgctgctgcacgccgccagacctcaggtgcagctcgtg | |
| | | cagagcggcgctgaggtgaagaaacctggcagcagcgtgaaggtgagctg | |
| | | caaggcctccggctacaccttcaccgactacaacatgcactgggtgaggcaa | |
| | | gcccctggccagggactggagtggatcggctacatctacccttacaacggcg | |
| | | gcacaggctacaaccagaagttcaagtccaaggccaccatcaccgccgatga | |
| | | gtccaccaataccgcctacatggagctcagcagcctgaggtccgaggacaca | |
| | | gccgtctactactgcgccaggggcaggcccgctatggactactggggccag | |
| | | ggcaccctggtgacagtgagctctggtggcggcggatccggcggcggcgg | |
| | | cagcggcggcggcggctccgacattcagatgacccagagccctagcagcct | |
| | | gagcgcttccgtgggagacagggtgaccatcacatgcagggcctccgagag | |
| | | cgtggacaattacggcatcagcttcatgaactggttccagcagaagcccggca | |
| | | aggcccccaaactgctgatctatgccgccagcaatcagggctccggcgtgcc | |
| | | tagcaggttttccggcagcggcagcggcaccgactttaccctgaccatctcca | |
| | | gcctgcagcctgacgatttcgccacctactactgccagcagagcaaggaggt | |
| | | gccttggacctttggacagggcacaaaggtggagatcaagtccggagccgc | |
| | | cgccatcgaagtgatgtacccccctccctacctggataacgagaagagcaac | |
| | | ggcaccatcatccacgtgaagggaaagcacctgtgtcccagccccctgtttcc | |
| | | cggccctagcaagcccttctgggtgctggtggtggtcggcggagtgctggcc | |
| | | tgctacagcctcctggtgaccgtggccttcatcatcttctgggtgaggagcaag | |
| | | aggtccaggctgctgcacagcgactacatgaatatgacccccagaaggcccg | |
| | | gccccaccagaaagcactatcagccctacgccccccccagggactttgccgc | |
| | | ctacaggagcagggtgaagttcagcagatccgccgatgcccctgcttaccag | |
| | | cagggccagaaccagctgtataacgagctgaacctgggcaggagggagga | |
| | | atacgacgtgctggataagaggaggggaagggaccccgagatgggcggaa | |
| | | agcccaggaggaagaacccccaggagggcctgtacaatgagctgcagaaa | |
| | | gacaagatggccgaggcctacagcgagatcggcatgaagggcgagaggag | |
| | | gaggggcaagggccatgacggcctgtaccaaggcctgtccaccgccaccaa | |
| | | ggatacctacgacgccctgcacatgcaggccctgcctcccaggggatcctga | |
| | | taaAATGAATGCAATTGTTGTTGTTAATAAAGGA | |
| | | AATTTATTTTCATTGCAATAGTGTGTTGGAATTT | |
| | | TTTGTGTCTCTCACGACGAGTACGACTACCTCTT | |
| | | TAAAGGTGAGGCCATGGGCTCTCGCACTCTACA | |
| | | CAGTCCTCGTTCGGGGACCCGGGCCACTCCCGG | |
| | | TGGACCCTCGTGCCGGCCACCCCTGCACTGATA | |
| | | TAGGCCTCCCTCAGCCCTTCCTTTTTGTGCGGTT | |
| | | CCGTCTCCTACCCAGCTCAGCCTCTTCTCCCCCG | |
| | | CTCAGACAGGGGTCCCCATCACATGCCGCTCTC | |
| | | TGAGCGACCTCTCCATAGGCCTTCGCTGGCCTC | |
| | | AGAGCCCCTCCCTGCGTGTCCTTCCCCTGGCGG | |
| | | ACTGCCTTCTCCCACATCGTCGAATTCCTTTCCC | |
| | | CGGGTTCTACGGCCCCGCCGCTCCTCCCACCAT | |
| | | CTCTCTTTTCGGGTGTAGCGCCCCCTCCCCCTCG | |
| | | GCGTACACCCTTCCCAGCTCGCGTCCTCTCCCG | |
| | | AAGCCCCTCTGACGGGTTCTTCGCTTCCCTCTTG | |
| | | GCCTTGCCTTCGGTGCAGACTCCCATTACAGGT | |
| | | CTTTTTCTTATC | |
| CT11 | 500bpHA- | TAGGGACAGGATTGGTGACAGAAAAGCCCCAT | 112 |
| | AAVS1- | CCTTAGGCCTCCTCCTTCCTAGTCTCCTGATATT | |
| | SFFV- | GGGTCTAACCCCCACCTCCTGTTAGGCAGATTC | |
| | CD33CAR | CTTATCTGGTGACACACCCCCATTTCCTGGAGC | |
| | | CATCTCTCTCCTTGCCAGAACCTCTAAGGTTTGC | |
| | | TTACGATGGAGCCAGAGAGGATCCTGGGAGGG | |
| | | AGAGCTTGGCAGGGGGTGGGAGGGAAGGGGGG | |
| | | GATGCGTGACCTGCCCGGTTCTCAGTGGCCACC | |
| | | CTGCGCTACCCTCTCCCAGAACCTGAGCTGCTC | |
| | | TGACGCGGCTGTCTGGTGCGTTTCACTGATCCT | |
| | | GGTGCTGCAGCTTCCTTACACTTCCCAAGAGGA | |
| | | GAAGCAGTTTGGAAAAACAAAATCAGAATAAG | |
| | | TTGGTCCTGAGTTCTAACTTTGGCTCTTCACCTT | |
| | | TCTAGTCCCCAATTTATATTGTTCCTCCGTGCGT | |
| | | CAGTTTTACCTGTGAGATAAGGCCAGTAGCCAG | |
| | | CCCCGGTAACGCCATTTTGCAAGGCATGGAAAA | |
| | | ATACCAAACCAAGAATAGAGAAGTTCAGATCA | |
| | | AGGGCGGGTACATGAAAATAGCTAACGTTGGG | |
| | | CCAAACAGGATATCTGCGGTGAGCAGTTTCGGC | |
| | | CCCGGCCCGGGGCCAAGAACAGATGGTCACCG | |
| | | CAGTTTCGGCCCCGGCCCGAGGCCAAGAACAG | |
| | | ATGGTCCCCAGATATGGCCCAACCCTCAGCAGT | |
| | | TTCTTAAGACCCATCAGATGTTTCCAGGCTCCC | |
| | | CCAAGGACCTGAAATGACCCTGCGCCTTATTTG | |
| | | AATTAACCAATCAGCCTGCTTCTCGCTTCTGTTC | |
| | | GCGCGCTTCTGCTTCCCGAGCTCTATAAAAGAG | |
| | | CTCACAACCCCTCACTCGGCGCGCCAGTCCTCC | |
| | | GACAGACTGAGTCGCCCGGGCCGCGGCCGCGG | |
| | | GCTAGCGGATCCCCACCGGTCGCCACCatggctctgc | |
| | | ccgtcacagctctgctgctgcctctggccctgctgctgcacgccgccagacct | |
| | | caggtgcagctcgtgcagagcggcgctgaggtgaagaaacctggcagcag | |
| | | cgtgaaggtgagctgcaaggcctccggctacaccttcaccgactacaacatg | |
| | | cactgggtgaggcaagcccctggccagggactggagtggatcggctacatct | |
| | | acccttacaacggcggcacaggctacaaccagaagttcaagtccaaggccac | |
| | | catcaccgccgatgagtccaccaataccgcctacatggagctcagcagcctg | |
| | | aggtccgaggacacagccgtctactactgcgccaggggcaggcccgctatg | |
| | | gactactggggccagggcaccctggtgacagtgagctctggtggcggcgga | |
| | | tccggcggcggcggcagcggcggcggcggctccgacattcagatgaccca | |
| | | gagccctagcagcctgagcgcttccgtgggagacagggtgaccatcacatgc | |
| | | agggcctccgagagcgtggacaattacggcatcagcttcatgaactggttcca | |
| | | gcagaagcccggcaaggcccccaaactgctgatctatgccgccagcaatca | |
| | | gggctccggcgtgcctagcaggttttccggcagcggcagcggcaccgacttt | |
| | | accctgaccatctccagcctgcagcctgacgatttcgccacctactactgccag | |
| | | cagagcaaggaggtgccttggacctttggacagggcacaaaggtggagatc | |
| | | aagtccggagccgccgccatcgaagtgatgtacccccctccctacctggataa | |
| | | cgagaagagcaacggcaccatcatccacgtgaagggaaagcacctgtgtcc | |
| | | cagccccctgtttcccggccctagcaagcccttctgggtgctggtggtggtcg | |
| | | gcggagtgctggcctgctacagcctcctggtgaccgtggccttcatcatcttct | |
| | | gggtgaggagcaagaggtccaggctgctgcacagcgactacatgaatatga | |
| | | cccccagaaggcccggccccaccagaaagcactatcagccctacgcccccc | |
| | | ccagggactttgccgcctacaggagcagggtgaagttcagcagatccgccga | |
| | | tgcccctgcttaccagcagggccagaaccagctgtataacgagctgaacctg | |
| | | ggcaggagggaggaatacgacgtgctggataagaggaggggaagggacc | |
| | | ccgagatgggcggaaagcccaggaggaagaacccccaggagggcctgta | |
| | | caatgagctgcagaaagacaagatggccgaggcctacagcgagatcggcat | |
| | | gaagggcgagaggaggaggggcaagggccatgacggcctgtaccaaggc | |
| | | ctgtccaccgccaccaaggatacctacgacgccctgcacatgcaggccctgc | |
| | | ctcccaggggatcctgataaAATGAATGCAATTGTTGTTGT | |
| | | TAATAAAGGAAATTTATTTTCATTGCAATAGTG | |
| | | TGTTGGAATTTTTTGTGTCTCTCATCCTGGCAGG | |
| | | GCTGTGGTGAGGAGGGGGGTGTCCGTGTGGAA | |
| | | AACTCCCTTTGTGAGAATGGTGCGTCCTAGGTG | |
| | | TTCACCAGGTCGTGGCCGCCTCTACTCCCTTTCT | |
| | | CTTTCTCCATCCTTCTTTCCTTAAAGAGTCCCCA | |
| | | GTGCTATCTGGGACATATTCCTCCGCCCAGAGC | |
| | | AGGGTCCCGCTTCCCTAAGGCCCTGCTCTGGGC | |
| | | TTCTGGGTTTGAGTCCTTGGCAAGCCCAGGAGA | |
| | | GGCGCTCAGGCTTCCCTGTCCCCCTTCCTCGTCC | |
| | | ACCATCTCATGCCCCTGGCTCTCCTGCCCCTTCC | |
| | | CTACAGGGGTTCCTGGCTCTGCTCTTCAGACTG | |
| | | AGCCCCGTTCCCCTGCATCCCCGTTCCCCTGCAT | |
| | | CCCCCTTCCCCTGCATCCCCCAGAGGCCCCAGG | |
| | | CCACCTACTTGGCCTGGACCCCACGAGAGGCCA | |
| | | CCCCAGCCCTGTCTACCAGGCTGCCTTTTGGGT | |
| | | GGATTCTCCTCCAACTGTGGGGTG | |
| CT12 | 500bpHA- | TTTGGTTTTGGCTTTCACTGGAGTCTGCAACAA | 113 |
| | g1-CD5- | GAACTGGCATCATGCTGCCCATTTCCCGCCTCT | |
| | CAR- | CCCCACCCAGACCCCTGCCTCAGGGACGCCTGT | |
| | P2A- | CCTCAGCCCAGCCCTCAGCTGCAGCCAGGCCTT | |
| | EGFP | CAGCCTCCGTAACCCCCGCTCAGGGTCCCCACC | |
| | | CCCTGCAGCCCTGTCCCTCCAGGATGCATGGCC | |
| | | TTGTCCTGTGTGGGGGTGGCCGAGAGCACTGCC | |
| | | CCAGCCCTGGGTACCTTGGGCAGGAAGCTGGCA | |
| | | GAGGCCAGGGCTGCCATTCAAACAGGGGCAGG | |
| | | TGGTTTTGCCAGGAGGAAGTTGACAGTTCAACT | |
| | | TCAAACATGGGTGACGCAGGCCCCACACTGCCT | |
| | | GCTCCCCGTCCCACCCCTCCCTGAGCACGCCAC | |
| | | CCCGCCCTCTCCCTCTCTGAGAGCGAGATACCC | |
| | | GGCCAGACACCCTCACCTGCGGTGCCCAGCTGC | |
| | | CCAGGCTGAGGCAAGAGAAGGCCAGAAACCAT | |
| | | GCCCATGTAAATAAATAAggctccggtgcccgtcagtgggc | |
| | | agagcgcacatcgcccacagtccccgagaagttggggggaggggtcggca | |
| | | attgatccggtgcctagagaaggtggcgcggggtaaactgggaaagtgatgt | |
| | | cgtgtactggctccgcctttttcccgagggtgggggagaaccgtatataagtgc | |
| | | agtagtcgccgtgaacgttctttttcgcaacgggtttgccgccagaacacaggt | |
| | | aagtgccgtgtgtggttcccgcgggcctggcctctttacgggttatggcccttg | |
| | | cgtgccttgaattacttccacctggctgcagtacgtgattcttgatcccgagcttc | |
| | | gggttggaagtgggtgggagagttcgaggccttgcgcttaaggagccccttc | |
| | | gcctcgtgcttgagttgaggcctggcctgggcgctggggccgccgcgtgcga | |
| | | atctggtggcaccttcgcgcctgtctcgctgctttcgataagtctctagccattta | |
| | | aaatttttgatgacctgctgcgacgctttttttctggcaagatagtcttgtaaatgc | |
| | | gggccaagatctgcacactggtatttcggtttttggggccgcgggcggcgacg | |
| | | gggcccgtgcgtcccagcgcacatgttcggcgaggcggggcctgcgagcg | |
| | | cggccaccgagaatcggacgggggtagtctcaagctggccggcctgctctg | |
| | | gtgcctggcctcgcgccgccgtgtatcgccccgccctgggcggcaaggctg | |
| | | gcccggtcggcaccagttgcgtgagcggaaagatggccgcttcccggccct | |
| | | gctgcagggagctcaaaatggaggacgcggcgctcgggagagcgggcgg | |
| | | gtgagtcacccacacaaaggaaaagggcctttccgtcctcagccgtcgcttca | |
| | | tgtgactccacggagtaccgggcgccgtccaggcacctcgattagttctcgag | |
| | | cttttggagtacgtcgtctttaggttggggggaggggttttatgcgatggagtttc | |
| | | cccacactgagtgggtggagactgaagttaggccagcttggcacttgatgtaa | |
| | | ttctccttggaatttgccctttttgagtttggatcttggttcattctcaagcctcagac | |
| | | agtggttcaaagtttttttcttccatttcaggtgtcgtgacgtacgGAATTCG | |
| | | ACGCCACCATGGAGTTCGGCCTGAGCTGGCTGT | |
| | | TCCTGGTGGCCATCCTGAAGGGCGTGCAGTGCA | |
| | | TCGACGCCATGGGCAACATCCAGCTGGTGCAGA | |
| | | GCGGCCCCGAGCTGAAGAAGCCCGGCGAGACC | |
| | | GTGAAGATCAGCTGCAAGGCCAGCGGCTACAC | |
| | | CTTCACCAACTACGGCATGAACTGGGTGAAGCA | |
| | | GGCCCCCGGCAAGGGCCTGAGGTGGATGGGCT | |
| | | GGATCAACACCCACACCGGCGAGCCCACCTAC | |
| | | GCCGACGACTTCAAGGGCAGGTTCGCCTTCAGC | |
| | | CTGGAGACCAGCGCCAGCACCGCCTACCTGCAG | |
| | | ATCAACAACCTGAAGAACGAGGACACCGCCAC | |
| | | CTACTTCTGCACCAGGAGGGGCTACGACTGGTA | |
| | | CTTCGACGTGTGGGGCGCCGGCACCACCGTGAC | |
| | | CGTGAGCAGCGGCGGCGGCGGCAGCGGCGGCG | |
| | | GCGGCAGCGGCGGCGGCGGCAGCGACATCAAG | |
| | | ATGACCCAGAGCCCCAGCAGCATGTACGCCAG | |
| | | CCTGGGCGAGAGGGTGACCATCACCTGCAAGG | |
| | | CCAGCCAGGACATCAACAGCTACCTGAGCTGGT | |
| | | TCCACCACAAGCCCGGCAAGAGCCCCAAGACC | |
| | | CTGATCTACAGGGCCAACAGGCTGGTGGACGG | |
| | | CGTGCCCAGCAGGTTCAGCGGCAGCGGCAGCG | |
| | | GCCAGGACTACAGCCTGACCATCAGCAGCCTGG | |
| | | ACTACGAGGACATGGGCATCTACTACTGCCAGC | |
| | | AGTACGACGAGAGCCCCTGGACCTTCGGCGGC | |
| | | GGCACCAAGCTGGAGATGAAGGGCAGCGGCGA | |
| | | CCCCGCCGAGCCCAAGAGCCCCGACAAGACCC | |
| | | ACACCTGCCCCCCCTGCCCCGCCCCCGAGCTGC | |
| | | TGGGCGGCCCCAGCGTGTTCCTGTTCCCCCCCA | |
| | | AGCCCAAGGACACCCTGATGATCAGCAGGACC | |
| | | CCCGAGGTGACCTGCGTGGTGGTGGACGTGAGC | |
| | | CACGAGGACCCCGAGGTGAAGTTCAACTGGTA | |
| | | CGTGGACGGCGTGGAGGTGCACAACGCCAAGA | |
| | | CCAAGCCCAGGGAGGAGCAGTACAACAGCACC | |
| | | TACAGGGTGGTGAGCGTGCTGACCGTGCTGCAC | |
| | | CAGGACTGGCTGAACGGCAAGGAGTACAAGTG | |
| | | CAAGGTGAGCAACAAGGCCCTGCCCGCCCCCAT | |
| | | CGAGAAGACCATCAGCAAGGCCAAGGGCCAGC | |
| | | CCAGGGAGCCCCAGGTGTACACCCTGCCCCCCA | |
| | | GCAGGGACGAGCTGACCAAGAACCAGGTGAGC | |
| | | CTGACCTGCCTGGTGAAGGGCTTCTACCCCAGC | |
| | | GACATCGCCGTGGAGTGGGAGAGCAACGGCCA | |
| | | GCCCGAGAACAACTACAAGACCACCCCCCCCGT | |
| | | GCTGGACAGCGACGGCAGCTTCTTCCTGTACAG | |
| | | CAAGCTGACCGTGGACAAGAGCAGGTGGCAGC | |
| | | AGGGCAACGTGTTCAGCTGCAGCGTGATGCACG | |
| | | AGGCCCTGCACAACCACTACACCCAGAAGAGC | |
| | | CTGAGCCTGAGCCCCGGCAAGAAGGACCCCAA | |
| | | GTTCTGGGTGCTGGTGGTGGTGGGCGGCGTGCT | |
| | | GGCCTGCTACAGCCTGCTGGTGACCGTGGCCTT | |
| | | CATCATCTTCTGGGTGAGGAGCAAGAGGAGCA | |
| | | GGCTGCTGCACAGCGACTACATGAACATGACCC | |
| | | CCAGGAGGCCCGGCCCCACCAGGAAGCACTAC | |
| | | CAGCCCTACGCCCCCCCCAGGGACTTCGCCGCC | |
| | | TACAGGAGCAGGGTGAAGTTCAGCAGGAGCGC | |
| | | CGACGCCCCCGCCTACCAGCAGGGCCAGAACC | |
| | | AGCTGTACAACGAGCTGAACCTGGGCAGGAGG | |
| | | GAGGAGTACGACGTGCTGGACAAGAGGAGGGG | |
| | | CAGGGACCCCGAGATGGGCGGCAAGCCCAGGA | |
| | | GGAAGAACCCCCAGGAGGGCCTGTACAACGAG | |
| | | CTGCAGAAGGACAAGATGGCCGAGGCCTACAG | |
| | | CGAGATCGGCATGAAGGGCGAGAGGAGGAGGG | |
| | | GCAAGGGCCACGACGGCCTGTACCAGGGCCTG | |
| | | AGCACCGCCACCAAGGACACCTACGACGCCCT | |
| | | GCACATGCAGGCCCTGCCCCCCAGGGCCACGA | |
| | | ACTTCTCTCTGTTAAAGCAAGCAGGAGACGTGG | |
| | | AAGAAAACCCCGGTCCTATGGTGAGCAAGGGC | |
| | | GAGGAGCTGTTCACCGGGGTGGTGCCCATCCTG | |
| | | GTCGAGCTGGACGGCGACGTAAACGGCCACAA | |
| | | GTTCAGCGTGTCCGGCGAGGGCGAGGGCGATG | |
| | | CCACCTACGGCAAGCTGACCCTGAAGTTCATCT | |
| | | GCACCACCGGCAAGCTGCCCGTGCCCTGGCCCA | |
| | | CCCTCGTGACCACCCTGACCTACGGCGTGCAGT | |
| | | GCTTCAGCCGCTACCCCGACCACATGAAGCAGC | |
| | | ACGACTTCTTCAAGTCCGCCATGCCCGAAGGCT | |
| | | ACGTCCAGGAGCGCACCATCTTCTTCAAGGACG | |
| | | ACGGCAACTACAAGACCCGCGCCGAGGTGAAG | |
| | | TTCGAGGGCGACACCCTGGTGAACCGCATCGAG | |
| | | CTGAAGGGCATCGACTTCAAGGAGGACGGCAA | |
| | | CATCCTGGGGCACAAGCTGGAGTACAACTACA | |
| | | ACAGCCACAACGTCTATATCATGGCCGACAAGC | |
| | | AGAAGAACGGCATCAAGGTGAACTTCAAGATC | |
| | | CGCCACAACATCGAGGACGGCAGCGTGCAGCT | |
| | | CGCCGACCACTACCAGCAGAACACCCCCATCGG | |
| | | CGACGGCCCCGTGCTGCTGCCCGACAACCACTA | |
| | | CCTGAGCACCCAGTCCGCCCTGAGCAAAGACCC | |
| | | CAACGAGAAGCGCGATCACATGGTCCTGCTGG | |
| | | AGTTCGTGACCGCCGCCGGGATCACTCTCGGCA | |
| | | TGGACGAGCTGTACAAGTAAAATGAATGCAATT | |
| | | GTTGTTGTTAATAAAGGAAATTTATTTTCATTGC | |
| | | AATAGTGTGTTGGAATTTTTTGTGTCTCTCAGGG | |
| | | TCTCTGCAACCGCTGGCCACCTTGTACCTGCTG | |
| | | GGGATGCTGGGTGAGTACCCCTCCCAGGTGTCC | |
| | | TGCGAACACCCGGGCTCGCTCCAGTGCAAGGA | |
| | | AGGAGTTCCCAGTTTTACCCAAGGCTGACTCTG | |
| | | GGATCCACATGTCAGCCCTCTGGAGCGTTGTGG | |
| | | AGATTTGGGGCCACTGGGATCCCTGCCTGCCCC | |
| | | CACTAAGCCGCAGCTTGGCCCTCTGTCCTGCAT | |
| | | GTCCCACCCGCCAGGAGCACAACCTTGCCTCTC | |
| | | TCATGCGCTGTTGAGAACCCTGCTTTACCCTTCC | |
| | | AGTGCAAGAGAGACTGCAGGGGGGACCCGCAT | |
| | | TTGATGGGGCCCAGACAACTTGATTCCTAGGCT | |
| | | GAGTTGGATTTTAGCAGAGCATTCAGGCCTCCC | |
| | | TCTGCGAGGTCCCCCACTGACAGCCCAGCCTTT | |
| | | ACTTGGTCGCCTCCAGAGACATGGAAACTCGCC | |
| | | GTCTCCGAGGCAGCTCTGATGATGCTCTGGACA | |
| | | GAC | |
| CT13 | 500bpHA- | TTGAGGCTGCAGTGAGCTGTGATCATGCCACTG | 114 |
| | g2-CD5- | CACTCCAGCCTGGGTGAGGAGAGTGATATCCTG | |
| | CAR- | TCTCAAAAAGTAATAATAATAATAATTGATGGT | |
| | P2A- | TACATTATTACAAAGGTTAGCATGAGGGAATTT | |
| | EGFP | GAGGTAGGGAGTAATGGAACTGGTGTATACCTT | |
| | | GATTGTGATGGTGGTCACACACATCTATATATG | |
| | | TGACATTCACGGAATCATGCAGTAAAGAAAAA | |
| | | TCAATTTCACGTCTGTTCATTTTAAAAGTAACGT | |
| | | TTTTTAAGAAGAAAAAAAATCGATAGTTGCAGC | |
| | | CCACTAGATAGAATTCATATCACTCAGGGGTTC | |
| | | CAACCTGGAGTATGAAAATTCCTGTCCCTAAAA | |
| | | CCCATGATAGTGGATAGGGGGAGGCAGAAAGG | |
| | | GCCATTGCTCGGGCTGTGGGTGGGTGAGCTGGG | |
| | | GAGAAGGGAGAGAGTGGGAGGTTTCACTTCCT | |
| | | GACCCTCCTCTCTTCTTTCTGCAGTCGCTTCCTG | |
| | | CCTCGGTAAATAAATAAggctccggtgcccgtcagtgggcag | |
| | | agcgcacatcgcccacagtccccgagaagttggggggaggggtcggcaatt | |
| | | gatccggtgcctagagaaggtggcgcggggtaaactgggaaagtgatgtcgt | |
| | | gtactggctccgcctttttcccgagggtgggggagaaccgtatataagtgcagt | |
| | | agtcgccgtgaacgttctttttcgcaacgggtttgccgccagaacacaggtaag | |
| | | tgccgtgtgtggttcccgcgggcctggcctctttacgggttatggcccttgcgtg | |
| | | ccttgaattacttccacctggctgcagtacgtgattcttgatcccgagcttcgggt | |
| | | tggaagtgggtgggagagttcgaggccttgcgcttaaggagccccttcgcctc | |
| | | gtgcttgagttgaggcctggcctgggcgctggggccgccgcgtgcgaatctg | |
| | | gtggcaccttcgcgcctgtctcgctgctttcgataagtctctagccatttaaaattt | |
| | | ttgatgacctgctgcgacgctttttttctggcaagatagtcttgtaaatgcgggcc | |
| | | aagatctgcacactggtatttcggtttttggggccgcgggcggcgacggggcc | |
| | | cgtgcgtcccagcgcacatgttcggcgaggcggggcctgcgagcgcggcc | |
| | | accgagaatcggacgggggtagtctcaagctggccggcctgctctggtgcct | |
| | | ggcctcgcgccgccgtgtatcgccccgccctgggggcaaggctggcccg | |
| | | gtcggcaccagttgcgtgagcggaaagatggccgcttcccggccctgctgca | |
| | | gggagctcaaaatggaggacgcggcgctcgggagagcgggcgggtgagt | |
| | | cacccacacaaaggaaaagggcctttccgtcctcagccgtcgcttcatgtgac | |
| | | tccacggagtaccgggcgccgtccaggcacctcgattagttctcgagcttttgg | |
| | | agtacgtcgtctttaggttggggggaggggttttatgcgatggagtttccccaca | |
| | | ctgagtgggtggagactgaagttaggccagcttggcacttgatgtaattctcctt | |
| | | ggaatttgccctttttgagtttggatcttggttcattctcaagcctcagacagtggtt | |
| | | caaagtttttttcttccatttcaggtgtcgtgacgtacgGAATTCGACGC | |
| | | CACCATGGAGTTCGGCCTGAGCTGGCTGTTCCT | |
| | | GGTGGCCATCCTGAAGGGCGTGCAGTGCATCGA | |
| | | CGCCATGGGCAACATCCAGCTGGTGCAGAGCG | |
| | | GCCCCGAGCTGAAGAAGCCCGGCGAGACCGTG | |
| | | AAGATCAGCTGCAAGGCCAGCGGCTACACCTTC | |
| | | ACCAACTACGGCATGAACTGGGTGAAGCAGGC | |
| | | CCCCGGCAAGGGCCTGAGGTGGATGGGCTGGA | |
| | | TCAACACCCACACCGGCGAGCCCACCTACGCCG | |
| | | ACGACTTCAAGGGCAGGTTCGCCTTCAGCCTGG | |
| | | AGACCAGCGCCAGCACCGCCTACCTGCAGATCA | |
| | | ACAACCTGAAGAACGAGGACACCGCCACCTAC | |
| | | TTCTGCACCAGGAGGGGCTACGACTGGTACTTC | |
| | | GACGTGTGGGGCGCCGGCACCACCGTGACCGT | |
| | | GAGCAGCGGCGGCGGCGGCAGCGGCGGCGGCG | |
| | | GCAGCGGCGGCGGCGGCAGCGACATCAAGATG | |
| | | ACCCAGAGCCCCAGCAGCATGTACGCCAGCCTG | |
| | | GGCGAGAGGGTGACCATCACCTGCAAGGCCAG | |
| | | CCAGGACATCAACAGCTACCTGAGCTGGTTCCA | |
| | | CCACAAGCCCGGCAAGAGCCCCAAGACCCTGA | |
| | | TCTACAGGGCCAACAGGCTGGTGGACGGCGTG | |
| | | CCCAGCAGGTTCAGCGGCAGCGGCAGCGGCCA | |
| | | GGACTACAGCCTGACCATCAGCAGCCTGGACTA | |
| | | CGAGGACATGGGCATCTACTACTGCCAGCAGTA | |
| | | CGACGAGAGCCCCTGGACCTTCGGCGGCGGCA | |
| | | CCAAGCTGGAGATGAAGGGCAGCGGCGACCCC | |
| | | GCCGAGCCCAAGAGCCCCGACAAGACCCACAC | |
| | | CTGCCCCCCCTGCCCCGCCCCCGAGCTGCTGGG | |
| | | CGGCCCCAGCGTGTTCCTGTTCCCCCCCAAGCC | |
| | | CAAGGACACCCTGATGATCAGCAGGACCCCCG | |
| | | AGGTGACCTGCGTGGTGGTGGACGTGAGCCAC | |
| | | GAGGACCCCGAGGTGAAGTTCAACTGGTACGT | |
| | | GGACGGCGTGGAGGTGCACAACGCCAAGACCA | |
| | | AGCCCAGGGAGGAGCAGTACAACAGCACCTAC | |
| | | AGGGTGGTGAGCGTGCTGACCGTGCTGCACCAG | |
| | | GACTGGCTGAACGGCAAGGAGTACAAGTGCAA | |
| | | GGTGAGCAACAAGGCCCTGCCCGCCCCCATCGA | |
| | | GAAGACCATCAGCAAGGCCAAGGGCCAGCCCA | |
| | | GGGAGCCCCAGGTGTACACCCTGCCCCCCAGCA | |
| | | GGGACGAGCTGACCAAGAACCAGGTGAGCCTG | |
| | | ACCTGCCTGGTGAAGGGCTTCTACCCCAGCGAC | |
| | | ATCGCCGTGGAGTGGGAGAGCAACGGCCAGCC | |
| | | CGAGAACAACTACAAGACCACCCCCCCCGTGCT | |
| | | GGACAGCGACGGCAGCTTCTTCCTGTACAGCAA | |
| | | GCTGACCGTGGACAAGAGCAGGTGGCAGCAGG | |
| | | GCAACGTGTTCAGCTGCAGCGTGATGCACGAGG | |
| | | CCCTGCACAACCACTACACCCAGAAGAGCCTGA | |
| | | GCCTGAGCCCCGGCAAGAAGGACCCCAAGTTCT | |
| | | GGGTGCTGGTGGTGGTGGGCGGCGTGCTGGCCT | |
| | | GCTACAGCCTGCTGGTGACCGTGGCCTTCATCA | |
| | | TCTTCTGGGTGAGGAGCAAGAGGAGCAGGCTG | |
| | | CTGCACAGCGACTACATGAACATGACCCCCAGG | |
| | | AGGCCCGGCCCCACCAGGAAGCACTACCAGCC | |
| | | CTACGCCCCCCCCAGGGACTTCGCCGCCTACAG | |
| | | GAGCAGGGTGAAGTTCAGCAGGAGCGCCGACG | |
| | | CCCCCGCCTACCAGCAGGGCCAGAACCAGCTGT | |
| | | ACAACGAGCTGAACCTGGGCAGGAGGGAGGAG | |
| | | TACGACGTGCTGGACAAGAGGAGGGGCAGGGA | |
| | | CCCCGAGATGGGCGGCAAGCCCAGGAGGAAGA | |
| | | ACCCCCAGGAGGGCCTGTACAACGAGCTGCAG | |
| | | AAGGACAAGATGGCCGAGGCCTACAGCGAGAT | |
| | | CGGCATGAAGGGCGAGAGGAGGAGGGGCAAG | |
| | | GGCCACGACGGCCTGTACCAGGGCCTGAGCAC | |
| | | CGCCACCAAGGACACCTACGACGCCCTGCACAT | |
| | | GCAGGCCCTGCCCCCCAGGGCCACGAACTTCTC | |
| | | TCTGTTAAAGCAAGCAGGAGACGTGGAAGAAA | |
| | | ACCCCGGTCCTATGGTGAGCAAGGGCGAGGAG | |
| | | CTGTTCACCGGGGTGGTGCCCATCCTGGTCGAG | |
| | | CTGGACGGCGACGTAAACGGCCACAAGTTCAG | |
| | | CGTGTCCGGCGAGGGCGAGGGCGATGCCACCT | |
| | | ACGGCAAGCTGACCCTGAAGTTCATCTGCACCA | |
| | | CCGGCAAGCTGCCCGTGCCCTGGCCCACCCTCG | |
| | | TGACCACCCTGACCTACGGCGTGCAGTGCTTCA | |
| | | GCCGCTACCCCGACCACATGAAGCAGCACGACT | |
| | | TCTTCAAGTCCGCCATGCCCGAAGGCTACGTCC | |
| | | AGGAGCGCACCATCTTCTTCAAGGACGACGGCA | |
| | | ACTACAAGACCCGCGCCGAGGTGAAGTTCGAG | |
| | | GGCGACACCCTGGTGAACCGCATCGAGCTGAA | |
| | | GGGCATCGACTTCAAGGAGGACGGCAACATCC | |
| | | TGGGGCACAAGCTGGAGTACAACTACAACAGC | |
| | | CACAACGTCTATATCATGGCCGACAAGCAGAA | |
| | | GAACGGCATCAAGGTGAACTTCAAGATCCGCC | |
| | | ACAACATCGAGGACGGCAGCGTGCAGCTCGCC | |
| | | GACCACTACCAGCAGAACACCCCCATCGGCGA | |
| | | CGGCCCCGTGCTGCTGCCCGACAACCACTACCT | |
| | | GAGCACCCAGTCCGCCCTGAGCAAAGACCCCA | |
| | | ACGAGAAGCGCGATCACATGGTCCTGCTGGAGT | |
| | | TCGTGACCGCCGCCGGGATCACTCTCGGCATGG | |
| | | ACGAGCTGTACAAGTAAAATGAATGCAATTGTT | |
| | | GTTGTTAATAAAGGAAATTTATTTTCATTGCAA | |
| | | TAGTGTGTTGGAATTTTTTGTGTCTCTCAACGGC | |
| | | TCAGCTGGTATGACCCAGGTAAGGAAGAGCCA | |
| | | CATGGAGAAAGGCCTGGGGCAGGGGGAGAGTG | |
| | | GGGCTGTGGTTTCATCAGGCCATCGGGGACCTC | |
| | | TCGATGAAGCCATCACTTCTGCCAGAGTGAACC | |
| | | CCACCCTATAGAGAGAGTGAACCCCAGCATAC | |
| | | ACACAGGCACATAGATGCAGACACTGCACATT | |
| | | AAGATGCTCACATGCAGGTGGGTGCCCTCGACA | |
| | | GCCGTAAATCACCCACAAATGCCAGATCTCATG | |
| | | ATAATTATTATGACCCGCTCACCATGCACAGAA | |
| | | GACATCCCAGCTCATAAATGTACCTTGCAAAGT | |
| | | CTTATTTCCCACCCAATCCTGACAGATGCTCCAT | |
| | | GGTCAAAGATGTTTAGAGCGGAGTCTGCAGAG | |
| | | AGAGGCCGCAGACTGATGGTAAAGTGTGTGGA | |
| | | ACGTCCAGCCTTAGACGTTGGAGTTTAGTCGTA | |
| | | GAGGCTGTTTCCCAAATAGGGTTCCATGGAGCA | |
| | | TGTTG | |
| CT14 | 500bpHA- | GCACTCATGCCAGGAGCTCCTGGTCCTCTCAAG | 115 |
| | g3-CD5- | GCTGCTGGCTGCCCCCGGCCCTCCCCACACCAC | |
| | CAR- | CCATTCCTCCCTCACCAGAGTGTCTCATTGCAG | |
| | P2A- | AACCCCAGAAGACAACACCTCCAACGACAAGG | |
| | EGFP | CCCCCGCCCACCACAACTCCAGAGCCCACAGGT | |
| | | AAGAGGATTCTGAACCCCCCACAGGGAGTCAG | |
| | | AGCTAGCAAATAAAAACCCAGGATGCCCAGTT | |
| | | ACATTGGAATTTCTGACAAAGGTGGAAATGTTT | |
| | | AGTATTGGTGTGTTCTACGCAATATTTGGGACC | |
| | | CCATCACCTCCCAAGGCTAAGCGTTAGTCAGTA | |
| | | GTTGTCCACAAGTTGGGGCCAAACAGCAAGGA | |
| | | GTGCCCAGGAAGCCCTCGGCGCTCAGGGTGGCT | |
| | | CCCCCTCCTGCTCTCTCCTCTCCTAGCTCCTCCC | |
| | | AGGCTGCAGCTGGTGGCACAGTCTGGCGGCCA | |
| | | GCACTGTGCCGGCGTGGTGGAGTTCTACAGCGG | |
| | | CAGCCTGGGTAAATAAATAAggctccggtgcccgtcagtg | |
| | | ggcagagcgcacatcgcccacagtccccgagaagttggggggaggggtcg | |
| | | gcaattgatccggtgcctagagaaggtggcgcggggtaaactgggaaagtg | |
| | | atgtcgtgtactggctccgcctttttcccgaggggggggagaaccgtatataa | |
| | | gtgcagtagtcgccgtgaacgttctttttcgcaacgggtttgccgccagaacac | |
| | | aggtaagtgccgtgtgtggttcccgcgggcctggcctctttacgggttatggcc | |
| | | cttgcgtgccttgaattacttccacctggctgcagtacgtgattcttgatcccgag | |
| | | cttcgggttggaagtgggtgggagagttcgaggccttgcgcttaaggagccc | |
| | | cttcgcctcgtgcttgagttgaggcctggcctgggcgctggggccgccgcgt | |
| | | gcgaatctggtggcaccttcgcgcctgtctcgctgctttcgataagtctctagcc | |
| | | atttaaaatttttgatgacctgctgcgacgctttttttctggcaagatagtcttgtaa | |
| | | atgcgggccaagatctgcacactggtatttcggtttttggggccgcgggcggc | |
| | | gacggggcccgtgcgtcccagcgcacatgttcggcgaggcggggcctgcg | |
| | | agcgcggccaccgagaatcggacgggggtagtctcaagctggccggcctg | |
| | | ctctggtgcctggcctcgcgccgccgtgtatcgccccgccctgggcggcaag | |
| | | gctggcccggtcggcaccagttgcgtgagcggaaagatggccgcttcccgg | |
| | | ccctgctgcagggagctcaaaatggaggacgcggcgctcgggagagcggg | |
| | | cgggtgagtcacccacacaaaggaaaagggcctttccgtcctcagccgtcgc | |
| | | ttcatgtgactccacggagtaccgggcgccgtccaggcacctcgattagttctc | |
| | | gagcttttggagtacgtcgtctttaggttggggggaggggttttatgcgatgga | |
| | | gtttccccacactgagtgggtggagactgaagttaggccagcttggcacttgat | |
| | | gtaattctccttggaatttgccctttttgagtttggatcttggttcattctcaagcctc | |
| | | agacagtggttcaaagtttttttcttccatttcaggtgtcgtgacgtacgGAAT | |
| | | TCGACGCCACCATGGAGTTCGGCCTGAGCTGGC | |
| | | TGTTCCTGGTGGCCATCCTGAAGGGCGTGCAGT | |
| | | GCATCGACGCCATGGGCAACATCCAGCTGGTGC | |
| | | AGAGCGGCCCCGAGCTGAAGAAGCCCGGCGAG | |
| | | ACCGTGAAGATCAGCTGCAAGGCCAGCGGCTA | |
| | | CACCTTCACCAACTACGGCATGAACTGGGTGAA | |
| | | GCAGGCCCCCGGCAAGGGCCTGAGGTGGATGG | |
| | | GCTGGATCAACACCCACACCGGCGAGCCCACCT | |
| | | ACGCCGACGACTTCAAGGGCAGGTTCGCCTTCA | |
| | | GCCTGGAGACCAGCGCCAGCACCGCCTACCTGC | |
| | | AGATCAACAACCTGAAGAACGAGGACACCGCC | |
| | | ACCTACTTCTGCACCAGGAGGGGCTACGACTGG | |
| | | TACTTCGACGTGTGGGGCGCCGGCACCACCGTG | |
| | | ACCGTGAGCAGCGGCGGCGGCGGCAGCGGCGG | |
| | | CGGCGGCAGCGGCGGCGGCGGCAGCGACATCA | |
| | | AGATGACCCAGAGCCCCAGCAGCATGTACGCC | |
| | | AGCCTGGGCGAGAGGGTGACCATCACCTGCAA | |
| | | GGCCAGCCAGGACATCAACAGCTACCTGAGCT | |
| | | GGTTCCACCACAAGCCCGGCAAGAGCCCCAAG | |
| | | ACCCTGATCTACAGGGCCAACAGGCTGGTGGAC | |
| | | GGCGTGCCCAGCAGGTTCAGCGGCAGCGGCAG | |
| | | CGGCCAGGACTACAGCCTGACCATCAGCAGCCT | |
| | | GGACTACGAGGACATGGGCATCTACTACTGCCA | |
| | | GCAGTACGACGAGAGCCCCTGGACCTTCGGCG | |
| | | GCGGCACCAAGCTGGAGATGAAGGGCAGCGGC | |
| | | GACCCCGCCGAGCCCAAGAGCCCCGACAAGAC | |
| | | CCACACCTGCCCCCCCTGCCCCGCCCCCGAGCT | |
| | | GCTGGGCGGCCCCAGCGTGTTCCTGTTCCCCCC | |
| | | CAAGCCCAAGGACACCCTGATGATCAGCAGGA | |
| | | CCCCCGAGGTGACCTGCGTGGTGGTGGACGTGA | |
| | | GCCACGAGGACCCCGAGGTGAAGTTCAACTGG | |
| | | TACGTGGACGGCGTGGAGGTGCACAACGCCAA | |
| | | GACCAAGCCCAGGGAGGAGCAGTACAACAGCA | |
| | | CCTACAGGGTGGTGAGCGTGCTGACCGTGCTGC | |
| | | ACCAGGACTGGCTGAACGGCAAGGAGTACAAG | |
| | | TGCAAGGTGAGCAACAAGGCCCTGCCCGCCCCC | |
| | | ATCGAGAAGACCATCAGCAAGGCCAAGGGCCA | |
| | | GCCCAGGGAGCCCCAGGTGTACACCCTGCCCCC | |
| | | CAGCAGGGACGAGCTGACCAAGAACCAGGTGA | |
| | | GCCTGACCTGCCTGGTGAAGGGCTTCTACCCCA | |
| | | GCGACATCGCCGTGGAGTGGGAGAGCAACGGC | |
| | | CAGCCCGAGAACAACTACAAGACCACCCCCCC | |
| | | CGTGCTGGACAGCGACGGCAGCTTCTTCCTGTA | |
| | | CAGCAAGCTGACCGTGGACAAGAGCAGGTGGC | |
| | | AGCAGGGCAACGTGTTCAGCTGCAGCGTGATGC | |
| | | ACGAGGCCCTGCACAACCACTACACCCAGAAG | |
| | | AGCCTGAGCCTGAGCCCCGGCAAGAAGGACCC | |
| | | CAAGTTCTGGGTGCTGGTGGTGGTGGGCGGCGT | |
| | | GCTGGCCTGCTACAGCCTGCTGGTGACCGTGGC | |
| | | CTTCATCATCTTCTGGGTGAGGAGCAAGAGGAG | |
| | | CAGGCTGCTGCACAGCGACTACATGAACATGAC | |
| | | CCCCAGGAGGCCCGGCCCCACCAGGAAGCACT | |
| | | ACCAGCCCTACGCCCCCCCCAGGGACTTCGCCG | |
| | | CCTACAGGAGCAGGGTGAAGTTCAGCAGGAGC | |
| | | GCCGACGCCCCCGCCTACCAGCAGGGCCAGAA | |
| | | CCAGCTGTACAACGAGCTGAACCTGGGCAGGA | |
| | | GGGAGGAGTACGACGTGCTGGACAAGAGGAGG | |
| | | GGCAGGGACCCCGAGATGGGCGGCAAGCCCAG | |
| | | GAGGAAGAACCCCCAGGAGGGCCTGTACAACG | |
| | | AGCTGCAGAAGGACAAGATGGCCGAGGCCTAC | |
| | | AGCGAGATCGGCATGAAGGGCGAGAGGAGGAG | |
| | | GGGCAAGGGCCACGACGGCCTGTACCAGGGCC | |
| | | TGAGCACCGCCACCAAGGACACCTACGACGCC | |
| | | CTGCACATGCAGGCCCTGCCCCCCAGGGCCACG | |
| | | AACTTCTCTCTGTTAAAGCAAGCAGGAGACGTG | |
| | | GAAGAAAACCCCGGTCCTATGGTGAGCAAGGG | |
| | | CGAGGAGCTGTTCACCGGGGTGGTGCCCATCCT | |
| | | GGTCGAGCTGGACGGCGACGTAAACGGCCACA | |
| | | AGTTCAGCGTGTCCGGCGAGGGCGAGGGCGAT | |
| | | GCCACCTACGGCAAGCTGACCCTGAAGTTCATC | |
| | | TGCACCACCGGCAAGCTGCCCGTGCCCTGGCCC | |
| | | ACCCTCGTGACCACCCTGACCTACGGCGTGCAG | |
| | | TGCTTCAGCCGCTACCCCGACCACATGAAGCAG | |
| | | CACGACTTCTTCAAGTCCGCCATGCCCGAAGGC | |
| | | TACGTCCAGGAGCGCACCATCTTCTTCAAGGAC | |
| | | GACGGCAACTACAAGACCCGCGCCGAGGTGAA | |
| | | GTTCGAGGGCGACACCCTGGTGAACCGCATCGA | |
| | | GCTGAAGGGCATCGACTTCAAGGAGGACGGCA | |
| | | ACATCCTGGGGCACAAGCTGGAGTACAACTAC | |
| | | AACAGCCACAACGTCTATATCATGGCCGACAAG | |
| | | CAGAAGAACGGCATCAAGGTGAACTTCAAGAT | |
| | | CCGCCACAACATCGAGGACGGCAGCGTGCAGC | |
| | | TCGCCGACCACTACCAGCAGAACACCCCCATCG | |
| | | GCGACGGCCCCGTGCTGCTGCCCGACAACCACT | |
| | | ACCTGAGCACCCAGTCCGCCCTGAGCAAAGACC | |
| | | CCAACGAGAAGCGCGATCACATGGTCCTGCTGG | |
| | | AGTTCGTGACCGCCGCCGGGATCACTCTCGGCA | |
| | | TGGACGAGCTGTACAAGTAAAATGAATGCAATT | |
| | | GTTGTTGTTAATAAAGGAAATTTATTTTCATTGC | |
| | | AATAGTGTGTTGGAATTTTTTGTGTCTCTCAGGG | |
| | | TACCATCAGCTATGAGGCCCAGGACAAGACCC | |
| | | AGGACCTGGAGAACTTCCTCTGCAACAACCTCC | |
| | | AGTGTGGCTCCTTCTTGAAGCATCTGCCAGAGA | |
| | | CTGAGGCAGGCAGAGCCCAAGACCCAGGGGAG | |
| | | CCACGGGAACACCAGCCCTTGCCAATCCAATGG | |
| | | AAGATCCAGAACTCAAGCTGTACCTCCCTGGAG | |
| | | CATTGCTTCAGGAAAATCAAGCCCCAGAAAAGT | |
| | | GGCCGAGTTCTTGCCCTCCTTTGCTCAGGTAAG | |
| | | TGAGACCTGGCCAAGCCCCATGACACCTTCTGC | |
| | | TGCCCTAGGTGGGGTCACAGAGCATCCCAGAA | |
| | | GGTCAGGGAACATGTGTGCAGCACAGGGCACT | |
| | | ATGGAGAATACAAGGGAAGTGGAGGCCTGGTC | |
| | | TTGGCCTCTAAGAGGTAACAAGGGTTGGGGTGG | |
| | | GGAGGATGCATCCACACTCAATGCCTTGGTAAT | |
| | | CTCTGCAAAGCTACACACCCCAAGCCCAAAGG | |
| | | AACCGCTG | |
| CT15 | 500BpHA- | ATGTGATAGATTTCCCAACTTAATGCCAACATA | 116 |
| | SG17- | CCATAAACCTCCCATTCTGCTAATGCCCAGCCT | |
| | TRAC- | AAGTTGGGGAGACCACTCCAGATTCCAAGATGT | |
| | SSFV- | ACAGTTTGCTTTGCTGGGCCTTTTTCCCATGCCT | |
| | EGFP | GCCTTTACTCTGCCAGAGTTATATTGCTGGGGTT | |
| | | TTGAAGAAGATCCTATTAAATAAAAGAATAAG | |
| | | CAGTATTATTAAGTAGCCCTGCATTTCAGGTTTC | |
| | | CTTGAGTGGCAGGCCAGGCCTGGCCGTGAACGT | |
| | | TCACTGAAATCATGGCCTCTTGGCCAAGATTGA | |
| | | TAGCTTGTGCCTGTCCCTGAGTCCCAGTCCATC | |
| | | ACGAGCAGCTGGTTTCTAAGATGCTATTTCCCG | |
| | | TATAAAGCATGAGACCGTGACTTGCCAGCCCCA | |
| | | CAGAGCCCCGCCCTTGTCCATCACTGGCATCTG | |
| | | GACTCCAGCCTGGGTTGGGGCAAAGAGGGAAA | |
| | | TGAGATCATGTCCTAACCCTGATCCTCTTGTCCC | |
| | | ACAGTAACGCCATTTTGCAAGGCATGGAAAAAT | |
| | | ACCAAACCAAGAATAGAGAAGTTCAGATCAAG | |
| | | GGCGGGTACATGAAAATAGCTAACGTTGGGCC | |
| | | AAACAGGATATCTGCGGTGAGCAGTTTCGGCCC | |
| | | CGGCCCGGGGCCAAGAACAGATGGTCACCGCA | |
| | | GTTTCGGCCCCGGCCCGAGGCCAAGAACAGAT | |
| | | GGTCCCCAGATATGGCCCAACCCTCAGCAGTTT | |
| | | CTTAAGACCCATCAGATGTTTCCAGGCTCCCCC | |
| | | AAGGACCTGAAATGACCCTGCGCCTTATTTGAA | |
| | | TTAACCAATCAGCCTGCTTCTCGCTTCTGTTCGC | |
| | | GCGCTTCTGCTTCCCGAGCTCTATAAAAGAGCT | |
| | | CACAACCCCTCACTCGGCGCGCCAGTCCTCCGA | |
| | | CAGACTGAGTCGCCCGGGCCGCGGCCGCGGGC | |
| | | TAGCGGATCCCCACCGGTCGCCACCATGGTGAG | |
| | | CAAGGGCGAGGAGCTGTTCACCGGGGTGGTGC | |
| | | CCATCCTGGTCGAGCTGGACGGCGACGTAAACG | |
| | | GCCACAAGTTCAGCGTGTCCGGCGAGGGCGAG | |
| | | GGCGATGCCACCTACGGCAAGCTGACCCTGAA | |
| | | GTTCATCTGCACCACCGGCAAGCTGCCCGTGCC | |
| | | CTGGCCCACCCTCGTGACCACCCTGACCTACGG | |
| | | CGTGCAGTGCTTCAGCCGCTACCCCGACCACAT | |
| | | GAAGCAGCACGACTTCTTCAAGTCCGCCATGCC | |
| | | CGAAGGCTACGTCCAGGAGCGCACCATCTTCTT | |
| | | CAAGGACGACGGCAACTACAAGACCCGCGCCG | |
| | | AGGTGAAGTTCGAGGGCGACACCCTGGTGAAC | |
| | | CGCATCGAGCTGAAGGGCATCGACTTCAAGGA | |
| | | GGACGGCAACATCCTGGGGCACAAGCTGGAGT | |
| | | ACAACTACAACAGCCACAACGTCTATATCATGG | |
| | | CCGACAAGCAGAAGAACGGCATCAAGGTGAAC | |
| | | TTCAAGATCCGCCACAACATCGAGGACGGCAG | |
| | | CGTGCAGCTCGCCGACCACTACCAGCAGAACAC | |
| | | CCCCATCGGCGACGGCCCCGTGCTGCTGCCCGA | |
| | | CAACCACTACCTGAGCACCCAGTCCGCCCTGAG | |
| | | CAAAGACCCCAACGAGAAGCGCGATCACATGG | |
| | | TCCTGCTGGAGTTCGTGACCGCCGCCGGGATCA | |
| | | CTCTCGGCATGGACGAGCTGTACAAGTAAAATG | |
| | | AATGCAATTGTTGTTGTTAATAAAGGAAATTTA | |
| | | TTTTCATTGCAATAGTGTGTTGGAATTTTTTGTG | |
| | | TCTCTCAGATATCCAGAACCCTGACCCTGCCGT | |
| | | GTACCAGCTGAGAGACTCTAAATCCAGTGACAA | |
| | | GTCTGTCTGCCTATTCACCGATTTTGATTCTCAA | |
| | | ACAAATGTGTCACAAAGTAAGGATTCTGATGTG | |
| | | TATATCACAGACAAAACTGTGCTAGACATGAGG | |
| | | TCTATGGACTTCAAGAGCAACAGTGCTGTGGCC | |
| | | TGGAGCAACAAATCTGACTTTGCATGTGCAAAC | |
| | | GCCTTCAACAACAGCATTATTCCAGAAGACACC | |
| | | TTCTTCCCCAGCCCAGGTAAGGGCAGCTTTGGT | |
| | | GCCTTCGCAGGCTGTTTCCTTGCTTCAGGAATG | |
| | | GCCAGGTTCTGCCCAGAGCTCTGGTCAATGATG | |
| | | TCTAAAACTCCTCTGATTGGTGGTCTCGGCCTTA | |
| | | TCCATTGCCACCAAAACCCTCTTTTTACTAAGA | |
| | | AACAGTGAGCCTTGTTCTGGCAGTCCAGAGAAT | |
| | | GACACGGGAAAAAAGCAGATGAAGAGAAGGTG | |
| | | GCAGGAGAGGG | |
| CT16 | 500BpHA- | ATGTGATAGATTTCCCAACTTAATGCCAACATA | 117 |
| | SG17- | CCATAAACCTCCCATTCTGCTAATGCCCAGCCT | |
| | TRAC- | AAGTTGGGGAGACCACTCCAGATTCCAAGATGT | |
| | Ef1a- | ACAGTTTGCTTTGCTGGGCCTTTTTCCCATGCCT | |
| | EGFP | GCCTTTACTCTGCCAGAGTTATATTGCTGGGGTT | |
| | | TTGAAGAAGATCCTATTAAATAAAAGAATAAG | |
| | | CAGTATTATTAAGTAGCCCTGCATTTCAGGTTTC | |
| | | CTTGAGTGGCAGGCCAGGCCTGGCCGTGAACGT | |
| | | TCACTGAAATCATGGCCTCTTGGCCAAGATTGA | |
| | | TAGCTTGTGCCTGTCCCTGAGTCCCAGTCCATC | |
| | | ACGAGCAGCTGGTTTCTAAGATGCTATTTCCCG | |
| | | TATAAAGCATGAGACCGTGACTTGCCAGCCCCA | |
| | | CAGAGCCCCGCCCTTGTCCATCACTGGCATCTG | |
| | | GACTCCAGCCTGGGTTGGGGCAAAGAGGGAAA | |
| | | TGAGATCATGTCCTAACCCTGATCCTCTTGTCCC | |
| | | ACAggctccggtgcccgtcagtgggcagagcgcacatcgcccacagtcc | |
| | | ccgagaagttggggggaggggtcggcaattgaaccggtgcctagagaaggt | |
| | | ggcgcggggtaaactgggaaagtgatgtcgtgtactggctccgcctttttcccg | |
| | | agggtgggggagaaccgtatataagtgcagtagtcgccgtgaacgttctttttc | |
| | | gcaacgggtttgccgccagaacacaggtaagtgccgtgtgtggttcccgcgg | |
| | | gcctggcctctttacgggttatggcccttgcgtgccttgaattacttccacctggc | |
| | | tgcagtacgtgattcttgatcccgagcttcgggttggaagtgggtgggagagtt | |
| | | cgaggccttgcgcttaaggagccccttcgcctcgtgcttgagttgaggcctgg | |
| | | cctgggcgctggggccgccgcgtgcgaatctggtggcaccttcgcgcctgtc | |
| | | tcgctgctttcgataagtctctagccatttaaaatttttgatgacctgctgcgacgc | |
| | | tttttttctggcaagatagtcttgtaaatgcgggccaagatctgcacactggtattt | |
| | | cggtttttggggccgcgggcggcgacggggcccgtgcgtcccagcgcacat | |
| | | gttcggcgaggcggggcctgcgagcgcggccaccgagaatcggacgggg | |
| | | gtagtctcaagctggccggcctgctctggtgcctggcctcgcgccgccgtgta | |
| | | tcgccccgccctgggcggcaaggctggcccggtcggcaccagttgcgtgag | |
| | | cggaaagatggccgcttcccggccctgctgcagggagctcaaaatggagga | |
| | | cgcggcgctcgggagagcgggcgggtgagtcacccacacaaaggaaaag | |
| | | ggcctttccgtcctcagccgtcgcttcatgtgactccactgagtaccgggcgcc | |
| | | gtccaggcacctcgattagttctcgtgcttttggagtacgtcgtctttaggttggg | |
| | | gggaggggttttatgcgatggagtttccccacactgagtgggtggagactgaa | |
| | | gttaggccagcttggcacttgatgtaattctccttggaatttgccctttttgagtttg | |
| | | gatcttggttcattctcaagcctcagacagtggttcaaagtttttttcttccatttca | |
| | | ggtgtcgtgaGCCACCATGGTGAGCAAGGGCGAGGA | |
| | | GCTGTTCACCGGGGTGGTGCCCATCCTGGTCGA | |
| | | GCTGGACGGCGACGTAAACGGCCACAAGTTCA | |
| | | GCGTGTCCGGCGAGGGCGAGGGCGATGCCACC | |
| | | TACGGCAAGCTGACCCTGAAGTTCATCTGCACC | |
| | | ACCGGCAAGCTGCCCGTGCCCTGGCCCACCCTC | |
| | | GTGACCACCCTGACCTACGGCGTGCAGTGCTTC | |
| | | AGCCGCTACCCCGACCACATGAAGCAGCACGA | |
| | | CTTCTTCAAGTCCGCCATGCCCGAAGGCTACGT | |
| | | CCAGGAGCGCACCATCTTCTTCAAGGACGACGG | |
| | | CAACTACAAGACCCGCGCCGAGGTGAAGTTCG | |
| | | AGGGCGACACCCTGGTGAACCGCATCGAGCTG | |
| | | AAGGGCATCGACTTCAAGGAGGACGGCAACAT | |
| | | CCTGGGGCACAAGCTGGAGTACAACTACAACA | |
| | | GCCACAACGTCTATATCATGGCCGACAAGCAGA | |
| | | AGAACGGCATCAAGGTGAACTTCAAGATCCGC | |
| | | CACAACATCGAGGACGGCAGCGTGCAGCTCGC | |
| | | CGACCACTACCAGCAGAACACCCCCATCGGCG | |
| | | ACGGCCCCGTGCTGCTGCCCGACAACCACTACC | |
| | | TGAGCACCCAGTCCGCCCTGAGCAAAGACCCCA | |
| | | ACGAGAAGCGCGATCACATGGTCCTGCTGGAGT | |
| | | TCGTGACCGCCGCCGGGATCACTCTCGGCATGG | |
| | | ACGAGCTGTACAAGTAAAATGAATGCAATTGTT | |
| | | GTTGTTAATAAAGGAAATTTATTTTCATTGCAA | |
| | | TAGTGTGTTGGAATTTTTTGTGTCTCTCAGATAT | |
| | | CCAGAACCCTGACCCTGCCGTGTACCAGCTGAG | |
| | | AGACTCTAAATCCAGTGACAAGTCTGTCTGCCT | |
| | | ATTCACCGATTTTGATTCTCAAACAAATGTGTC | |
| | | ACAAAGTAAGGATTCTGATGTGTATATCACAGA | |
| | | CAAAACTGTGCTAGACATGAGGTCTATGGACTT | |
| | | CAAGAGCAACAGTGCTGTGGCCTGGAGCAACA | |
| | | AATCTGACTTTGCATGTGCAAACGCCTTCAACA | |
| | | ACAGCATTATTCCAGAAGACACCTTCTTCCCCA | |
| | | GCCCAGGTAAGGGCAGCTTTGGTGCCTTCGCAG | |
| | | GCTGTTTCCTTGCTTCAGGAATGGCCAGGTTCT | |
| | | GCCCAGAGCTCTGGTCAATGATGTCTAAAACTC | |
| | | CTCTGATTGGTGGTCTCGGCCTTATCCATTGCCA | |
| | | CCAAAACCCTCTTTTTACTAAGAAACAGTGAGC | |
| | | CTTGTTCTGGCAGTCCAGAGAATGACACGGGAA | |
| | | AAAAGCAGATGAAGAGAAGGTGGCAGGAGAG | |
| | | GG | |
| CT17 | 500BpHA- | AACATACCATAAACCTCCCATTCTGCTAATGCC | 118 |
| | SG18- | CAGCCTAAGTTGGGGAGACCACTCCAGATTCCA | |
| | TRAC- | AGATGTACAGTTTGCTTTGCTGGGCCTTTTTCCC | |
| | SSFV- | ATGCCTGCCTTTACTCTGCCAGAGTTATATTGCT | |
| | EGFP | GGGGTTTTGAAGAAGATCCTATTAAATAAAAGA | |
| | | ATAAGCAGTATTATTAAGTAGCCCTGCATTTCA | |
| | | GGTTTCCTTGAGTGGCAGGCCAGGCCTGGCCGT | |
| | | GAACGTTCACTGAAATCATGGCCTCTTGGCCAA | |
| | | GATTGATAGCTTGTGCCTGTCCCTGAGTCCCAG | |
| | | TCCATCACGAGCAGCTGGTTTCTAAGATGCTAT | |
| | | TTCCCGTATAAAGCATGAGACCGTGACTTGCCA | |
| | | GCCCCACAGAGCCCCGCCCTTGTCCATCACTGG | |
| | | CATCTGGACTCCAGCCTGGGTTGGGGCAAAGAG | |
| | | GGAAATGAGATCATGTCCTAACCCTGATCCTCT | |
| | | TGTCCCACAGATATCCAGAACCCTGACCCTGCC | |
| | | GTGGTAACGCCATTTTGCAAGGCATGGAAAAAT | |
| | | ACCAAACCAAGAATAGAGAAGTTCAGATCAAG | |
| | | GGCGGGTACATGAAAATAGCTAACGTTGGGCC | |
| | | AAACAGGATATCTGCGGTGAGCAGTTTCGGCCC | |
| | | CGGCCCGGGGCCAAGAACAGATGGTCACCGCA | |
| | | GTTTCGGCCCCGGCCCGAGGCCAAGAACAGAT | |
| | | GGTCCCCAGATATGGCCCAACCCTCAGCAGTTT | |
| | | CTTAAGACCCATCAGATGTTTCCAGGCTCCCCC | |
| | | AAGGACCTGAAATGACCCTGCGCCTTATTTGAA | |
| | | TTAACCAATCAGCCTGCTTCTCGCTTCTGTTCGC | |
| | | GCGCTTCTGCTTCCCGAGCTCTATAAAAGAGCT | |
| | | CACAACCCCTCACTCGGCGCGCCAGTCCTCCGA | |
| | | CAGACTGAGTCGCCCGGGCCGCGGCCGCGGGC | |
| | | TAGCGGATCCCCACCGGTCGCCACCATGGTGAG | |
| | | CAAGGGCGAGGAGCTGTTCACCGGGGTGGTGC | |
| | | CCATCCTGGTCGAGCTGGACGGCGACGTAAACG | |
| | | GCCACAAGTTCAGCGTGTCCGGCGAGGGCGAG | |
| | | GGCGATGCCACCTACGGCAAGCTGACCCTGAA | |
| | | GTTCATCTGCACCACCGGCAAGCTGCCCGTGCC | |
| | | CTGGCCCACCCTCGTGACCACCCTGACCTACGG | |
| | | CGTGCAGTGCTTCAGCCGCTACCCCGACCACAT | |
| | | GAAGCAGCACGACTTCTTCAAGTCCGCCATGCC | |
| | | CGAAGGCTACGTCCAGGAGCGCACCATCTTCTT | |
| | | CAAGGACGACGGCAACTACAAGACCCGCGCCG | |
| | | AGGTGAAGTTCGAGGGCGACACCCTGGTGAAC | |
| | | CGCATCGAGCTGAAGGGCATCGACTTCAAGGA | |
| | | GGACGGCAACATCCTGGGGCACAAGCTGGAGT | |
| | | ACAACTACAACAGCCACAACGTCTATATCATGG | |
| | | CCGACAAGCAGAAGAACGGCATCAAGGTGAAC | |
| | | TTCAAGATCCGCCACAACATCGAGGACGGCAG | |
| | | CGTGCAGCTCGCCGACCACTACCAGCAGAACAC | |
| | | CCCCATCGGCGACGGCCCCGTGCTGCTGCCCGA | |
| | | CAACCACTACCTGAGCACCCAGTCCGCCCTGAG | |
| | | CAAAGACCCCAACGAGAAGCGCGATCACATGG | |
| | | TCCTGCTGGAGTTCGTGACCGCCGCCGGGATCA | |
| | | CTCTCGGCATGGACGAGCTGTACAAGTAAAATG | |
| | | AATGCAATTGTTGTTGTTAATAAAGGAAATTTA | |
| | | TTTTCATTGCAATAGTGTGTTGGAATTTTTTGTG | |
| | | TCTCTCATACCAGCTGAGAGACTCTAAATCCAG | |
| | | TGACAAGTCTGTCTGCCTATTCACCGATTTTGAT | |
| | | TCTCAAACAAATGTGTCACAAAGTAAGGATTCT | |
| | | GATGTGTATATCACAGACAAAACTGTGCTAGAC | |
| | | ATGAGGTCTATGGACTTCAAGAGCAACAGTGCT | |
| | | GTGGCCTGGAGCAACAAATCTGACTTTGCATGT | |
| | | GCAAACGCCTTCAACAACAGCATTATTCCAGAA | |
| | | GACACCTTCTTCCCCAGCCCAGGTAAGGGCAGC | |
| | | TTTGGTGCCTTCGCAGGCTGTTTCCTTGCTTCAG | |
| | | GAATGGCCAGGTTCTGCCCAGAGCTCTGGTCAA | |
| | | TGATGTCTAAAACTCCTCTGATTGGTGGTCTCG | |
| | | GCCTTATCCATTGCCACCAAAACCCTCTTTTTAC | |
| | | TAAGAAACAGTGAGCCTTGTTCTGGCAGTCCAG | |
| | | AGAATGACACGGGAAAAAAGCAGATGAAGAGA | |
| | | AGGTGGCAGGAGAGGGCACGTGGCCCAGCCTC | |
| | | AGTCTCTCCAA | |
| CT18 | 500BpHA- | AACATACCATAAACCTCCCATTCTGCTAATGCC | 119 |
| | SG18- | CAGCCTAAGTTGGGGAGACCACTCCAGATTCCA | |
| | TRAC- | AGATGTACAGTTTGCTTTGCTGGGCCTTTTTCCC | |
| | Ef1a- | ATGCCTGCCTTTACTCTGCCAGAGTTATATTGCT | |
| | EGFP | GGGGTTTTGAAGAAGATCCTATTAAATAAAAGA | |
| | | ATAAGCAGTATTATTAAGTAGCCCTGCATTTCA | |
| | | GGTTTCCTTGAGTGGCAGGCCAGGCCTGGCCGT | |
| | | GAACGTTCACTGAAATCATGGCCTCTTGGCCAA | |
| | | GATTGATAGCTTGTGCCTGTCCCTGAGTCCCAG | |
| | | TCCATCACGAGCAGCTGGTTTCTAAGATGCTAT | |
| | | TTCCCGTATAAAGCATGAGACCGTGACTTGCCA | |
| | | GCCCCACAGAGCCCCGCCCTTGTCCATCACTGG | |
| | | CATCTGGACTCCAGCCTGGGTTGGGGCAAAGAG | |
| | | GGAAATGAGATCATGTCCTAACCCTGATCCTCT | |
| | | TGTCCCACAGATATCCAGAACCCTGACCCTGCC | |
| | | GTGggctccggtgcccgtcagtgggcagagcgcacatcgcccacagtcc | |
| | | ccgagaagttggggggaggggtcggcaattgaaccggtgcctagagaaggt | |
| | | ggcgcggggtaaactgggaaagtgatgtcgtgtactggctccgcctttttcccg | |
| | | agggtgggggagaaccgtatataagtgcagtagtcgccgtgaacgttctttttc | |
| | | gcaacgggtttgccgccagaacacaggtaagtgccgtgtgtggttcccgcgg | |
| | | gcctggcctctttacgggttatggcccttgcgtgccttgaattacttccacctggc | |
| | | tgcagtacgtgattcttgatcccgagcttcgggttggaagtgggtgggagagtt | |
| | | cgaggccttgcgcttaaggagccccttcgcctcgtgcttgagttgaggcctgg | |
| | | cctgggcgctggggccgccgcgtgcgaatctggtggcaccttcgcgcctgtc | |
| | | tcgctgctttcgataagtctctagccatttaaaatttttgatgacctgctgcgacgc | |
| | | tttttttctggcaagatagtcttgtaaatgcgggccaagatctgcacactggtattt | |
| | | cggtttttggggccgcgggcggcgacggggcccgtgcgtcccagcgcacat | |
| | | gttcggcgaggcggggcctgcgagcgcggccaccgagaatcggacgggg | |
| | | gtagtctcaagctggccggcctgctctggtgcctggcctcgcgccgccgtgta | |
| | | tcgccccgccctgggcggcaaggctggcccggtcggcaccagttgcgtgag | |
| | | cggaaagatggccgcttcccggccctgctgcagggagctcaaaatggagga | |
| | | cgcggcgctcgggagagcgggcgggtgagtcacccacacaaaggaaaag | |
| | | ggcctttccgtcctcagccgtcgcttcatgtgactccactgagtaccggggcc | |
| | | gtccaggcacctcgattagttctcgtgcttttggagtacgtcgtctttaggttggg | |
| | | gggaggggttttatgcgatggagtttccccacactgagtgggtggagactgaa | |
| | | gttaggccagcttggcacttgatgtaattctccttggaatttgccctttttgagtttg | |
| | | gatcttggttcattctcaagcctcagacagtggttcaaagtttttttcttccatttca | |
| | | ggtgtcgtgaGCCACCATGGTGAGCAAGGGCGAGGA | |
| | | GCTGTTCACCGGGGTGGTGCCCATCCTGGTCGA | |
| | | GCTGGACGGCGACGTAAACGGCCACAAGTTCA | |
| | | GCGTGTCCGGCGAGGGCGAGGGCGATGCCACC | |
| | | TACGGCAAGCTGACCCTGAAGTTCATCTGCACC | |
| | | ACCGGCAAGCTGCCCGTGCCCTGGCCCACCCTC | |
| | | GTGACCACCCTGACCTACGGCGTGCAGTGCTTC | |
| | | AGCCGCTACCCCGACCACATGAAGCAGCACGA | |
| | | CTTCTTCAAGTCCGCCATGCCCGAAGGCTACGT | |
| | | CCAGGAGCGCACCATCTTCTTCAAGGACGACGG | |
| | | CAACTACAAGACCCGCGCCGAGGTGAAGTTCG | |
| | | AGGGCGACACCCTGGTGAACCGCATCGAGCTG | |
| | | AAGGGCATCGACTTCAAGGAGGACGGCAACAT | |
| | | CCTGGGGCACAAGCTGGAGTACAACTACAACA | |
| | | GCCACAACGTCTATATCATGGCCGACAAGCAGA | |
| | | AGAACGGCATCAAGGTGAACTTCAAGATCCGC | |
| | | CACAACATCGAGGACGGCAGCGTGCAGCTCGC | |
| | | CGACCACTACCAGCAGAACACCCCCATCGGCG | |
| | | ACGGCCCCGTGCTGCTGCCCGACAACCACTACC | |
| | | TGAGCACCCAGTCCGCCCTGAGCAAAGACCCCA | |
| | | ACGAGAAGCGCGATCACATGGTCCTGCTGGAGT | |
| | | TCGTGACCGCCGCCGGGATCACTCTCGGCATGG | |
| | | ACGAGCTGTACAAGTAAAATGAATGCAATTGTT | |
| | | GTTGTTAATAAAGGAAATTTATTTTCATTGCAA | |
| | | TAGTGTGTTGGAATTTTTTGTGTCTCTCATACCA | |
| | | GCTGAGAGACTCTAAATCCAGTGACAAGTCTGT | |
| | | CTGCCTATTCACCGATTTTGATTCTCAAACAAAT | |
| | | GTGTCACAAAGTAAGGATTCTGATGTGTATATC | |
| | | ACAGACAAAACTGTGCTAGACATGAGGTCTATG | |
| | | GACTTCAAGAGCAACAGTGCTGTGGCCTGGAGC | |
| | | AACAAATCTGACTTTGCATGTGCAAACGCCTTC | |
| | | AACAACAGCATTATTCCAGAAGACACCTTCTTC | |
| | | CCCAGCCCAGGTAAGGGCAGCTTTGGTGCCTTC | |
| | | GCAGGCTGTTTCCTTGCTTCAGGAATGGCCAGG | |
| | | TTCTGCCCAGAGCTCTGGTCAATGATGTCTAAA | |
| | | ACTCCTCTGATTGGTGGTCTCGGCCTTATCCATT | |
| | | GCCACCAAAACCCTCTTTTTACTAAGAAACAGT | |
| | | GAGCCTTGTTCTGGCAGTCCAGAGAATGACACG | |
| | | GGAAAAAAGCAGATGAAGAGAAGGTGGCAGG | |
| | | AGAGGGCACGTGGCCCAGCCTCAGTCTCTCCAA | |
| AAV6 | sg089 | GCCAGTAGCCAGCCCCGTCC | 94 |
| | (AAVS1-T) | | |
| pAV1 | AAV6- | CAGTCTGGTCTATCTGCCTGGCCCTGGCCATTGT | 120 |
| | 01- | CACTTTGCGCTGCCCTCCTCTCGCCCCCGAGTGC | |
| | SG16- | CCTTGCTGTGCCGCCGGAACTCTGCCCTCTAAC | |
| | AAVS1- | GCTGCCGTCTCTCTCCTGAGTCCGGACCACTTTG | |
| | SFFV- | AGCTCTACTGGCTTCTGCGCCGCCTCTGGCCCA | |
| | EGFP-B | CTGTTTCCCCTTCCCAGGCAGGTCCTGCTTTCTC | |
| | | TGACCTGCATTCTCTCCCCTGGGCCTGTGCCGCT | |
| | | TTCTGTCTGCAGCTTGTGGCCTGGGTCACCTCTA | |
| | | CGGCTGGCCCAGATCCTTCCCTGCCGCCTCCTTC | |
| | | AGGTTCCGTCTTCCTCCACTCCCTCTTCCCCTTG | |
| | | CTCTCTGCTGTGTTGCTGCCCAAGGATGCTCTTT | |
| | | CCGGAGCACTTCCTTCTCGGCGCTGCACCACGT | |
| | | GATGTCCTCTGAGCGGATCCTCCCCGTGTCTGG | |
| | | GTCCTCTCCGGGCATCTCTCCTCCCTCACCCAAC | |
| | | CCCATGCCGTCTTCACTCGCTGGGTTCCCTTTTC | |
| | | CTTCTCCTTCTGGGGCCTGTGCCATCTCTCGTTT | |
| | | CTTAGGATGGCCTTCTCCGACGGATGTCTCCCTT | |
| | | GCGTCCCGCCTCCCCTTCTTGTAGGCCTGCATCA | |
| | | TCACCGTTTTTCTGGACAACCCCAAAGTACCCC | |
| | | GTCTCCCTGGCTTTAGCCACCTCTCCATCCTCTT | |
| | | GCTTTCTTTGCCTGGACACCCCGTTCTCCTGTGG | |
| | | ATTCGGGTCACCTCTCACTCCTTTCATTTGGGCA | |
| | | GCTCCCCTACCCCCCTTACCTCTCTAGTCTGTGC | |
| | | TAGCTCTTCCAGCCCCCTGTCATGGCATCTTCCA | |
| | | GGGGTCCGAGAGCTCAGCTAGTCTTCTTCCTCC | |
| | | AACCCGGGCCCCTATGTCCACTTCAGGACAGCA | |
| | | TGTTTGCTGCCTCCAGGGATCCTGTGTCCCCGA | |
| | | GCTGGGACCACCTTATATTCCCAGGGCCGGTTA | |
| | | ATGTGGCTCTGGTTCTGGGTACTTTTATCTGTCC | |
| | | CCTCCACCCCACAGTGGGGCCACTAGGGACAG | |
| | | GATTGGTGACAGAAAAGCCCCATCCTTAGGCCT | |
| | | CCTCCTTCCTAGTCTCCTGATATTGGGTCTAACC | |
| | | CCCACCTCCTGTTAGGCAGATTCCTTATCTGGTG | |
| | | ACACACCCCCATTTCCTGGAGCCATCTCTCTCCT | |
| | | TGCCAGAACCTCTAAGGTTTGCTTACGATGGAG | |
| | | CCAGAGAGGATCCTGGGAGGGAGAGCTTGGCA | |
| | | GGGGGTGGGAGGGAAGGGGGGGATGCGTGACC | |
| | | TGCCCGGTTCTCAGTGGCCACCCTGCGCTACCC | |
| | | TCTCCCAGAACCTGAGCTGCTCTGACGCGGCTG | |
| | | TCTGGTGCGTTTCACTGATCCTGGTGCTGCAGCT | |
| | | TCCTTACACTTCCCAAGAGGAGAAGCAGTTTGG | |
| | | AAAAACAAAATCAGAATAAGTTGGTCCTGAGTT | |
| | | CTAACTTTGGCTCTTCACCTTTCTAGTCCCCAAT | |
| | | TTATATTGTTCCTCCGTGCGTCAGTTTTACCTGT | |
| | | GAGATAAGGCCAGTAGCCAGCCCCGGTAACGC | |
| | | CATTTTGCAAGGCATGGAAAAATACCAAACCA | |
| | | AGAATAGAGAAGTTCAGATCAAGGGCGGGTAC | |
| | | ATGAAAATAGCTAACGTTGGGCCAAACAGGAT | |
| | | ATCTGCGGTGAGCAGTTTCGGCCCCGGCCCGGG | |
| | | GCCAAGAACAGATGGTCACCGCAGTTTCGGCCC | |
| | | CGGCCCGAGGCCAAGAACAGATGGTCCCCAGA | |
| | | TATGGCCCAACCCTCAGCAGTTTCTTAAGACCC | |
| | | ATCAGATGTTTCCAGGCTCCCCCAAGGACCTGA | |
| | | AATGACCCTGCGCCTTATTTGAATTAACCAATC | |
| | | AGCCTGCTTCTCGCTTCTGTTCGCGCGCTTCTGC | |
| | | TTCCCGAGCTCTATAAAAGAGCTCACAACCCCT | |
| | | CACTCGGCGCGCCAGTCCTCCGACAGACTGAGT | |
| | | CGCCCGGGCCGCGGCCGCGGGCTAGCGGATCC | |
| | | CCACCGGTCGCCACCATGGTGAGCAAGGGCGA | |
| | | GGAGCTGTTCACCGGGGTGGTGCCCATCCTGGT | |
| | | CGAGCTGGACGGCGACGTAAACGGCCACAAGT | |
| | | TCAGCGTGTCCGGCGAGGGCGAGGGCGATGCC | |
| | | ACCTACGGCAAGCTGACCCTGAAGTTCATCTGC | |
| | | ACCACCGGCAAGCTGCCCGTGCCCTGGCCCACC | |
| | | CTCGTGACCACCCTGACCTACGGCGTGCAGTGC | |
| | | TTCAGCCGCTACCCCGACCACATGAAGCAGCAC | |
| | | GACTTCTTCAAGTCCGCCATGCCCGAAGGCTAC | |
| | | GTCCAGGAGCGCACCATCTTCTTCAAGGACGAC | |
| | | GGCAACTACAAGACCCGCGCCGAGGTGAAGTT | |
| | | CGAGGGCGACACCCTGGTGAACCGCATCGAGC | |
| | | TGAAGGGCATCGACTTCAAGGAGGACGGCAAC | |
| | | ATCCTGGGGCACAAGCTGGAGTACAACTACAA | |
| | | CAGCCACAACGTCTATATCATGGCCGACAAGCA | |
| | | GAAGAACGGCATCAAGGTGAACTTCAAGATCC | |
| | | GCCACAACATCGAGGACGGCAGCGTGCAGCTC | |
| | | GCCGACCACTACCAGCAGAACACCCCCATCGGC | |
| | | GACGGCCCCGTGCTGCTGCCCGACAACCACTAC | |
| | | CTGAGCACCCAGTCCGCCCTGAGCAAAGACCCC | |
| | | AACGAGAAGCGCGATCACATGGTCCTGCTGGA | |
| | | GTTCGTGACCGCCGCCGGGATCACTCTCGGCAT | |
| | | GGACGAGCTGTACAAGTAAAATGAATGCAATT | |
| | | GTTGTTGTTAATAAAGGAAATTTATTTTCATTGC | |
| | | AATAGTGTGTTGGAATTTTTTGTGTCTCTCATCC | |
| | | TGGCAGGGCTGTGGTGAGGAGGGGGGTGTCCG | |
| | | TGTGGAAAACTCCCTTTGTGAGAATGGTGCGTC | |
| | | CTAGGTGTTCACCAGGTCGTGGCCGCCTCTACT | |
| | | CCCTTTCTCTTTCTCCATCCTTCTTTCCTTAAAG | |
| | | AGTCCCCAGTGCTATCTGGGACATATTCCTCCG | |
| | | CCCAGAGCAGGGTCCCGCTTCCCTAAGGCCCTG | |
| | | CTCTGGGCTTCTGGGTTTGAGTCCTTGGCAAGC | |
| | | CCAGGAGAGGCGCTCAGGCTTCCCTGTCCCCCT | |
| | | TCCTCGTCCACCATCTCATGCCCCTGGCTCTCCT | |
| | | GCCCCTTCCCTACAGGGGTTCCTGGCTCTGCTCT | |
| | | TCAGACTGAGCCCCGTTCCCCTGCATCCCCGTT | |
| | | CCCCTGCATCCCCCTTCCCCTGCATCCCCCAGA | |
| | | GGCCCCAGGCCACCTACTTGGCCTGGACCCCAC | |
| | | GAGAGGCCACCCCAGCCCTGTCTACCAGGCTGC | |
| | | CTTTTGGGTGGATTCTCCTCCAACTGTGGGGTG | |
| | | ACTGCTTGGCAAACTCACTCTTCGGGGTATCCC | |
| | | AGGAGGCCTGGAGCATTGGGGTGGGCTGGGGT | |
| | | TCAGAGAGGAGGGATTCCCTTCTCAGGTTACGT | |
| | | GGCCAAGAAGCAGGGGAGCTGGGTTTGGGTCA | |
| | | GGTCTGGGTGTGGGGTGACCAGCTTATGCTGTT | |
| | | TGCCCAGGACAGCCTAGTTTTAGCGCTGAAACC | |
| | | CTCAGTCCTAGGAAAACAGGGATGGTTGGTCAC | |
| | | TGTCTCTGGGTGACTCTTGATTCCCGGCCAGTTT | |
| | | CTCCACCTGGGGCTGTGTTTCTCGTCCTGCATCC | |
| | | TTCTCCAGGCAGGTCCCCAAGCATCGCCCCCCT | |
| | | GCTGTGGCTGTTCCCAAGTTCTTAGGGTACCCC | |
| | | ACGTGGGTTTATCAACCACTTGGTGAGGCTGGT | |
| | | ACCCTGCCCCCATTCCTGCACCCCAATTGCCTTA | |
| | | GTGGCTGGGGGGTTGGGGGCTAGAGTAGGAGG | |
| | | GGCTGGAGCCAGGATTCTTAGGGCTGAACAGA | |
| | | GAAGAGCTGGGGGCCTGGGCTCCTGGGTTTGAG | |
| | | AGAGGAGGGGCTGGGGCCTGGACTCCTGGGTC | |
| | | CGAGGGAGGAGGGGCTGGGGCCTGGACTCCTG | |
| | | GGTCTGAGGGTGGAGGGACTGGGGGCCTGGAC | |
| | | TCCTGGGTCCGAGGGAGGAGGGGCTGGGGCCT | |
| | | GGACTCGTGGGTCTGAGGGAGGAGGGGCTGGG | |
| | | GGCCTGGACTTCTGGGTCTTAGGGAGGCGGGGC | |
| | | TGGGCCTGGACCCCTGGGTCTGAATGGGGAGA | |
| | | GGCTGGGGGCCTGGACTCCTTCATCTGAGGGCG | |
| | | GAAGGGCTGGGGCCTGGCCTCCTGGGTTGAATG | |
| | | GGGAGGGGTTGGGCCTGGACTCTGGAGTCCCTG | |
| | | GTGCCCAGGCCTCAGGCATCTTTCACAGGGATG | |
| | | CCTGTACTGGGCAGGTCCTTGAAAGGGAAAGG | |
| | | CCCATTGCTCTCCTTGCCCCCCTCCCCTATCGCC | |
| | | ATGACAACTGGGTGGAAATAAACGAGCCGAGT | |
| | | TCATCCCGTTCCCAGGGC | |
| Nano | Sg9 | tgacatcaattattatacat | 31 |
| Plasmid/ | (CCR5) | | |
| Mini | | | |
| Plasmid | | | |
| | Sg16 | GCCAGTAGCCAGCCCCGTCC | 94 |
| | (AAVS1-T) | | |
| PT1 | PT1 | AAGGCGATTAAGTTGGGTAACGCCAGGGTTTGA | 121 |
| | (CCR5- | CATCAATTATTATACATCGGATAGGGCGAATTG | |
| | sg9-2C- | GAGCTCGGATCCCTATACAGTTGAAGTCGGAAG | |
| | SFFV- | TTTACATACACTTAAGTTGGAGTCATTAAAACT | |
| | EGFP) | CGTTTTTCAACTACTCCACAAATTTCTTGTTAAC | |
| | | AAACAATAGTTTTGGCAAGTCAGTTAGGACATC | |
| | | TACTTTGTGCATGACACAAGTCATTTTTCCAAC | |
| | | AATTGTTTACAGACAGATTATTTCACTTATAATT | |
| | | CACTGTATCACAATTCCAGTGGGTCAGAAGTTT | |
| | | ACATACACTAAGTTGACTGTGCCTTTAAACAGC | |
| | | TTGGAAAATTGTAACGCCATTTTGCAAGGCATG | |
| | | GAAAAATACCAAACCAAGAATAGAGAAGTTCA | |
| | | GATCAAGGGCGGGTACATGAAAATAGCTAACG | |
| | | TTGGGCCAAACAGGATATCTGCGGTGAGCAGTT | |
| | | TCGGCCCCGGCCCGGGGCCAAGAACAGATGGT | |
| | | CACCGCAGTTTCGGCCCCGGCCCGAGGCCAAGA | |
| | | ACAGATGGTCCCCAGATATGGCCCAACCCTCAG | |
| | | CAGTTTCTTAAGACCCATCAGATGTTTCCAGGC | |
| | | TCCCCCAAGGACCTGAAATGACCCTGCGCCTTA | |
| | | TTTGAATTAACCAATCAGCCTGCTTCTCGCTTCT | |
| | | GTTCGCGCGCTTCTGCTTCCCGAGCTCTATAAA | |
| | | AGAGCTCACAACCCCTCACTCGGCGCGCCAGTC | |
| | | CTCCGACAGACTGAGTCGCCCGGGCCGCGGCCG | |
| | | CGGGCTAGCGGATCCCCACCGGTCGCCACCATG | |
| | | GTGAGCAAGGGCGAGGAGCTGTTCACCGGGGT | |
| | | GGTGCCCATCCTGGTCGAGCTGGACGGCGACGT | |
| | | AAACGGCCACAAGTTCAGCGTGTCCGGCGAGG | |
| | | GCGAGGGCGATGCCACCTACGGCAAGCTGACC | |
| | | CTGAAGTTCATCTGCACCACCGGCAAGCTGCCC | |
| | | GTGCCCTGGCCCACCCTCGTGACCACCCTGACC | |
| | | TACGGCGTGCAGTGCTTCAGCCGCTACCCCGAC | |
| | | CACATGAAGCAGCACGACTTCTTCAAGTCCGCC | |
| | | ATGCCCGAAGGCTACGTCCAGGAGCGCACCATC | |
| | | TTCTTCAAGGACGACGGCAACTACAAGACCCGC | |
| | | GCCGAGGTGAAGTTCGAGGGCGACACCCTGGT | |
| | | GAACCGCATCGAGCTGAAGGGCATCGACTTCA | |
| | | AGGAGGACGGCAACATCCTGGGGCACAAGCTG | |
| | | GAGTACAACTACAACAGCCACAACGTCTATATC | |
| | | ATGGCCGACAAGCAGAAGAACGGCATCAAGGT | |
| | | GAACTTCAAGATCCGCCACAACATCGAGGACG | |
| | | GCAGCGTGCAGCTCGCCGACCACTACCAGCAG | |
| | | AACACCCCCATCGGCGACGGCCCCGTGCTGCTG | |
| | | CCCGACAACCACTACCTGAGCACCCAGTCCGCC | |
| | | CTGAGCAAAGACCCCAACGAGAAGCGCGATCA | |
| | | CATGGTCCTGCTGGAGTTCGTGACCGCCGCCGG | |
| | | GATCACTCTCGGCATGGACGAGCTGTACAAGTA | |
| | | AAATGAATGCAATTGTTGTTGTTAATAAAGGAA | |
| | | ATTTATTTTCATTGCAATAGTGTGTTGGAATTTT | |
| | | TTGTGTCTCTCAACAATTTAAAGGCAATGCTAC | |
| | | CAAATACTAATTGAGTGTATGTAAACTTCTGAC | |
| | | CCACTGGGAATGTGATGAAAGAAATAAAAGCT | |
| | | GAAATGAATCATTCTCTCTACTATTATTCTGATA | |
| | | TTTCACATTCTTAAAATAAAGTGGTGATCCTAA | |
| | | CTGACCTAAGACAGGGAATTTTTACTAGGATTA | |
| | | AATGTCAGGAATTGTGAAAAAGTGAGTTTAAAT | |
| | | GTATTTGGCTAAGGTGTATGTAAACTTCCGACT | |
| | | TCAACTGTATAGGGATCCTCTAGCTAGAGTCGA | |
| | | CCTCTGACATCAATTATTATACATCGGAAGATC | |
| | | CTTTGATCTTTTCTACGGGGTCTG | |
| PT2 | PT2 | AAGGCGATTAAGTTGGGTAACGCCAGGGTTTGA | 122 |
| | (CCR5- | CATCAATTATTATACATCGGATAGGGCGAATTG | |
| | sg9-1C- | GAGCTCGGATCCCTATACAGTTGAAGTCGGAAG | |
| | SFFV- | TTTACATACACTTAAGTTGGAGTCATTAAAACT | |
| | EGFP) | CGTTTTTCAACTACTCCACAAATTTCTTGTTAAC | |
| | | AAACAATAGTTTTGGCAAGTCAGTTAGGACATC | |
| | | TACTTTGTGCATGACACAAGTCATTTTTCCAAC | |
| | | AATTGTTTACAGACAGATTATTTCACTTATAATT | |
| | | CACTGTATCACAATTCCAGTGGGTCAGAAGTTT | |
| | | ACATACACTAAGTTGACTGTGCCTTTAAACAGC | |
| | | TTGGAAAATTGTAACGCCATTTTGCAAGGCATG | |
| | | GAAAAATACCAAACCAAGAATAGAGAAGTTCA | |
| | | GATCAAGGGCGGGTACATGAAAATAGCTAACG | |
| | | TTGGGCCAAACAGGATATCTGCGGTGAGCAGTT | |
| | | TCGGCCCCGGCCCGGGGCCAAGAACAGATGGT | |
| | | CACCGCAGTTTCGGCCCCGGCCCGAGGCCAAGA | |
| | | ACAGATGGTCCCCAGATATGGCCCAACCCTCAG | |
| | | CAGTTTCTTAAGACCCATCAGATGTTTCCAGGC | |
| | | TCCCCCAAGGACCTGAAATGACCCTGCGCCTTA | |
| | | TTTGAATTAACCAATCAGCCTGCTTCTCGCTTCT | |
| | | GTTCGCGCGCTTCTGCTTCCCGAGCTCTATAAA | |
| | | AGAGCTCACAACCCCTCACTCGGCGCGCCAGTC | |
| | | CTCCGACAGACTGAGTCGCCCGGGCCGCGGCCG | |
| | | CGGGCTAGCGGATCCCCACCGGTCGCCACCATG | |
| | | GTGAGCAAGGGCGAGGAGCTGTTCACCGGGGT | |
| | | GGTGCCCATCCTGGTCGAGCTGGACGGCGACGT | |
| | | AAACGGCCACAAGTTCAGCGTGTCCGGCGAGG | |
| | | GCGAGGGCGATGCCACCTACGGCAAGCTGACC | |
| | | CTGAAGTTCATCTGCACCACCGGCAAGCTGCCC | |
| | | GTGCCCTGGCCCACCCTCGTGACCACCCTGACC | |
| | | TACGGCGTGCAGTGCTTCAGCCGCTACCCCGAC | |
| | | CACATGAAGCAGCACGACTTCTTCAAGTCCGCC | |
| | | ATGCCCGAAGGCTACGTCCAGGAGCGCACCATC | |
| | | TTCTTCAAGGACGACGGCAACTACAAGACCCGC | |
| | | GCCGAGGTGAAGTTCGAGGGCGACACCCTGGT | |
| | | GAACCGCATCGAGCTGAAGGGCATCGACTTCA | |
| | | AGGAGGACGGCAACATCCTGGGGCACAAGCTG | |
| | | GAGTACAACTACAACAGCCACAACGTCTATATC | |
| | | ATGGCCGACAAGCAGAAGAACGGCATCAAGGT | |
| | | GAACTTCAAGATCCGCCACAACATCGAGGACG | |
| | | GCAGCGTGCAGCTCGCCGACCACTACCAGCAG | |
| | | AACACCCCCATCGGCGACGGCCCCGTGCTGCTG | |
| | | CCCGACAACCACTACCTGAGCACCCAGTCCGCC | |
| | | CTGAGCAAAGACCCCAACGAGAAGCGCGATCA | |
| | | CATGGTCCTGCTGGAGTTCGTGACCGCCGCCGG | |
| | | GATCACTCTCGGCATGGACGAGCTGTACAAGTA | |
| | | AAATGAATGCAATTGTTGTTGTTAATAAAGGAA | |
| | | ATTTATTTTCATTGCAATAGTGTGTTGGAATTTT | |
| | | TTGTGTCTCTCAACAATTTAAAGGCAATGCTAC | |
| | | CAAATACTAATTGAGTGTATGTAAACTTCTGAC | |
| | | CCACTGGGAATGTGATGAAAGAAATAAAAGCT | |
| | | GAAATGAATCATTCTCTCTACTATTATTCTGATA | |
| | | TTTCACATTCTTAAAATAAAGTGGTGATCCTAA | |
| | | CTGACCTAAGACAGGGAATTTTTACTAGGATTA | |
| | | AATGTCAGGAATTGTGAAAAAGTGAGTTTAAAT | |
| | | GTATTTGGCTAAGGTGTATGTAAACTTCCGACT | |
| | | TCAACTGTATAGGGATCCTCTAGCTAGAGTCGA | |
| | | CCTC | |
| PT3 | PT3 | AAGGCGATTAAGTTGGGTAACGCCAGGGTTGCC | 123 |
| | (AAVS1- | AGTAGCCAGCCCCGTCCTGGATAGGGCGAATTG | |
| | SG16- | GAGCTCGGATCCCTATACAGTTGAAGTCGGAAG | |
| | 2C- | TTTACATACACTTAAGTTGGAGTCATTAAAACT | |
| | SFFV- | CGTTTTTCAACTACTCCACAAATTTCTTGTTAAC | |
| | EGFP) | AAACAATAGTTTTGGCAAGTCAGTTAGGACATC | |
| | | TACTTTGTGCATGACACAAGTCATTTTTCCAAC | |
| | | AATTGTTTACAGACAGATTATTTCACTTATAATT | |
| | | CACTGTATCACAATTCCAGTGGGTCAGAAGTTT | |
| | | ACATACACTAAGTTGACTGTGCCTTTAAACAGC | |
| | | TTGGAAAATTGTAACGCCATTTTGCAAGGCATG | |
| | | GAAAAATACCAAACCAAGAATAGAGAAGTTCA | |
| | | GATCAAGGGCGGGTACATGAAAATAGCTAACG | |
| | | TTGGGCCAAACAGGATATCTGCGGTGAGCAGTT | |
| | | TCGGCCCCGGCCCGGGGCCAAGAACAGATGGT | |
| | | CACCGCAGTTTCGGCCCCGGCCCGAGGCCAAGA | |
| | | ACAGATGGTCCCCAGATATGGCCCAACCCTCAG | |
| | | CAGTTTCTTAAGACCCATCAGATGTTTCCAGGC | |
| | | TCCCCCAAGGACCTGAAATGACCCTGCGCCTTA | |
| | | TTTGAATTAACCAATCAGCCTGCTTCTCGCTTCT | |
| | | GTTCGCGCGCTTCTGCTTCCCGAGCTCTATAAA | |
| | | AGAGCTCACAACCCCTCACTCGGCGCGCCAGTC | |
| | | CTCCGACAGACTGAGTCGCCCGGGCCGCGGCCG | |
| | | CGGGCTAGCGGATCCCCACCGGTCGCCACCATG | |
| | | GTGAGCAAGGGCGAGGAGCTGTTCACCGGGGT | |
| | | GGTGCCCATCCTGGTCGAGCTGGACGGCGACGT | |
| | | AAACGGCCACAAGTTCAGCGTGTCCGGCGAGG | |
| | | GCGAGGGCGATGCCACCTACGGCAAGCTGACC | |
| | | CTGAAGTTCATCTGCACCACCGGCAAGCTGCCC | |
| | | GTGCCCTGGCCCACCCTCGTGACCACCCTGACC | |
| | | TACGGCGTGCAGTGCTTCAGCCGCTACCCCGAC | |
| | | CACATGAAGCAGCACGACTTCTTCAAGTCCGCC | |
| | | ATGCCCGAAGGCTACGTCCAGGAGCGCACCATC | |
| | | TTCTTCAAGGACGACGGCAACTACAAGACCCGC | |
| | | GCCGAGGTGAAGTTCGAGGGCGACACCCTGGT | |
| | | GAACCGCATCGAGCTGAAGGGCATCGACTTCA | |
| | | AGGAGGACGGCAACATCCTGGGGCACAAGCTG | |
| | | GAGTACAACTACAACAGCCACAACGTCTATATC | |
| | | ATGGCCGACAAGCAGAAGAACGGCATCAAGGT | |
| | | GAACTTCAAGATCCGCCACAACATCGAGGACG | |
| | | GCAGCGTGCAGCTCGCCGACCACTACCAGCAG | |
| | | AACACCCCCATCGGCGACGGCCCCGTGCTGCTG | |
| | | CCCGACAACCACTACCTGAGCACCCAGTCCGCC | |
| | | CTGAGCAAAGACCCCAACGAGAAGCGCGATCA | |
| | | CATGGTCCTGCTGGAGTTCGTGACCGCCGCCGG | |
| | | GATCACTCTCGGCATGGACGAGCTGTACAAGTA | |
| | | AAATGAATGCAATTGTTGTTGTTAATAAAGGAA | |
| | | ATTTATTTTCATTGCAATAGTGTGTTGGAATTTT | |
| | | TTGTGTCTCTCAACAATTTAAAGGCAATGCTAC | |
| | | CAAATACTAATTGAGTGTATGTAAACTTCTGAC | |
| | | CCACTGGGAATGTGATGAAAGAAATAAAAGCT | |
| | | GAAATGAATCATTCTCTCTACTATTATTCTGATA | |
| | | TTTCACATTCTTAAAATAAAGTGGTGATCCTAA | |
| | | CTGACCTAAGACAGGGAATTTTTACTAGGATTA | |
| | | AATGTCAGGAATTGTGAAAAAGTGAGTTTAAAT | |
| | | GTATTTGGCTAAGGTGTATGTAAACTTCCGACT | |
| | | TCAACTGTATAGGGATCCTCTAGCTAGAGTCGA | |
| | | CCTCGCCAGTAGCCAGCCCCGTCCTGGAAGATC | |
| | | CTTTGATCTTTTCTACGGGGTCTG | |
| PT4 | PT4 | AAGGCGATTAAGTTGGGTAACGCCAGGGTTGCC | 124 |
| | (AAVS1- | AGTAGCCAGCCCCGTCCTGGATAGGGCGAATTG | |
| | SG16- | GAGCTCGGATCCCTATACAGTTGAAGTCGGAAG | |
| | 1C- | TTTACATACACTTAAGTTGGAGTCATTAAAACT | |
| | SFFV- | CGTTTTTCAACTACTCCACAAATTTCTTGTTAAC | |
| | EGFP) | AAACAATAGTTTTGGCAAGTCAGTTAGGACATC | |
| | | TACTTTGTGCATGACACAAGTCATTTTTCCAAC | |
| | | AATTGTTTACAGACAGATTATTTCACTTATAATT | |
| | | CACTGTATCACAATTCCAGTGGGTCAGAAGTTT | |
| | | ACATACACTAAGTTGACTGTGCCTTTAAACAGC | |
| | | TTGGAAAATTGTAACGCCATTTTGCAAGGCATG | |
| | | GAAAAATACCAAACCAAGAATAGAGAAGTTCA | |
| | | GATCAAGGGCGGGTACATGAAAATAGCTAACG | |
| | | TTGGGCCAAACAGGATATCTGCGGTGAGCAGTT | |
| | | TCGGCCCCGGCCCGGGGCCAAGAACAGATGGT | |
| | | CACCGCAGTTTCGGCCCCGGCCCGAGGCCAAGA | |
| | | ACAGATGGTCCCCAGATATGGCCCAACCCTCAG | |
| | | CAGTTTCTTAAGACCCATCAGATGTTTCCAGGC | |
| | | TCCCCCAAGGACCTGAAATGACCCTGCGCCTTA | |
| | | TTTGAATTAACCAATCAGCCTGCTTCTCGCTTCT | |
| | | GTTCGCGCGCTTCTGCTTCCCGAGCTCTATAAA | |
| | | AGAGCTCACAACCCCTCACTCGGCGCGCCAGTC | |
| | | CTCCGACAGACTGAGTCGCCCGGGCCGCGGCCG | |
| | | CGGGCTAGCGGATCCCCACCGGTCGCCACCATG | |
| | | GTGAGCAAGGGCGAGGAGCTGTTCACCGGGGT | |
| | | GGTGCCCATCCTGGTCGAGCTGGACGGCGACGT | |
| | | AAACGGCCACAAGTTCAGCGTGTCCGGCGAGG | |
| | | GCGAGGGCGATGCCACCTACGGCAAGCTGACC | |
| | | CTGAAGTTCATCTGCACCACCGGCAAGCTGCCC | |
| | | GTGCCCTGGCCCACCCTCGTGACCACCCTGACC | |
| | | TACGGCGTGCAGTGCTTCAGCCGCTACCCCGAC | |
| | | CACATGAAGCAGCACGACTTCTTCAAGTCCGCC | |
| | | ATGCCCGAAGGCTACGTCCAGGAGCGCACCATC | |
| | | TTCTTCAAGGACGACGGCAACTACAAGACCCGC | |
| | | GCCGAGGTGAAGTTCGAGGGCGACACCCTGGT | |
| | | GAACCGCATCGAGCTGAAGGGCATCGACTTCA | |
| | | AGGAGGACGGCAACATCCTGGGGCACAAGCTG | |
| | | GAGTACAACTACAACAGCCACAACGTCTATATC | |
| | | ATGGCCGACAAGCAGAAGAACGGCATCAAGGT | |
| | | GAACTTCAAGATCCGCCACAACATCGAGGACG | |
| | | GCAGCGTGCAGCTCGCCGACCACTACCAGCAG | |
| | | AACACCCCCATCGGCGACGGCCCCGTGCTGCTG | |
| | | CCCGACAACCACTACCTGAGCACCCAGTCCGCC | |
| | | CTGAGCAAAGACCCCAACGAGAAGCGCGATCA | |
| | | CATGGTCCTGCTGGAGTTCGTGACCGCCGCCGG | |
| | | GATCACTCTCGGCATGGACGAGCTGTACAAGTA | |
| | | AAATGAATGCAATTGTTGTTGTTAATAAAGGAA | |
| | | ATTTATTTTCATTGCAATAGTGTGTTGGAATTTT | |
| | | TTGTGTCTCTCAACAATTTAAAGGCAATGCTAC | |
| | | CAAATACTAATTGAGTGTATGTAAACTTCTGAC | |
| | | CCACTGGGAATGTGATGAAAGAAATAAAAGCT | |
| | | GAAATGAATCATTCTCTCTACTATTATTCTGATA | |
| | | TTTCACATTCTTAAAATAAAGTGGTGATCCTAA | |
| | | CTGACCTAAGACAGGGAATTTTTACTAGGATTA | |
| | | AATGTCAGGAATTGTGAAAAAGTGAGTTTAAAT | |
| | | GTATTTGGCTAAGGTGTATGTAAACTTCCGACT | |
| | | TCAACTGTATAGGGATCCTCTAGCTAGAGTCGA | |
| | | CCTC | |
| PT5 | PT5 | AAGGCGATTAAGTTGGGTAACGCCAGGGTTTGA | 125 |
| | (CCR5-sg9- | CATCAATTATTATACATCGGATAGGGCGAATTG | |
| | 2C- | GAGCTCGGATCCCTATACAGTTGAAGTCGGAAG | |
| | SFFV- | TTTACATACACTTAAGTTGGAGTCATTAAAACT | |
| | EGFP) | CGTTTTTCAACTACTCCACAAATTTCTTGTTAAC | |
| | | AAACAATAGTTTTGGCAAGTCAGTTAGGACATC | |
| | | TACTTTGTGCATGACACAAGTCATTTTTCCAAC | |
| | | AATTGTTTACAGACAGATTATTTCACTTATAATT | |
| | | CACTGTATCACAATTCCAGTGGGTCAGAAGTTT | |
| | | ACATACACTAAGTTGACTGTGCCTTTAAACAGC | |
| | | TTGGAAAATTGTAACGCCATTTTGCAAGGCATG | |
| | | GAAAAATACCAAACCAAGAATAGAGAAGTTCA | |
| | | GATCAAGGGCGGGTACATGAAAATAGCTAACG | |
| | | TTGGGCCAAACAGGATATCTGCGGTGAGCAGTT | |
| | | TCGGCCCCGGCCCGGGGCCAAGAACAGATGGT | |
| | | CACCGCAGTTTCGGCCCCGGCCCGAGGCCAAGA | |
| | | ACAGATGGTCCCCAGATATGGCCCAACCCTCAG | |
| | | CAGTTTCTTAAGACCCATCAGATGTTTCCAGGC | |
| | | TCCCCCAAGGACCTGAAATGACCCTGCGCCTTA | |
| | | TTTGAATTAACCAATCAGCCTGCTTCTCGCTTCT | |
| | | GTTCGCGCGCTTCTGCTTCCCGAGCTCTATAAA | |
| | | AGAGCTCACAACCCCTCACTCGGCGCGCCAGTC | |
| | | CTCCGACAGACTGAGTCGCCCGGGCCGCGGCCG | |
| | | CGGGCTAGCGGATCCCCACCGGTCGCCACCATG | |
| | | GTGAGCAAGGGCGAGGAGCTGTTCACCGGGGT | |
| | | GGTGCCCATCCTGGTCGAGCTGGACGGCGACGT | |
| | | AAACGGCCACAAGTTCAGCGTGTCCGGCGAGG | |
| | | GCGAGGGCGATGCCACCTACGGCAAGCTGACC | |
| | | CTGAAGTTCATCTGCACCACCGGCAAGCTGCCC | |
| | | GTGCCCTGGCCCACCCTCGTGACCACCCTGACC | |
| | | TACGGCGTGCAGTGCTTCAGCCGCTACCCCGAC | |
| | | CACATGAAGCAGCACGACTTCTTCAAGTCCGCC | |
| | | ATGCCCGAAGGCTACGTCCAGGAGCGCACCATC | |
| | | TTCTTCAAGGACGACGGCAACTACAAGACCCGC | |
| | | GCCGAGGTGAAGTTCGAGGGCGACACCCTGGT | |
| | | GAACCGCATCGAGCTGAAGGGCATCGACTTCA | |
| | | AGGAGGACGGCAACATCCTGGGGCACAAGCTG | |
| | | GAGTACAACTACAACAGCCACAACGTCTATATC | |
| | | ATGGCCGACAAGCAGAAGAACGGCATCAAGGT | |
| | | GAACTTCAAGATCCGCCACAACATCGAGGACG | |
| | | GCAGCGTGCAGCTCGCCGACCACTACCAGCAG | |
| | | AACACCCCCATCGGCGACGGCCCCGTGCTGCTG | |
| | | CCCGACAACCACTACCTGAGCACCCAGTCCGCC | |
| | | CTGAGCAAAGACCCCAACGAGAAGCGCGATCA | |
| | | CATGGTCCTGCTGGAGTTCGTGACCGCCGCCGG | |
| | | GATCACTCTCGGCATGGACGAGCTGTACAAGTA | |
| | | AAATGAATGCAATTGTTGTTGTTAATAAAGGAA | |
| | | ATTTATTTTCATTGCAATAGTGTGTTGGAATTTT | |
| | | TTGTGTCTCTCAACAATTTAAAGGCAATGCTAC | |
| | | CAAATACTAATTGAGTGTATGTAAACTTCTGAC | |
| | | CCACTGGGAATGTGATGAAAGAAATAAAAGCT | |
| | | GAAATGAATCATTCTCTCTACTATTATTCTGATA | |
| | | TTTCACATTCTTAAAATAAAGTGGTGATCCTAA | |
| | | CTGACCTAAGACAGGGAATTTTTACTAGGATTA | |
| | | AATGTCAGGAATTGTGAAAAAGTGAGTTTAAAT | |
| | | GTATTTGGCTAAGGTGTATGTAAACTTCCGACT | |
| | | TCAACTGTATAGGGATCCTCTAGCTAGAGTCGA | |
| | | CCTCTGACATCAATTATTATACATCGGAAGATC | |
| | | CTTTGATCTTTTCTACGGGGTCTG | |
| PT6 | PT6 | AAGGCGATTAAGTTGGGTAACGCCAGGGTTTGA | 126 |
| | (CCR5- | CATCAATTATTATACATCGGATAGGGCGAATTG | |
| | sg9-1C- | GAGCTCGGATCCCTATACAGTTGAAGTCGGAAG | |
| | SFFV- | TTTACATACACTTAAGTTGGAGTCATTAAAACT | |
| | EGFP) | CGTTTTTCAACTACTCCACAAATTTCTTGTTAAC | |
| | | AAACAATAGTTTTGGCAAGTCAGTTAGGACATC | |
| | | TACTTTGTGCATGACACAAGTCATTTTTCCAAC | |
| | | AATTGTTTACAGACAGATTATTTCACTTATAATT | |
| | | CACTGTATCACAATTCCAGTGGGTCAGAAGTTT | |
| | | ACATACACTAAGTTGACTGTGCCTTTAAACAGC | |
| | | TTGGAAAATTGTAACGCCATTTTGCAAGGCATG | |
| | | GAAAAATACCAAACCAAGAATAGAGAAGTTCA | |
| | | GATCAAGGGCGGGTACATGAAAATAGCTAACG | |
| | | TTGGGCCAAACAGGATATCTGCGGTGAGCAGTT | |
| | | TCGGCCCCGGCCCGGGGCCAAGAACAGATGGT | |
| | | CACCGCAGTTTCGGCCCCGGCCCGAGGCCAAGA | |
| | | ACAGATGGTCCCCAGATATGGCCCAACCCTCAG | |
| | | CAGTTTCTTAAGACCCATCAGATGTTTCCAGGC | |
| | | TCCCCCAAGGACCTGAAATGACCCTGCGCCTTA | |
| | | TTTGAATTAACCAATCAGCCTGCTTCTCGCTTCT | |
| | | GTTCGCGCGCTTCTGCTTCCCGAGCTCTATAAA | |
| | | AGAGCTCACAACCCCTCACTCGGCGCGCCAGTC | |
| | | CTCCGACAGACTGAGTCGCCCGGGCCGCGGCCG | |
| | | CGGGCTAGCGGATCCCCACCGGTCGCCACCATG | |
| | | GTGAGCAAGGGCGAGGAGCTGTTCACCGGGGT | |
| | | GGTGCCCATCCTGGTCGAGCTGGACGGCGACGT | |
| | | AAACGGCCACAAGTTCAGCGTGTCCGGCGAGG | |
| | | GCGAGGGCGATGCCACCTACGGCAAGCTGACC | |
| | | CTGAAGTTCATCTGCACCACCGGCAAGCTGCCC | |
| | | GTGCCCTGGCCCACCCTCGTGACCACCCTGACC | |
| | | TACGGCGTGCAGTGCTTCAGCCGCTACCCCGAC | |
| | | CACATGAAGCAGCACGACTTCTTCAAGTCCGCC | |
| | | ATGCCCGAAGGCTACGTCCAGGAGCGCACCATC | |
| | | TTCTTCAAGGACGACGGCAACTACAAGACCCGC | |
| | | GCCGAGGTGAAGTTCGAGGGCGACACCCTGGT | |
| | | GAACCGCATCGAGCTGAAGGGCATCGACTTCA | |
| | | AGGAGGACGGCAACATCCTGGGGCACAAGCTG | |
| | | GAGTACAACTACAACAGCCACAACGTCTATATC | |
| | | ATGGCCGACAAGCAGAAGAACGGCATCAAGGT | |
| | | GAACTTCAAGATCCGCCACAACATCGAGGACG | |
| | | GCAGCGTGCAGCTCGCCGACCACTACCAGCAG | |
| | | AACACCCCCATCGGCGACGGCCCCGTGCTGCTG | |
| | | CCCGACAACCACTACCTGAGCACCCAGTCCGCC | |
| | | CTGAGCAAAGACCCCAACGAGAAGCGCGATCA | |
| | | CATGGTCCTGCTGGAGTTCGTGACCGCCGCCGG | |
| | | GATCACTCTCGGCATGGACGAGCTGTACAAGTA | |
| | | AAATGAATGCAATTGTTGTTGTTAATAAAGGAA | |
| | | ATTTATTTTCATTGCAATAGTGTGTTGGAATTTT | |
| | | TTGTGTCTCTCAACAATTTAAAGGCAATGCTAC | |
| | | CAAATACTAATTGAGTGTATGTAAACTTCTGAC | |
| | | CCACTGGGAATGTGATGAAAGAAATAAAAGCT | |
| | | GAAATGAATCATTCTCTCTACTATTATTCTGATA | |
| | | TTTCACATTCTTAAAATAAAGTGGTGATCCTAA | |
| | | CTGACCTAAGACAGGGAATTTTTACTAGGATTA | |
| | | AATGTCAGGAATTGTGAAAAAGTGAGTTTAAAT | |
| | | GTATTTGGCTAAGGTGTATGTAAACTTCCGACT | |
| | | TCAACTGTATAGGGATCCTCTAGCTAGAGTCGA | |
| | | CCTC | |
| PT7 | PT7 | AAGGCGATTAAGTTGGGTAACGCCAGGGTTGCC | 127 |
| | (AAVS1- | AGTAGCCAGCCCCGTCCTGGATAGGGCGAATTG | |
| | SG16- | GAGCTCGGATCCCTATACAGTTGAAGTCGGAAG | |
| | 2C- | TTTACATACACTTAAGTTGGAGTCATTAAAACT | |
| | SFFV- | CGTTTTTCAACTACTCCACAAATTTCTTGTTAAC | |
| | EGFP) | AAACAATAGTTTTGGCAAGTCAGTTAGGACATC | |
| | | TACTTTGTGCATGACACAAGTCATTTTTCCAAC | |
| | | AATTGTTTACAGACAGATTATTTCACTTATAATT | |
| | | CACTGTATCACAATTCCAGTGGGTCAGAAGTTT | |
| | | ACATACACTAAGTTGACTGTGCCTTTAAACAGC | |
| | | TTGGAAAATTGTAACGCCATTTTGCAAGGCATG | |
| | | GAAAAATACCAAACCAAGAATAGAGAAGTTCA | |
| | | GATCAAGGGCGGGTACATGAAAATAGCTAACG | |
| | | TTGGGCCAAACAGGATATCTGCGGTGAGCAGTT | |
| | | TCGGCCCCGGCCCGGGGCCAAGAACAGATGGT | |
| | | CACCGCAGTTTCGGCCCCGGCCCGAGGCCAAGA | |
| | | ACAGATGGTCCCCAGATATGGCCCAACCCTCAG | |
| | | CAGTTTCTTAAGACCCATCAGATGTTTCCAGGC | |
| | | TCCCCCAAGGACCTGAAATGACCCTGCGCCTTA | |
| | | TTTGAATTAACCAATCAGCCTGCTTCTCGCTTCT | |
| | | GTTCGCGCGCTTCTGCTTCCCGAGCTCTATAAA | |
| | | AGAGCTCACAACCCCTCACTCGGCGCGCCAGTC | |
| | | CTCCGACAGACTGAGTCGCCCGGGCCGCGGCCG | |
| | | CGGGCTAGCGGATCCCCACCGGTCGCCACCATG | |
| | | GTGAGCAAGGGCGAGGAGCTGTTCACCGGGGT | |
| | | GGTGCCCATCCTGGTCGAGCTGGACGGCGACGT | |
| | | AAACGGCCACAAGTTCAGCGTGTCCGGCGAGG | |
| | | GCGAGGGCGATGCCACCTACGGCAAGCTGACC | |
| | | CTGAAGTTCATCTGCACCACCGGCAAGCTGCCC | |
| | | GTGCCCTGGCCCACCCTCGTGACCACCCTGACC | |
| | | TACGGCGTGCAGTGCTTCAGCCGCTACCCCGAC | |
| | | CACATGAAGCAGCACGACTTCTTCAAGTCCGCC | |
| | | ATGCCCGAAGGCTACGTCCAGGAGCGCACCATC | |
| | | TTCTTCAAGGACGACGGCAACTACAAGACCCGC | |
| | | GCCGAGGTGAAGTTCGAGGGCGACACCCTGGT | |
| | | GAACCGCATCGAGCTGAAGGGCATCGACTTCA | |
| | | AGGAGGACGGCAACATCCTGGGGCACAAGCTG | |
| | | GAGTACAACTACAACAGCCACAACGTCTATATC | |
| | | ATGGCCGACAAGCAGAAGAACGGCATCAAGGT | |
| | | GAACTTCAAGATCCGCCACAACATCGAGGACG | |
| | | GCAGCGTGCAGCTCGCCGACCACTACCAGCAG | |
| | | AACACCCCCATCGGCGACGGCCCCGTGCTGCTG | |
| | | CCCGACAACCACTACCTGAGCACCCAGTCCGCC | |
| | | CTGAGCAAAGACCCCAACGAGAAGCGCGATCA | |
| | | CATGGTCCTGCTGGAGTTCGTGACCGCCGCCGG | |
| | | GATCACTCTCGGCATGGACGAGCTGTACAAGTA | |
| | | AAATGAATGCAATTGTTGTTGTTAATAAAGGAA | |
| | | ATTTATTTTCATTGCAATAGTGTGTTGGAATTTT | |
| | | TTGTGTCTCTCAACAATTTAAAGGCAATGCTAC | |
| | | CAAATACTAATTGAGTGTATGTAAACTTCTGAC | |
| | | CCACTGGGAATGTGATGAAAGAAATAAAAGCT | |
| | | GAAATGAATCATTCTCTCTACTATTATTCTGATA | |
| | | TTTCACATTCTTAAAATAAAGTGGTGATCCTAA | |
| | | CTGACCTAAGACAGGGAATTTTTACTAGGATTA | |
| | | AATGTCAGGAATTGTGAAAAAGTGAGTTTAAAT | |
| | | GTATTTGGCTAAGGTGTATGTAAACTTCCGACT | |
| | | TCAACTGTATAGGGATCCTCTAGCTAGAGTCGA | |
| | | CCTCGCCAGTAGCCAGCCCCGTCCTGGAAGATC | |
| | | CTTTGATCTTTTCTACGGGGTCTG | |
| PT8 | PT8 | AAGGCGATTAAGTTGGGTAACGCCAGGGTTGCC | 128 |
| | (AAVS1- | AGTAGCCAGCCCCGTCCTGGATAGGGCGAATTG | |
| | SG16- | GAGCTCGGATCCCTATACAGTTGAAGTCGGAAG | |
| | 1C- | TTTACATACACTTAAGTTGGAGTCATTAAAACT | |
| | SFFV- | CGTTTTTCAACTACTCCACAAATTTCTTGTTAAC | |
| | EGFP) | AAACAATAGTTTTGGCAAGTCAGTTAGGACATC | |
| | | TACTTTGTGCATGACACAAGTCATTTTTCCAAC | |
| | | AATTGTTTACAGACAGATTATTTCACTTATAATT | |
| | | CACTGTATCACAATTCCAGTGGGTCAGAAGTTT | |
| | | ACATACACTAAGTTGACTGTGCCTTTAAACAGC | |
| | | TTGGAAAATTGTAACGCCATTTTGCAAGGCATG | |
| | | GAAAAATACCAAACCAAGAATAGAGAAGTTCA | |
| | | GATCAAGGGCGGGTACATGAAAATAGCTAACG | |
| | | TTGGGCCAAACAGGATATCTGCGGTGAGCAGTT | |
| | | TCGGCCCCGGCCCGGGGCCAAGAACAGATGGT | |
| | | CACCGCAGTTTCGGCCCCGGCCCGAGGCCAAGA | |
| | | ACAGATGGTCCCCAGATATGGCCCAACCCTCAG | |
| | | CAGTTTCTTAAGACCCATCAGATGTTTCCAGGC | |
| | | TCCCCCAAGGACCTGAAATGACCCTGCGCCTTA | |
| | | TTTGAATTAACCAATCAGCCTGCTTCTCGCTTCT | |
| | | GTTCGCGCGCTTCTGCTTCCCGAGCTCTATAAA | |
| | | AGAGCTCACAACCCCTCACTCGGCGCGCCAGTC | |
| | | CTCCGACAGACTGAGTCGCCCGGGCCGCGGCCG | |
| | | CGGGCTAGCGGATCCCCACCGGTCGCCACCATG | |
| | | GTGAGCAAGGGCGAGGAGCTGTTCACCGGGGT | |
| | | GGTGCCCATCCTGGTCGAGCTGGACGGCGACGT | |
| | | AAACGGCCACAAGTTCAGCGTGTCCGGCGAGG | |
| | | GCGAGGGCGATGCCACCTACGGCAAGCTGACC | |
| | | CTGAAGTTCATCTGCACCACCGGCAAGCTGCCC | |
| | | GTGCCCTGGCCCACCCTCGTGACCACCCTGACC | |
| | | TACGGCGTGCAGTGCTTCAGCCGCTACCCCGAC | |
| | | CACATGAAGCAGCACGACTTCTTCAAGTCCGCC | |
| | | ATGCCCGAAGGCTACGTCCAGGAGCGCACCATC | |
| | | TTCTTCAAGGACGACGGCAACTACAAGACCCGC | |
| | | GCCGAGGTGAAGTTCGAGGGCGACACCCTGGT | |
| | | GAACCGCATCGAGCTGAAGGGCATCGACTTCA | |
| | | AGGAGGACGGCAACATCCTGGGGCACAAGCTG | |
| | | GAGTACAACTACAACAGCCACAACGTCTATATC | |
| | | ATGGCCGACAAGCAGAAGAACGGCATCAAGGT | |
| | | GAACTTCAAGATCCGCCACAACATCGAGGACG | |
| | | GCAGCGTGCAGCTCGCCGACCACTACCAGCAG | |
| | | AACACCCCCATCGGCGACGGCCCCGTGCTGCTG | |
| | | CCCGACAACCACTACCTGAGCACCCAGTCCGCC | |
| | | CTGAGCAAAGACCCCAACGAGAAGCGCGATCA | |
| | | CATGGTCCTGCTGGAGTTCGTGACCGCCGCCGG | |
| | | GATCACTCTCGGCATGGACGAGCTGTACAAGTA | |
| | | AAATGAATGCAATTGTTGTTGTTAATAAAGGAA | |
| | | ATTTATTTTCATTGCAATAGTGTGTTGGAATTTT | |
| | | TTGTGTCTCTCAACAATTTAAAGGCAATGCTAC | |
| | | CAAATACTAATTGAGTGTATGTAAACTTCTGAC | |
| | | CCACTGGGAATGTGATGAAAGAAATAAAAGCT | |
| | | GAAATGAATCATTCTCTCTACTATTATTCTGATA | |
| | | TTTCACATTCTTAAAATAAAGTGGTGATCCTAA | |
| | | CTGACCTAAGACAGGGAATTTTTACTAGGATTA | |
| | | AATGTCAGGAATTGTGAAAAAGTGAGTTTAAAT | |
| | | GTATTTGGCTAAGGTGTATGTAAACTTCCGACT | |
| | | TCAACTGTATAGGGATCCTCTAGCTAGAGTCGA | |
| | | CCTC | |

### EQUIVALENTS AND SCOPE

Those skilled in the art will recognize, or be able to ascertain using no more than routine experimentation, many equivalents of the embodiments described herein. The scope of the present disclosure is not intended to be limited to the above description, but rather is as set forth in the appended claims.

Articles such as “a,” “an,” and “the” may mean one or more than one unless indicated to the contrary or otherwise evident from the context. Claims or descriptions that include “or” between two or more members of a group are considered satisfied if one, more than one, or all of the group members are present, unless indicated to the contrary or otherwise evident from the context. The disclosure of a group that includes “or” between two or more group members provides embodiments in which exactly one member of the group is present, embodiments in which two or more members of the group are present, and embodiments in which all of the group members are present. For purposes of brevity those embodiments have not been individually spelled out herein, but it will be understood that each of these embodiments is provided herein and may be specifically claimed or disclaimed.

It is to be understood that the invention encompasses all variations, combinations, and permutations in which one or more limitation, element, clause, or descriptive term, from one or more of the claims or from one or more relevant portion of the description, is introduced into another claim. For example, a claim that is dependent on another claim can be modified to include one or more of the limitations found in any other claim that is dependent on the same base claim. Furthermore, where the claims recite a composition, it is to be understood that methods of making or using the composition according to any of the methods of making or using disclosed herein or according to methods known in the art, if any, are included, unless otherwise indicated or unless it would be evident to one of ordinary skill in the art that a contradiction or inconsistency would arise.

Where elements are presented as lists, e.g., in Markush group format, it is to be understood that every possible subgroup of the elements is also disclosed, and that any element or subgroup of elements can be removed from the group. It is also noted that the term “comprising” is intended to be open and permits the inclusion of additional elements or steps. It should be understood that, in general, where an embodiment, product, or method is referred to as comprising particular elements, features, or steps, embodiments, products, or methods that consist, or consist essentially of, such elements, features, or steps, are provided as well. For purposes of brevity those embodiments have not been individually spelled out herein, but it will be understood that each of these embodiments is provided herein and may be specifically claimed or disclaimed.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in some embodiments, to the tenth of the unit of the lower limit of the range, unless the context clearly dictates otherwise. For purposes of brevity, the values in each range have not been individually spelled out herein, but it will be understood that each of these values is provided herein and may be specifically claimed or disclaimed. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Where ranges are given, any value within the range may explicitly be excluded from any one or more of the claims. Any embodiment, element, feature, application, or aspect of the compositions and/or methods described herein, can be excluded from any one or more claims. For purposes of brevity, all of the embodiments in which one or more elements, features, purposes, or aspects is excluded are not set forth.

What is claimed is:

1. A method comprising:
contacting a hematopoietic cell with:
(a) a Clustered Regularly Interspaced Short Palindromic Repeats (CRISPR)-CRISPR associated (Cas) (CRISPR/Cas) system comprising a Cas nuclease and a guide RNA (gRNA) comprising a nucleotide sequence that hybridizes to a target DNA in the genome of the hematopoietic cell; and
(b) a template polynucleotide.

2. The method of claim 1, wherein contacting also comprises contacting the hematopoietic cell with: (c) one or both of:
an expansion agent;
a homology-directed repair (HDR) promoting agent.

3. The method of either one of claim 1 or 2, wherein the CRISPR/Cas system creates a double-stranded break (DSB) in the target DNA in the genome of the hematopoietic cell.

4. The method of claims 1-3, wherein the template polynucleotide is a single-stranded donor oligonucleotide (ssODN) or a double-stranded donor oligonucleotide (dsODN).

5. The method of either one of claims 2-4, wherein the template polynucleotide hybridizes to a genomic sequence flanking the DSB in the target DNA and integrates into the target DNA.

6. The method of any one of claims 2-5, wherein the template polynucleotide comprises a donor sequence, a first flanking sequence which is homologous to a genomic sequence upstream of the DSB in the target DNA and a second flanking sequence which is homologous to a genomic sequence downstream of the DSB in the target DNA.

7. The method of claim 6, wherein the donor sequence of the template polynucleotide is integrated into the genome of the hematopoietic cell by homology-directed repair (HDR).

8. The method of any one of claims 1-7, wherein the template polynucleotide is a template for homology-directed repair (HDR) of a prior mutation in the target DNA.

9. The method of any one of claims 1-7, wherein the template polynucleotide is a template for homology-directed repair (HDR) insertion of a gene in the target DNA.

10. The method of any one of claims 1-9, wherein contacting comprises contacting a population of hematopoietic cells.

11. The method of claim 10, further comprising sorting the population of hematopoietic cells.

12. The method of claim 11, wherein sorting comprises selecting for viable hematopoietic cells.

13. The method of either one of claim 11 or 12, wherein sorting comprises selecting for hematopoietic cells that integrated the donor sequence into their genome.

14. The method of any one of claims 11-13, wherein sorting comprises Fluorescence Activated Cell Sorting (FACS).

15. The method of any one of claims 11-14, wherein sorting comprises selecting for viable long term engrafting HSCs.

16. The method of any one of claims 10-15, wherein the editing efficiency in the population of hematopoietic cells is at least 30, at least 40, at least 50, at least 60, at least 70, at least 80, at least 90, at least 95, or at least 99%.

17. The method of any one of claims 10-16, wherein the percent viability in the population of hematopoietic cells is at least 50, at least 60, at least 70, at least 80, at least 90, at least 95, or at least 99%.

18. The method of any one of claims 10-17, wherein the efficiency of HDR is 50% or higher.

19. The method of any one of claims 10-17, wherein the efficiency of HDR is 60% or higher.

20. The method of any one of claims 10-17, wherein the efficiency of HDR is 80% or higher.

21. The method of any one of claims 2-20, wherein the expansion agent comprises at least one of StemRegenin (SR1), UM171, and IL-6.

22. The method of any one of claims 2-21, wherein the expansion agent comprises SR1 and UM171.

23. The method of any one of claims 2-22, wherein the HDR promoting agent comprises at least one of SCR7, NU7441, Rucaparib, and RS-1.

24. The method of any one of claims 2-23, wherein the HDR promoting agent comprises at least two of SCR7, NU7441, Rucaparib, and RS-1.

25. The method of any one of claims 2-24, wherein the HDR promoting agent comprises at least three of SCR7, NU7441, Rucaparib, and RS-1.

26. The method of any one of claims 2-25, wherein the HDR promoting agent comprises SCR7, NU7441, Rucaparib, and RS-1.

27. The method of claim 21 or 22, wherein the SR1 is present at a concentration of 0.1-1.5, 0.3-1.5, 0.5-1.5, 0.7-1.5, 1-1.5, 1.2-1.5, 0.1-1, 0.3-1, 0.5-1, 0.7-1, 0.1-0.8, 0.3-0.8, 0.5-0.8, 0.7-0.8, 0.1-0.5, 0.3-0.5, or 0.1-0.3 μM.

28. The method of claim 21, 22, or 27, wherein the UM171 is present at a concentration of 1-100, 1-80, 1-60, 1-40, 1-20, 1-10, 20-100, 20-80, 20-60, 20-40, 30-100, 30-80, 30-60, 30-40, 50-100, 50-80, 50-60, or 80-100 nM.

29. The method of any one of claims 23-26, wherein the SCR7 is present at a concentration of 0.1-20, 0.1-15, 0.1-10, 0.1-8, 0.1-6, 0.1-5, 0.1-4, 0.1-3, 0.1-2, 0.1-1, 1-20, 1-15, 1-10, 1-8, 1-6, 1-5, 1-4, 1-3, 1-2, 3-20, 3-15, 3-10, 3-8, 3-6, 3-5, 3-4, 5-20, 5-15, 5-10, 5-8, 5-6, 8-20, 8-15, 8-10, 10-15, 10-20, or 15-20 μM.

30. The method of any one of claim 23-26 or 29, wherein the NU7441 is present at a concentration of 0.05-10, 0.05-8, 0.05-6, 0.05-5, 0.05-4, 0.05-3, 0.05-2, 0.05-1, 0.05-0.1, 0.1-20, 0.1-15, 0.1-10, 0.1-8, 0.1-6, 0.1-5, 0.1-4, 0.1-3, 0.1-2, 0.1-1, 1-20, 1-15, 1-10, 1-8, 1-6, 1-5, 1-4, 1-3, 1-2, 3-20, 3-15, 3-10, 3-8, 3-6, 3-5, 3-4, 5-20, 5-15, 5-10, 5-8, 5-6, 8-20, 8-15, 8-10, 10-15, 10-20, or 15-20 μM.

31. The method of any one of claim 23-26, 29, or 30, wherein the RS-1 is present at a concentration of 0.1-50, 0.1-20, 0.1-15, 0.1-10, 0.1-8, 0.1-6, 0.1-5, 0.1-4, 0.1-3, 0.1-2, 0.1-1, 1-50, 1-20, 1-15, 1-10, 1-8, 1-6, 1-5, 1-4, 1-3, 1-2, 3-50 3-20, 3-15, 3-10, 3-8, 3-6, 3-5, 3-4, 5-50, 5-20, 5-15, 5-10, 5-8, 5-6, 8-50, 8-20, 8-15, 8-10, 10-50, 10-15, 10-20, 15-50, 15-20, or 20-50 μM.

32. The method of any one of claim 23-26 or 29-31, wherein Rucaparib is present at a concentration of 0.05-10, 0.05-8, 0.05-6, 0.05-5, 0.05-4, 0.05-3, 0.05-2, 0.05-1, 0.05-0.1, 0.1-20, 0.1-15, 0.1-10, 0.1-8, 0.1-6, 0.1-5, 0.1-4, 0.1-3, 0.1-2, 0.1-1, 1-20, 1-15, 1-10, 1-8, 1-6, 1-5, 1-4, 1-3, 1-2, 3-20, 3-15, 3-10, 3-8, 3-6, 3-5, 3-4, 5-20, 5-15, 5-10, 5-8, 5-6, 8-20, 8-15, 8-10, 10-15, 10-20, or 15-20 μM.

33. The method of any one of claims 1-32, wherein the hematopoietic cell is a hematopoietic stem cell (HSC).

34. The method of any one of claims 1-33, wherein the hematopoietic cell is a CD34+ cell.

35. The method of any one of claims 1-34, wherein the hematopoietic cell is obtained from bone marrow, blood, umbilical cord, or peripheral blood mononuclear cells (PBMCs).

36. The method of any one of claims 1-35, wherein the hematopoietic cell is human.

37. The method of any one of claims 1-36, wherein contacting also comprises contacting the hematopoietic cell with growth media.

38. The method of claim 37, wherein the growth media is a Stromal Cell Growth Media (SCGM™), e.g., as available from Lonza Bioscience), or serum- and feeder-free media (SFFM).

39. The method of either one of claim 37 or 38, wherein the growth media comprises one or more cytokines.

40. The method of claim 39, wherein the one or more cytokines are selected from one, two, or all of human stem cell factor (hSCF), Fms-like tyrosine kinase 3 ligand (FLT3-L), or thrombopoietin (TPO).

41. The method of any one of claims 1-40, wherein the hematopoietic cell is capable of long-term engraftment into a human recipient.

42. The method of any one of claims 33-41, wherein the hematopoietic cell is capable of reconstituting the hematopoietic system in a human recipient after engraftment.

43. The method of any one of claims 1-42, wherein the target DNA comprises a portion of a glucosylceramidase beta (GBA) gene.

44. The method of claim 43, wherein the template polynucleotide comprises a first flanking sequence which is homologous to a first portion of the GBA gene and a second flanking sequence which is homologous to a second portion of the GBA gene.

45. A method comprising:
contacting a hematopoietic cell with:
(a) a Clustered Regularly Interspaced Short Palindromic Repeats (CRISPR)-CRISPR associated (Cas) (CRISPR/Cas) system comprising a Cas nuclease and a guide RNA (gRNA) comprising a nucleotide sequence that hybridizes to a target DNA in a glucosylceramidase beta (GBA) gene in the genome of the hematopoietic cell, wherein the CRISPR/Cas system creates a double-stranded break (DSB) in the GBA gene; and
(b) a template polynucleotide comprising a donor sequence, a first flanking sequence which is homologous to a first portion of the GBA gene and a second flanking sequence which is homologous to a second portion of the GBA gene.

46. The method of either one of claim 44 or 45, wherein the first portion of the GBA gene comprises a portion of exon 9 or a sequence proximal thereto.

47. The method of claim 46, wherein the second portion of the GBA gene comprises a portion of exon 9 or a sequence proximal thereto, wherein the first portion and second portion are not identical.

48. The method of either one of claim 44 or 45, wherein the first portion of the GBA gene comprises a portion of exon 10 or a sequence proximal thereto.

49. The method of claim 48, wherein the second portion of the GBA gene comprises a portion of exon 10 or a sequence proximal thereto, wherein the first portion and second portion are not identical.

50. The method of any one of claims 43-49, wherein the donor sequence comprises a sequence corresponding to the codon encoding N409 or L483 in a wildtype GBA gene.

51. The method of claim 50, wherein the wildtype GBA gene comprises the sequence of SEQ ID NO: 47.

52. The method of claim 50, wherein the sequence corresponding to the codon encoding N409 in the wildtype GBA gene encodes an asparagine.

53. The method of claim 52, wherein the template polynucleotide comprises the sequence of any one of SEQ ID NOs: 51-54.

54. The method of claim 50, wherein the sequence corresponding to the codon encoding N409 in the wildtype GBA gene encodes a serine.

55. The method of claim 54, wherein the template polynucleotide comprises the sequence of SEQ ID NOs: 25-28.

56. The method of claim 50, wherein the sequence corresponding to the codon encoding L483 in the wildtype GBA gene encodes a leucine.

57. The method of claim 56, wherein the template polynucleotide comprises the sequence of any one of SEQ ID NOs: 55-57.

58. The method of claim 50, wherein the sequence corresponding to the codon encoding L483 in the wildtype GBA gene encodes a proline.

59. The method of claim 58, wherein the template polynucleotide comprises the sequence of SEQ ID NOs: 29-30.

60. The method of any one of claims 43-59, wherein the first flanking sequence comprises a flanking sequence set forth in any one of SEQ ID NOs: 25-30 or 51-57.

61. The method of any one of claims 43-60, wherein the second flanking sequence comprises a flanking sequence set forth in any one of SEQ ID NOs: 25-30 or 51-57.

62. The method of any one of claims 43-61, wherein the donor sequence comprises a donor sequence selected from any one of SEQ ID NOs: 25-30 or 51-57.

63. The method of any one of claims 43-62, wherein the template polynucleotide comprises the sequence of SEQ ID NOs: 25-30 or 51-57.

64. The method of any one of claims 1-63, wherein the donor sequence comprises a restriction site or a unique sequence tag.

65. The method of claim 64, wherein the sequence comprising the restriction site or unique sequence tag is an insertion relative to the target DNA.

66. The method of claim 64, wherein the sequence comprising the restriction site or unique sequence tag is not an insertion relative to the target DNA.

67. The method of claim 64 or 66, wherein the sequence comprising the restriction site or unique sequence tag does not alter an amino acid sequence encoded by the target DNA.

68. The method of any one of claims 64-67, wherein the first flanking sequence, second flanking sequence, or both comprise a PAM site sequence or a sequence complementary to the PAM site sequence.

69. The method of claim 68, wherein the restriction site is no more than 20, no more than 15, no more than 10, no more than 9, no more than 8, no more than 7, no more than 6, no more than 5, no more than 4, no more than 3, no more than 2, or no more than 1 nucleotides from the PAM site sequence or the sequence complementary to the PAM site sequence.

70. The method of any one of claims 8-69, wherein the donor sequence comprises a second mutation relative to the target DNA.

71. The method of claim 70, wherein the second mutation is a silent mutation.

72. The method of either one of claim 70 or 71, wherein the second mutation is situated in a codon that is contiguous with the HDR mutation or HDR insertion.

73. The method of any one of claims 4-72, wherein the ssODN comprises, from 5′ to 3′, the first flanking sequence, the donor sequence, and the second flanking sequence.

74. The method of any one of claims 4-73, wherein the first flanking sequence is 50-200, 50-180, 50-160, 50-140, 50-120, 50-100, 50-80, 50-60, 70-200, 70-180, 70-160, 70-140, 70-120, 70-100, 70-80, 100-200, 100-180, 100-160, 100-140, 100-120, 120-200, 120-180, 120-160, 120-140, 150-200, 150-180, or 150-160 nucleotides in length.

75. The method of any one of claims 4-74, wherein the second flanking sequence is 50-200, 50-180, 50-160, 50-140, 50-120, 50-100, 50-80, 50-60, 70-200, 70-180, 70-160, 70-140, 70-120, 70-100, 70-80, 100-200, 100-180, 100-160, 100-140, 100-120, 120-200, 120-180, 120-160, 120-140, 150-200, 150-180, or 150-160 nucleotides in length.

76. The method of any one of claims 4-75, wherein the donor sequence is 1-100, 1-80, 1-60, 1-40, 1-20, 1-15, 1-10, 1-9, 1-8, 1-7, 1-6, 1-5, 1-4, 1-3, 1-2, 5-100, 5-80, 5-60, 5-40, 5-20, 5-15, 5-10, 5-9, 5-8, 5-7, 5-6, 10-100, 10-80, 10-60, 10-40, 10-20, 10-15, 20-100, 20-80, 20-60, 20-40, 60-100, or 60-80 nucleotides in length.

77. The method of any one of claims 1-76, wherein the CRISPR/Cas system comprises a guide nucleic acid comprising a sequence chosen from any one of SEQ ID NOs: 3, 6, 9, 12, 15, 18, 21, 24, 33, 36, 39, and 42, or a sequence having no more than 1, no more than 2, no more than 3, no more than 4, or no more than 5 substitutions relative to any thereof.

78. The method of any one of claims 4-77, wherein the donor sequence is integrated into the genome of the hematopoietic stem cell by homology-directed repair (HDR).

79. The method of any one of claims 1-78, wherein the method is a method of producing a genetically modified hematopoietic cell or population of genetically modified hematopoietic cells.

80. A method comprising:
providing a genetically modified hematopoietic cell wherein the hematopoietic cell was genetically modified to comprise one, two, or three of:
(a) an endogenous glucosylceramidase beta (GBA) gene that encodes an asparagine at a position corresponding to position 409 of a wildtype GBA gene;
(b) an endogenous GBA gene that encodes a leucine at a position corresponding to position 409 of a wildtype GBA gene; or
(c) a heterologous copy of a GBA gene that encodes an asparagine at a position corresponding to position 409 of a wildtype GBA gene and a leucine at a position corresponding to position 409 of a wildtype GBA gene, and
administering the genetically modified hematopoietic cell to a subject.

81. The method of claim 80, wherein the method is a method of treating Gaucher disease in the subject.

82. The method of either of claim 80 or 81, wherein the genetically modified hematopoietic cell is a genetically modified hematopoietic stem cell.

83. The method of claim 80, wherein providing comprises genetically modifying the hematopoietic cell by contacting the cell with:
(a) a Clustered Regularly Interspaced Short Palindromic Repeats (CRISPR)-CRISPR associated (Cas) (CRISPR/Cas) system comprising a Cas nuclease and a guide RNA (gRNA) comprising a nucleotide sequence that hybridizes to a target DNA in a glucosylceramidase beta (GBA) gene in the genome of the hematopoietic cell, wherein the CRISPR/Cas system creates a double-stranded break (DSB) in the GBA gene; and
(b) a template polynucleotide comprising a donor sequence, a first flanking sequence which is homologous to a first portion of the GBA gene and a second flanking sequence which is homologous to a second portion of the GBA gene.

84. The method of any one of claims 80-83, wherein the genetically modified hematopoietic cell was produced by a method of any one of claims 1-79.

85. The method of any one of claims 80-84, wherein the genetically modified hematopoietic stem cell is autologous to the subject.

86. A template polynucleotide comprising a nucleic acid single-strand that comprises, from 5′ to 3′:
a first flanking sequence complementary to a first portion of a glucosylceramidase beta (GBA) gene,
a donor sequence, and
a second flanking sequence complementary to a second portion of the GBA gene.

87. The template polynucleotide of claim 86, wherein the template polynucleotide is a single-strand donor oligonucleotide (ssODN) or a double-stranded oligonucleotide (dsODN) donor.

88. The template polynucleotide of either one of claim 86 or 87, wherein the template polynucleotide is a template for homology-directed repair (HDR) of a mutation in the GBA gene.

89. The template polynucleotide of any one of claims 86-88, wherein the template polynucleotide is a template for homology-directed repair (HDR) insertion of a GBA gene or portion thereof.

90. The template polynucleotide of either one of claim 86 or 87, wherein the first portion of the GBA gene comprises a portion of exon 9 or a sequence proximal thereto.

91. The template polynucleotide of either one of claim 86 or 90, wherein the second portion of the GBA gene comprises a portion of exon 9 or a sequence proximal thereto, wherein the first portion and second portion are not identical.

92. The template polynucleotide of claim 86, wherein the first portion of the GBA gene comprises a portion of exon 10 or a sequence proximal thereto.

93. The template polynucleotide of either one of claim 86 or 92, wherein the second portion of the GBA gene comprises a portion of exon 10 or a sequence proximal thereto, wherein the first portion and second portion are not identical.

94. The template polynucleotide of any one of claims 86-93, wherein the donor sequence comprises a sequence corresponding to the codon encoding N409 or L483 in a wildtype GBA gene.

95. The template polynucleotide of claim 94, wherein the wildtype GBA gene comprises the sequence of SEQ ID NO: 47.

96. The template polynucleotide of claim 94, wherein the sequence corresponding to the codon encoding N409 in the wildtype GBA gene encodes an asparagine.

97. The template polynucleotide of claim 94, comprising the sequence of any one of SEQ ID NOs: 51-54.

98. The template polynucleotide of claim 94, wherein the sequence corresponding to the codon encoding N409 in the wildtype GBA gene encodes a serine.

99. The template polynucleotide of claim 98, wherein the donor sequence comprises the sequence of SEQ ID NOs: 25-28.

100. The template polynucleotide of claim 94, wherein the sequence corresponding to the codon encoding L483 in the wildtype GBA gene encodes a leucine.

101. The template polynucleotide of claim 100, comprising the sequence of any one of SEQ ID NOs: 55-57.

102. The template polynucleotide of claim 94, wherein the sequence corresponding to the codon encoding L483 in the wildtype GBA gene encodes a proline.

103. The template polynucleotide of claim 102, comprising the sequence of SEQ ID NOS:
29-30.

104. The template polynucleotide of any one of claims 86-103, wherein the first flanking sequence comprises a flanking sequence as set forth in any one of SEQ ID NOs: 25-30 or 51-57.

105. The template polynucleotide of any one of claims 86-104, wherein the second flanking sequence comprises a flanking sequence as set forth in any one of SEQ ID NOs: 25-30 or 51-57.

106. The template polynucleotide of any one of claims 86-105, wherein the donor sequence comprises a donor sequence of any one of SEQ ID NOs: 25-30 or 51-57.

107. The template polynucleotide of any one of claims 86-106, wherein the template polynucleotide comprises the sequence of SEQ ID NO: 25-30 or 51-57.

108. The template polynucleotide of any one of claims 86-107, wherein the donor sequence comprises a restriction site or a unique sequence tag.

109. The template polynucleotide of claim 108, wherein the sequence comprising the restriction site or unique sequence tag is an insertion relative to a target site in the GBA gene.

110. The template polynucleotide of claim 108, wherein the sequence comprising the restriction site or unique sequence tag is not an insertion relative to a target site in the GBA gene.

111. The template polynucleotide of either one of claim 109 or 110, wherein the sequence comprising the restriction site or unique sequence tag does not alter an amino acid sequence encoded by the target site.

112. The template polynucleotide of any one of claims 86-111, wherein the first flanking sequence, second flanking sequence, or both comprise a PAM site sequence or a sequence complementary to a PAM site sequence present in the GBA gene.

113. The template polynucleotide of claim 112, wherein the restriction site or unique sequence tag is no more than 20, no more than 15, no more than 10, no more than 9, no more than 8, no more than 7, no more than 6, no more than 5, no more than 4, no more than 3, no more than 2, or no more than 1 nucleotides from the PAM site sequence or the sequence complementary to a PAM site sequence.

114. The template polynucleotide of any one of claims 88-113, wherein the donor sequence comprises a second mutation relative to the target DNA.

115. The template polynucleotide of claim 114, wherein the second mutation is a silent mutation.

116. The template polynucleotide of either one of claim 114 or 115, wherein the second mutation is situated in a codon that is contiguous with the HDR mutation or HDR insertion.

117. The template polynucleotide of any one of claims 108-116, wherein the first flanking sequence comprises a flanking sequence as set forth in any one of SEQ ID NOs: 25-30 or 51-57.

118. The template polynucleotide of any one of claims 108-117, wherein the second flanking sequence comprises a flanking sequence as set forth in any one of SEQ ID NOs: 25-30 or 51-57.

119. The template polynucleotide of any one of claims 108-118, wherein the donor sequence comprises a sequence selected from any one of SEQ ID NOs: 25-30 or 51-57.

120. The template polynucleotide of any one of claims 108-119, wherein the template polynucleotide comprises the sequence of SEQ ID NOs: 25-30 or 51-57.

121. A guide nucleic acid comprising a sequence complementary to a portion of the glucosylceramidase beta (GBA) gene, wherein the portion comprises a portion of exon 9 or exon 10 and a PAM site sequence.

122. A guide nucleic acid comprising the sequence of any one of SEQ ID NOs: 3, 6, 9, 12, 15, 18, 21, 24, 33, 36, 39, or 42, or a sequence having no more than 1, no more than 2, no more than 3, no more than 4, or no more than 5 substitutions relative to any thereof.

123. A mixture comprising:
(a) a template polynucleotide comprising a nucleic acid single-strand that comprises a donor sequence, a first flanking sequence and a second flanking sequence,
(b) a Clustered Regularly Interspaced Short Palindromic Repeats (CRISPR)-CRISPR associated (Cas) (CRISPR/Cas) system comprising a Cas nuclease and a guide RNA (gRNA) comprising a nucleotide sequence that hybridizes to a target DNA in the genome of the hematopoietic cell, and
(c) one or both of:
an expansion agent selected from at least one of StemRegenin 1 (SR1), and UM171, and
a homology-directed repair (HDR) promoting agent selected from at least one of SCR7, NU7441, Rucaparib, and RS-1.

124. A kit comprising:
(a) a template polynucleotide comprising a nucleic acid single-strand that comprises a donor sequence, a first flanking sequence and a second flanking sequence,
(b) a Clustered Regularly Interspaced Short Palindromic Repeats (CRISPR)-CRISPR associated (Cas) (CRISPR/Cas) system comprising a Cas nuclease and a guide RNA (gRNA) comprising a nucleotide sequence that hybridizes to a target DNA in the genome of the hematopoietic cell, and
(c) one or both of:
an expansion agent selected from at least one of StemRegenin 1 (SR1), and UM171, and
a homology-directed repair (HDR) promoting agent selected from at least one of SCR7, NU7441, Rucaparib, and RS-1.

125. The kit of claim 124, comprising one or more containers comprising (a), (b), and/or (c).

126. The kit of either one of claim 124 or 125, comprising instructions for producing a genetically modified hematopoietic stem cell.

127. The kit of any one of claims 124-126, comprising instructions to perform a method of any one of claims 1-76.

128. The mixture or kit of any one of claims 123-127, wherein the template polynucleotide is a template polynucleotide of any one of claims 86-120.

129. The mixture or kit of any one of claims 123-128, wherein (c) comprises expansion agents comprising at StemRegenin 1 (SR1) and UM171.

130. The mixture or kit of any one of claims 123-128, wherein (c) comprises HDR promoting agents comprising at least two of SCR7, NU7441, Rucaparib, and RS-1.

131. The mixture or kit of any one of claims 123-130, wherein (c) comprises HDR promoting agents comprising at least three of SCR7, NU7441, Rucaparib, and RS-1.

132. The mixture or kit of any one of claims 123-131, wherein (c) comprises HDR promoting agents comprising SCR7, NU7441, Rucaparib, and RS-1.

133. The mixture or kit of any one of claims 123-132, wherein the SR1 is present at a concentration of 0.1-1.5, 0.3-1.5, 0.5-1.5, 0.7-1.5, 1-1.5, 1.2-1.5, 0.1-1, 0.3-1, 0.5-1, 0.7-1, 0.1-0.8, 0.3-0.8, 0.5-0.8, 0.7-0.8, 0.1-0.5, 0.3-0.5, or 0.1-0.3 μM.

134. The mixture or kit of any one of claims 123-133, wherein the UM171 is present at a concentration of 1-100, 1-80, 1-60, 1-40, 1-20, 1-10, 20-100, 20-80, 20-60, 20-40, 30-100, 30-80, 30-60, 30-40, 50-100, 50-80, 50-60, or 80-100 nM.

135. The mixture or kit of any one of claims 123-134, wherein the SCR7 is present at a concentration of 0.1-20, 0.1-15, 0.1-10, 0.1-8, 0.1-6, 0.1-5, 0.1-4, 0.1-3, 0.1-2, 0.1-1, 1-20, 1-15, 1-10, 1-8, 1-6, 1-5, 1-4, 1-3, 1-2, 3-20, 3-15, 3-10, 3-8, 3-6, 3-5, 3-4, 5-20, 5-15, 5-10, 5-8, 5-6, 8-20, 8-15, 8-10, 10-15, 10-20, or 15-20 μM.

136. The mixture or kit of any one of claims 123-135, wherein the NU7441 is present at a concentration of 0.05-10, 0.05-8, 0.05-6, 0.05-5, 0.05-4, 0.05-3, 0.05-2, 0.05-1, 0.05-0.1, 0.1-20, 0.1-15, 0.1-10, 0.1-8, 0.1-6, 0.1-5, 0.1-4, 0.1-3, 0.1-2, 0.1-1, 1-20, 1-15, 1-10, 1-8, 1-6, 1-5, 1-4, 1-3, 1-2, 3-20, 3-15, 3-10, 3-8, 3-6, 3-5, 3-4, 5-20, 5-15, 5-10, 5-8, 5-6, 8-20, 8-15, 8-10, 10-15, 10-20, or 15-20 μM.

137. The mixture or kit of any one of claims 123-136, wherein the RS-1 is present at a concentration of 0.1-50, 0.1-20, 0.1-15, 0.1-10, 0.1-8, 0.1-6, 0.1-5, 0.1-4, 0.1-3, 0.1-2, 0.1-1, 1-50, 1-20, 1-15, 1-10, 1-8, 1-6, 1-5, 1-4, 1-3, 1-2, 3-50 3-20, 3-15, 3-10, 3-8, 3-6, 3-5, 3-4, 5-50, 5-20, 5-15, 5-10, 5-8, 5-6, 8-50, 8-20, 8-15, 8-10, 10-50, 10-15, 10-20, 15-50, 15-20, or 20-50 M.

138. The mixture or kit of any one of claims 123-137, wherein the Rucaparib is present at a concentration of 0.05-10, 0.05-8, 0.05-6, 0.05-5, 0.05-4, 0.05-3, 0.05-2, 0.05-1, 0.05-0.1, 0.1-20, 0.1-15, 0.1-10, 0.1-8, 0.1-6, 0.1-5, 0.1-4, 0.1-3, 0.1-2, 0.1-1, 1-20, 1-15, 1-10, 1-8, 1-6, 1-5, 1-4, 1-3, 1-2, 3-20, 3-15, 3-10, 3-8, 3-6, 3-5, 3-4, 5-20, 5-15, 5-10, 5-8, 5-6, 8-20, 8-15, 8-10, 10-15, 10-20, or 15-20 μM.

139. The method, mixture, or kit of any one of claims 1-138, wherein the Cas nuclease is Cas9.

140. The method, mixture, or kit of any one of claims 1-139, wherein the Cas nuclease is Streptococcus pyogenes Cas9 (spCas9).

141. The method, mixture, or kit of any one of claims 1-139, wherein the Cas nuclease is Staphylococcus aureus Cas9 (saCas9).

142. The method, mixture, or kit of any one of claims 1-138, wherein the Cas nuclease is Cas12a.

143. The method, mixture, or kit of any one of claims 1-138, wherein the Cas nuclease is Cas12b.

144. The method, mixture, or kit of any one of claims 1-138, wherein the Cas nuclease is Cas13.

145. The method of any one of claim 1-79 or 139-144, wherein the contacting comprises introducing the CRISPR/Cas system into the cell in the form of a pre-formed ribonucleoprotein (RNP) complex.

146. The method of claim 145, wherein the pre-formed RNP complex is introduced into the cell via electroporation.

147. The method of either one of claim 145 or 146, wherein the contacting comprises introducing the template polynucleotide into the cell via electroporation.

148. The method of claim 147, wherein the template polynucleotide and CRISPR/Cas system are electroporated into the cell simultaneously.

149. The method of any one of claims 145-148, wherein the CRISPR/Cas system is introduced into the hematopoietic cell within 0, 1, or 2 days after culturing the hematopoietic cell.

150. The method, mixture, or kit of any one of claim 1-79, 83-85, or 123-149, wherein the CRISPR/Cas system comprises a guide nucleic acid which comprises one or more nucleotide residues that are chemically modified.

151. The method, mixture, or kit of claim 150, wherein the chemically modified nucleotide residues comprise 2′O-methyl moieties.

152. The method, mixture, or kit of either one of claim 150 or 151, wherein the chemically modified nucleotide residues comprise phosphorothioate moieties.

153. The method, mixture, or kit of any one of claims 150-152, wherein the chemically modified nucleotide residues comprise thioPACE moieties.

154. The method of any one of claim 1-85 or 139-53, wherein the genetically modified hematopoietic stem cell has reduced or eliminated expression of a lineage-specific cell-surface antigen relative to a wildtype hematopoietic stem cell.

155. The method of claim 154, wherein the lineage-specific cell-surface antigen is selected from the group consisting of CD33, CD19, CD123, CLL-1, CD30, CD5, CD6, CD7, CD38, and BCMA.

156. A genetically modified hematopoietic stem cell, or descendant thereof, produced by a method of any one of claims 1-79.

157. A cell population comprising a plurality of cells obtained by or obtainable by the method of any of any one of claims 1-79, or a plurality of cells of claim 156.

158. A pharmaceutical composition comprising the cell, or a descendant thereof, of claim 156 or the cell population of claim 157.

159. A template polynucleotide comprising from 5′ to 3′-a first flanking sequence, a donor sequence, and a second flanking sequence, wherein the first and second flanking sequences are at least 500 nucleotides in length.

160. The template polynucleotide of claim 159, wherein the template polynucleotide is a single-stranded donor oligonucleotide (ssODN), a double-stranded donor oligonucleotide (dsODN), a minicircle plasmid, or a nanoplasmid.

161. The template polynucleotide of claim 159 or 160, wherein the first and second flanking sequence comprises 500-2000, 600-2000, 700-2000, 800-2000, 900-2000, 1000-2000, 1100-2000, 1200-2000, 1300-2000, 1400-2000, 1500-2000, 1600-2000, 1700-2000, 1800-2000, or 1900-2000 nucleotides in length.

162. The template polynucleotide of any one of claims 159-161, wherein the donor sequence is 200-2000, 200-1900, 200-1800, 200-1700, 200-1600, 200-1500, 200-1400, 200-1300, 200-1200, 200-1100, 100-1000, 100-900, 100-800, 100-700, 100-600, 100-500, 100-400, 100-300, or 100-200 nucleotides in length.

163. The template polynucleotide of any one of claims 159-162, wherein the template polynucleotide comprises the sequence of any one of SEQ ID NOs: 25-30, 51-57, 72-73, 93, 95-96, or 102-128.

164. The template polynucleotide of any one of claims 159-163, wherein the donor sequence encodes a chimeric antigen receptor (CAR).

165. The template polynucleotide of claim 164, wherein the CAR binds to a lineage-specific cell-surface antigen.

166. The template polynucleotide of claim 165, wherein the lineage-specific cell-surface antigen is CD33.

167. The template polynucleotide of any one of claims 159-166, wherein the first flanking sequence is homologous to first portion of the RAB11a, AAVS1, TRAC, CCR5, or GBA genes and the second flanking sequence is homologous to a second portion of the RAB11a, AAVS1, TRAC, CCR5, or GBA genes.

168. A transgene comprising the template polynucleotide of any one of claims 159-167, wherein the template polynucleotide is flanked by a first and second recombinant adeno-associated virus (rAAV) inverted terminal repeat (ITR).

169. The transgene of claim 168, wherein the transgene comprises from 5′ to 3′ the first ITR, the first flanking sequence, a promoter, a Kozak sequence, the donor sequence, a poly(A) signal, the second flanking sequence, and the second ITR.

170. The transgene of claim 169, wherein the promoter is an SFFV promoter.

171. The transgene of claim 169 or 170, wherein the poly(A) signal is a β-globin poly(A) signal.

172. The transgene of any one of claims 168-171, wherein the transgene comprises the nucleic acid sequence set forth in any one of SEQ ID NOs: 25-30, 51-57, 72-73, 93, 95-96, or 120.

173. A vector comprising the template polynucleotide of any one of claims 159-167 or the transgene of any one of claims 168-172.

174. The vector of claim 173, wherein the vector is a plasmid.

175. The vector of claim 173 or 174, wherein the vector is a pAV1 plasmid.

176. The vector of any one of claims 173-175, wherein the vector comprises the nucleic acid sequence set forth in any one of SEQ ID NOs: 25-30, 51-57, 72-73, 93, 95-96, or 102-128.

177. A recombinant adeno-associated virus (rAAV) comprising the transgene of any one of claims 168-172 or the vector of any one of claims 173-176 and at least one AAV capsid protein.

178. The rAAV of claim 177, wherein the at least one capsid protein is an AAV1, AAV2, AAV3, AAV4, AAV5, AAV6, AAV7, AAV8, or AAV9.

179. A method comprising:
contacting a cell with:
(a) a Clustered Regularly Interspaced Short Palindromic Repeats (CRISPR)-CRISPR associated (Cas) (CRISPR/Cas) system comprising a Cas nuclease and a guide RNA (gRNA) comprising a nucleotide sequence that hybridizes to a target DNA in the genome of the cell; and
(b) the template polynucleotide of any one of claims 159-167, the transgene of any one of claims 171-175, the vector of any one of claims 168-172, or the rAAV of claim 177-178.

180. The method of claim 179, wherein the CRISPR/Cas system creates a double-stranded break (DSB) in the target DNA in the genome of the cell.

181. The method of claim 179 or 180, wherein the template polynucleotide hybridizes to a genomic sequence flanking the DSB in the target DNA and integrates into the target DNA.

182. The method of any one of claims 179-181, wherein the donor sequence of the template polynucleotide is integrated into the genome of the cell by homology-directed repair (HDR).

183. The method of any one of claims 179-182, wherein the cell is a T cell or a hematopoietic cell.

184. The method of claim 183, wherein the cell is a human cell.

185. The method of any one of claims 183-184, wherein the cell is a T cell and the method further comprises contacting the T cells with IL-2, IL-7, and IL-15 prior to contacting the T cells with the CRISPR/Cas system.

186. A cell population comprising a plurality of cells produced by the methods of any one of claims 179-185.

187. The cell population of claim 186, wherein the plurality of cells comprise genetically engineered hematopoietic cells.

188. The cell population of claim 188, wherein the plurality of cells comprise genetically engineered T cells.
